(19) United States
(12) Patent Application Publication
(10) Pub. No.: US 2021/0188843 A1
(43) Pub. Date: Jun. 24, 2021

(54) QUINOLINE COMPOUNDS AND THEIR PREPARATION AND USE AS ANTIMALARIAL AGENTS

(71) Applicants: The United States of America,as represented by the Secretary,Department of Health and Human Services, Bethesda, MD (US); The Henry M. Jackson Foundation for the Advancement of Military Medicine, Inc., Bethesda, MD (US)

(72) Inventors: Wenwei Huang, Rockville, MD (US); Hao Li, Rockville, MD (US); Wei Zheng, Potomac, MD (US); Wei Sun, Germantown, MD (US); Xiuli Huang, Potomac, MD (US); Xiao Lu, Rockville, MD (US); Philip E. Sanderson, Bethesda, MD (US); Xin Xu, Rockville, MD (US); Pranav Shah, Gaithersburg, MD (US); Kim C. Williamson, Bethesda, MD (US); Zhiji Luo, Rockville, MD (US); Gregory J. Tawa, Doylestown, PA (US)

(73) Assignees: The United States of America,as represented by the Secretary,Department of Health and Human Services, Bethesda, MD (US); The Henry M. Jackson Foundation for the Advancment of Military Medicine, Inc., Bethesda, MD (US)

(21) Appl. No.: 17/054,614

(22) PCT Filed: May 13, 2019
(86) PCT No.: PCT/US2019/031987

§ 371 Date: Nov. 11, 2020

Related U.S. Application Data
(60) Provisional application No. 62/670,351, filed on May 11, 2018.

(51) Int. Cl. C07D 471/04; A61P 33/06; C07D 519/00

(52) U.S. Cl. CPC C07D 471/04; C07D 519/00; A61P 33/06

(57) ABSTRACT

Disclosed are compounds of formula (I) wherein R¹, R², R³, A, and B are as defined herein, and a method of preparing such compounds. Also disclosed are pharmaceutical compositions containing the compound of formula (I) and a method of blocking transmission of a Plasmodium parasite and a method of treating or preventing malaria by killing or arresting the growth of Plasmodium organisms in a mammal, wherein the Plasmodium organisms are in a liver stage, an asexual stage, or gametocytes, comprising administering to an animal an effective amount of a compound of formula (I).

### CROSS-REFERENCE TO A RELATED APPLICATION

This patent application claims the benefit of U.S. Provisional Patent Application No. 62/670,351, filed May 11, 2018, the disclosure of which is incorporated herein by reference in its entirety for all purposes.

### BACKGROUND OF THE INVENTION

Malaria cases and deaths have dropped 50% in 29 countries since 2000 due to the combined effects of long-lasting insecticidal bed nets, indoor residual spraying, and artemisinin-based combination therapies (ACTs) [Kilama W. et al., Lancet, 2009, 374: 1480-1482]. This success has raised hopes for malaria eradication and consequently stimulated interest in developing new reagents that block gametocyte transmission, such as novel and safe gametocytocidal drugs [Buchholz K. et al., The Journal ofInfectious Diseases, 2011, 203: 1445-1453]. Previous drug development efforts have focused primarily on the asexual parasites that cause symptoms but not malaria transmission. To be transmitted from person to person via mosquitoes, the parasites must switch from asexual to sexual development and produce male and female gametocytes. Once gametocytes are taken up in a blood meal by a mosquito, fertilization is stimulated and the resulting zygote differentiates into a motile ookinete that migrates across the midgut epithelium of the mosquito and forms an oocyst. Over the course of the next 2 weeks, tens of thousands of infectious sporozoites are generated and sequestered in the mosquito salivary glands until released into a vertebrate host for transmission during the next blood meal.

Sexual stage P. falciparum gametocytes have a lifespan of over 3 weeks and are not cleared effectively by current antimalarial agents, except primaquine (PQ) [Sweeney A W et al., American Journal of Tropical Medicine and Hygiene, 2004, 71: 187-189; Peatey C L et al., Journal ofInfectious Diseases, 2009, 200: 1518-1521] which is not widely used because it causes hemolytic anemia in patients with glucose-6-phosphate dehydrogenase deficiency [Baird J K et al., Trends in Parasitology, 2011, 27: 11-16]. Consequently, treatment with current antimalarial drugs often results in asymptomatic carriers who remain infectious for weeks after the clearance of asexual parasites. Despite the risks of PQ, its efficacy with artemisinin combination therapy (ACT) in reducing malaria transmission in the PQ-tolerant patients was recently demonstrated in test regions. Other than PQ, the only other gametocytocidal candidate being tested is methylene blue.

Thus, a new generation of antimalarial agents with potent activities against both sexual and asexual parasites is urgently needed for better therapeutic effect and eradication of malarial infection globally.

### BRIEF SUMMARY OF THE INVENTION

The invention provides a compound of formula (I):

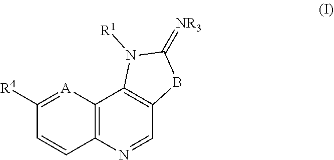

wherein A is CR⁵ or N,

B is CR⁸═CR⁹ or NR²,

R⁸ and R⁹ are independently selected from hydrogen, hydroxyl, OR¹⁰, halogen, optionally substituted C₆₋₁₀ aryl, and optionally substituted C₁₋₆ alkyl,

R¹⁰ is hydrogen, C₁₋₁₂ alkyl, C₃₋₈ cycloalkyl, CH₂COOR¹³, or H₂N(CH₂)ₙ— wherein n is an integer of 2-6,

R¹ is C₆₋₁₀ aryl or heteroaryl optionally substituted with at least one substituent selected from —CN, halo, —CF₃, —CONH₂, —OCF₃, C₁-C₆ alkyl, C₁-C₆ alkylcarbonyl, —OH, C₁-C₆ alkylaminocarbonyl, C₃-C₈ cycloalkylaminocarbonyl, C₁-C₆ alkylaminoalkyl, cyanomethyl, piperazinomethyl, C₁-C₆ alkylsulfonyl, C₁-C₆ alkylsulfonylamino, dialkylaminoalkylamino, optionally substituted piperazinylcarbonyl, and C₁-C₆ alkoxy, a heterocyclyl group selected from the group consisting of piperazin-1-yl, 4-(C₁-C₆ alkylcarbonyl)piperazin-1-yl, morpholinyl optionally substituted with C₁-C₆ alkyl, azetidin-1-yl, pyrrolidin-1-yl, piperidin-1-yl, octahydropyrrolo[3,4-b]pyrrolyl, and 2-oxa-6-azaspiro[3.3]heptyl, C₃-C₈ cycloalkyl or C₃-C₈ azacycloalkyl, each optionally substituted with morpholino, optionally substituted piperidinyl, or optionally substituted piperazinyl,

R² is C₁-C₆ alkyl, hydroxyl C₁-C₆ alkyl, C₁-C₆ alkylcarbonyl, C₁-C₆ alkylsulfonyl, or optionally substituted benzyl,

R³ is H, —CN, C₁-C₆ alkylcarbonyl, C₁-C₆ alkoxycarbonyl, phenylsulfonyl, 4-methylphenylsulfonyl, C₁-C₆ alkylsulfonyl, aminocarbonyl, aminosulfonyl, optionally substituted benzyl, —OH, —OR, —SR, —(S═O)R (R═C₁-C₆ alkyl), guanidino, or pyrimidin-5-yl,

R⁴ is phenyl, heteroaryl, 1-phenyl-2-ethynyl, or heterocyclyl, wherein the phenyl, heteroaryl, heterocyclyl, or phenyl of the 1-phenyl-2-ethynyl is optionally substituted with one or more substituents selected from the group consisting of halo, C₁-C₆ alkyl, amino, oxo, dialkylaminoalkyl, dialkylaminoalkoxy, —CN, aminocarbonyl, —OR⁶, CF₃, and C₁-C₆ alkylsulfonyl,

R⁶ is H or C₁-C₆ alkyl,

R⁵ is hydrogen, C₁-C₆ alkyl, C₆-C₁₀ aryl, halogen, hydroxyl, or OR⁷,

R⁷ is C₁-C₆ alkyl, formyl, C₁-C₆ acyl, or C₆-C₁₀ aryl,

or a pharmaceutically acceptable salt thereof.

The invention further provides a pharmaceutical composition comprising a compound or salt of formula (I) and a pharmaceutically acceptable carrier.

The invention additionally provides a method of blocking transmission of a Plasmodium parasite comprising administering to a mammal in need of such treatment, a therapeutically effective amount of a first compound of formula (I).

The invention also provides a method of treating malaria by killing or arresting the growth of Plasmodium organisms in a mammal, wherein the Plasmodium organisms are in a liver stage or an asexual stage, the method comprising administering to a mammal a therapeutically effective amount of a compound of formula (I) or (II).

Advantageously, compounds and methods in accordance with embodiments of the invention kill all stages of malaria parasites.

### DETAILED DESCRIPTION OF THE INVENTION

In an embodiment, the invention provides a compound of formula (I):

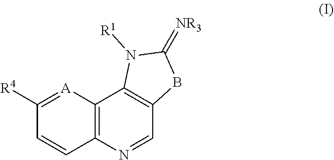

wherein A is CR⁵ or N,

B is CR⁸═CR⁹ or NR²,

R⁸ and R⁹ are independently selected from hydrogen, hydroxyl, OR¹⁰, halogen, optionally substituted C₆₋₁₀ aryl, and optionally substituted C₁₋₆ alkyl,

R¹⁰ is hydrogen, C₁₋₁₂ alkyl, C₃₋₈ cycloalkyl, CH₂COOR¹³, or H₂N(CH₂)ₙ— wherein n is an integer of 2-6,

R¹ is C₆₋₁₀ aryl or heteroaryl optionally substituted with at least one substituent selected from —CN, halo, —CF₃, —CONH₂, —OCF₃, C₁-C₆ alkyl, C₁-C₆ alkylcarbonyl, —OH, C₁-C₆ alkylaminocarbonyl, C₃-C₈ cycloalkylaminocarbonyl, C₁-C₆ alkylaminoalkyl, cyanomethyl, piperazinomethyl, C₁-C₆ alkylsulfonyl, C₁-C₆ alkylsulfonylamino, dialkylaminoalkylamino, optionally substituted piperazinylcarbonyl, and C₁-C₆ alkoxy, a heterocyclyl group selected from the group consisting of piperazin-1-yl, 4-(C₁-C₆ alkylcarbonyl)piperazin-1-yl, morpholinyl optionally substituted with C₁-C₆ alkyl, azetidin-1-yl, pyrrolidin-1-yl, piperidin-1-yl, octahydropyrrolo[3,4-b]pyrrolyl, and 2-oxa-6-azaspiro[3.3]heptyl, C₃-C₈ cycloalkyl or C₃-C₈ azacycloalkyl, each optionally substituted with morpholino, optionally substituted piperidinyl, or optionally substituted piperazinyl,

R² is C₁-C₆ alkyl, hydroxyl C₁-C₆ alkyl, C₁-C₆ alkylcarbonyl, C₁-C₆ alkylsulfonyl, or optionally substituted benzyl,

R³ is H, —CN, C₁-C₆ alkylcarbonyl, C₁-C₆ alkoxycarbonyl, phenylsulfonyl, 4-methylphenylsulfonyl, C₁-C₆ alkylsulfonyl, aminocarbonyl, aminosulfonyl, optionally substituted benzyl, —OH, —OR, —SR, —(S═O)R (R═C₁-C₆ alkyl), guanidino, or pyrimidin-5-yl,

R⁴ is phenyl, heteroaryl, 1-phenyl-2-ethynyl, or heterocyclyl, wherein the phenyl, heteroaryl, heterocyclyl, or phenyl of the 1-phenyl-2-ethynyl is optionally substituted with one or more substituents selected from the group consisting of halo, C₁-C₆ alkyl, amino, oxo, dialkylaminoalkyl, dialkylaminoalkoxy, —CN, aminocarbonyl, —OR⁶, CF₃, and C₁-C₆ alkylsulfonyl,

R⁶ is H or C₁-C₆ alkyl,

R⁵ is hydrogen, C₁-C₆ alkyl, C₆-C₁₀ aryl, halogen, hydroxyl, or OR⁷,

R⁷ is C₁-C₆ alkyl, formyl, C₁-C₆ acyl, or C₆-C₁₀ aryl,

or a pharmaceutically acceptable salt thereof.

Referring now to terminology used generically herein, the term “alkyl” means a straight-chain or branched alkyl substituent containing from, for example, 1 to about 12 carbon atoms, preferably from 1 to about 6 carbon atoms, more preferably from 1 to 4 carbon atoms. Examples of such substituents include methyl, ethyl, propyl, isopropyl, n-butyl, sec-butyl, isobutyl, tert-butyl, pentyl, isoamyl, hexyl, and the like.

The term “aryl” refers to an unsubstituted or substituted aromatic carbocyclic substituent, as commonly understood in the art, and the term “C₆-C₁₀ aryl” includes phenyl and naphthyl. It is understood that the term aryl applies to cyclic substituents that are planar and comprise 4n+2 π electrons, according to Hickel's Rule. The aryl group is optionally substituted with 1, 2, 3, 4, or 5 substituents as recited herein such as with alkyl groups such as methyl groups, ethyl groups, and the like, halo, dihaloalkyl, trihaloalkyl, nitro, hydroxy, alkoxy, aryloxy, amino, alkylamino, dialkylamino, acylamino, acylalkylamino, alkylcarbonyl, alkoxycarbonyl, arylcarbonyl, aryloxycarbonyl, cyanomethyl, alkylsulfonyl, aminosulfonyl, alkylaminosulfonyl, dialkylaminosulfonyl, aminocarbonyl, alkylaminocarbonyl, dialkylaminocarbonyl, thio, alkylthio, arylthio, and the like, wherein the optional substituent can be present at any open position on the aryl group.

The term “heteroaryl” refers to a monocyclic or bicyclic 5 to 10-membered ring system as described herein, wherein the heteroaryl group is unsaturated and satisfies Hickel's rule, and wherein the heteroaryl contains 1-4 heteroatoms independently selected from nitrogen, oxygen and sulfur. Non-limiting examples of suitable heteroaryl groups include furanyl, thiopheneyl, pyrrolyl, pyrazolyl, imidazolyl, 1,2,3-triazolyl, 1,2,4-triazolyl, isoxazolyl, oxazolyl, isothiazolyl, thiazolyl, 1,3,4-oxadiazol-2-yl, 1,2,4-oxadiazol-2-yl, 5-methyl-1,3,4-oxadiazole, 3-methyl-1,2,4-oxadiazole, pyridinyl, pyrimidinyl, pyrazinyl, triazinyl, benzofuranyl, benzothiopheneyl, indolyl, indazolyl, imidazolyl, quinolinyl, isoquinolinyl, benzimidazolyl, benzoxazolinyl, benzothiazolinyl, and quinazolinyl. The term “pyridinyl” is synonymous with the term “pyridyl” and both terms refer to an optionally substituted pyridine group. The heteroaryl groups can be attached at any open position on the heteroaryl groups. The terms “heterocyclic” or “heterocyclyl” refer to a 4 to 12-membered heterocyclic ring system as described herein, wherein the heterocycle contains 1 or 2 heteroatoms independently selected from nitrogen, oxygen, and sulfur, wherein the heterocycle is saturated or monounsaturated. The heterocyclyl or heteroaryl group is optionally substituted with 1, 2, 3, 4, or 5 substituents as recited herein such as with alkyl groups such as methyl groups, ethyl groups, and the like, with hydroxyalkyl groups such as hydroxyl ethyl, or with aryl groups such as phenyl groups, naphthyl groups and the like, wherein the aryl groups can be further substituted with, for example, halo, dihaloalkyl, trihaloalkyl, nitro, hydroxy, alkoxy, aryloxy, amino, alkylamino, dialkylamino, acylamino, acylalkylamino, alkylcarbonyl, alkoxycarbonyl, arylcarbonyl, aryloxycarbonyl, cyanomethyl, alkylsulfonyl, aminosulfonyl, alkylaminosulfonyl, dialkylaminosulfonyl, aminocarbonyl, alkylaminocarbonyl, dialkylaminocarbonyl, thio, alkylthio, arylthio, and the like, wherein the optional substituent can be present at any open position on the heterocyclyl or heteroaryl group.

The term “acyl” refers to an alkylcarbonyl (R—C(═O)—) substituent. The term “aminosulfonyl” refers to a group of the structure: H₂NSO₂—. The term “alkylsulfonyl” refers to a group of the structure: alkyl-SO₂—. The term “aminocarbonyl” refers to a group of the structure: R¹R²NC(═O)— wherein R¹ and R² are independently hydrogen, alkyl, or aryl. The term “alkylsulfonylamino” refers to a group of the structure: alkyl-SO₂—NH—. The term “guanidino” refers to a group of the structure: —C(═NH)NH₂. The term “azacycloalkyl” refers to a cycloalkyl ring having one or more ring carbon atoms replaced with nitrogen. Non-limiting examples of suitable azacycloalkyl groups include azetidinyl, pyrrolidinyl, piperidinyl, piperazinyl, morpholinyl, azepanyl, and the like.

In certain embodiments, R¹ is C₆₋₁₀ aryl or heteroaryl substituted with at least one substituent selected from —CN, halo, —CF₃, —CONH₂, —OCF₃, C₁-C₆ alkyl, C₁-C₆ alkylcarbonyl, —OH, C₁-C₆ alkylaminocarbonyl, C₃-C₈ cycloalkylaminocarbonyl, C₁-C₆ alkylaminoalkyl, cyanomethyl, piperazinomethyl, C₁-C₆ alkylsulfonyl, C₁-C₆ alkylsulfonylamino, dialkylaminoalkylamino, optionally substituted piperazinylcarbonyl, and C₁-C₆ alkoxy, a heterocyclyl group selected from the group consisting of morpholinyl optionally substituted with C₁-C₆ alkyl, azetidin-1-yl, pyrrolidin-1-yl, piperidin-1-yl, octahydropyrrolo[3,4-b]pyrrolyl, and 2-oxa-6-azaspiro[3.3]heptyl, C₃-C₈ cycloalkyl or C₃-C₈ azacycloalkyl substituted with morpholino, optionally substituted piperidinyl, or optionally substituted piperazinyl,

R³ is H, —CN, C₁-C₆ alkylcarbonyl, C₁-C₆ alkoxycarbonyl, phenylsulfonyl, 4-methylphenylsulfonyl, C₁-C₆ alkylsulfonyl, aminocarbonyl, aminosulfonyl, optionally substituted benzyl, —OH, —OR, —SR, —(S═O)R (R═C₁-C₆ alkyl), or guanidino, and R⁴ is phenyl, heteroaryl, 1-phenyl-2-ethynyl, or heterocyclyl, wherein the phenyl, heteroaryl, or heterocyclyl is optionally substituted with one or more substituents selected from the group consisting of halo, C₁-C₆ alkyl, amino, oxo, dialkylaminoalkyl, dialkylaminoalkoxy, —CN, aminocarbonyl, and —OR⁶.

In certain embodiments, B is NR² and R¹ is selected from the group consisting of

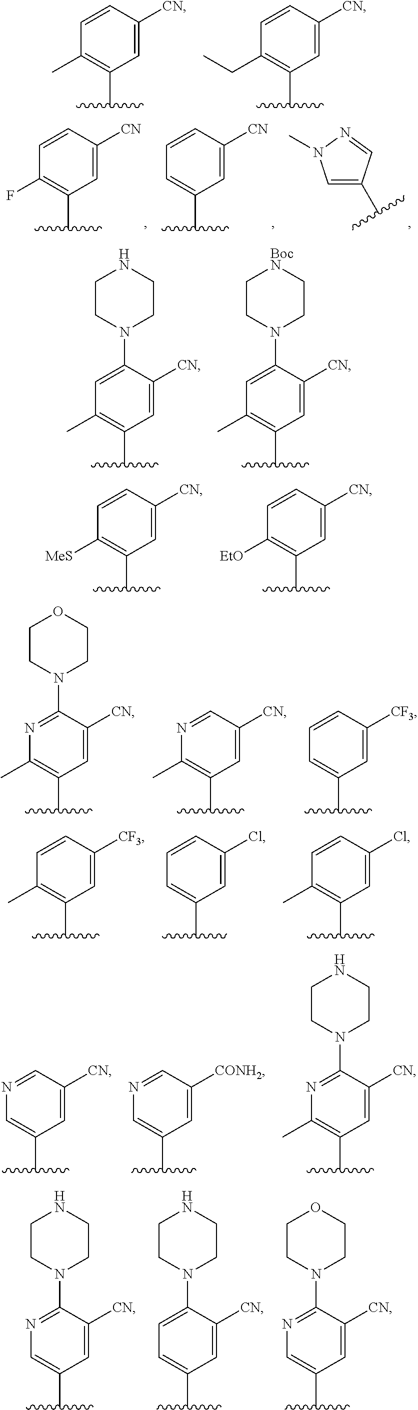

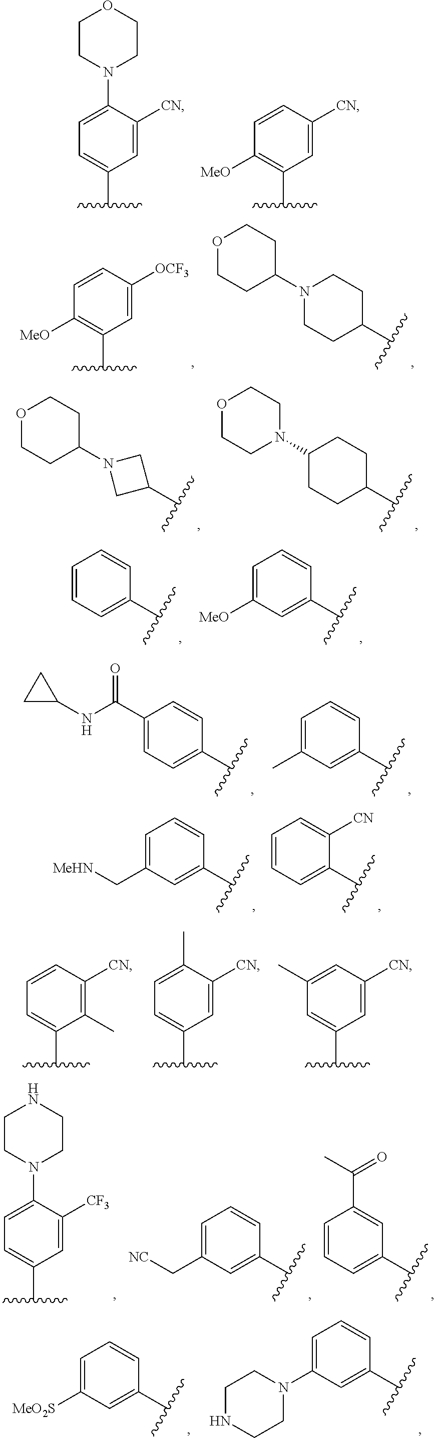

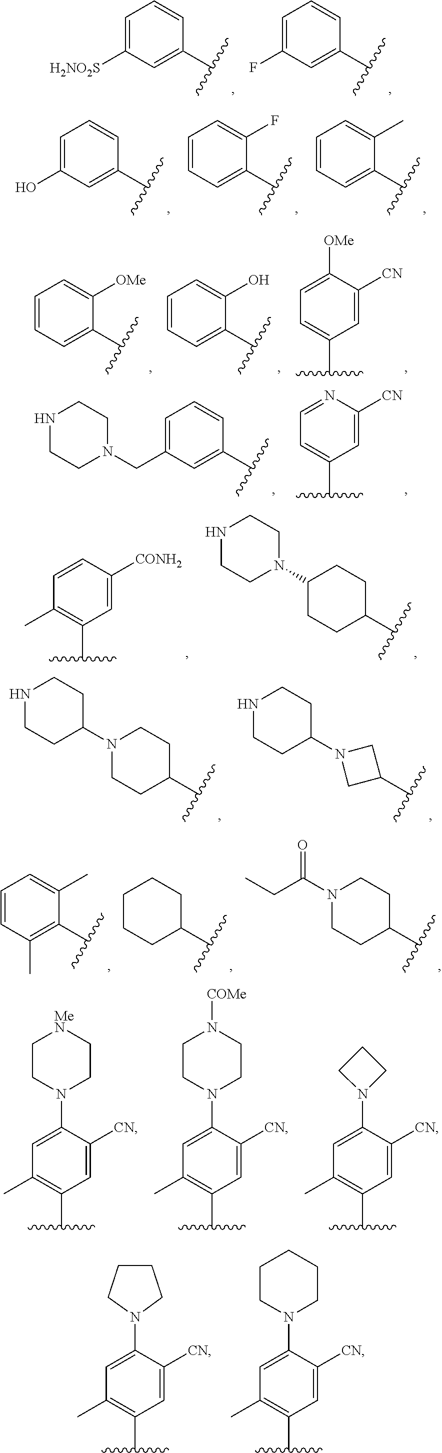

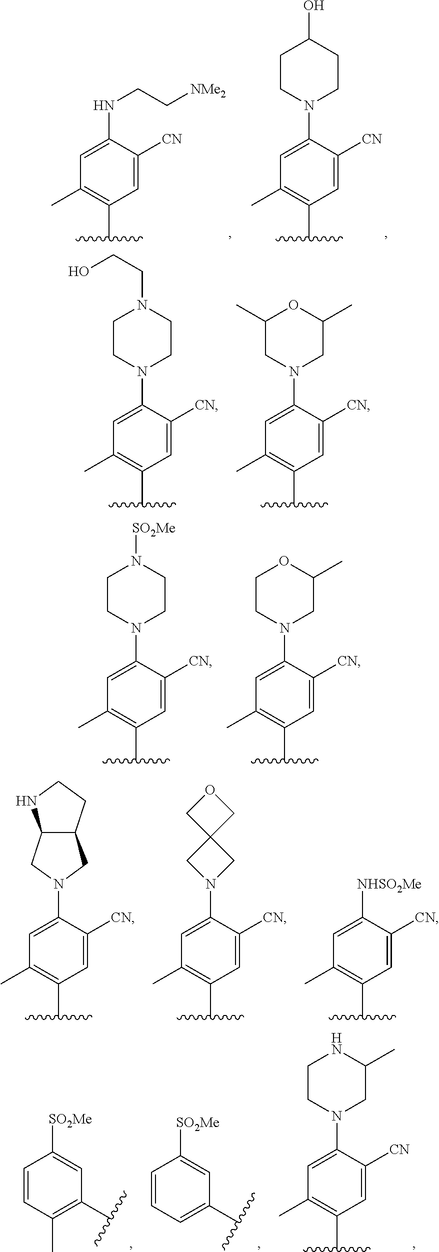

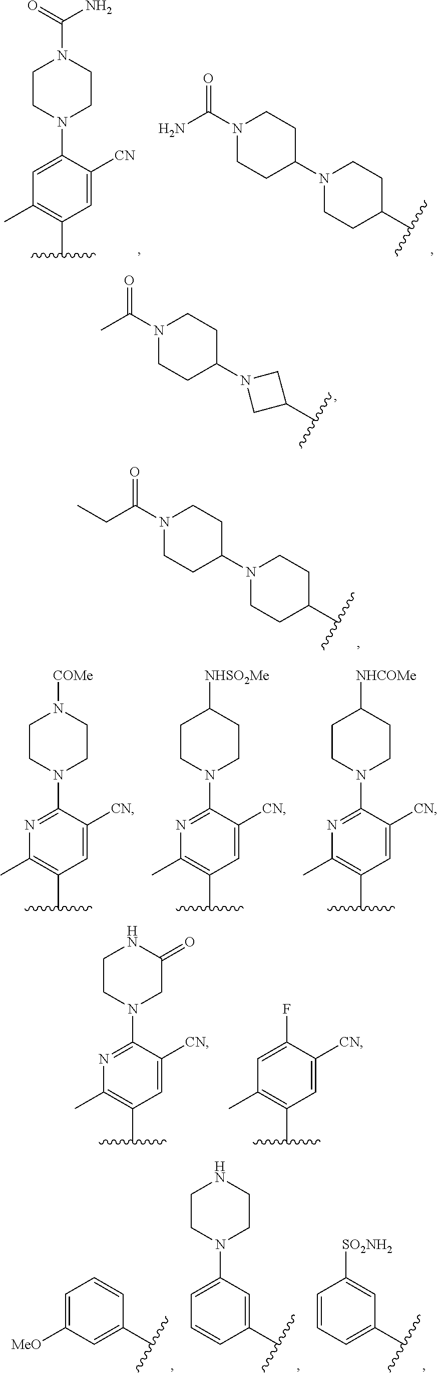

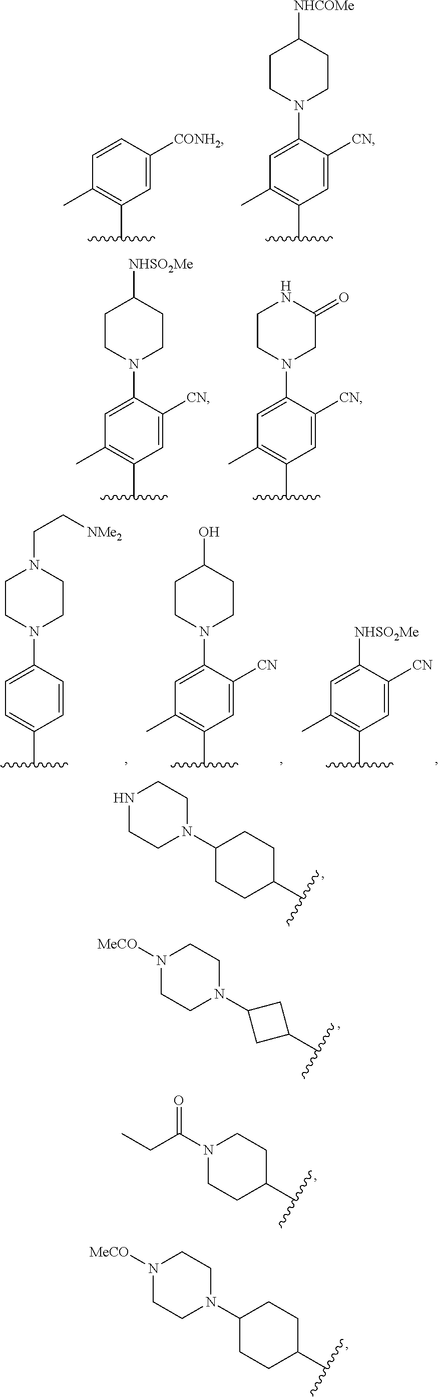

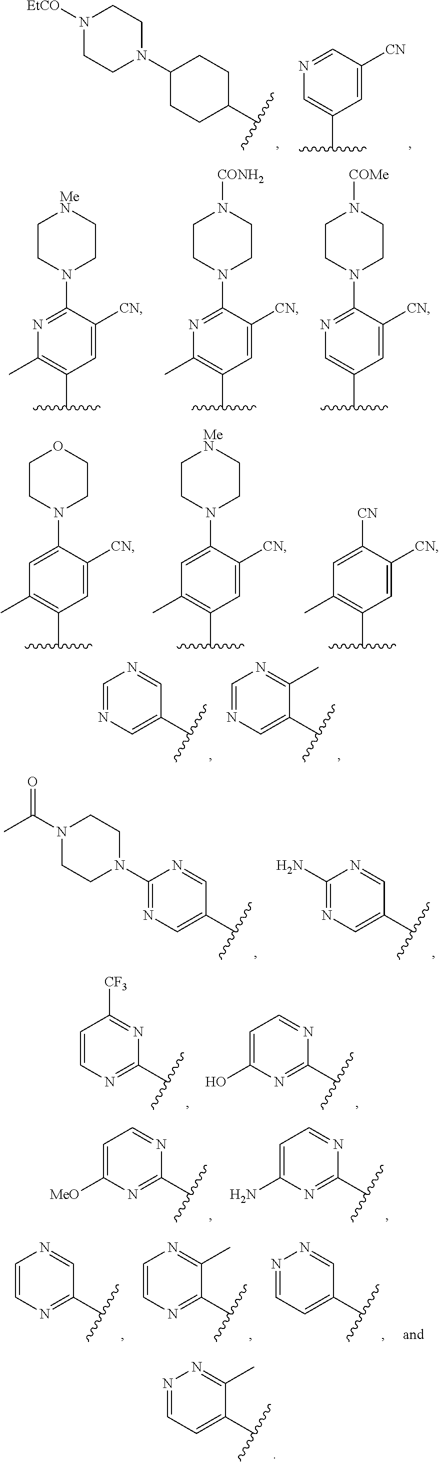

In certain embodiments, A is CH, R² is methyl, and R³ is H.

In certain embodiments, R² is methyl, R³ is H, and R⁴ is 4-chlorophenyl.

In certain of these embodiments, R² is methyl, R³ is H, R¹ is

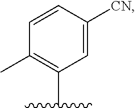 [SMILES: *c1cc(C#N)ccc1C]

### and R⁴ is

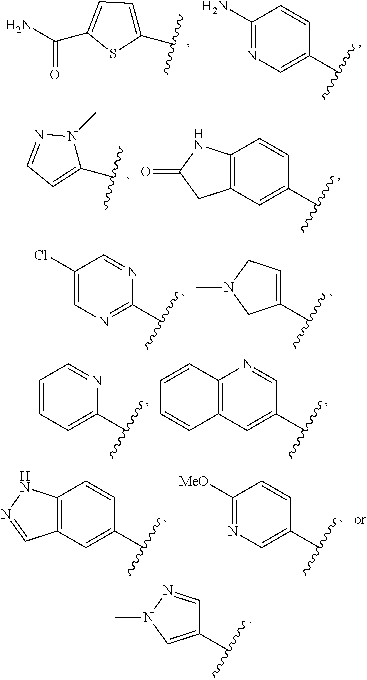

In certain embodiments, R² is methyl, R³ is H, R¹ is

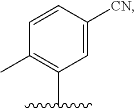 [SMILES: *c1cc(C#N)ccc1C]

and R⁴ is 4-fluorophenyl, 3-dimethylaminommthylphenyl, 3-cyanophenyl, 3-cyano-4-chlorophenyl, 3-methoxy-4-chlorophenyl, 3-chlorophenyl, 3-fluorophenyl, phenyl, 3-methylphenyl, 3-hydroxyphenyl, 3-aminophenyl, 3-hydroxy-4-fluorophenyl, 3,4-dimethoxyphenyl, 3,4-dichlorophenyl, 3,4-difluorophenyl, 2-methylphenyl, 2-chlorophenyl, or 2-hydroxyphenyl.

In certain embodiments, R² is methyl, R³ is H, R⁴ is 4-chlorophenyl, and R¹ is

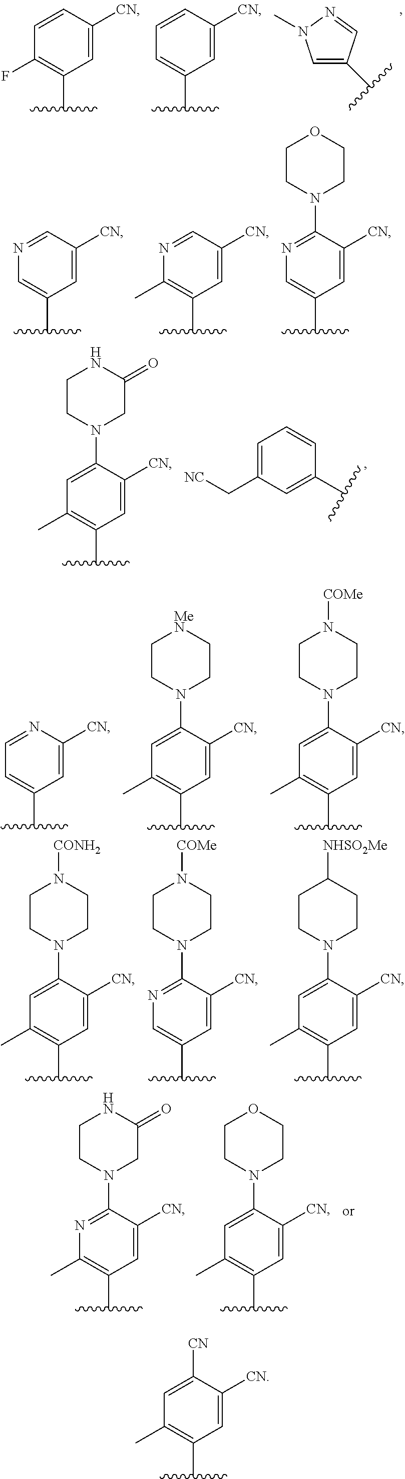

In certain embodiments, R² is methyl, R¹ is

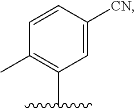 [SMILES: *c1cc(C#N)ccc1C]

R⁴ is 4-chlorophenyl, and R³ is —CN, ethylcarbonyl, 4-methylphenylsulfonyl, methyl, n-butyl, methylsulfonyl, guanidine, methoxycarbonyl, t-butyloxycarbonyl, n-butyloxycarbonyl, or aminosulfonyl.

In certain particular embodiments, R¹ is

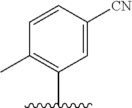 [SMILES: *c1cc(C#N)ccc1C]

and R² is methyl, R⁴ is 4-fluorophenyl, and R³ is —CN; R² is methyl, R⁴ is 4-fluorophenyl and R³ is or guanidine; R² is methyl, R³ is aminosulfonyl, and R⁴ is 4-chlorophenyl; R² is ethyl, R³ is H, and R⁴ is 4-chlorophenyl; R² is benzyl or 2-hydroxyethyl, R³ is H, and R⁴ is 4-chlorophenyl; R² is 2-hydroxyethyl, R³ is H, and R⁴ is 4-chlorophenyl, R² is methyl, R³ is methoxycarbonyl, and R⁴ is 2-methylpyridin-5-yl, or R² is methyl, R³ is methoxycarbonyl, and R⁴ is 4-chlorophenyl.

In certain particular embodiments, R² is methyl, R³ is aminosulfonyl or methoxycarbonyl, R⁴ is 2-amino-5-pyridyl, and R¹ is

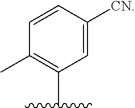 [SMILES: *c1cc(C#N)ccc1C]

In a particular embodiment, R² is methyl, R³ is H, R⁴ is 4-chlorophenyl, and R¹ is

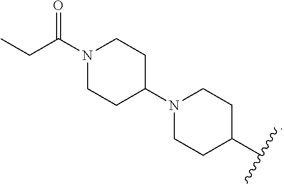 [SMILES: *C1CCN(C2CCN(C(=O)CC)CC2)CC1]

In certain particular embodiments, R² is methyl, R³ is H, R⁴ is 3-fluorophenyl, and R¹ is

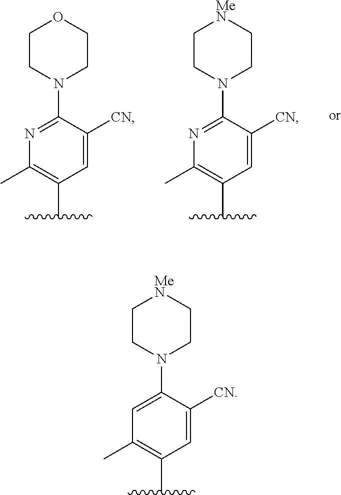 [SMILES: *c1cc(C#N)c(N2CCOCC2)nc1C ; *c1cc(C#N)c(N2CCN(C)CC2)nc1C ; "or" ; *c1cc(C#N)c(N2CCN(C)CC2)cc1C]

In certain particular embodiments, R² is methoxycarbonyl, R³ is H, R⁴ is 3-fluorophenyl or 4-methylphenyl, and R¹ is

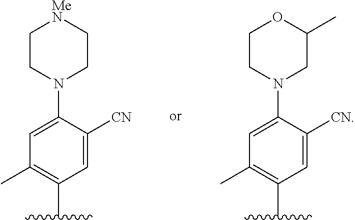 [SMILES: *c1cc(C#N)c(N2CCN(C)CC2)cc1C ; "or" ; *c1cc(C#N)c(N2CCOC(C)C2)cc1C]

In certain embodiments, B is CR⁸═CR⁹, A is CH, and R⁸ and R⁹ are both H.

In certain embodiments, R¹ is

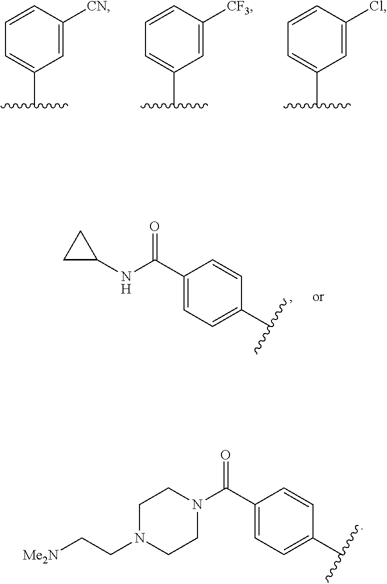 [SMILES: *c1cccc(C#N)c1 ; *c1cccc(C(F)(F)F)c1 ; *c1cccc(Cl)c1 ; *c1ccc(C(=O)NC2CC2)cc1 ; *c1ccc(C(=O)N2CCN(CCN(C)C)CC2)cc1]

In certain embodiments, R⁴ is 4-chlorophenyl or 2-amino-5-pyridyl.

In certain particular embodiments, R³ is H, R⁴ is 4-chlorophenyl, and R¹ is 3-trifluorophenyl, 3-chlorophenyl, or

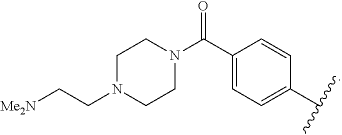 [SMILES: *c1ccc(C(=O)N2CCN(CCN(C)C)CC2)cc1]

In a particular embodiment, R³ is —CN, R⁴ is 4-chlorophenyl, and R¹ is

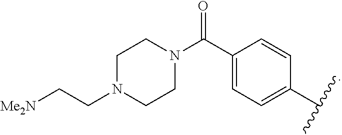 [SMILES: *c1ccc(C(=O)N2CCN(CCN(C)C)CC2)cc1]

In a particular embodiment, R³ is 4-methylphenylsulfonyl, R⁴ is 4-chlorophenyl, and R¹ is

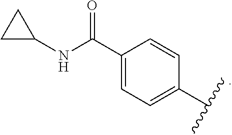 [SMILES: *c1ccc(C(=O)NC2CC2)cc1]

In certain particular embodiments, R³ is propionyl or methyl, R⁴ is 4-chlorophenyl, and R¹ is

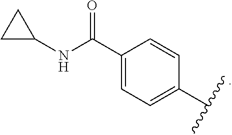 [SMILES: *c1ccc(C(=O)NC2CC2)cc1]

In certain embodiments, A is CH, B is NR², and R¹ is selected from the group consisting of

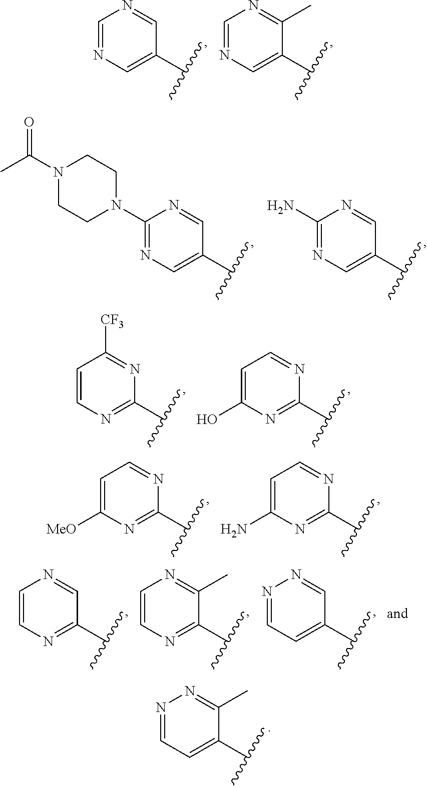

In certain embodiments, R¹ is

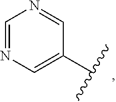 [SMILES: *c1cncnc1]

R² is methyl, R³ is H, and R⁴ is 4-chlorophenyl, 4-fluorophenyl, 2-aminopyrid-5-yl, 4-methylphenyl, 3-fluorophenyl,

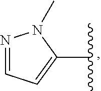 [SMILES: *c1ccnn1C]

3,4-dimethoxyphenyl, 3-cyano-4-chlorophenyl, 3-trifluoromethoxy-4-chlorophenyl, 2-trifluoromethylpyrid-5-yl, 2-methylpyrid-5-yl, or 2-methoxypyrid-5-yl.

In certain embodiments, R¹ is

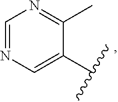 [SMILES: *c1cncnc1C]

R² is methyl, R³ is H, and R⁴ is 4-chlorophenyl, 4-fluorophenyl, 2-aminopyrid-5-yl, 4-methylphenyl, 3-fluorophenyl, 3-methoxy-4-chlorophenyl, 3-fluoro-4-methoxyphenyl, 3-methoxy-4-fluorophenyl, 3-trifluoromethyl-4-chlorophenyl, 3-trifluoromethoxy-4-chlorophenyl, 3,4-dimethoxylphenyl, 3,5-dimethoxylphenyl, 3,4-dimethoxy-5-fluorophenyl, 3,4-dimethoxy-5-chlorophenyl, and 3,4 5-trimethoxyphenyl.

In certain particular embodiments, R² is methyl, R³ is H, and wherein:

R¹ is

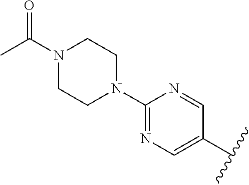 [SMILES: *c1cnc(N2CCN(C(C)=O)CC2)nc1]

and R⁴ is 4-chlorophenyl,

R¹ is

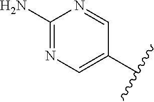 [SMILES: *c1cnc(N)nc1]

and R⁴ is 3-fluorophenyl,

R¹ is

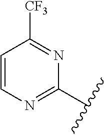 [SMILES: *c1nccc(C(F)(F)F)n1]

and R⁴ is 4-chlorophenyl or 3-fluororophenyl,

R¹ is

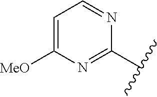 [SMILES: *c1nccc(OC)n1]

and R⁴ is 4-chlorophenyl or 3-fluororophenyl,

R¹ is

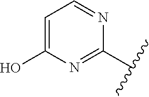 [SMILES: *c1nccc(O)n1]

and R⁴ is 4-chlorophenyl or 3-fluororophenyl,

R¹ is

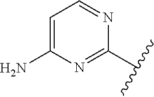 [SMILES: *c1nccc(N)n1]

and R⁴ is 4-chlorophenyl or 3-fluoro, or

R¹ is

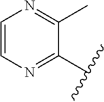 [SMILES: *c1nccnc1C]

and R⁴ is 3,4-dimethoxyphenyl.

In certain embodiments, R¹ is

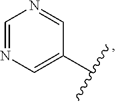 [SMILES: *c1cncnc1]

R² is methyl, R³ is

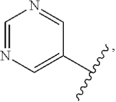 [SMILES: *c1cncnc1]

and R⁴ is 3-fluorophenyl, or

### R¹ is

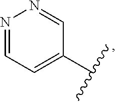 [SMILES: *c1ccnnc1]

R² is H, R³ is H, and R⁴ is 3,4-dimethoxyphenyl.

Chemistry

The compounds of the invention can be synthesized using any suitable route. In an embodiment, the compounds of formula (I) wherein A is CH and B is NR² can be synthesized by the route shown in Scheme 1.

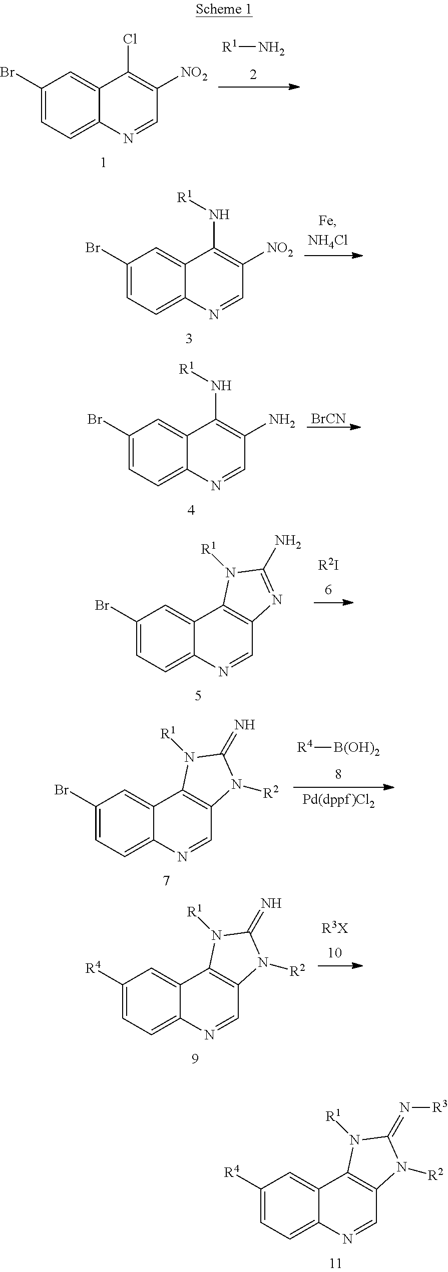

Compound 1 can be reacted with amine 2 in the presence of HC/dioxane in DMF at an elevated temperature such as 100° C. to give compound 3. Compound 3 can be reacted with Fe and NH₄Cl in EtOH and H₂O at a temperature such as 85° C. to provide Compound 4. Cyanation of compound 4 can be effected by reaction with carbononitridic bromide (BrCN) in a solvent such as EtOH at a temperature such as 80° C.-90° C. to give compound 5. Alkylation of compound 5 with 6 can be accomplished in DMF at a temperature such as 20-25° C. to afford compound 7. Suzuki coupling of compound 7 and boronic acid/ester 8 in the presence of a base such as Na₂CO₃ and a catalyst such as Pd(dppf)Cl₂ in a solvent such as a mixture of dioxane and H₂O at a temperature such as 100° C. gives compound 9, which can be reacted with reagent 10 to form compound 11.

In an embodiment, the compounds of formula (I) wherein A is CH and B is CR⁸═CR⁹ can be synthesized by the route shown in Scheme 2.

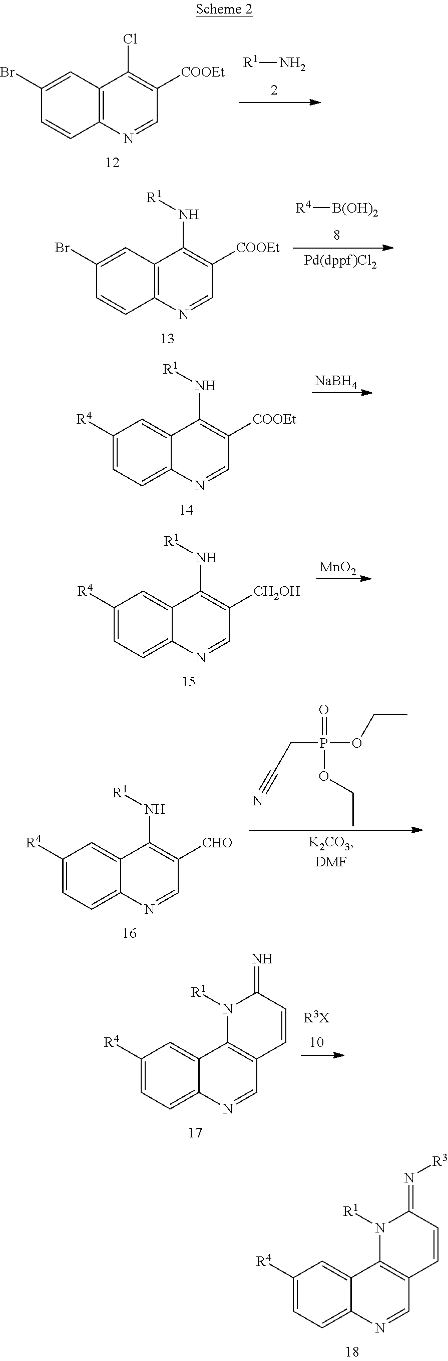

Compound 12 can be reacted with amine 2 in the presence of HCl/dioxane in a solvent such as DMF at a temperature such as 100° C. to give compound 13. Suzuki coupling of compound 13 and boronic acid/ester 8 in the presence of a base such as Na₂CO₃ and a catalyst such as Pd(dppf)Cl₂ in a mixture of dioxane and H₂O at a temperature such as 100° C. affords compound 14. Reduction of compound 14 with an agent such as NaBH₄ in a mixture of EtOH and THF at a temperature such as 20-25° C. gives compound 15. Oxidation of compound 15 with an oxidizing agent such as MnO₂ in a solvent such as DCM at a temperature such as 20-25° C. affords compound 16. Olefination of compound 16 with 2-diethoxyphosphorylacetonitrile and a base such as K₂CO₃ in a solvent such as DMF at a temperature such as 100° C. gives compound 17, which can be reacted with reagent 10 to form compound 18.

In another embodiment, the compounds of formula (I) wherein A is CH and B is NR² can be synthesized via a one-pot two step synthesis by the route shown in Scheme 3.

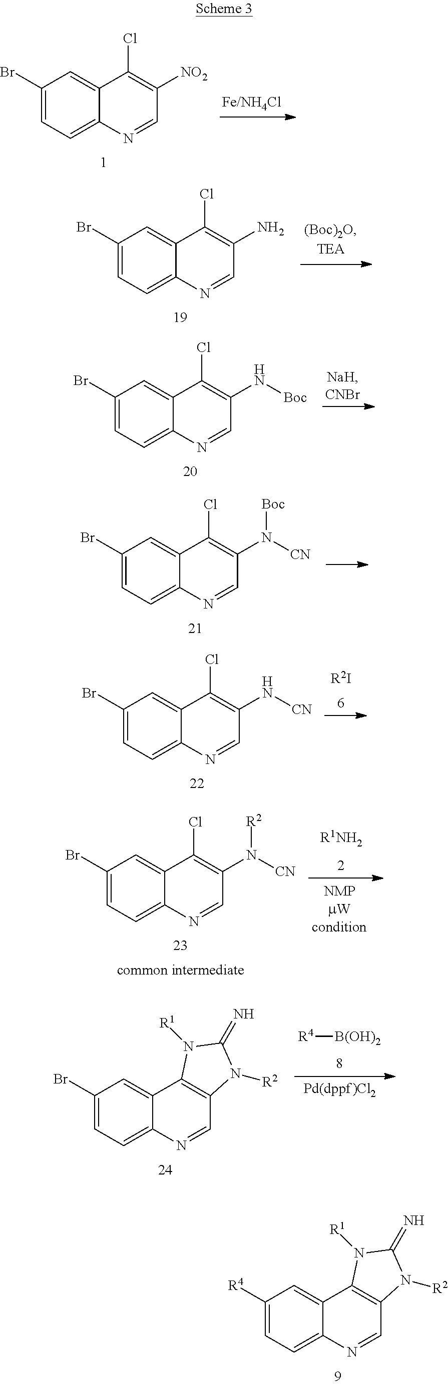

Reduction of compound 1 with Fe and NH₄Cl in EtOH and H₂O gave compound 19. Boc protection of compound 19 followed by cyanation afforded compound 21. Deprotection of compound 21 followed by alkylation gave the common intermediate 23. A mixture of compound 23 and amine 2 in NMP can be reacted under microwave irradiation at a temperature such as 180° C. (R¹=alkyl), or at a temperature such as 150° C. (R¹=aryl) to form compound 24. Suzuki coupling of compound 24 by addition of a solvent such DMF and a base/buffer such as 1M K₃PO₄ followed by boronic acid/ester 8 and a catalyst such as Pd(dppf)Cl₂ at a temperature such as 150° C. afforded compound 9.

In an embodiment, the invention provides a method of synthesizing a compound of formula 102, comprising the steps of

(a) providing a compound of formula 100:

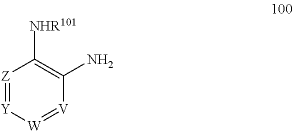

wherein R¹⁰¹ is C₆₋₁₀ aryl or heteroaryl substituted with at least one substituent selected from —CN, halo, —CF₃, —CONH₂, —OCF₃, C₁-C₆ alkyl, C₁-C₆ alkylcarbonyl, —OH, C₁-C₆ alkylaminocarbonyl, C₃-C₈ cycloalkylaminocarbonyl, C₁-C₆ alkylaminoalkyl, cyanomethyl, piperazinomethyl, C₁-C₆ alkylsulfonyl, C₁-C₆ alkylsulfonylamino, dialkylaminoalkylamino, optionally substituted piperazinylcarbonyl, and C₁-C₆ alkoxy, optionally further in combination with one or more substituents selected from halo, C₁₋₁₂ alkyl, a heterocyclyl group selected from the group consisting of optionally substituted piperazinyl, morpholinyl optionally substituted with C₁-C₆ alkyl, azetidin-1-yl, pyrrolidin-1-yl, piperidin-1-yl, octahydropyrrolo[3,4-b]pyrrolyl, 2-oxa-6-azaspiro[3.3]heptyl, or is a C₃-C₈ cycloalkyl or C₃-C₈ azacycloalkyl substituted with morpholino, optionally substituted piperidinyl, or optionally substituted piperazinyl,

### V is CR¹⁰³ or N,

### W is CR¹⁰⁴ or N,

### Y is CR¹⁰⁵ or N,

### Z is CR¹⁰⁶ or N,

wherein R¹⁰³-R¹⁰⁶ are independently hydrogen, optionally substituted alkyl, optionally substituted aryl, or optionally substituted heteroaryl, or when Y is CR¹⁰⁶ and Z is CR¹⁰⁷, R¹⁰⁶ and R¹⁰⁶, taken together with the carbons to which they are bound, form an optionally substituted fused 5- to 8-membered carbocyclic, aryl, heterocyclyl, or heteroaryl ring, wherein the heterocyclyl contains one or more atoms selected from N, O, and S, wherein at least one of V, W, Y, and Z is N,

(b) reacting the compound of formula 100 with a cyanation reagent to provide a compound of formula 101:

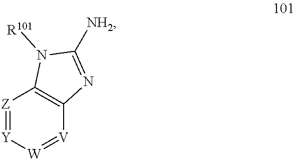

and
(c) reacting the compound of formula 101 with an alkylating agent of the formula:

R¹⁰²-LG

wherein R¹⁰² is C₁-C₆ alkyl, hydroxyl C₁-C₆ alkyl, C₁-C₆ alkylcarbonyl, C₁-C₆ alkylsulfonyl, or optionally substituted benzyl and LG is a leaving group selected from halo, alkylsulfonate, and arylsulfonate to give the compound of formula 102:

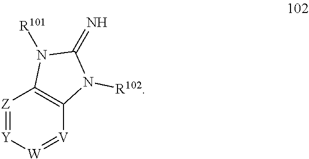

In another embodiment, the invention provides a method of synthesizing a compound of formula 102, comprising the steps of:

(a) providing a compound of formula 103:

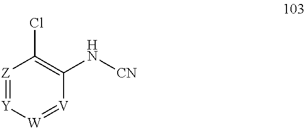

wherein V is CR¹⁰³ or N,

### W is CR¹⁰⁴ or N,

### Y is CR¹⁰⁵ or N,

### Z is CR¹⁰⁶ or N,

wherein R¹⁰³-R¹⁰⁶ are independently hydrogen, optionally substituted alkyl, optionally substituted aryl, or optionally substituted heteroaryl, or when Y is CR¹⁰⁶ and Z is CR¹⁰⁷, R¹⁰⁶ and R¹⁰⁶, taken together with the carbons to which they are bound, form an optionally substituted fused 5- to 8-membered carbocyclic, aryl, heterocyclyl, or heteroaryl ring, wherein the heterocyclyl contains one or more atoms selected from N, O, and S, wherein at least one of V, W, Y, and Z is N,

(b) reacting the compound of formula 103 with an alkylating agent of the formula:

R¹⁰²-LG

wherein R¹⁰² is C₁-C₆ alkyl, hydroxyl C₁-C₆ alkyl, C₁-C₆ alkylcarbonyl, C₁-C₆ alkylsulfonyl, or optionally substituted benzyl and LG is a leaving group selected from halo, alkylsulfonate, and arylsulfonate to give a compound of formula 104:

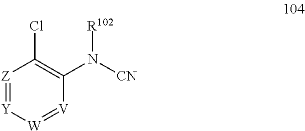

and
(c) reacting the compound of formula 104 with an amine of the formula R¹⁰¹—NH₂ to give the compound of formula 102:

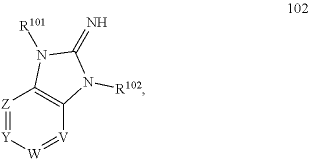

wherein R¹⁰¹ is C₆₋₁₀ aryl or heteroaryl substituted with at least one substituent selected from —CN, halo, —CF₃, —CONH₂, —OCF₃, C₁-C₆ alkyl, C₁-C₆ alkylcarbonyl, —OH, C₁-C₆ alkylaminocarbonyl, C₃-C₈ cycloalkylaminocarbonyl, C₁-C₆ alkylaminoalkyl, cyanomethyl, piperazinomethyl, C₁-C₆ alkylsulfonyl, C₁-C₆ alkylsulfonylamino, dialkylaminoalkylamino, optionally substituted piperazinylcarbonyl, and C₁-C₆ alkoxy, optionally further in combination with one or more substituents selected from halo, C₁₋₁₂ alkyl, a heterocyclyl group selected from the group consisting of optionally substituted piperazinyl, morpholinyl optionally substituted with C₁-C₆ alkyl, azetidin-1-yl, pyrrolidin-1-yl, piperidin-1-yl, octahydropyrrolo[3,4-b]pyrrolyl, 2-oxa-6-azaspiro[3.3]heptyl, or is a C₃-C₈ cycloalkyl or C₃-C₈ azacycloalkyl substituted with morpholino, optionally substituted piperidinyl, or optionally substituted piperazinyl.

In a further embodiment, the invention provides a method of synthesizing a compound of formula 106, comprising the steps of:

(a) providing a compound of formula 105:

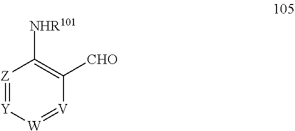

wherein R¹⁰¹ is C₆₋₁₀ aryl or heteroaryl substituted with at least one substituent selected from —CN, halo, —CF₃, —CONH₂, —OCF₃, C₁-C₆ alkyl, C₁-C₆ alkylcarbonyl, —OH, C₁-C₆ alkylaminocarbonyl, C₃-C₈ cycloalkylaminocarbonyl, C₁-C₆ alkylaminoalkyl, cyanomethyl, piperazinomethyl, C₁-C₆ alkylsulfonyl, C₁-C₆ alkylsulfonylamino, dialkylaminoalkylamino, optionally substituted piperazinylcarbonyl, and C₁-C₆ alkoxy, optionally further in combination with one or more substituents selected from halo, C₁₋₁₂ alkyl, a heterocyclyl group selected from the group consisting of optionally substituted piperazinyl, morpholinyl optionally substituted with C₁-C₆ alkyl, azetidin-1-yl, pyrrolidin-1-yl, piperidin-1-yl, octahydropyrrolo[3,4-b]pyrrolyl, 2-oxa-6-azaspiro[3.3]heptyl, or is a C₃-C₈ cycloalkyl or C₃-C₈ azacycloalkyl substituted with morpholino, optionally substituted piperidinyl, or optionally substituted piperazinyl, and
(b) reacting the compound of formula 105 with a di(C₁-C₆)alkyl cyanomethylphosphonate to give the compound of formula 106:

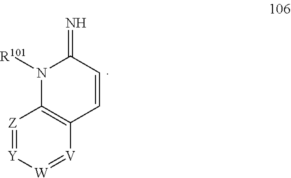

In accordance with an embodiment of the invention, any of the compounds or salts thereof can be administered in the form of a pharmaceutical composition comprising the compound or salt and a pharmaceutically acceptable carrier.

The pharmaceutically acceptable carriers described herein, for example, vehicles, adjuvants, excipients, or diluents, are well known to those who are skilled in the art and are readily available to the public. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, intraperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The compounds of the present invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Additionally, the compounds of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable carriers and their formulations are further described in A. R. Gennaro, ed., Remington: The Science and Practice of Pharmacy (19th ed.), Mack Publishing Company, Easton, Pa. (1995).

The compound of the invention or a composition thereof can be administered as a pharmaceutically acceptable acid-addition, base neutralized or addition salt, formed by reaction with inorganic acids, such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base, such as sodium hydroxide, ammonium hydroxide, potassium hydroxide, and organic bases, such as mono-, di-, trialkyl, and aryl amines and substituted ethanolamines. The conversion to a salt is accomplished by treatment of the base compound with at least a stoichiometric amount of an appropriate acid. Typically, the free base is dissolved in an inert organic solvent such as diethyl ether, ethyl acetate, chloroform, ethanol, methanol, and the like, and the acid is added in a similar solvent. The mixture is maintained at a suitable temperature (e.g., between 0° C. and 50° C.). The resulting salt precipitates spontaneously or can be brought out of solution with a less polar solvent.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

It should be recognized that the particular counterion forming a part of any salt of this invention is usually not of a critical nature, so long as the salt as a whole is pharmacologically acceptable and as long as the counterion does not contribute undesired qualities to the salt as a whole.

It is further understood that the above compounds and salts may form solvates, or exist in a substantially uncomplexed form, such as the anhydrous form. As used herein, the term “solvate” refers to a molecular complex wherein the solvent molecule, such as the crystallizing solvent, is incorporated into the crystal lattice. When the solvent incorporated in the solvate is water, the molecular complex is called a hydrate. Pharmaceutically acceptable solvates include hydrates, alcoholates such as methanolates and ethanolates, acetonitrilates and the like. These compounds can also exist in polymorphic forms.

In an embodiment, the invention provides a method of blocking transmission of a Plasmodium parasite comprising administering to a mammal in need of such treatment, a therapeutically effective amount of a compound of the invention. In another embodiment, the invention provides a method of treating malaria by killing or arresting the growth of Plasmodium organisms in a mammal, wherein the Plasmodium organisms are in a liver stage or an asexual stage, the method comprising administering to a mammal a therapeutically effective amount of a compound of the invention.

The Plasmodium parasite can be any suitable Plasmodium parasite. Non-limiting examples of suitable Plasmodium parasites include Plasmodium falciparum, Plasmodium vivax, Plasmodium malariae, Plasmodium ovale, and Plasmodium knowlesi. In a preferred embodiment, the Plasmodium parasite is Plasmodium falciparum.

In an embodiment, the Plasmodium parasite is a Plasmodium gametocyte.

In embodiments, the Plasmodium gametocyte is a mature stage II-V gametocyte. In a preferred embodiment, the Plasmodium gametocyte is a stage III-V gametocyte, e.g., a mature stage III-V gametocyte. In another preferred embodiment, the Plasmodium gametocyte is a mature stage V gametocyte.

In certain preferred embodiments, the compound effectively kills Plasmodium gametocytes.

In embodiments, the Plasmodium parasite is a drug-resistant strain. Examples of drug-resistant strains of Plasmodium are described in Kun, J. F. J. et al., Antimicrob Agents Chemother., 1999 September; 43(9): 2205-2208, and references cited therein.

In embodiments, the Plasmodium parasite is in an asexual stage. For example, the Plasmodium parasite can be a sporozoite, a liver stage parasite, a merozoite, an asexual erythrocyte-stage parasite, a zygote, an ookinete, or an oocyst.

The amount or dose of a compound of the invention or a salt thereof, or a composition thereof should be sufficient to effect a therapeutic or prophylactic response in the mammal. The appropriate dose will depend upon several factors. For instance, the dose also will be determined by the existence, nature and extent of any adverse side effects that might accompany the administration of a particular compound or salt. Ultimately, the attending physician will decide the dosage of the compound of the present invention with which to treat each individual patient, taking into consideration a variety of factors, such as age, body weight, general health, diet, sex, compound or salt to be administered, route of administration, and the severity of the condition being treated. By way of example and not intending to limit the invention, the dose of the compound(s) described herein can be about 0.1 mg to about 1 g daily, for example, about 5 mg to about 500 mg daily. Further examples of doses include but are not limited to: 0.1 mg, 0.15 mg, 0.2 mg, 0.25 mg, 0.5 mg, 0.6 mg, 0.75 mg, 1 mg, 1.5 mg, 2 mg, 3 mg, 4 mg, 5 mg, 6 mg, 7 mg, 8 mg, 9 mg, 10 mg, 12 mg, 15 mg, 17 mg, 20 mg, 25 mg, 30 mg, 35 mg, 40 mg, 45 mg, 50 mg, 55 mg, 60 mg, 70 mg, 75 mg, 80 mg, 85 mg, 90 mg, 95 mg, 100 mg, 125 mg, 140 mg, 150 mg, 175 mg, 200 mg, 225 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg/kg body weight per day.

In certain embodiments, the method further comprises administering to the mammal at least one additional antimalarial compound. Any suitable antimalarial compound can be used, many of which are well known in the art. Non-limiting examples of suitable antimalarial compounds include primaquine, bulaquine, artemisinin and derivatives thereof, chloroquine, mefloquine, amodiaquine, piperaquine, pyronaridine, atovaquone, tafenoquine, methylene blue, trioxaquines, endoperoxides such as OZ 439 and OZ 277, decoquinate, 9-anilinoacridines, HIV-protease inhibitors, and natural products such as neem, epoxomicin, harmonine, and riboflavin. In certain preferred embodiments, the compound of the invention is administered in combination with elesclomol, NSC174938, NVP-AUY922, maduramicin, narasin, alvespimycin, omacetaxine, thiram, zinc pyrithione, phanquinone, bortezomib, salinomycin sodium, monensin sodium, dipyrithione, dicyclopentamethylene-thiuram disulfide, YM155, withaferin A, adriamycin, romidepsin, AZD-1152-HQPA, CAY10581, plicamycin, CUDC-101, auranofin, trametinib, GSK-458, afatinib, panobinostat, or any combination thereof.

### ILLUSTRATIVE EXAMPLES OF EMBODIMENTS

The invention contains at least the following embodiments:

1. A compound of formula (I):

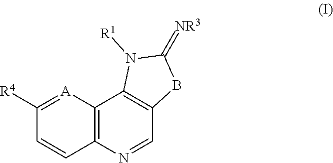

wherein A is CR⁵ or N,

B is CR⁸═CR⁹ or NR²,

R⁸ and R⁹ are independently selected from hydrogen, hydroxyl, OR¹⁰, halogen, optionally substituted C₆₋₁₀ aryl, and optionally substituted C₁₋₆ alkyl,

R¹⁰ is hydrogen, C₁₋₁₂ alkyl, C₃₋₈ cycloalkyl, CH₂COOR¹³, or H₂N(CH₂)ₙ— wherein n is an integer of 2-6,

R¹ is C₆₋₁₀ aryl or heteroaryl optionally substituted with at least one substituent selected from —CN, halo, —CF₃, —CONH₂, —OCF₃, C₁-C₆ alkyl, C₁-C₆ alkylcarbonyl, —OH, C₁-C₆ alkylaminocarbonyl, C₃-C₈ cycloalkylaminocarbonyl, C₁-C₆ alkylaminoalkyl, cyanomethyl, piperazinomethyl, C₁-C₆ alkylsulfonyl, C₁-C₆ alkylsulfonylamino, dialkylaminoalkylamino, optionally substituted piperazinylcarbonyl, and C₁-C₆ alkoxy, a heterocyclyl group selected from the group consisting of 4-(C₁-C₆ alkylcarbonyl)piperazin-1-yl, morpholinyl optionally substituted with C₁-C₆ alkyl, azetidin-1-yl, pyrrolidin-1-yl, piperidin-1-yl, octahydropyrrolo[3,4-b]pyrrolyl, and 2-oxa-6-azaspiro[3.3]heptyl, C₃-C₈ cycloalkyl or C₃-C₈ azacycloalkyl, each substituted with morpholino, optionally substituted piperidinyl, or optionally substituted piperazinyl,

R² is C₁-C₆ alkyl, hydroxyl C₁-C₆ alkyl, C₁-C₆ alkylcarbonyl, C₁-C₆ alkylsulfonyl, or optionally substituted benzyl,

R³ is H, —CN, C₁-C₆ alkylcarbonyl, C₁-C₆ alkoxycarbonyl, phenylsulfonyl, 4-methylphenylsulfonyl, C₁-C₆ alkylsulfonyl, aminocarbonyl, aminosulfonyl, optionally substituted benzyl, —OH, —OR, —SR, —(S═O)R (R═C₁-C₆ alkyl), guanidino, or pyrimidin-5-yl,

R⁴ is phenyl, heteroaryl, 1-phenyl-2-ethynyl, or heterocyclyl, wherein the phenyl, heteroaryl, heterocyclyl, or phenyl of the 1-phenyl-2-ethynyl is optionally substituted with one or more substituents selected from the group consisting of halo, C₁-C₆ alkyl, amino, oxo, dialkylaminoalkyl, dialkylaminoalkoxy, —CN, aminocarbonyl, —OR⁶, CF₃, and C₁-C₆ alkylsulfonyl,

R⁶ is H or C₁-C₆ alkyl,

R⁵ is hydrogen, C₁-C₆ alkyl, C₆-C₁₀ aryl, halogen, hydroxyl, or OR⁷,

R⁷ is C₁-C₆ alkyl, formyl, C₁-C₆ acyl, or C₆-C₁₀ aryl,

or a pharmaceutically acceptable salt thereof.

2. The compound or salt of embodiment 1, wherein:

R¹ is C₆₋₁₀ aryl or heteroaryl substituted with at least one substituent selected from —CN, halo, —CF₃, —CONH₂, —OCF₃, C₁-C₆ alkyl, C₁-C₆ alkylcarbonyl, —OH, C₁-C₆ alkylaminocarbonyl, C₃-C₈ cycloalkylaminocarbonyl, C₁-C₆ alkylaminoalkyl, cyanomethyl, piperazinomethyl, C₁-C₆ alkylsulfonyl, C₁-C₆ alkylsulfonylamino, dialkylaminoalkylamino, optionally substituted piperazinylcarbonyl, and C₁-C₆ alkoxy, a heterocyclyl group selected from the group consisting of piperazin-1-yl, 4-(C₁-C₆ alkylcarbonyl)piperazin-1-yl, morpholinyl optionally substituted with C₁-C₆ alkyl, azetidin-1-yl, pyrrolidin-1-yl, piperidin-1-yl, octahydropyrrolo[3,4-b]pyrrolyl, and 2-oxa-6-azaspiro[3.3]heptyl, C₃-C₈ cycloalkyl or C₃-C₈ azacycloalkyl substituted with morpholino, optionally substituted piperidinyl, or optionally substituted piperazinyl,

R³ is H, —CN, C₁-C₆ alkylcarbonyl, C₁-C₆ alkoxycarbonyl, phenylsulfonyl, 4-methylphenylsulfonyl, C₁-C₆ alkylsulfonyl, aminocarbonyl, aminosulfonyl, optionally substituted benzyl, —OH, —OR, —SR, —(S═O)R (R═C₁-C₆ alkyl), or guanidino, and

R⁴ is phenyl, heteroaryl, 1-phenyl-2-ethynyl, or heterocyclyl, wherein the phenyl, heteroaryl, heterocyclyl, or phenyl of the 1-phenyl-2-ethynyl is optionally substituted with one or more substituents selected from the group consisting of halo, C₁-C₆ alkyl, amino, oxo, dialkylaminoalkyl, dialkylaminoalkoxy, —CN, aminocarbonyl, and —OR⁶,

3. The compound or salt of embodiment 1 or 2, wherein B is NR².

4, The compound or salt of embodiment 3, wherein R¹ is selected from the group consisting of

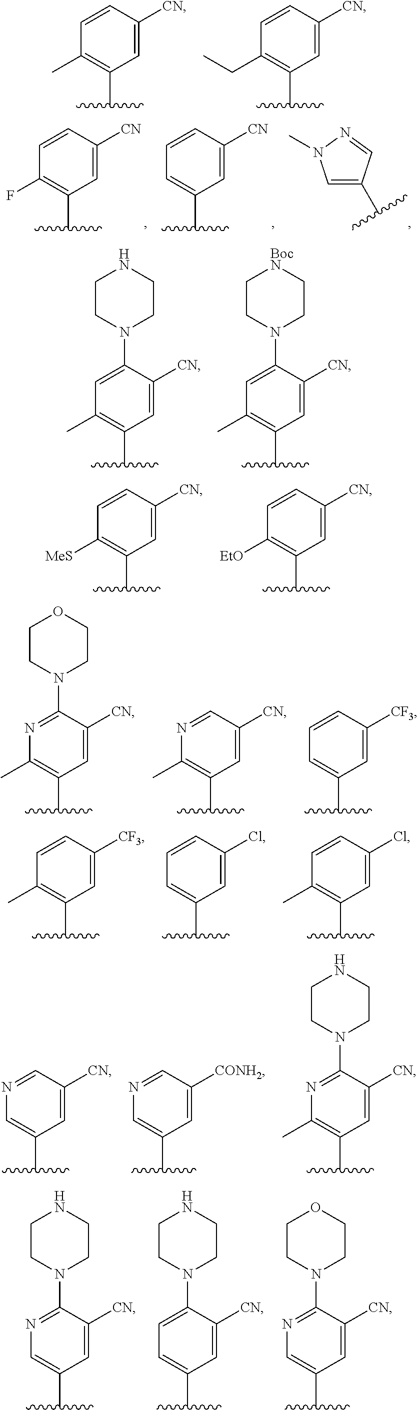

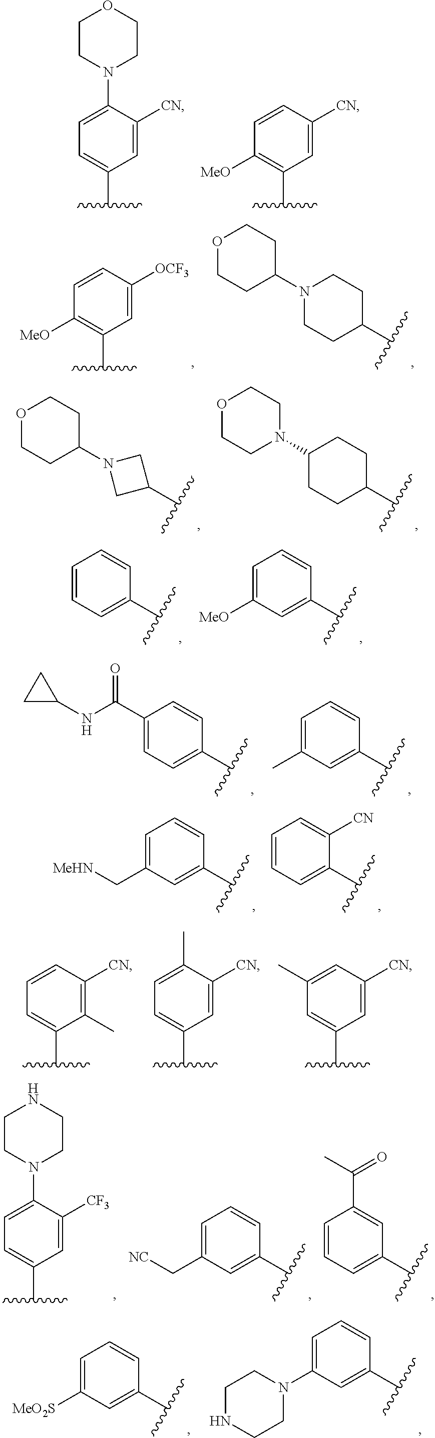

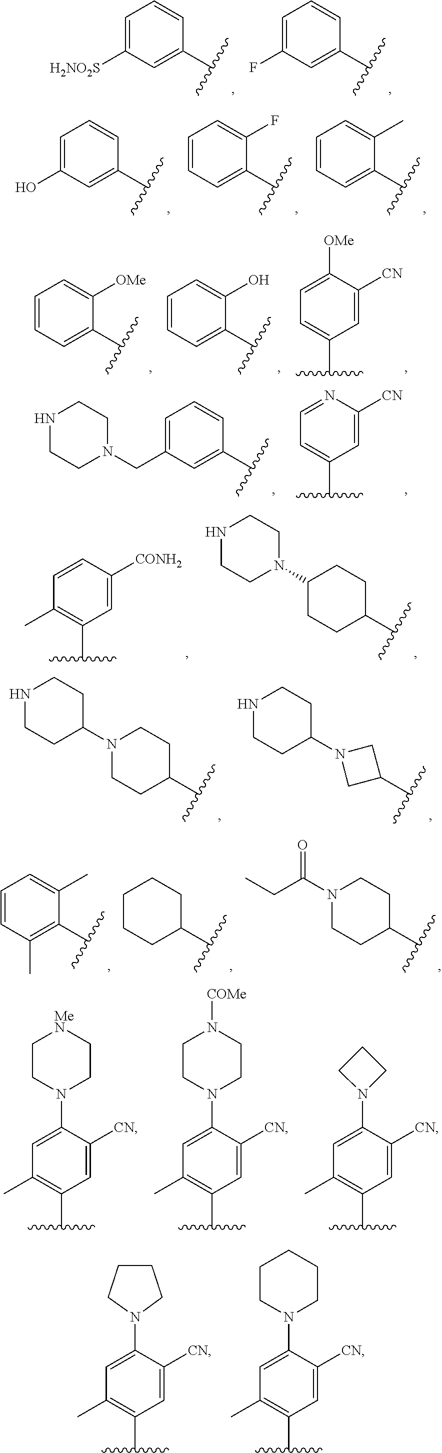

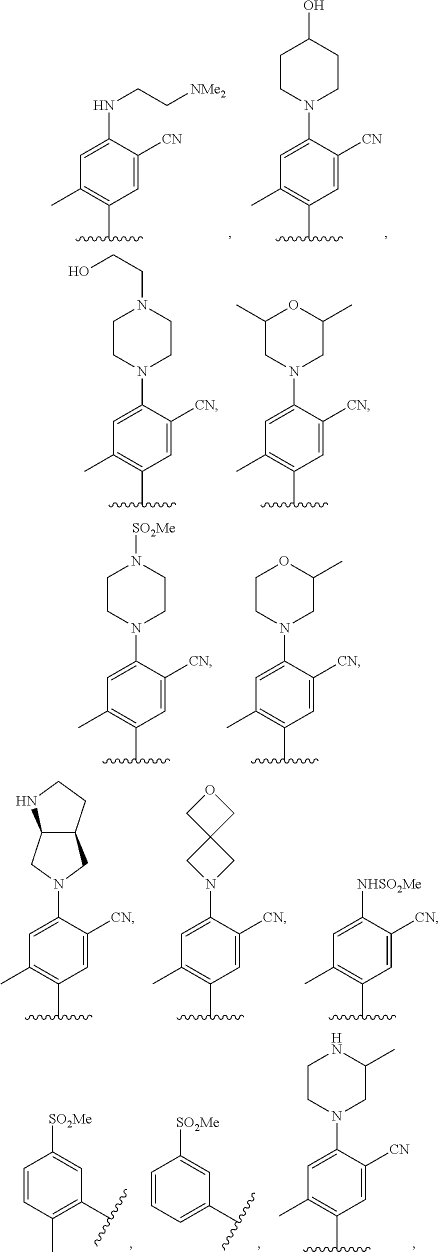

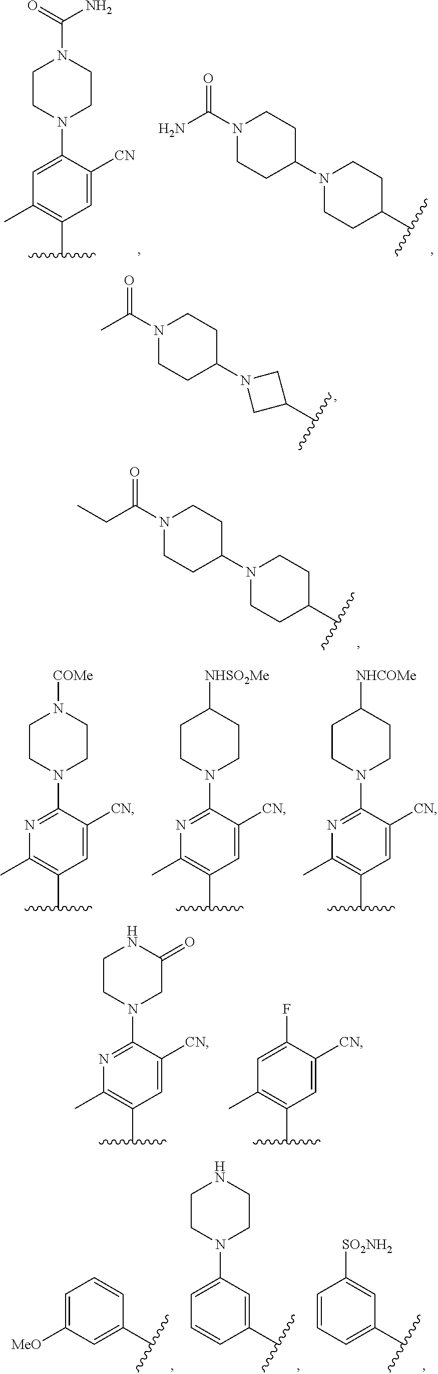

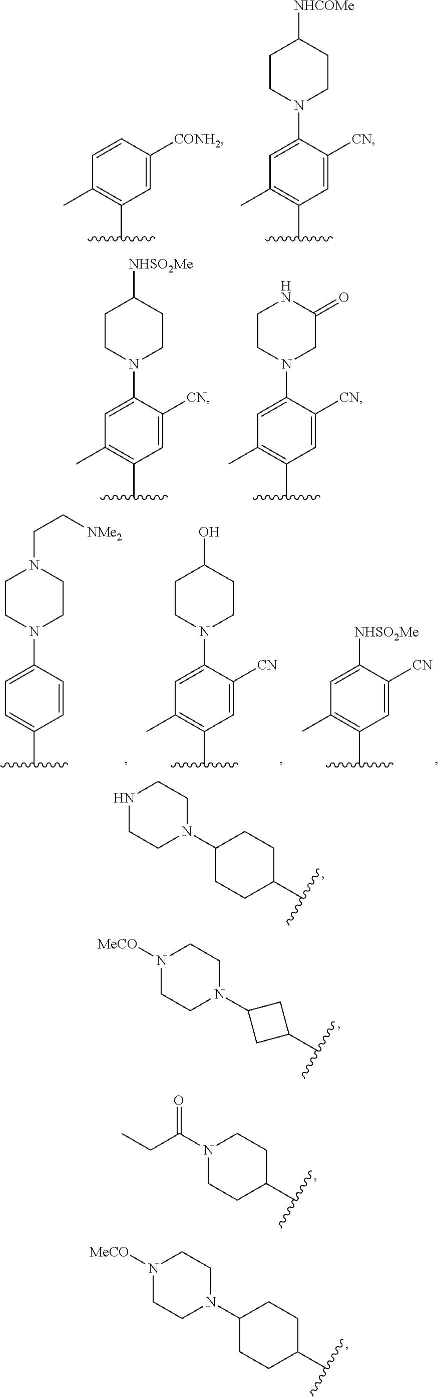

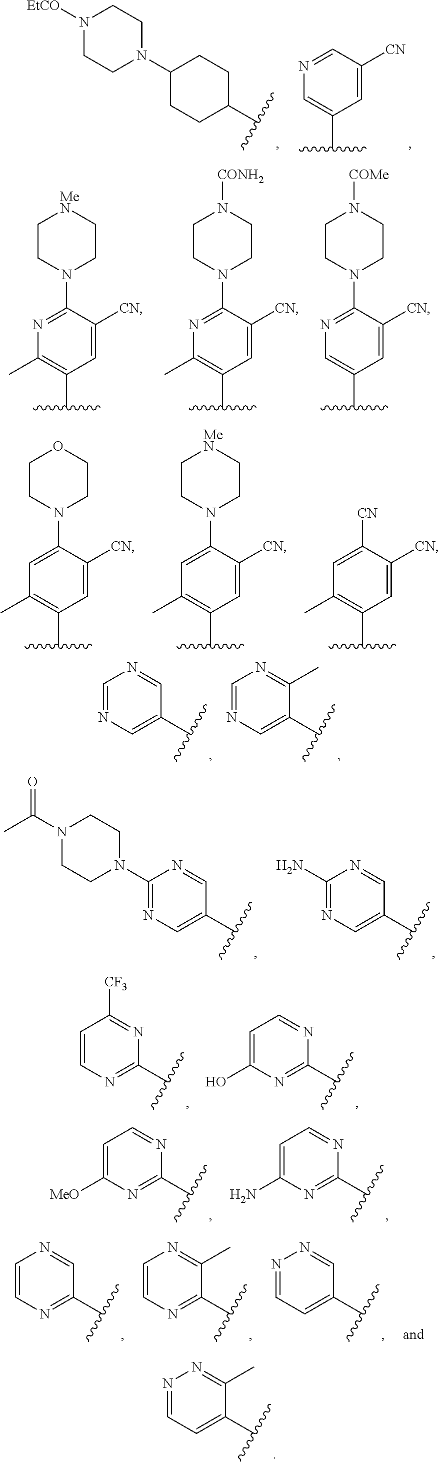

5. The compound or salt of embodiment 4, wherein A is CH.

6. The compound or salt of embodiment 4 or 5, wherein R² is methyl.

7. The compound or salt of any one of embodiments 4-6, wherein R³ is H.

8. The compound or salt of any one of embodiments 4-7, wherein R² is methyl,

R³ is H, and R⁴ is 4-chlorophenyl.

9. The compound or salt of any one of embodiments 4-8, wherein R² is methyl, R³ is H, R¹ is

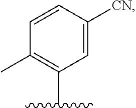 [SMILES: *c1cc(C#N)ccc1C]

### and R⁴ is

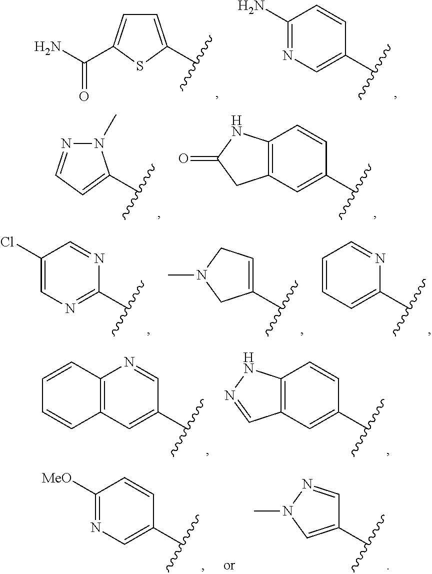

10. The compound or salt of any one of embodiments 4-7, wherein R² is methyl, R³ is H, R¹ is

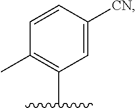 [SMILES: *c1cc(C#N)ccc1C]

and R⁴ is 4-fluorophenyl, 3-dimethylaminomethylphenyl, 3-cyanophenyl, 3-cyano-4-chlorophenyl, 3-methoxy-4-chlorophenyl, 3-chlorophenyl, 3-fluorophenyl, phenyl, 3-methylphenyl, 3-hydroxyphenyl, 3-aminophenyl, 3-hydroxy-4-fluorophenyl, 3,4-dimethoxyphenyl, 3,4-dichlorophenyl, 3,4-difluorophenyl, 2-methylphenyl, 2-chlorophenyl, or 2-hydroxyphenyl.

11. The compound or salt of any one of embodiments 4-7, wherein R² is methyl, R³ is H, R⁴ is 4-chlorophenyl, and R¹ is

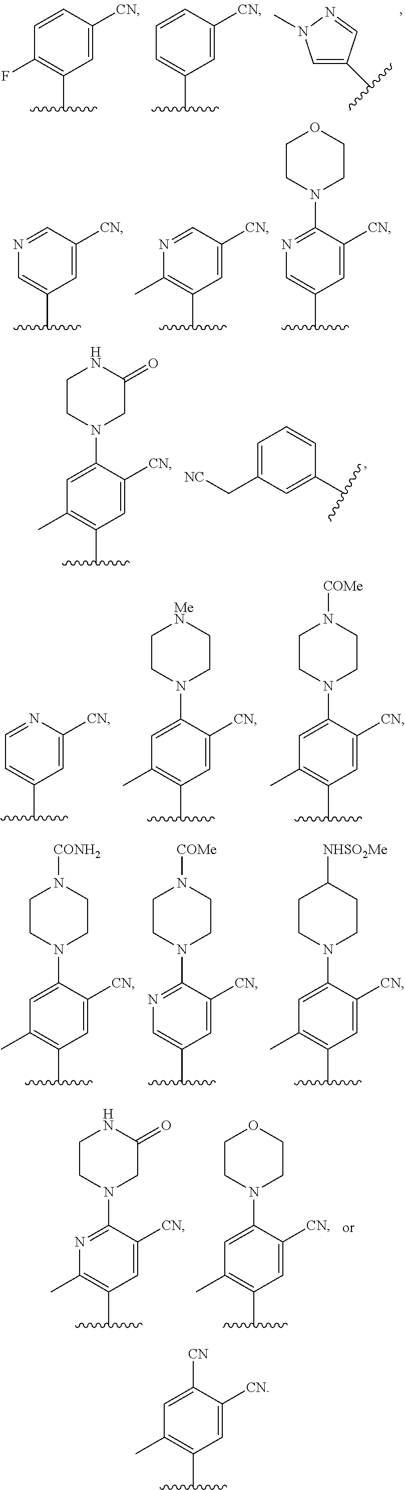

12. The compound or salt of any one of embodiments 4-6, wherein R² is methyl, R¹ is

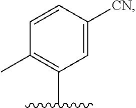 [SMILES: *c1cc(C#N)ccc1C]

R⁴ is 4-chlorophenyl, and R³ is —CN, ethylcarbonyl, 4-methylphenylsulfonyl, methyl, n-butyl, methylsulfonyl, guanidine, methoxycarbonyl, t-butyloxycarbonyl, n-butyloxycarbonyl, or aminosulfonyl.

13. The compound or salt of any one of embodiments 4-6, wherein R² is methyl, R¹ is

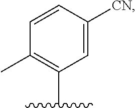 [SMILES: *c1cc(C#N)ccc1C]

R⁴ is 4-fluorophenyl, and R³ is —CN or guanidino.

14. The compound or salt of any one of embodiments 4-6, wherein R² is methyl, R¹ is

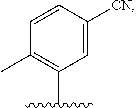 [SMILES: *c1cc(C#N)ccc1C]

R³ is aminosulfonyl, and R⁴ is 4-chlorophenyl.

15. The compound or salt of any one of embodiments 4-7, wherein R¹ is

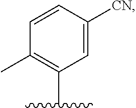 [SMILES: *c1cc(C#N)ccc1C]

R³ is H, R⁴ is 4-chlorophenyl, and R² is ethyl, benzyl, or 2-hydroxyethyl.

16. The compound or salt of any one of embodiments 4-6, wherein R² is methyl, R¹ is

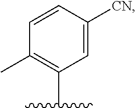 [SMILES: *c1cc(C#N)ccc1C]

R³ is methoxycarbonyl, and R⁴ is 2-methylpyridin-5-yl or 4-chlorophenyl.

17. The compound or salt of any one of embodiments 4-6, wherein R³ is methyl, R³ is aminosulfonyl or methoxycarbonyl, R⁴ is 2-amino-5-pyridyl, and R¹ is

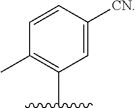 [SMILES: *c1cc(C#N)ccc1C]

18. The compound or salt of any one of embodiments 4-6, wherein R² is methyl, R³ is H, R⁴ is 4-chlorophenyl, and R¹ is

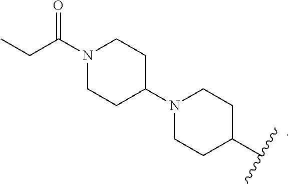 [SMILES: *C1CCN(C2CCN(C(=O)CC)CC2)CC1]

19. The compound or salt of any one of embodiments 4-6, wherein R² is methyl, R³ is H, R⁴ is 3-fluorophenyl, and R¹ is

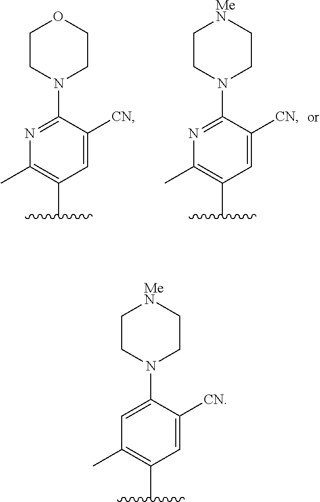 [SMILES: *c1cc(C#N)c(N2CCOCC2)nc1C ; *c1cc(C#N)c(N2CCN(C)CC2)nc1C ; *c1cc(C#N)c(N2CCN(C)CC2)cc1C]

20. The compound or salt of any one of embodiments 4-6, wherein R² is methoxycarbonyl, R³ is H, R⁴ is 3-fluorophenyl or 4-methylphenyl, and R¹ is

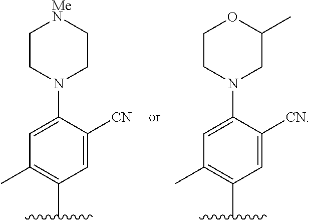 [SMILES: *c1cc(C#N)c(N2CCN(C)CC2)cc1C ; *c1cc(C#N)c(N2CCOC(C)C2)cc1C]

21. The compound or salt of embodiment 1 or 2, wherein B is CR⁸═CR⁹ and A is CH.

22. The compound or salt of embodiment 21, wherein R⁸ and R⁹ are both H.

23. The compound or salt of embodiment 21 or 22, wherein R¹ is

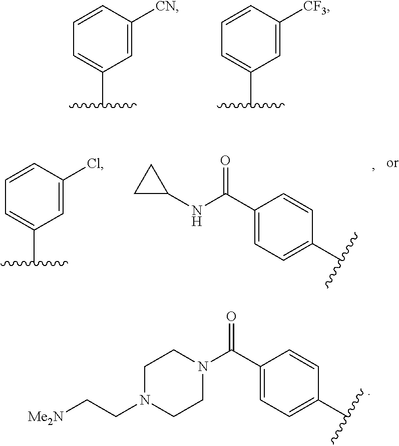 [SMILES: *c1cccc(C#N)c1 ; *c1cccc(C(F)(F)F)c1 ; *c1cccc(Cl)c1 ; *c1ccc(C(=O)NC2CC2)cc1 ; *c1ccc(C(=O)N2CCN(CCN(C)C)CC2)cc1]

24. The compound or salt of any one of embodiments 21-23, wherein R⁴ is 4-chlorophenyl or 2-amino-5-pyridyl.

25. The compound or salt of any one of embodiments 21-24, wherein R³ is H, R⁴ is 4-chlorophenyl, and R¹ is 3-trifluorophenyl, 3-chlorophenyl, or

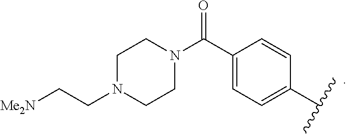 [SMILES: *c1ccc(C(=O)N2CCN(CCN(C)C)CC2)cc1]

26. The compound or salt of any one of embodiments 21-24, wherein R³ is —CN, R⁴ is 4-chlorophenyl, and R¹ is

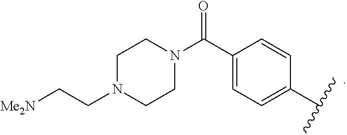 [SMILES: *c1ccc(C(=O)N2CCN(CCN(C)C)CC2)cc1]

27. The compound or salt of any one of embodiments 21-24, wherein R³ is 4-methylphenylsulfonyl, R⁴ is 4-chlorophenyl, and R¹ is

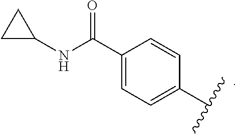 [SMILES: *c1ccc(C(=O)NC2CC2)cc1]

28. The compound or salt of any one of embodiments 21-24, wherein R³ is propionyl or methyl, R⁴ is 4-chlorophenyl, and R¹ is

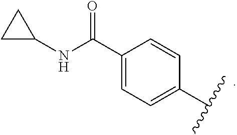 [SMILES: *c1ccc(C(=O)NC2CC2)cc1]

29. The compound or salt of embodiment 1, wherein A is CH, B is NR², and R¹ is selected from the group consisting of

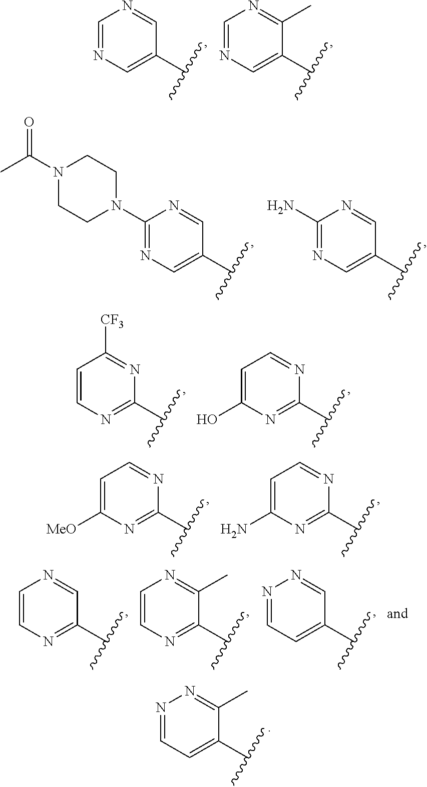

30. The compound or salt of embodiment 29, wherein R¹ is

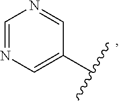 [SMILES: *c1cncnc1]

R² is methyl, R³ is H, and R⁴ is 4-chlorophenyl, 4-fluorophenyl, 2-aminopyrid-5-yl, 4-methylphenyl, 3-fluorophenyl,

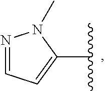 [SMILES: *c1ccnn1C]

3,4-dimethoxyphenyl, 3-cyano-4-chlorophenyl, 3-trifluoromethoxy-4-chlorophenyl, 2-trifluoromethylpyrid-5-yl, 2-methylpyrid-5-yl, or 2-methoxypyrid-5-yl.

31. The compound or salt of embodiment 29, wherein R¹ is

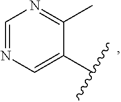 [SMILES: *c1cncnc1C]

R² is methyl, R³ is H, and R⁴ is 4-chlorophenyl, 4-fluorophenyl, 2-aminopyrid-5-yl, 4-methylphenyl, 3-fluorophenyl, 3-methoxy-4-chlorophenyl, 3-fluoro-4-methoxyphenyl, 3-methoxy-4-fluorophenyl, 3-trifluoromethyl-4-chlorophenyl, 3-trifluoromethoxy-4-chlorophenyl, 3,4-dimethoxylphenyl, 3,5-dimethoxylphenyl, 3,4-dimethoxy-5-fluorophenyl, 3,4-dimethoxy-5-chlorophenyl, and 3,4 5-trimethoxyphenyl.

32. The compound or salt of embodiment 29, wherein:

R² is methyl, R³ is H, and wherein:

R¹ is

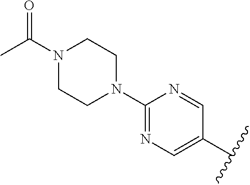 [SMILES: *c1cnc(N2CCN(C(C)=O)CC2)nc1]

and R⁴ is 4-chlorophenyl,

R¹ is

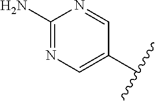 [SMILES: *c1cnc(N)nc1]

and R⁴ is 3-fluorophenyl,

R¹ is

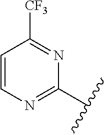 [SMILES: *c1nccc(C(F)(F)F)n1]

and R⁴ is 4-chlorophenyl or 3-fluorophenyl,

R¹ is

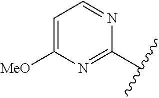 [SMILES: *c1nccc(OC)n1]

and R⁴ is 4-chlorophenyl or 3-fluororophenyl,

R¹ is

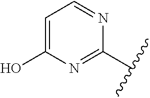 [SMILES: *c1nccc(O)n1]

and R⁴ is 4-chlorophenyl or 3-fluororophenyl,

R¹ is

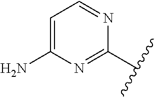 [SMILES: *c1nccc(N)n1]

and R⁴ is 4-chlorophenyl or 3-fluoro, or

R¹ is

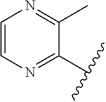 [SMILES: *c1nccnc1C]

and R⁴ is 3,4-dimethoxyphenyl.

33. The compound or salt of embodiment 29, wherein:

R¹ is

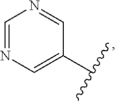 [SMILES: *c1cncnc1]

R² is methyl, R³ is

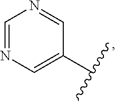 [SMILES: *c1cncnc1]

and R⁴ is 3-fluorophenyl, or

R¹ is

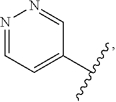 [SMILES: *c1ccnnc1]

R² is H, R³ is H, and R⁴ is 3,4-dimethoxyphenyl.

34. A pharmaceutical composition comprising a compound or salt of any one of embodiments 1-33 and a pharmaceutically acceptable carrier.

35. A method of blocking transmission of a Plasmodium parasite comprising administering to a mammal in need of such treatment, a therapeutically effective amount of a first compound of any one of embodiments 1-33.

36. A method of treating or preventing malaria by killing or arresting the growth of Plasmodium organisms in a mammal, wherein the Plasmodium organisms are in a liver stage or an asexual stage, the method comprising administering to a mammal a therapeutically effective amount of a first compound of any one of embodiments 1-33.

37. A compound or salt of any one of embodiments 1-33 for use in blocking transmission of a Plasmodium parasite in a mammal in need of thereof.

38. A compound or salt of any one of embodiments 1-33 for use in killing or arresting the growth of Plasmodium organisms in a mammal, wherein the Plasmodium organisms are in a liver stage or an asexual stage.

### EXAMPLES

The following examples further illustrate the invention but, of course, should not be construed as in any way limiting its scope.

Materials and Methods

Cell culture. Asexual parasites of P. falciparum strain 3D7 were cultured as described previously [Trager W, et al., Journal of Parasitology 2005, 91: 484-486]. Stage III-V gametocytes were selected and enriched with 3-day treatment with 50 mM N-acetylglucosamine (NAG) and the following Percoll density gradient centrifugation after gametocyte production [Tanaka T Q, et al., Molecular and Biochemical Parasitology, 2011, 177: 160-163]. Gametocytes of HB3 and Dd2 strains were produced and then set up for assay in a similar process. HepG2 cells (ATCC, cat. no. 77400) were cultured in 175-cm² tissue culture flasks with 30 ml growth medium at 37° C. in a 5% CO₂ humidified atmosphere. Growth medium was made with Dulbecco's Modified Eagle Medium with 10% fetal bovine serum (FBS). Growth medium was replaced every other day and cells were passed at 75% confluence.

Compound library and gametocyte assay screen. The approved drug library was collected with 4,265 compounds from traditional chemical suppliers, specialty collections, pharmacies and custom synthesis [Huang R et al., Science Translational Medicine, 2011, 3: 80ps16] that included 49% drugs approved for human or animal use by the US Food and Drug Administration (FDA), 23% approved in Canada/UK/EU/Japan, and the remaining 28% either in clinical trials or research tool compounds. The Malaria Box contained 400 drugs or tool compounds with the confirmed activities on blood-staged P. falciparum and assessed cytotoxicity against mammalian cells [Gamo F J et al., Nature, 2010 465: 305-U356; Guiguemde W A et al., Nature, 2010, 465: 311-315]. The MIPE library was an internal collection of 550 kinase inhibitors, which contain approved drugs and drug candidates in preclinical and clinical stages [Mathews L A et al., Journal of Biomolecular Screening, 2012, 17: 1231-1242]. Compounds from all libraries were obtained as powder samples and dissolved in DMSO as 10 mM stock solutions, except several hundreds from the approved drug library that were prepared as 4.47 mM stock solutions due to solubility limitations.

Compound screening experiments were performed as previously described [Tanaka T Q et al., Molecular and Biochemical Parasitology, 2013, 3188: 20-25]. Briefly, 2.5 μl/well incomplete medium was dispensed into each well of 1,536-well plates using the Multidrop Combi followed by 23 nl compound transferring using the NX-TR Pintool (WAKO Scientific Solutions, San Diego, Calif.). 2.5 μl/well of gametocytes was dispensed with a seeding density of 20,000 cells/well using the Multidrop Combi. The assay plates were incubated for 72 h at 37° C. with 5% CO₂. After addition of 5 μl/well of 2×AlamarBlue dye (Life Technologies, cat. no. DAL1100), the plates were incubated for 24 h at 37° C. with 5% CO₂ and were read in a fluorescence detection mode (Ex=525 nm, Em=598 nm) on a ViewLux plate reader (PerkinElmer).

Small molecule pull-down. Affinity matrix: To make a bead-connected affinity probe of Torin 2, a tetraethylene glycol linker was attached to 1-(piperazin-1-yl)propan-1-one of HWW030 and then coupled to Affi-Gel 10 resin (Bio-Rad Laboratories, cat. no. 153-6046) under mild basic conditions to afford Torin 2 matrix (T2M). Torin 1 was similarly immobilized to resin and used as a negative control (TIM). The resultant affinities probes were incubated with gametocyte lysates, the bound proteins were eluted from resin by boiling in SDS-PAGE sample loading buffer. The eluted fractions were separated by SDS-PAGE and visualized by silver staining. RBC infected with gametocytes (3D7 strain: Stage III-V) were washed 3 times with PBS and then lysed by 0.05% saponin treatment in PBS for 5 min at room temperature. The prepared gametocytes were washed 3 times with PBS and frozen at −80° C. The affinity precipitation experiment was processed as previously described [Zhang Q et al., Proceedings of the National Academy of Sciences of the United States of America, 2007, 104: 7444-7448; Arastu-Kapur S et al., Nature Chemical Biology, 2008, 4: 203-213]. The frozen samples were lysed with homogenization buffer (60 mM glycerophosphate, 15 mM p-nitrophenyl phosphate, 25 mM MOPS (pH 7.2), 15 mM EGTA, 15 mM MgCl2, 1 mM DTT, protease inhibitors (Roche Diagnostics, cat. no. 11836170001), and 0.5% Nonidet P-40). Cell lysates were centrifuged at 16,000×g for 20 min at 4° C., and the supernatant was collected. Protein concentration in the supernatant was determined by using a BCA protein assay kit (Pierce Chemical, cat. no. 23225). The lysate (0.5 mg) was then added to the packed affinity matrix, and bead buffer (50 mM Tris HCl (pH 7.4), 5 mM NaF, 250 mM NaCl, 5 mM EDTA, 5 mM EGTA, protease inhibitors, and 0.1% Nonidet P-40) was added to a final volume of 1 ml. After rotating at 4° C. for 2 h, the mixture was centrifuged at 16,000×g for 2 min at 4° C., and the supernatant was removed. The affinity matrix was then washed (six times) with cold bead buffer and eluted by boiling with SDS-PAGE sample loading buffer at 95° C. for 5 min. Supernatants were separated on a 10% Bis-Tris gel (Life Technologies, cat. no. NPO315BOX) and visualized by silver staining using a Pierce Silver Stain Kit for Mass Spectrometry (Pierce Chemical, cat. no. 24600).

DARTS (drug affinity responsive target stability). The 3D7 gametocytes were lysed with M-PER supplemented with protease and phosphatase inhibitors as previously described [Lomenick B et al., Proceedings of the National Academy of Sciences of the United States of America, 2009, 106: 21984-21989]. After centrifugation at 16,000×g for 20 min, protein concentration in the supernatant was quantified and 2 μg/μl proteins were treated with 600 nM of Torin 2 or 600 nM of Torin 1 for 2 h at room temperature. The samples were treated with 46 μg/ml pronase (Sigma-Aldrich, cat. no. P6911) for 30 min at room temperature. The digestion was stopped by adding the SDS-PAGE sample loading buffer and boiled at 70° C. for 10 min. The samples were separated on a 10% Bis-Tris gel and visualized by silver staining.

Malaria Mouse Model. Plasmodium berghei ANKA (Pb) parasites were maintained by serial passage by intraperitoneal (i.p.) injection in outbred mice. Two days before feeding, female mice were infected i.p. with 200-400 μl whole blood from a Pb-infected mouse with >10% parasitemia. On the day of feeding, the mice were checked for exflagellation and injected intravenously (i.v.) with drug vehicle alone (10% N-methylpyrrolidnone, 40% PEG 400 in water), or (a) 2-4 mg/kg Torin 2 (one or two doses), (b) 8 mg/kg NVP-AUY922 (two doses), or (c) 8 mg/kg Alvespimycin (two doses). Two hours post treatment, mice were anesthetized and Anopheles stephensi mosquitoes were allowed to feed on infected mice for 15 minutes. Parasitemia, gametocytemia, and presence of exflagellation were examined as described previously [Blagborough A M et al., Nature Communications, 2013, 4: 1812]. Mosquitoes were maintained on 5% (w/v) glucose at 19° C. and 80% relative humidity. At day 10 post feeding, mosquito midguts were dissected and transmission was measured by staining mosquito midguts with 0.2% mercurochrome and counting the numbers of oocysts per midgut.

Data analysis. The primary screen data was analyzed using customized software developed internally [Wang Y et al., Current Chemical Genomics 2010, 4: 57-66]. IC₅₀ values were calculated using the Prism software (Graphpad Software, Inc. San Diego, Calif.). Data were presented as means SEM with n=3 independent experiments.

General experimental details. All commercially available reagents, compounds, and solvents were purchased and used without further purification. Column chromatography on silica gel was performed on RediSep column using the Teledyne ISCO combiflash Rf system. Preparative purification was performed on a Waters semi-preparative HPLC. The column used was a Phenomenex Luna C18 (5 micron, 30×75 mm) at a flow rate of 45 mL/min. The mobile phase consisted of acetonitrile and water (each containing 0.1% trifluoroacetic acid). A gradient of 10% to 50% acetonitrile over 8 minutes was used during the purification. Fraction collection was triggered by UVdetection (220 nm).

¹H spectra were recorded using an 400 MHz spectrometer (Varian & Bruker). Samples were analyzed on Shimadzu 20 series LC/MS using Xtimate C18 (2.1*30 mm, 3 um) column and a flow rate of 0.8 mL/min. The mobile phase was a mixture of acetonitrile (Mobile Phase B, containing 0.01875% trifluoroacetic acid) and H₂O (Mobile Phase A, containing 0.0375% trifluoroacetic acid). A gradient of 10% to 80% acetonitrile over 6 minutes and holding at 80% for 0.5 minutes was used during analytical analysis.

### Example 1

This example illustrates a method of assaying an in vitro drug activity on gametocytes.

Stage III-V gametocytes (blood stage P. falciparum parasites) were enriched with treatment with 50 mM N-acetylglucosamine (NAG) and Percoll density gradient centrifugation as described previously¹. Briefly, 2.5 μl/well complete medium was dispensed into each well of 1,536-well plates using the Multidrop Combi followed by 23 nl compound transfer using the NX-TR Pintool (WAKO Scientific Solutions, San Diego, Calif.). Then, 2.5 μl/well of gametocytes was dispensed with a seeding density of 20,000 cells/well using the Multidrop Combi. The assay plates were incubated for 72 h at 37° C. with 5% CO₂. After addition of 5 μl/well of 2×AlamarBlue dye (Life Technologies, cat. no. DAL1100), the plates were incubated for 24 h at 37° C. with 5% CO₂ and then were read in a fluorescence detection mode (Ex=525 nm, Em=598 nm) on a ViewLux plate reader (PerkinElmer).

### Example 2

This example illustrates a method of assaying the in vitro drug activity on asexual parasites in accordance with an embodiment of the invention.

Asexual parasites of P. falciparum strain 3D7 were cultured as described previously (Trager, W. et al., J. Parasitol. 2005, 91(3): 484-486). Drug activity on asexual stage parasites was tested using a SYBR Green assay as described previously (Eastman, R. T. et al., Antimicrob. Agents Chemother. 2013, 57(1): 425-435; Smilkstein, M. et al., Antimicrob. Agents Chemother. 2004, 48(5): 1803-1806). Briefly, parasites were diluted to 0.5% parasitemia in complete culture medium with 2% hematocrit and drugs diluted in DMSO (<0.5%) and were loaded into a 96-well plate (200 μl/well). No drug and RBC alone wells were included as positive and background controls, respectively, and each testing condition was examined in duplicated. After 72 h incubation under the standard culture condition and a freeze-thaw lysis step at −80° C. and room temperature, 100 μl/well of lysis buffer containing SYBR Green I was added to the parasite culture and incubated for 30 min at room temperature. The fluorescence of each well was measured at 520 nm following excitation at 490 nm using a FLUOstar Optima™ microplate reader (BMG Labtech).

### Example 3

This example demonstrates syntheses of compounds, in accordance with embodiments of the invention.

General procedure A (scheme 1). A mixture of compound 1, amine 2 and HCl/dioxane in DMF was stirred at 100° C. to give compound 3. Then compound 3 was mixed with Fe, and NH₄Cl in EtOH and H₂O and stirred at 85° C. to afford Compound 4. Cyanation of compound 4 with carbononitridic bromide in EtOH at 80° C.-90° C. gave compound 5. Alkylation of compound 5 with 6 in DMF at 20-25° C. afforded compound 7. Suzuki coupling of compound 7 and boronic acid/ester 8 in the presence of Na₂CO₃ and Pd(dppf)Cl₂ in dioxane and H₂O at 100° C. gave compound 9, which reacted with reagent 10 to form compound 11.

General procedure B (scheme 2). A mixture of compound 12, amine 2 and HCl/dioxane in DMF was stirred at 100° C. to give compound 13. Suzuki coupling of compound 13 and boronic acid/ester 8 in the presence of Na₂CO₃ and Pd(dppf)Cl₂ in dioxane and H₂O at 100° C. afforded compound 14. Reduction of compound 14 with NaBH₄ in EtOH and THF at 20-25° C. gave compound 15. Oxidation of compound 15 with MnO₂ in DCM at 20-25° C. afford compound 16. Olefination of compound 16 with 2-diethoxyphosphorylacetonitrile and K₂CO₃ in DMF at 100° C. gave compound 17, which reacted with reagent 10 to form compound 18.

General procedure C (scheme 3). A mixture of compound 23 and amine 2 in NMP was stirred under microwave irradiation at 180° C. (R¹=alkyl), or at 150° C. (R¹=aryl) to form compound 24. Then DMF and 1M K₃PO₄ was added followed by boronic acid/ester 8 and Pd(dppf)Cl₂. The Suzuki coupling of compound 24 at 150° C. afforded compound 9.

### 5-(1-(5-cyano-2-methylphenyl)-2-imino-3-methyl-2,3-dihydro-1H-imidazo[4,5-c]quinolin-8-yl)thiophene-2-carboxamide (1)

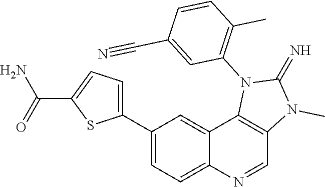 [SMILES: Cc1ccc(C#N)cc1-n1c(=N)n(C)c2cnc3ccc(-c4ccc(C(N)=O)s4)cc3c21]

LCMS: t_{R}=2.733 min in 0-60AB_7 min_220&254 (Xtimate 3 um, C18, 2.1*30 mm), MS (ESI) m/z 439.1 [M+H]⁺.

¹H NMR (400 MHz, DMSO-d₆) δ=9.35 (s, 1H), 9.10 (s, 2H), 8.36 (d, J=1.5 Hz, 1H), 8.29 (dd, J=1.7, 8.0 Hz, 1H), 8.20 (d, J=9.0 Hz, 1H), 8.07 (dd, J=2.0, 8.8 Hz, 1H), 8.04-7.97 (m, 2H), 7.72-7.66 (m, 1H), 7.48 (br s, 1H), 7.34 (d, J=3.7 Hz, 1H), 6.90 (d, J=1.8 Hz, 1H), 3.93 (s, 3H), 2.22 (s, 3H).

### 3-(8-(6-aminopyridin-3-yl)-2-imino-3-methyl-2,3-dihydro-H-imidazo[4,5-c]quinolin-1-yl)-4-methylbenzonitrile (2)

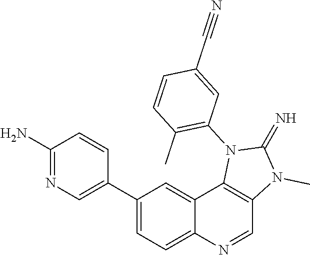 [SMILES: Cc1ccc(C#N)cc1-n1c(=N)n(C)c2cnc3ccc(-c4ccc(N)nc4)cc3c21]

LCMS: R=2.423 min in 10-80CD_7 MIN_220&254 (XBridge Shield RP18 2.1*50 mm, 5 um), MS (ESI) m/z 406.2 [M+H]⁺.

¹H NMR (400 MHz, DMSO-d₆) δ=9.35 (s, 1H), 9.06 (br s, 2H), 8.37-8.30 (m, 1H), 8.26-8.18 (m, 2H), 7.98 (dd, J=1.9, 8.9 Hz, 1H), 7.95-7.89 (m, 2H), 7.66-7.60 (m, 1H), 6.84 (br d, J=8.4 Hz, 1H), 6.74 (d, J=1.8 Hz, 1H), 3.92 (s, 3H), 2.19 (s, 3H).

### 3-(2-imino-3-methyl-8-(2-oxoindolin-5-yl)-2,3-dihydro-1H-imidazo[4,5-c]quinolin-1-yl)-4-methylbenzonitrile (3)

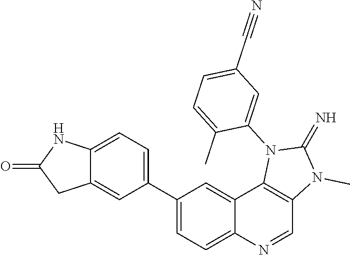 [SMILES: Cc1ccc(C#N)cc1-n1c(=N)n(C)c2cnc3ccc(-c4ccc5c(c4)CC(=O)N5)cc3c21]

LCMS: t_{R}=2.870 min in 0-60AB_7 min_220&254 (Xtimate 3 um, C18, 2.1*30 mm), MS (ESI) m/z 445.1 [M+H]⁺.

¹H NMR (400 MHz, DMSO-d₆) δ=10.54 (s, 1H), 9.31 (s, 1H), 9.03 (s, 2H), 8.38 (d, J=1.5 Hz, 1H), 8.31 (dd, J=1.7, 8.0 Hz, 1H), 8.18 (d, J=8.8 Hz, 1H), 7.97 (dd, J=2.1, 8.9 Hz, 1H), 7.94 (d, J=8.4 Hz, 1H), 7.13 (d, J=7.9 Hz, 1H), 7.10 (s, 1H), 6.82 (d, J=8.2 Hz, 1H), 6.74 (d, J=2.2 Hz, 1H), 3.91 (s, 3H), 3.51 (s, 2H), 2.19 (s, 3H).

### (E)-N-(8-(6-aminopyridin-3-yl)-1-(5-cyano-2-methylphenyl)-3-methyl-1,3-dihydro-2H-imidazo[4,5-c]quinolin-2-ylidene)cyanamide (4)

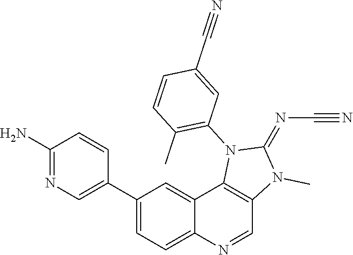 [SMILES: Cc1ccc(C#N)cc1-n1/c(=N/C#N)n(C)c2cnc3ccc(-c4ccc(N)nc4)cc3c21]

LCMS: t_{R}=2.627 min in 0-60AB_7 min_220&254 (Xtimate 3 um, C18, 2.1*30 mm), MS (ESI) m/z 431.0 [M+Na]⁺.

¹H NMR (400 MHz, DMSO-d₆) δ=9.16 (s, 1H), 8.39 (d, J=1.1 Hz, 1H), 8.19 (dd, J=1.5, 7.9 Hz, 1H), 8.10 (d, J=8.8 Hz, 1H), 7.88 (dd, J=1.8, 8.8 Hz, 1H), 7.85 (d, J=8.2 Hz, 1H), 7.81 (d, J=2.4 Hz, 1H), 7.35 (dd, J=2.5, 8.7 Hz, 1H), 6.72 (d, J=1.5 Hz, 1H), 6.44 (d, J=8.6 Hz, 1H), 6.24 (s, 2H), 3.84 (s, 3H), 2.20 (s, 3H).

### 3-(2-imino-3-methyl-8-(1-methyl-1H-pyrazol-5-yl)-2,3-dihydro-1H-imidazo[4,5-c]quinolin-1-yl)-4-methylbenzonitrile (5)

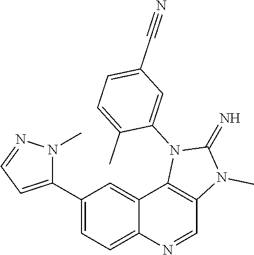 [SMILES: Cc1ccc(C#N)cc1-n1c(=N)n(C)c2cnc3ccc(-c4ccnn4C)cc3c21]

LCMS: t_{R}=2.738 min in 0-60AB_7 min 220&254 (Xtimate 3 um, C18, 2.1*30 mm), MS (ESI) m/z 394.1 [M+H]⁺.

¹H NMR (400 MHz, METHANOL-d₄) δ=9.32 (s, 1H), 8.31 (d, J=8.8 Hz, 1H), 8.21 (d, J=1.5 Hz, 1H), 8.12 (dd, J=1.7, 8.0 Hz, 1H), 7.95-7.82 (m, 2H), 7.47 (d, J=2.0 Hz, 1H), 6.95 (d, J=2.0 Hz, 1H), 6.28 (d, J=2.0 Hz, 1H), 4.02 (s, 3H), 3.65 (s, 3H), 2.28 (s, 3H).

### (E)-3-(8-(4-chlorophenyl)-3-methyl-2-(methylimino)-2,3-dihydro-1H-imidazo[4,5-c]quinolin-1-yl)-4-methylbenzonitrile (6)

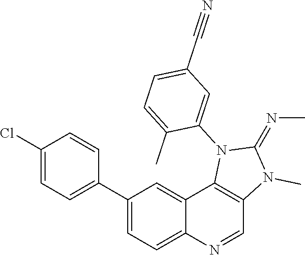 [SMILES: C/N=c1\n(C)c2cnc3ccc(-c4ccc(Cl)cc4)cc3c2n1-c1cc(C#N)ccc1C]

LCMS: t_{R}=4.136 min in 0-60AB_7 min_220&254 (Xtimate 3 um, C18, 2.1*30 mm), MS (ESI) m/z 438.1 [M+H]⁺.

¹H NMR (400 MHz, DMSO-d₆) δ=9.41 (s, 1H), 8.88 (br d, J=4.6 Hz, 1H), 8.43 (d, J=1.5 Hz, 1H), 8.33-8.17 (m, 2H), 8.02 (dd, J=2.0, 8.8 Hz, 1H), 7.93 (d, J=8.2 Hz, 1H), 7.55-7.43 (m, 2H), 7.36-7.25 (m, 2H), 6.73 (d, J=1.8 Hz, 1H), 4.04 (s, 3H), 2.83 (d, J=4.9 Hz, 3H), 2.26 (s, 3H).

### 3-(8-(4-chlorophenyl)-2-imino-3-methyl-2,3-dihydro-1H-imidazo[4,5-c]quinolin-1-yl)-4-ethylbenzonitrile (7)

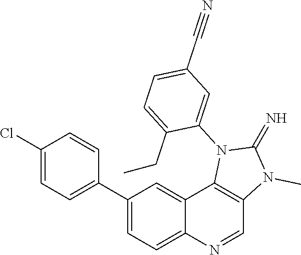 [SMILES: CCc1ccc(C#N)cc1-n1c(=N)n(C)c2cnc3ccc(-c4ccc(Cl)cc4)cc3c21]

LCMS: t_{R}=5.880 min in 0-60AB_7 min chromatography (Xtimate 3 um, C18, 2.1*30 mm), MS (ESI) m/z 438.1 [M+H]⁺.

### (E)-N-(8-(4-chlorophenyl)-1-(5-cyano-2-methylphenyl)-3-methyl-1,3-dihydro-2H-imidazo[4,5-c]quinolin-2-ylidene)cyanamide (8)

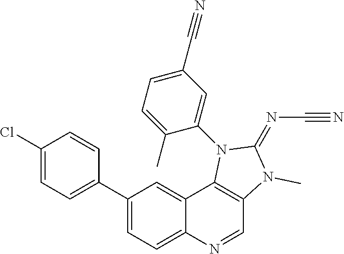 [SMILES: Cc1ccc(C#N)cc1-n1/c(=N/C#N)n(C)c2cnc3ccc(-c4ccc(Cl)cc4)cc3c21]

LCMS: t_{R}=5.027 min in 0-60AB_7 min_220&254 (Xtimate 3 um, C18, 2.1*30 mm), MS (ESI) m/z 449.1 [M+H]⁺.

¹H NMR (400 MHz, DMSO-d₆) δ=9.24 (s, 1H), 8.38 (d, J=1.3 Hz, 1H), 8.22-8.13 (m, 2H), 7.96 (dd, J=2.0, 8.8 Hz, 1H), 7.84 (d, J=8.2 Hz, 1H), 7.51-7.43 (m, 2H), 7.34-7.25 (m, 2H), 6.82 (d, J=1.8 Hz, 1H), 3.85 (s, 3H), 2.21 (s, 3H).

### (E)-N-(8-(4-chlorophenyl)-1-(5-cyano-2-methylphenyl)-3-methyl-1,3-dihydro-2H-imidazo[4,5-c]quinolin-2-ylidene)propionamide (9)

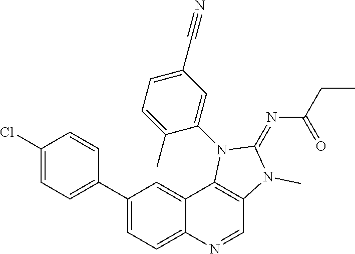 [SMILES: CCC(=O)/N=c1\n(C)c2cnc3ccc(-c4ccc(Cl)cc4)cc3c2n1-c1cc(C#N)ccc1C]

LCMS: t_{R}=3.361 min in 10-80AB_7 min_220&254 chromatography (Xtimate 3 um, C18, 2.1*30 mm), MS (ESI) m/z 480.0 [M+H]⁺.

¹H NMR (400 MHz, DMSO-d₆) δ=9.24 (s, 1H), 8.25-8.13 (m, 2H), 8.08 (dd, J=1.5, 7.9 Hz, 1H), 7.95 (dd, J=2.1, 8.9 Hz, 1H), 7.80 (d, J=8.2 Hz, 1H), 7.47 (d, J=8.6 Hz, 2H), 7.32 (d, J=8.6 Hz, 2H), 6.95 (d, J=1.8 Hz, 1H), 3.69 (s, 3H), 2.16 (s, 3H), 2.08 (q, J=7.6 Hz, 2H), 0.84 (t, J=7.5 Hz, 3H).

### 3-(8-(4-chlorophenyl)-2-imino-3-methyl-2,3-dihydro-1H-imidazo[4,5-c]quinolin-1-yl)-4-fluorobenzonitrile (10)

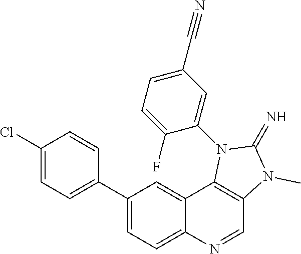 [SMILES: Cn1c(=N)n(-c2cc(C#N)ccc2F)c2c3cc(-c4ccc(Cl)cc4)ccc3ncc21]

LCMS: t_{R}=4.008 min in 0-60AB_7.0 min chromatography (Xtimate 3 um, C18, 2.1*30 mm), MS (ESI) m/z 428.0 [M+H]⁺.

¹H NMR: (DMSO-d₆ 400 MHz): δ 9.44-9.26 (m, 3H), 8.69 (dd, J=2.0, 6.8 Hz, 1H), 8.53 (dd, J=2.4, 8.9 Hz, 1H), 8.29 (d, J=8.8 Hz, 1H), 8.15-8.06 (m, 2H), 7.59-7.50 (m, 2H), 7.43 (d, J=8.5 Hz, 2H), 7.07 (s, 1H), 3.97 (s, 3H).

### 3-(8-(4-chlorophenyl)-2-imino-3-methyl-2,3-dihydro-1H-imidazo[4,5-c]quinolin-1-yl)benzonitrile (11)

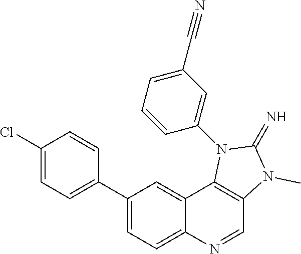 [SMILES: Cn1c(=N)n(-c2cccc(C#N)c2)c2c3cc(-c4ccc(Cl)cc4)ccc3ncc21]

LCMS: t_{R}=4.048 min in 0-60AB_7.0 MIN chromatography (Xtimate 3 um, C18, 2.1*30 mm), MS (ESI) m/z 410.0 [M+H]⁺.

¹H NMR (400 MHz, DMSO-d₆) δ=9.38 (s, 1H), 9.01 (s, 2H), 8.50 (s, 1H), 8.39 (d, J=7.7 Hz, 1H), 8.26 (d, J=8.8 Hz, 2H), 8.10-8.02 (m, 2H), 7.51 (d, J=8.6 Hz, 2H), 7.37 (d, J=8.6 Hz, 2H), 6.96 (d, J=1.8 Hz, 1H), 3.95 (s, 3H).

### 3-(8-(4-fluorophenyl)-2-imino-3-methyl-2,3-dihydro-1H-imidazo[4,5-c]quinolin-1-yl)-4-methylbenzonitrile (12)

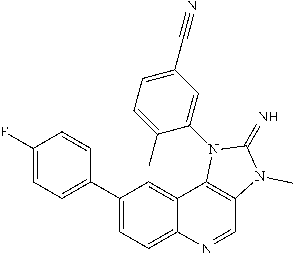 [SMILES: Cc1ccc(C#N)cc1-n1c(=N)n(C)c2cnc3ccc(-c4ccc(F)cc4)cc3c21]

LCMS: t_{R}=3.811 min in 0-60AB_7 min 220&254 (Xtimate 3 um, C18, 2.1*30 mm), MS (ESI) m/z 408.1 [M+H]⁺.

¹H NMR (400 MHz, METHANOL-d₄) δ=9.25 (s, 1H), 8.26 (d, J=9.0 Hz, 1H), 8.23 (d, J=1.8 Hz, 1H), 8.15 (dd, J=1.7, 8.0 Hz, 1H), 8.01 (dd, J=2.1, 8.9 Hz, 1H), 7.91 (d, J=8.2 Hz, 1H), 7.42-7.27 (m, 2H), 7.19-7.10 (m, 2H), 6.96 (d, J=1.8 Hz, 1H), 4.01 (s, 3H), 2.29 (s, 3H).

### 8-(4-chlorophenyl)-3-methyl-1-(1-methyl-1H-pyrazol-4-yl)-1,3-dihydro-2H-imidazo[4,5-c]quinolin-2-imine (13)

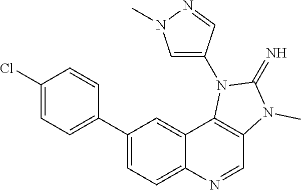 [SMILES: Cn1cc(-n2c(=N)n(C)c3cnc4ccc(-c5ccc(Cl)cc5)cc4c32)cn1]

LCMS: t_{R}=3.552 min in 0-60AB_7 min_220&254 (Xtimate 3 um, C18, 2.1*30 mm), MS (ESI) m/z 389.0 [M+H]⁺.

¹H NMR (400 MHz, METHANOL-d₄) δ=9.19 (s, 1H), 8.29 (s, 1H), 8.23 (d, J=9.0 Hz, 1H), 8.02 (br d, J=9.3 Hz, 1H), 7.96 (s, 1H), 7.55 (s, 1H), 7.46 (s, 4H), 4.11 (s, 3H), 3.95 (s, 3H).

### 5-(8-(4-chlorophenyl)-2-imino-3-methyl-2,3-dihydro-1H-imidazo[4,5-c]quinolin-1-yl)-4-methyl-2-(piperazin-1-yl)benzonitrile (14)

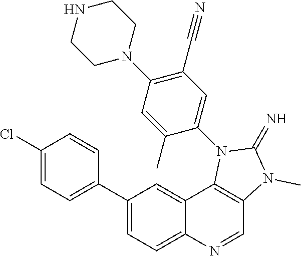 [SMILES: Cc1cc(N2CCNCC2)c(C#N)cc1-n1c(=N)n(C)c2cnc3ccc(-c4ccc(Cl)cc4)cc3c21]

LCMS: t_{R}=3.105 min in 0-60AB_7.0 MIN chromatography (Xtimate 3 um, C18, 2.1*30 mm), MS (ESI) m/z 508.2[M+H]⁺.

¹H NMR (400 MHz, DMSO-d₆) δ=9.38 (s, 1H), 9.11-8.93 (m, 4H), 8.30-8.23 (m, 2H), 8.08 (br d, J=8.6 Hz, 1H), 7.59 (s, 1H), 7.53 (br d, J=7.3 Hz, 2H), 7.41 (br d, J=7.5 Hz, 2H), 6.99 (s, 1H), 3.95 (s, 3H), 3.63 (br d, J=12.8 Hz, 2H), 3.48 (br s, 6H), 2.17 (s, 3H).

### tert-butyl 4-(4-(8-(4-chlorophenyl)-2-imino-3-methyl-2,3-dihydro-1H-imidazo[4,5-c]quinolin-1-yl)-2-cyano-5-methylphenyl)piperazine-1-carboxylate (15)

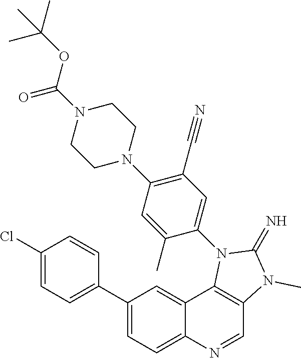 [SMILES: Cc1cc(N2CCN(C(=O)OC(C)(C)C)CC2)c(C#N)cc1-n1c(=N)n(C)c2cnc3ccc(-c4ccc(Cl)cc4)cc3c21]

LCMS: t_{R}=5.034 min in 0-60AB_7.0 MIN chromatography (Xtimate 3 um, C18, 2.1*30 mm), MS (ESI) m/z 608.2 [M+H]⁺.

¹H NMR (400 MHz, DMSO-d₆) δ=8.82 (br s, 1H), 8.03 (d, J=9.3 Hz, 2H), 7.81 (dd, J=2.0, 9.0 Hz, 1H), 7.46 (d, J=8.6 Hz, 2H), 7.40 (s, 1H), 7.34 (d, J=8.8 Hz, 2H), 6.92 (s, 1H), 3.58 (br s, 4H), 3.54 (s, 3H), 3.31 (s, 2H), 3.27-3.18 (m, 2H), 2.09 (br s, 3H), 1.46 (s, 9H).

### (E)-N-(8-(4-chlorophenyl)-1-(5-cyano-2-methylphenyl)-3-methyl-1,3-dihydro-2H-imidazo[4,5-c]quinolin-2-ylidene)-4-methylbenzenesulfonamide (16)

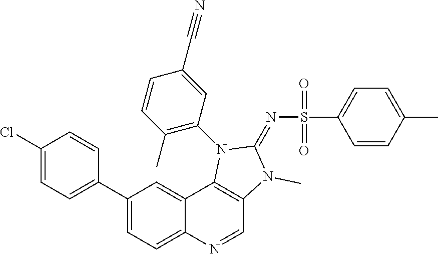 [SMILES: Cc1ccc(S(=O)(=O)/N=c2\n(C)c3cnc4ccc(-c5ccc(Cl)cc5)cc4c3n2-c2cc(C#N)ccc2C)cc1]

LCMS: t_{R}=4.347 min in 10-80AB_7 min_220&254 (Xtimate 3 um, C18, 2.1*30 mm), MS (ESI) m/z 578.1 [M+H]⁺.

¹H NMR (400 MHz, DMSO-d₆) δ=9.31 (s, 1H), 8.21 (d, J=8.8 Hz, 1H), 8.08-8.03 (m, 2H), 7.99 (dd, J=2.1, 8.9 Hz, 1H), 7.73 (d, J=8.6 Hz, 1H), 7.46 (d, J=8.6 Hz, 2H), 7.41 (d, J=8.2 Hz, 2H), 7.32-7.27 (m, 2H), 7.16 (d, J=8.2 Hz, 2H), 6.85 (d, J=1.8 Hz, 1H), 4.04 (s, 3H), 2.32 (s, 3H), 2.08 (s, 3H).

### (E)-3-(2-(butylimino)-8-(4-chlorophenyl)-3-methyl-2,3-dihydro-1H-imidazo[4,5-c]quinolin-1-yl)-4-methylbenzonitrile (17)

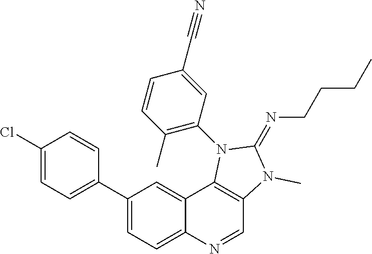 [SMILES: CCCC/N=c1\n(C)c2cnc3ccc(-c4ccc(Cl)cc4)cc3c2n1-c1cc(C#N)ccc1C]

LCMS: t_{R}=4.661 min in 0-60AB_7 min_220&254 (Xtimate 3 um, C18, 2.1*30 mm), MS (ESI) m/z 480.1 [M+H]⁺.

¹H NMR (400 MHz, DMSO-d₆) δ=9.41 (s, 1H), 8.74 (br t, J=5.8 Hz, 1H), 8.48 (s, 1H), 8.30 (d, J=8.2 Hz, 1H), 8.24 (d, J=8.8 Hz, 1H), 8.03 (dd, J=1.9, 8.9 Hz, 1H), 7.95 (d, J=7.9 Hz, 1H), 7.49 (d, J=8.6 Hz, 2H), 7.31 (d, J=8.6 Hz, 2H), 6.72 (d, J=1.8 Hz, 1H), 4.02 (s, 3H), 3.24-3.03 (m, 2H), 2.25 (s, 3H), 1.42 (quin, J=7.1 Hz, 2H), 1.23-1.00 (m, 2H), 0.76 (t, J=7.4 Hz, 3H).

### (E)-N-(8-(4-chlorophenyl)-1-(5-cyano-2-(methylthio)phenyl)-3-methyl-1,3-dihydro-2H-imidazo[4,5-c]quinolin-2-ylidene)cyanamide (18)

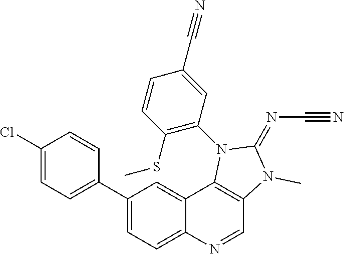 [SMILES: CSc1ccc(C#N)cc1-n1/c(=N/C#N)n(C)c2cnc3ccc(-c4ccc(Cl)cc4)cc3c21]

LCMS: t_{R}=4.955 min in 0-60AB_7 min chromatography (Xtimate 3 um, C18, 2.1*30 mm), MS (ESI) m/z 481.0 [M+H]⁺.

¹H NMR: (DMSO-d₆ 400 MHz): δ=9.25 (s, 1H), 8.42 (d, J=1.8 Hz, 1H), 8.23 (dd, J=1.8, 8.4 Hz, 1H), 8.18 (d, J=8.8 Hz, 1H), 7.98 (dd, J=1.9, 8.9 Hz, 1H), 7.77 (d, J=8.6 Hz, 1H), 7.48 (d, J=8.6 Hz, 2H), 7.32 (d, J=8.4 Hz, 2H), 6.95 (d, J=1.5 Hz, 1H), 3.83 (s, 3H), 2.49 (s, 3H).

### 3-(8-(4-chlorophenyl)-2-imino-3-methyl-2,3-dihydro-1H-imidazo[4,5-c]quinolin-1-yl)-4-ethoxybenzonitrile (19)

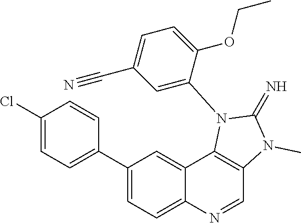 [SMILES: CCOc1ccc(C#N)cc1-n1c(=N)n(C)c2cnc3ccc(-c4ccc(Cl)cc4)cc3c21]

### (E)-N-(8-(4-chlorophenyl)-1-(5-cyano-2-methylphenyl)-3-methyl-1,3-dihydro-2H-imidazo[4,5-c]quinolin-2-ylidene)methanesulfonamide (20)

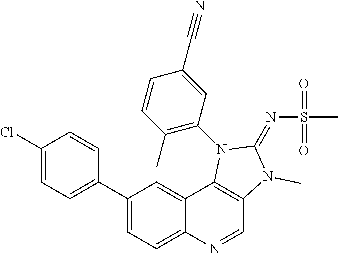 [SMILES: Cc1ccc(C#N)cc1-n1/c(=N/S(C)(=O)=O)n(C)c2cnc3ccc(-c4ccc(Cl)cc4)cc3c21]

LCMS: t_{R}=3.554 min in 10-80AB_7 min_220&254 (Xtimate 3 um, C18, 2.1*30 mm), MS (ESI) m/z 502.0 [M+H]⁺.

¹H NMR (400 MHz, DMSO-d₆) δ=9.32 (s, 1H), 8.32 (d, J=1.0 Hz, 1H), 8.23 (d, J=9.0 Hz, 1H), 8.16 (dd, J=1.3, 8.0 Hz, 1H), 8.03 (dd, J=1.9, 8.9 Hz, 1H), 7.85 (d, J=8.0 Hz, 1H), 7.51 (d, J=8.5 Hz, 2H), 7.34 (d, J=8.5 Hz, 2H), 6.89 (d, J=1.5 Hz, 1H), 4.04 (s, 3H), 2.85 (s, 3H), 2.18 (s, 3H).

### 5-(8-(4-chlorophenyl)-2-imino-3-methyl-2,3-dihydro-H-imidazo[4,5-c]quinolin-1-yl)-6-methyl-2-morpholinonicotinonitrile (21)

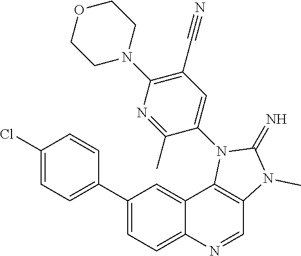 [SMILES: Cc1nc(N2CCOCC2)c(C#N)cc1-n1c(=N)n(C)c2cnc3ccc(-c4ccc(Cl)cc4)cc3c21]

LCMS: t_{R}=4.214 min in 0-60AB_7 min chromatography (Xtimate 3 um, C18, 2.1*30 mm), MS (ESI) m/z 510.1[M+H]⁺.

¹H NMR: (DMSO-d₆ 400 MHz): δ=9.37 (s, 1H), 9.20 (s, 2H), 8.50 (s, 1H), 8.26 (d, J=9.0 Hz, 1H), 8.06 (dd, J=2.0, 8.8 Hz, 1H), 7.55-7.51 (m, 2H), 7.49-7.46 (m, 2H), 7.18 (d, J=1.8 Hz, 1H), 3.95-3.90 (m, 7H), 3.89-3.75 (m, 4H), 2.24 (s, 3H).

### 5-(8-(4-chlorophenyl)-2-imino-3-methyl-2,3-dihydro-1H-imidazo[4,5-c]quinolin-1-yl)-6-methylnicotinonitrile (22)

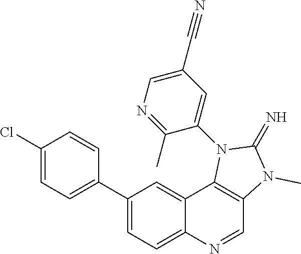 [SMILES: Cc1ncc(C#N)cc1-n1c(=N)n(C)c2cnc3ccc(-c4ccc(Cl)cc4)cc3c21]

LCMS: t_{R}=3.740 min in 0-60AB_7 min_220&254 (Xtimate 3 um, C18, 2.1*30 mm), MS (ESI) m/z 424.9 [M+H]⁺.

¹H NMR (400 MHz, DMSO-d₆) δ=9.45-9.39 (m, 2H), 9.35 (s, 2H), 8.87 (d, J=1.8 Hz, 1H), 8.29 (d, J=9.0 Hz, 1H), 8.07 (dd, J=2.0, 9.0 Hz, 1H), 7.54 (d, J=8.5 Hz, 2H), 7.40 (d, J=8.5 Hz, 2H), 6.90 (d, J=1.8 Hz, 1H), 4.00 (s, 3H), 2.48 (s, 3H).

### 5-(8-(4-chlorophenyl)-2-imino-3-methyl-2,3-dihydro-1H-imidazo[4,5-c]quinolin-1-yl)-4-methyl-2-morpholinobenzonitrile (23)

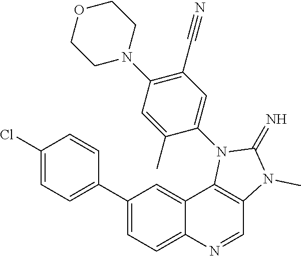 [SMILES: Cc1cc(N2CCOCC2)c(C#N)cc1-n1c(=N)n(C)c2cnc3ccc(-c4ccc(Cl)cc4)cc3c21]

LCMS: t_{R}=4.195 min in 0-60AB_7.0 MIN chromatography (Xtimate 3 um, C18, 2.1*30 mm), MS (ESI) m/z 509.1 [M+H]⁺.

¹H NMR (400 MHz, DMSO-d₆) δ=9.41 (s, 1H), 9.25 (s, 2H), 8.28 (d, J=8.8 Hz, 1H), 8.20 (s, 1H), 8.08 (dd, J=2.1, 8.9 Hz, 1H), 7.53-7.45 (m, 3H), 7.43-7.36 (m, 2H), 6.98 (d, J=1.5 Hz, 1H), 3.97 (s, 3H), 3.83 (t, J=4.6 Hz, 4H), 3.49-3.38 (m, 2H), 3.29 (td, J=4.7, 12.5 Hz, 2H), 2.14 (s, 3H).

### 3-(8-(3-((dimethylamino)methyl)phenyl)-2-imino-3-methyl-2,3-dihydro-1H-imidazo[4,5-c]quinolin-1-yl)-4-methylbenzonitrile (24)

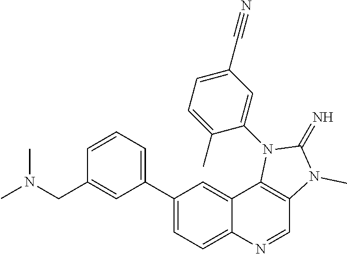

LCMS: t_{R}=2.332 min in 0-60AB_7.0 MIN chromatography (Xtimate 3 um, C18, 2.1*30 mm), MS (ESI) m/z 447.1 [M+H]⁺.

¹H NMR (400 MHz, DMSO-d₆) δ=10.45 (br s, 1H), 9.41 (s, 1H), 9.23 (br s, 2H), 8.41 (d, J=1.5 Hz, 1H), 8.37-8.22 (m, 2H), 8.07 (dd, J=1.9, 8.9 Hz, 1H), 8.01-7.91 (m, 1H), 7.60 (s, 1H), 7.57-7.51 (m, 2H), 7.36-7.28 (m, 1H), 6.93 (d, J=1.8 Hz, 1H), 4.39-4.25 (m, 2H), 3.99 (s, 3H), 2.78 (s, 6H), 2.25 (s, 3H).

### 3-(8-(3-cyanophenyl)-2-imino-3-methyl-2,3-dihydro-1H-imidazo[4,5-c]quinolin-1-yl)-4-methylbenzonitrile (25)

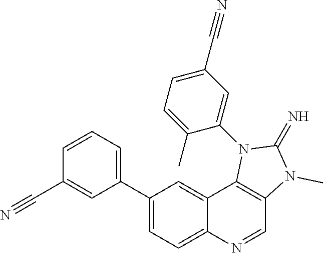 [SMILES: Cc1ccc(C#N)cc1-n1c(=N)n(C)c2cnc3ccc(-c4cccc(C#N)c4)cc3c21]

LCMS: t_{R}=3.438 min in 0-60AB_7.0 MIN chromatography (Xtimate 3 um, C18, 2.1*30 mm), MS (ESI) m/z 415.1 [M+H]⁺.

¹H NMR (400 MHz, DMSO-d₆) δ=9.41 (s, 1H), 9.18 (br s, 2H), 8.40 (d, J=0.9 Hz, 1H), 8.32-8.22 (m, 2H), 8.13 (dd, J=1.8, 8.8 Hz, 1H), 7.96 (d, J=8.2 Hz, 1H), 7.91-7.83 (m, 1H), 7.77 (s, 1H), 7.70-7.62 (m, 2H), 6.89 (d, J=1.5 Hz, 1H), 3.97 (s, 3H), 2.24 (s, 3H).

### 8-(4-chlorophenyl)-3-methyl-1-(3-(trifluoromethyl)phenyl)-1,3-dihydro-2H-imidazo[4,5-c]quinolin-2-imine (26)

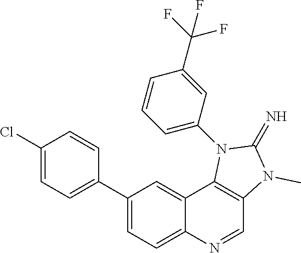 [SMILES: Cn1c(=N)n(-c2cccc(C(F)(F)F)c2)c2c3cc(-c4ccc(Cl)cc4)ccc3ncc21]

LCMS: ES7316-92-P1D t_{R}=4.491 min in 0-60AB_7 min_220&254 (Xtimate 3 um, C18, 2.1*30 mm), MS (ESI) m/z 453.0 [M+H]⁺.

¹H NMR (ES7316-92-P1A, 400 MHz, DMSO-d₆) δ=9.38 (s, 1H), 9.05 (s, 2H), 8.46 (s, 1H), 8.30 (br d, J=7.9 Hz, 1H), 8.28-8.20 (m, 2H), 8.13-8.07 (m, 1H), 8.04 (dd, J=1.9, 8.9 Hz, 1H), 7.47 (d, J=8.4 Hz, 2H), 7.31 (d, J=8.6 Hz, 2H), 6.86 (d, J=1.8 Hz, 1H), 3.96 (s, 3H).

### 8-(4-chlorophenyl)-3-methyl-1-(2-methyl-5-(trifluoromethyl)phenyl)-1,3-dihydro-2H-imidazo[4,5-c]quinolin-2-imine (27)

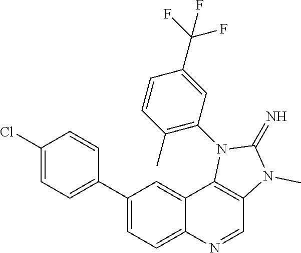 [SMILES: Cc1ccc(C(F)(F)F)cc1-n1c(=N)n(C)c2cnc3ccc(-c4ccc(Cl)cc4)cc3c21]

LCMS: t_{R}=4.538 min in 0-60AB_7 min chromatography (Xtimate 3 um, C18, 2.1*30 mm), MS (ESI) m/z 467.0 [M+H]⁺.

¹H NMR (400 MHz, DMSO-d₆) δ=9.39 (s, 1H), 9.17 (s, 2H), 8.35 (s, 1H), 8.26 (d, J=9.04 Hz, 1H), 8.19 (d, J=8.16 Hz, 1H), 8.03 (dd, J=8.82, 1.10 Hz, 1H), 7.97 (d, J=8.16 Hz, 1H), 7.46 (d, J=8.38 Hz, 2H), 7.29 (d, J=8.60 Hz, 2H), 6.78 (d, J=1.76 Hz, 1H), 3.97 (s, 3H), 2.24 (s, 3H).

### 1-(3-chlorophenyl)-8-(4-chlorophenyl)-3-methyl-1,3-dihydro-2H-imidazo[4,5-c]quinolin-2-imine (28)

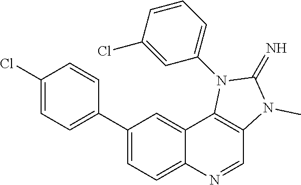 [SMILES: Cn1c(=N)n(-c2cccc(Cl)c2)c2c3cc(-c4ccc(Cl)cc4)ccc3ncc21]

LCMS: t_{R}=4.174 min in 0-60AB_7 min chromatography (Xtimate 3 um, C18, 2.1*30 mm), MS (ESI) m/z 419.0 [M+H]⁺.

¹H NMR: (DMSO-d₆ 400 MHz): δ=9.23 (s, 1H), 8.18 (d, J=8.8 Hz, 1H), 8.05 (s, 1H), 7.99-7.91 (m, 2H), 7.85-7.81 (m, 2H), 7.50-7.46 (m, 2H), 7.38-7.34 (m, 2H), 7.00 (d, J=1.8 Hz, 1H), 3.84 (s, 3H).

### 1-(5-chloro-2-methylphenyl)-8-(4-chlorophenyl)-3-methyl-1,3-dihydro-2H-imdazo[4,5-c]quinolin-2-imine (29)

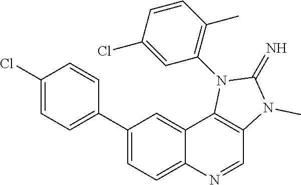 [SMILES: Cc1ccc(Cl)cc1-n1c(=N)n(C)c2cnc3ccc(-c4ccc(Cl)cc4)cc3c21]

LCMS: t_{R}=4.377 min in 0-60AB_7.0 MIN chromatography (Xtimate 3 um, C18, 2.1*30 mm), MS (ESI) m/z 433.0[M+H]⁺.

¹H NMR (400 MHz, DMSO-d₆) δ=9.36 (s, 1H), 9.01 (s, 2H), 8.24 (d, J=8.8 Hz, 1H), 8.06-7.97 (m, 2H), 7.91-7.84 (m, 1H), 7.76 (d, J=8.6 Hz, 1H), 7.53-7.46 (m, 2H), 7.38-7.32 (m, 2H), 6.90 (d, J=1.8 Hz, 1H), 3.93 (s, 3H), 2.11 (s, 3H).

### 2-chloro-5-(1-(5-cyano-2-methylphenyl)-2-imino-3-methyl-2,3-dihydro-1H-imidazo[4,5-c]quinolin-8-yl)benzonitrile (30)

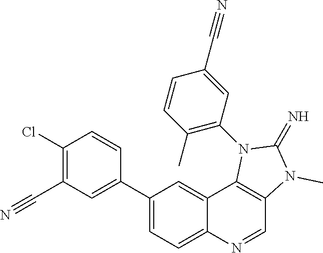 [SMILES: Cc1ccc(C#N)cc1-n1c(=N)n(C)c2cnc3ccc(-c4ccc(Cl)c(C#N)c4)cc3c21]

LCMS: t_{R}=3.823 min in 0-60 AB_7MIN_220&254 chromatography (Xtimate 3 um, C18, 2.1*30 mm), MS (ESI) m/z 449.0 [M+H]⁺.

¹H NMR: (400 MHz, DMSO-d₆) δ=9.42 (s, 1H), 9.20 (br s, 2H), 8.39 (d, J=1.5 Hz, 1H), 8.31-8.22 (m, 2H), 8.11 (dd, J=2.0, 8.8 Hz, 1H), 7.95 (d, J=8.2 Hz, 1H), 7.91 (d, J=2.4 Hz, 1H), 7.82 (d, J=8.6 Hz, 1H), 7.61 (dd, J=2.3, 8.5 Hz, 1H), 6.87 (d, J=1.8 Hz, 1H), 3.97 (s, 3H), 2.23 (s, 3H).

### 3-(8-(4-chloro-3-methoxyphenyl)-2-imino-3-methyl-2,3-dihydro-1H-imidazo[4,5-c]quinolin-1-yl)-4-methylbenzonitrile (31)

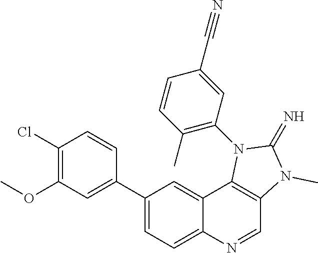

LCMS: t_{R}=4.016 min in 0-60 AB_7MIN_220&254 chromatography (Xtimate 3 um, C18, 2.1*30 mm), MS (ESI) m/z 454.1 [M+H]⁺.

¹H NMR: (400 MHz, DMSO-d₆) δ=9.38 (s, 1H), 9.08 (s, 2H), 8.38 (d, J=1.5 Hz, 1H), 8.30-8.22 (m, 2H), 8.07 (dd, J=2.0, 9.0 Hz, 1H), 7.93 (d, J=7.9 Hz, 1H), 7.47 (d, J=8.2 Hz, 1H), 6.98 (dd, J=2.0, 8.2 Hz, 1H), 6.91 (dd, J=1.9, 10.5 Hz, 2H), 3.94 (s, 3H), 3.87 (s, 3H), 2.21 (s, 3H).

### 3-(2-imino-8-(3-methoxyphenyl)-3-methyl-2,3-dihydro-H-imidazo[4,5-c]quinolin-1-yl)-4-methylbenzonitrile (32)

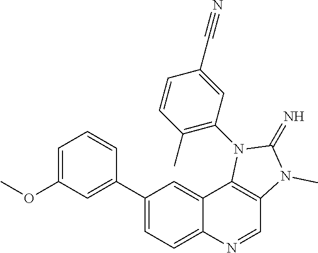

LCMS: t_{R}=3.648 min in 0-60 AB_7MIN_220&254 chromatography (Xtimate 3 um, C18, 2.1*30 mm), MS (ESI) m/z 420.1 [M+H]⁺.

¹H NMR: (400 MHz, DMSO-d₆) δ=9.39 (s, 1H), 9.07 (br s, 2H), 8.41 (d, J=1.3 Hz, 1H), 8.31-8.24 (m, 2H), 8.07 (dd, J=1.9, 8.9 Hz, 1H), 7.96 (d, J=8.2 Hz, 1H), 7.35 (t, J=7.9 Hz, 1H), 6.97-6.92 (m, 3H), 6.82 (s, 1H), 3.96 (s, 3H), 3.79 (s, 3H), 2.23 (s, 3H).

### 3-(8-(3-chlorophenyl)-2-imino-3-methyl-2,3-dihydro-1H-imidazo[4,5-c]quinolin-1-yl)-4-methylbenzonitrile (33)

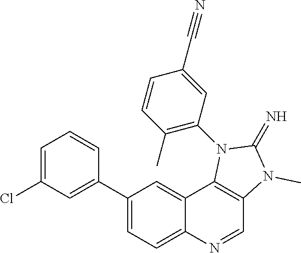 [SMILES: Cc1ccc(C#N)cc1-n1c(=N)n(C)c2cnc3ccc(-c4cccc(Cl)c4)cc3c21]

LCMS: t_{R}=3.922 min in 0-60 AB_7MIN_220&254 chromatography (Xtimate 3 um, C18, 2.1*30 mm), MS (ESI) m/z 424.1 [M+H]⁺.

¹H NMR: (400 MHz, DMSO-d₆) δ=9.41 (s, 1H), 9.14 (s, 2H), 8.43 (d, J=1.5 Hz, 1H), 8.32-8.25 (m, 2H), 8.11 (dd, J=2.1, 8.9 Hz, 1H), 7.98 (d, J=8.2 Hz, 1H), 7.50-7.45 (m, 2H), 7.40-7.37 (m, 1H), 7.30-7.28 (m, 1H), 6.88 (d, J=1.8 Hz, 1H), 3.97 (s, 3H), 2.24 (s, 3H).

### 3-(8-(3-fluorophenyl)-2-imino-3-methyl-2,3-dihydro-1H-imidazo[4,5-c]quinolin-1-yl)-4-methylbenzonitrile (34)

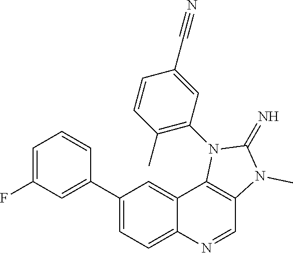 [SMILES: Cc1ccc(C#N)cc1-n1c(=N)n(C)c2cnc3ccc(-c4cccc(F)c4)cc3c21]

LCMS: t_{R}=3.722 min in 0-60 AB_7MIN_220&254 chromatography (Xtimate 3 um, C18, 2.1*30 mm), MS (ESI) m/z 408.1 [M+H]⁺.

¹H NMR: (400 MHz, DMSO-d₆) δ=9.40 (s, 1H), 9.07 (s, 2H), 8.40 (s, 1H), 8.31-8.25 (m, 2H), 8.09 (dd, J=2.1, 8.7 Hz, 1H), 7.97 (d, J=8.2 Hz, 1H), 7.52-7.45 (m, 1H), 7.27-7.21 (m, 1H), 7.19-7.13 (m, 2H), 6.90 (d, J=2.0 Hz, 1H), 3.96 (s, 3H), 2.23 (s, 3H).

### 3-(2-imino-3-methyl-8-phenyl-2,3-dihydro-1H-imidazo[4,5-c]quinolin-1-yl)-4-methylbenzonitrile (35)

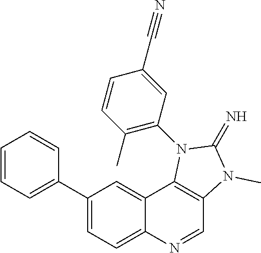 [SMILES: Cc1ccc(C#N)cc1-n1c(=N)n(C)c2cnc3ccc(-c4ccccc4)cc3c21]

LCMS: t_{R}=3.592 min in 0-60 AB_7MIN_220&254 chromatography (Xtimate 3 um, C18, 2.1*30 mm), MS (ESI) m/z 390.0[M+H]⁺.

¹H NMR: (400 MHz, DMSO-d₆) δ=9.36 (s, 1H), 9.02 (br s, 2H), 8.38 (s, 1H), 8.26 (dd, J=1.8, 8.2 Hz, 1H), 8.23 (d, J=9.0 Hz, 1H), 8.03 (dd, J=1.9, 8.9 Hz, 1H), 7.94 (br d, J=8.2 Hz, 1H), 7.44-7.35 (m, 3H), 7.31 (br d, J=6.6 Hz, 2H), 6.87 (br d, J=1.8 Hz, 1H), 3.93 (s, 3H), 2.21 (s, 3H).

### 3-(2-imino-3-methyl-8-(m-tolyl)-2,3-dihydro-1H-imidazo[4,5-c]quinolin-1-yl)-4-methylbenzonitrile (36)

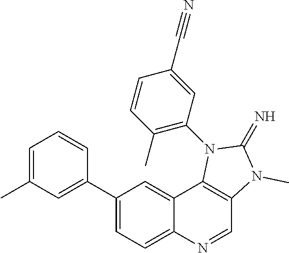

LCMS: t_{R}=3.863 min in 0-60 AB_7MIN_220&254 chromatography (Xtimate 3 um, C18, 2.1*30 mm), MS (ESI) m/z 449.1 [M+H]⁺.

¹H NMR: (400 MHz, DMSO-d₆) δ=9.38 (s, 1H), 9.11 (s, 2H), 8.44 (d, J=1.1 Hz, 1H), 8.32 (dd, J=1.4, 8.0 Hz, 1H), 8.24 (d, J=8.8 Hz, 1H), 8.06 (dd, J=1.9, 8.9 Hz, 1H), 7.98 (d, J=8.2 Hz, 1H), 7.36-7.30 (m, 1H), 7.26-7.19 (m, 2H), 7.04 (s, 1H), 6.88 (d, J=1.5 Hz, 1H), 3.96 (s, 3H), 2.35 (s, 3H), 2.23 (s, 3H).

### 3-(8-(3-hydroxyphenyl)-2-imino-3-methyl-2,3-dihydro-1H-imidazo[4,5-c]quinolin-1-yl)-4-methylbenzonitrile (37)

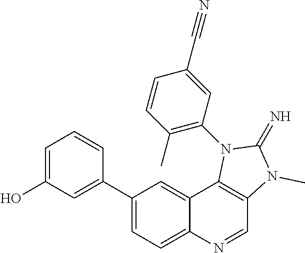 [SMILES: Cc1ccc(C#N)cc1-n1c(=N)n(C)c2cnc3ccc(-c4cccc(O)c4)cc3c21]

LCMS: t_{R}=2.979 min in 0-60 AB_7 min chromatography (Xtimate 3 um, C18, 2.1*30 mm), MS (ESI) m/z 406.1 [M+H]⁺.

¹H NMR: (400 MHz, DMSO-d₆) δ=9.54 (br s, 1H), 8.81 (s, 1H), 8.18 (s, 1H), 8.06 (br d, J=7.9 Hz, 1H), 7.99 (d, J=8.8 Hz, 1H), 7.81 (d, J=7.9 Hz, 1H), 7.71 (dd, J=2.0, 9.0 Hz, 1H), 7.15 (t, J=7.8 Hz, 1H), 6.83 (d, J=1.8 Hz, 1H), 6.75-6.66 (m, 2H), 6.62 (d, J=7.9 Hz, 1H), 5.88-5.62 (m, 1H), 3.53 (s, 3H), 2.16 (s, 3H).

### 3-(8-(3-aminophenyl)-2-imino-3-methyl-2,3-dihydro-1H-imidazo[4,5-c]quinolin-1-yl)-4-methylbenzonitrile (38)

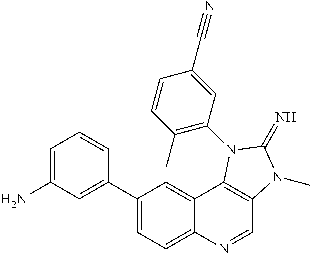 [SMILES: Cc1ccc(C#N)cc1-n1c(=N)n(C)c2cnc3ccc(-c4cccc(N)c4)cc3c21]

LCMS: t_{R}=2.391 min in 0-60 AB_7 min chromatography (Xtimate 3 um, C18, 2.1*30 mm), MS (ESI) m/z 405.0 [M+H]⁺.

¹H NMR: (400 MHz, DMSO-d₆) δ=8.81 (s, 1H), 8.19 (s, 1H), 8.09 (dd, J=1.5, 7.9 Hz, 1H), 7.99 (d, J=8.8 Hz, 1H), 7.84 (d, J=8.2 Hz, 1H), 7.68 (dd, J=2.0, 9.0 Hz, 1H), 7.00 (t, J=7.7 Hz, 1H), 6.82 (d, J=1.8 Hz, 1H), 6.58-6.49 (m, 2H), 6.33 (d, J=7.7 Hz, 1H), 5.75 (br s, 1H), 5.12 (s, 2H), 3.54 (s, 3H), 2.17 (s, 3H).

### 3-(8-(4-fluoro-3-hydroxyphenyl)-2-imino-3-methyl-2,3-dihydro-1H-imidazo[4,5-c]quinolin-1-yl)-4-methylbenzonitrile (39)

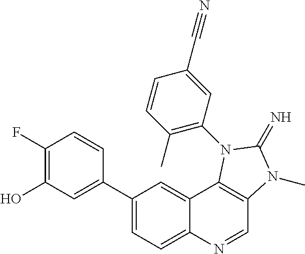 [SMILES: Cc1ccc(C#N)cc1-n1c(=N)n(C)c2cnc3ccc(-c4ccc(F)c(O)c4)cc3c21]

LCMS: t_{R}=3.081 min in 0-60 AB_7 min chromatography (Xtimate 3 um, C18, 2.1*30 mm), MS (ESI) m/z 424.1 [M+H]⁺.

¹H NMR: (400 MHz, DMSO-d₆) δ=10.02 (br s, 1H), 8.80 (s, 1H), 8.15 (s, 1H), 8.07-8.01 (m, 1H), 7.98 (d, J=9.0 Hz, 1H), 7.79 (d, J=7.9 Hz, 1H), 7.66 (dd, J=2.0, 8.8 Hz, 1H), 7.11 (dd, J=8.5, 11.1 Hz, 1H), 6.85 (dd, J=2.2, 8.4 Hz, 1H), 6.75 (d, J=1.8 Hz, 1H), 6.59 (ddd, J=2.3, 4.0, 8.4 Hz, 1H), 5.80 (br s, 1H), 3.51 (s, 3H), 2.14 (s, 3H).

### 3-(8-(3-amino-4-chlorophenyl)-2-imino-3-methyl-2,3-dihydro-1H-imidazo[4,5-c]quinolin-1-yl)-4-methylbenzonitrile (40)

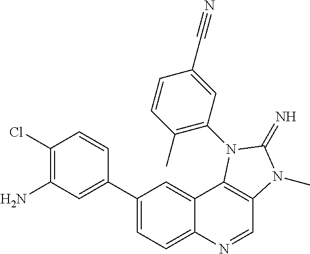 [SMILES: Cc1ccc(C#N)cc1-n1c(=N)n(C)c2cnc3ccc(-c4ccc(Cl)c(N)c4)cc3c21]

LCMS: t_{R}=3.600 min in 0-60AB_7 min_220&254 (Xtimate 3 um, C18, 2.1*30 mm), MS (ESI) m/z 439.1 [M+H]⁺.

¹H NMR (400 MHz, DMSO-d₆) δ=9.37 (s, 1H), 9.02 (s, 2H), 8.36 (d, J=1.8 Hz, 1H), 8.29-8.21 (m, 2H), 7.98 (d, J=8.2 Hz, 1H), 7.89 (dd, J=2.0, 9.0 Hz, 1H), 7.21 (d, J=8.2 Hz, 1H), 6.82 (t, J=2.3 Hz, 2H), 6.34 (dd, J=2.3, 8.3 Hz, 1H), 3.94 (s, 3H), 2.23 (s, 3H).

### 3-(8-(3,4-dimethoxyphenyl)-2-imino-3-methyl-2,3-dihydro-1H-imidazo[4,5-c]quinolin-1-yl)-4-methylbenzonitrile (41)

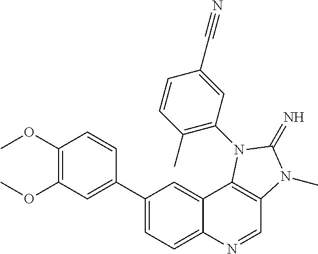

LCMS: t_{R}=3.333 min in 0-60 AB_7MIN_220&254 chromatography (Xtimate 3 um, C18, 2.1*30 mm), MS (ESI) m/z 450.1 [M+H]⁺.

¹H NMR: (400 MHz, DMSO-d₆) δ=9.36 (s, 1H), 9.04 (br s, 2H), 8.41 (d, J=1.5 Hz, 1H), 8.30 (dd, J=1.7, 8.0 Hz, 1H), 8.23 (d, J=8.8 Hz, 1H), 8.06 (dd, J=2.0, 8.8 Hz, 1H), 7.96 (d, J=8.2 Hz, 1H), 7.02 (s, 2H), 6.89 (d, J=1.8 Hz, 1H), 6.75 (s, 1H), 3.96 (s, 3H), 3.79 (d, J=4.4 Hz, 6H), 2.23 (s, 3H).

### 3-(8-(3,4-dichlorophenyl)-2-imino-3-methyl-2,3-dihydro-1H-imidazo[4,5-c]quinolin-1-yl)-4-methylbenzonitrile (42)

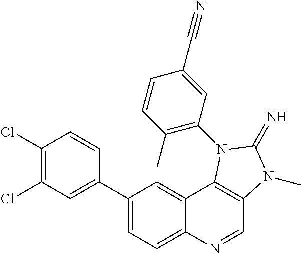 [SMILES: Cc1ccc(C#N)cc1-n1c(=N)n(C)c2cnc3ccc(-c4ccc(Cl)c(Cl)c4)cc3c21]

LCMS: t_{R}=4.328 min in 0-60 AB_7MIN_220&254 chromatography (Xtimate 3 um, C18, 2.1*30 mm), MS (ESI) m/z 458.0 [M+H]⁺.

¹H NMR: (400 MHz, DMSO-d₆) δ=9.41 (s, 1H), 9.08 (br s, 2H), 8.40 (s, 1H), 8.31-8.25 (m, 2H), 8.11 (br d, J=9.0 Hz, 1H), 7.96 (br d, J=7.9 Hz, 1H), 7.72 (br d, J=8.2 Hz, 1H), 7.48 (br d, J=2.2 Hz, 1H), 7.39 (br dd, J=2.3, 8.5 Hz, 1H), 6.85 (s, 1H), 3.95 (s, 3H), 2.22 (s, 3H).

### 3-(8-(3,4-difluorophenyl)-2-imino-3-methyl-2,3-dihydro-1H-imidazo[4,5-c]quinolin-1-yl)-4-methylbenzonitrile (43)

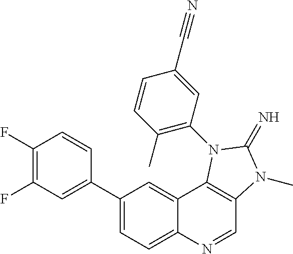 [SMILES: Cc1ccc(C#N)cc1-n1c(=N)n(C)c2cnc3ccc(-c4ccc(F)c(F)c4)cc3c21]

LCMS: t_{R}=3.837 min in 0-60 AB_7MIN_220&254 chromatography (Xtimate 3 um, C18, 2.1*30 mm), MS (ESI) m/z 426.0 [M+H]⁺.

¹H NMR: (400 MHz, DMSO-d₆) δ=9.41 (s, 1H), 9.09 (s, 2H), 8.39 (s, 1H), 8.31-8.24 (m, 2H), 8.06 (dd, J=1.9, 8.9 Hz, 1H), 7.96 (d, J=8.2 Hz, 1H), 7.57-7.49 (m, 1H), 7.46-7.38 (m, 1H), 7.16 (br d, J=8.6 Hz, 1H), 6.85 (d, J=1.5 Hz, 1H), 3.96 (s, 3H), 2.23 (s, 3H).

### 3-(8-(5-chloropyridin-2-yl)-2-imino-3-methyl-2,3-dihydro-1H-imidazo[4,5-c]quinolin-1-yl)-4-methylbenzonitrile (44)

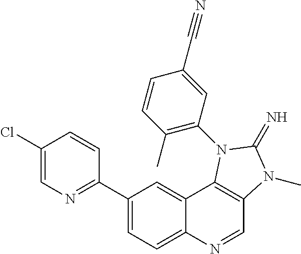 [SMILES: Cc1ccc(C#N)cc1-n1c(=N)n(C)c2cnc3ccc(-c4ccc(Cl)cn4)cc3c21]

LCMS: t_{R}=3.610 min in 0-60AB_7.0 min chromatography (Xtimate, 2.1*30 mm, 3 um), MS (ESI) m/z 425.0 [M+H]⁺.

¹H NMR: (400 MHz, DMSO-d₆): δ=9.91 (s, 2H), 9.74 (s, 1H), 8.55 (s, 1H), 8.50-8.48 (m, 2H), 8.42 (s, 1H), 8.30 (d, J=8.0 Hz, 1H), 8.01 (d, J=7.2 Hz, 1H), 7.94 (d, J=8.0 Hz, 1H), 7.76 (d, J=8.4 Hz, 1H), 7.58 (s, 1H), 4.08 (s, 3H), 2.22 (s, 3H).

### 5-(8-(4-chlorophenyl)-2-imino-3-methyl-2,3-dihydro-1H-imidazo[4,5-c]quinolin-1-yl)nicotinonitrile (45)

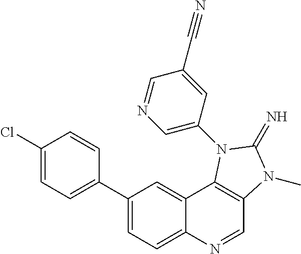 [SMILES: Cn1c(=N)n(-c2cncc(C#N)c2)c2c3cc(-c4ccc(Cl)cc4)ccc3ncc21]

LCMS: t_{R}=3.662 min in 0-60 AB_7 min chromatography (Xtimate 3 um, C18, 2.1*30 mm), MS (ESI) m/z 411.0 [M+H]⁺.

¹H NMR: (400 MHz, DMSO-d₆) δ=9.55 (br s, 2H), 9.49 (d, J=1.3 Hz, 1H), 9.44 (s, 1H), 9.37 (d, J=2.0 Hz, 1H), 9.00 (s, 1H), 8.30 (d, J=9.0 Hz, 1H), 8.07 (dd, J=1.5, 9.0 Hz, 1H), 7.50 (d, J=8.6 Hz, 2H), 7.39 (d, J=8.4 Hz, 2H), 6.97 (s, 1H), 4.01 (s, 3H).

### 5-(8-(4-chlorophenyl)-2-imino-3-methyl-2,3-dihydro-1H-imidazo[4,5-c]quinolin-1-yl)nicotinamide (46)

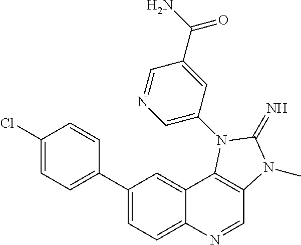 [SMILES: Cn1c(=N)n(-c2cncc(C(N)=O)c2)c2c3cc(-c4ccc(Cl)cc4)ccc3ncc21]

LCMS: t_{R}=3.181 min in 0-60 AB_7 min chromatography (Xtimate 3 um, C18, 2.1*30 mm), MS (ESI) m/z 429.0 [M+H]⁺.

¹H NMR: (400 MHz, DMSO-d₆) δ=9.25 (s, 1H), 9.00 (d, J=2.2 Hz, 1H), 8.84 (s, 1H), 8.51 (s, 1H), 8.34 (s, 1H), 8.03 (d, J=9.0 Hz, 1H), 7.85-7.76 (m, 2H), 7.46-7.38 (m, 2H), 7.36-7.28 (m, 2H), 6.98 (br s, 1H), 3.54 (s, 3H).

### 5-(8-(4-chlorophenyl)-2-imino-3-methyl-2,3-dihydro-1H-imidazo[4,5-c]quinolin-1-yl)-6-methyl-2-(piperazin-1-yl)nicotinonitrile (47)

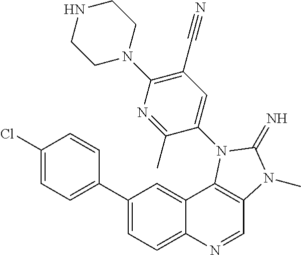 [SMILES: Cc1nc(N2CCNCC2)c(C#N)cc1-n1c(=N)n(C)c2cnc3ccc(-c4ccc(Cl)cc4)cc3c21]

LCMS: t_{R}=4.896 min in 0-30AB_7.0 min chromatography (Xtimate, 2.1*30 mm, 3 um), MS (ESI) m/z 509.1 [M+H]⁺.

¹H NMR: (400 MHz, DMSO-d₆): δ=9.78 (s, 2H), 9.52 (s, 2H), 9.44 (s, 1H), 8.61 (s, 1H), 8.32 (d, J=9.2 Hz, 1H), 8.13-8.10 (m, 1H), 7.58-7.56 (m, 2H), 7.48-7.46 (m, 2H), 7.22 (s, 1H), 4.07-4.04 (m, 4H), 3.99 (s, 3H), 3.32-3.29 (m, 4H), 2.28 (s, 3H).

### 5-(8-(4-chlorophenyl)-2-imino-3-methyl-2,3-dihydro-1H-imidazo[4,5-c]quinolin-1-yl)-2-(piperazin-1-yl)nicotinonitrile (48)

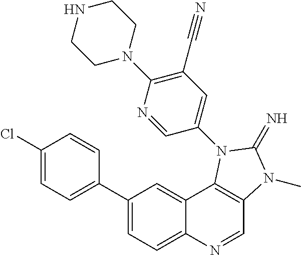 [SMILES: Cn1c(=N)n(-c2cnc(N3CCNCC3)c(C#N)c2)c2c3cc(-c4ccc(Cl)cc4)ccc3ncc21]

LCMS: t_{R}=3.867 min in 0-60AB_7 min chromatography (Xtimate 3 um, C18, 2.1*30 mm), MS (ESI) m/z 495.2 [M+H]⁺.

### 5-(8-(4-chlorophenyl)-2-imino-3-methyl-2,3-dihydro-1H-imidazo[4,5-c]quinolin-1-yl)-2-(piperazin-1-yl)benzonitrile (49)

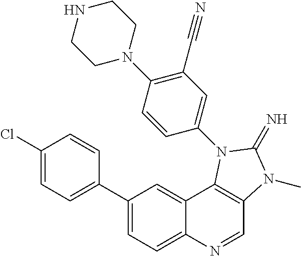 [SMILES: Cn1c(=N)n(-c2ccc(N3CCNCC3)c(C#N)c2)c2c3cc(-c4ccc(Cl)cc4)ccc3ncc21]

LCMS: t_{R}=3.044 mi in 0-60AB_7 min_220&254 (Xtimate 3 um, C18, 2.1*30 mm), MS (ESI) m/z 494.2 [M+H]⁺.

¹H NMR (400 MHz, DMSO-d₆) δ=9.33 (s, 1H), 9.14-8.94 (m, 4H), 8.34 (d, J=2.4 Hz, 1H), 8.23 (d, J=9.0 Hz, 1H), 8.06 (ddd, J=2.2, 8.8, 18.3 Hz, 2H), 7.64 (d, J=9.0 Hz, 1H), 7.53-7.46 (m, 2H), 7.44-7.36 (m, 2H), 7.04 (d, J=2.0 Hz, 1H), 3.91 (s, 3H), 3.49-3.43 (m, 4H), 3.38 (br s, 4H).

### 5-(8-(4-chlorophenyl)-2-imino-3-methyl-2,3-dihydro-1H-imidazo[4,5-c]quinolin-1-yl)-2-morpholinonicotinonitrile (50)

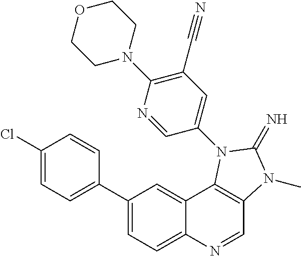 [SMILES: Cn1c(=N)n(-c2cnc(N3CCOCC3)c(C#N)c2)c2c3cc(-c4ccc(Cl)cc4)ccc3ncc21]

LCMS: t_{R}=3.865 min in 0-60AB_7 min chromatography (Xtimate 3 um, C18, 2.1*30 mm), MS (ESI) m/z 496.0 [M+H]⁺.

¹HNMR: (DMSO-d₆ 400 MHz): δ=9.38 (s, 1H), 9.23 (s, 1H), 9.27-9.18 (m, 1H), 8.84 (d, J=2.5 Hz, 1H), 8.67 (d, J=2.8 Hz, 1H), 8.29 (d, J=8.8 Hz, 1H), 8.10 (br d, J=2.0 Hz, 1H), 8.08 (d, J=1.8 Hz, 1H), 7.57-7.50 (m, 4H), 7.30 (d, J=1.8 Hz, 1H), 3.97 (s, 3H), 3.96-3.71 (m, 8H).

### 5-(8-(4-chlorophenyl)-2-imino-3-methyl-2,3-dihydro-1H-imidazo[4,5-c]quinolin-1-yl)-2-morpholinobenzonitrile (51)

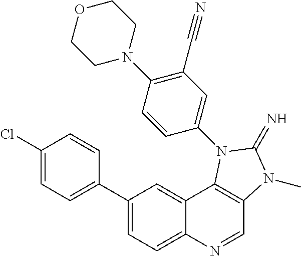 [SMILES: Cn1c(=N)n(-c2ccc(N3CCOCC3)c(C#N)c2)c2c3cc(-c4ccc(Cl)cc4)ccc3ncc21]

LCMS: t_{R}=4.089 min in 0-60AB_7 min_220&254 (Xtimate 3 um, C18, 2.1*30 mm), MS (ESI) m/z 495.1 [M+H]⁺.

¹H NMR (400 MHz, DMSO-d₆) δ=8.78 (s, 1H), 8.10 (br s, 1H), 7.99 (d, J=9.0 Hz, 1H), 7.87-7.82 (m, 1H), 7.77 (br d, J=8.6 Hz, 1H), 7.43 (br d, J=8.4 Hz, 3H), 7.34 (br d, J=8.4 Hz, 2H), 7.02 (br s, 1H), 3.82 (br s, 4H), 3.49 (s, 3H), 3.27-3.16 (m, 4H).

### 3-(8-(5-chloropyrimidin-2-yl)-2-imino-3-methyl-2,3-dihydro-1H-imidazo[4,5-c]quinolin-1-yl)-4-methylbenzonitrile (52)

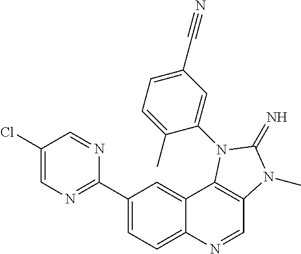 [SMILES: Cc1ccc(C#N)cc1-n1c(=N)n(C)c2cnc3ccc(-c4ncc(Cl)cn4)cc3c21]

LCMS: t_{R}=3.486 min in 0-60AB_7.0 min chromatography (Xtimate, 2.1*30 mm, 3 um), MS (ESI) m/z 426.0 [M+H]⁺.

¹H NMR: (400 MHz, DMSO-d₆): δ=9.43 (s, 1H), 9.22 (s, 2H), 8.91 (s, 2H), 8.56-8.54 (m, 1H), 8.35 (s, 1H), 8.29-8.26 (m, 2H), 7.94-7.92 (m, 1H), 7.90-7.89 (m, 1H), 3.97 (s, 3H), 2.18 (s, 3H).

### 3-(8-(4-chlorophenyl)-2-imino-3-methyl-2,3-dihydro-1H-imidazo[4,5-c]quinolin-1-yl)-4-methoxybenzonitrile (53)

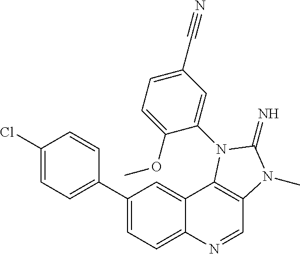 [SMILES: COc1ccc(C#N)cc1-n1c(=N)n(C)c2cnc3ccc(-c4ccc(Cl)cc4)cc3c21]

LCMS: t_{R}=3.982 min in 0-60AB_7.0 min chromatography (Xtimate, 2.1*30 mm, 3 um), MS (ESI) m/z 440.0 [M+H]⁺.

¹H NMR: (400 MHz, DMSO-d₆): δ=8.78-8.76 (m, 1H), 8.24-8.13 (m, 1H), 7.99 (d, J=9.2 Hz, 1H), 7.77-7.74 (m, 1H), 7.56-7.54 (m, 1H), 7.45 (d, J=8.8 Hz, 2H), 7.31 (d, J=8.4 Hz, 2H), 6.99-6.97 (m, 1H), 3.73 (s, 3H), 3.49 (s, 3H).

### 3-(8-(4-chlorophenyl)-2-imino-3-methyl-2,3-dihydro-1H-imidazo[4,5-c]quinolin-1-yl)-4-(trifluoromethoxy)benzonitrile (54)

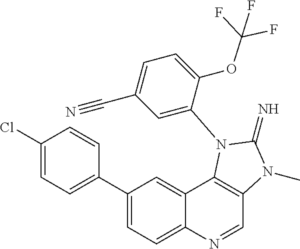 [SMILES: Cn1c(=N)n(-c2cc(C#N)ccc2OC(F)(F)F)c2c3cc(-c4ccc(Cl)cc4)ccc3ncc21]

LCMS: t_{R}=4.535 min in 10-60AB_7.0 min chromatography (Xtimate, 2.1*30 mm, 3 um), MS (ESI) m/z 493.9 [M+H]⁺. 1H NMR: (400 MHz, DMSO-d₆): δ=8.82 (s, 1H), 8.56 (s, 1H), 8.31 (s, 1H), 8.03-7.99 (m, 2H), 7.80-7.77 (m, 1H), 7.45 (d, J=8.8 Hz, 2H), 7.31 (d, J=8.8 Hz, 2H), 6.92 (s, 1H), 3.51 (s, 3H).

### (E)-1-(1-(5-cyano-2-methylphenyl)-8-(4-fluorophenyl)-3-methyl-1,3-dihydro-2H-imidazo[4,5-c]quinolin-2-ylidene)urea (55)

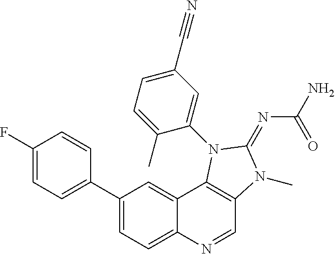 [SMILES: Cc1ccc(C#N)cc1-n1/c(=N/C(N)=O)n(C)c2cnc3ccc(-c4ccc(F)cc4)cc3c21]

LCMS: t_{R}=3.323 min in 10-80CD_7MIN_220&254 (XBrige Shield RP18 2.1*50 mm, 5 um), MS (ESI) m/z 451.1 [M+H]⁺. 1H NMR (400 MHz, DMSO-d₆) δ=9.09 (s, 1H), 8.21 (s, 1H), 8.11 (d, J=9.3 Hz, 1H), 8.05 (d, J=8.2 Hz, 1H), 7.88 (d, J=8.8 Hz, 1H), 7.77 (d, J=7.9 Hz, 1H), 7.38-7.29 (m, 2H), 7.28-7.19 (m, 2H), 6.86 (s, 1H), 6.50-5.54 (m, 2H), 3.67 (s, 3H), 2.17 (s, 3H).

### (E)-1-(1-(5-cyano-2-methylphenyl)-8-(4-fluorophenyl)-3-methyl-1,3-dihydro-2H-imidazo[4,5-c]quinolin-2-ylidene)guanidine (56)

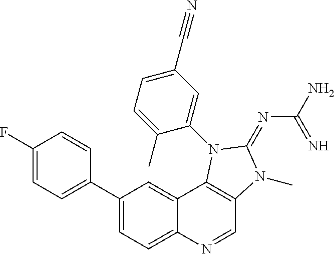 [SMILES: Cc1ccc(C#N)cc1-n1/c(=N/C(=N)N)n(C)c2cnc3ccc(-c4ccc(F)cc4)cc3c21]

LCMS: t_{R}=2.564 min in 10-80AB_7 min 220&254 (Xtimate C18 2.1*30 mm, 3 um), MS (ESI) m/z 450.0 [M+H]⁺.

¹H NMR (400 MHz, METHANOL-d₄) δ=9.22 (s, 1H), 8.24 (d, J=9.0 Hz, 1H), 8.10-8.07 (m, 1H), 8.06 (dd, J=1.4, 7.8 Hz, 1H), 8.01 (dd, J=1.9, 8.7 Hz, 1H), 7.82 (d, J=8.2 Hz, 1H), 7.39-7.32 (m, 2H), 7.15 (t, J=8.7 Hz, 2H), 7.09 (d, J=2.0 Hz, 1H), 3.90 (s, 3H), 2.27 (s, 3H).

### 8-(4-chlorophenyl)-3-methyl-1-(1-(tetrahydro-2H-pyran-4-yl)piperidin-4-yl)-1,3-dihydro-2H-imidazo[4,5-c]quinolin-2-imine (57)

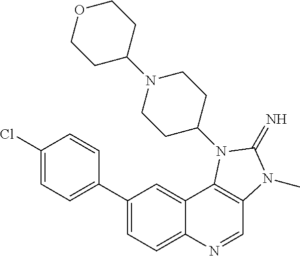 [SMILES: Cn1c(=N)n(C2CCN(C3CCOCC3)CC2)c2c3cc(-c4ccc(Cl)cc4)ccc3ncc21]

LCMS: t_{R}=4.299 min in 0-30AB_7 min_220&254 (Xtimate 3 um, C18, 2.1*30 mm), MS (ESI) m/z 476.0 [M+H]⁺.

¹H NMR (400 MHz, DMSO-d₆) δ=11.08 (br s, 1H), 9.35 (br d, J=15.1 Hz, 3H), 8.50 (s, 1H), 8.36 (d, J=8.8 Hz, 1H), 8.17 (br d, J=8.8 Hz, 1H), 8.00 (d, J=8.5 Hz, 2H), 7.64 (d, J=8.5 Hz, 2H), 5.79 (br d, J=5.3 Hz, 1H), 4.00-3.75 (m, 9H), 3.62 (br s, 1H), 3.51-3.18 (m, 6H), 2.10 (br d, J=10.8 Hz, 2H), 1.86-1.71 (m, 2H).

### 8-(4-chlorophenyl)-3-methyl-1-(3-morpholinocyclobutyl)-1,3-dihydro-2H-imidazo[4,5-c]quinolin-2-imine (58)

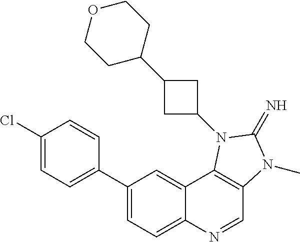 [SMILES: Cn1c(=N)n(C2CC(C3CCOCC3)C2)c2c3cc(-c4ccc(Cl)cc4)ccc3ncc21]

LCMS: t_{R}=4.483 min in 0-30AB_7 min_220&254 (Xtimate 3 um, C18, 2.1*30 mm), MS (ESI) m/z 448.0 [M+H]⁺.

¹H NMR (400 MHz, DMSO-d₆) δ=11.90 (br s, 1H), 9.36 (s, 1H), 9.29 (br s, 2H), 8.38 (d, J=1.5 Hz, 1H), 8.32 (d, J=8.8 Hz, 1H), 8.15 (dd, J=1.8, 8.8 Hz, 1H), 7.98 (d, J=8.5 Hz, 2H), 7.63 (d, J=8.5 Hz, 2H), 5.55 (quin, J=8.2 Hz, 1H), 3.99-3.85 (m, 5H), 3.78-3.60 (m, 3H), 3.36-3.30 (m, 2H), 3.26-3.11 (m, 4H), 2.95 (br d, J=10.8 Hz, 2H).

### 8-(4-chlorophenyl)-3-methyl-1-((1s,4s)-4-morpholinocyclohexyl)-1,3-dihydro-2H-imidazo[4,5-c]quinolin-2-imine (59)

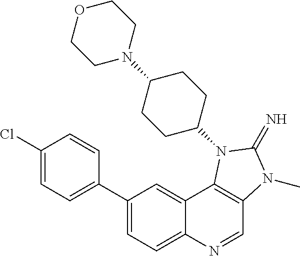

LCMS: t_{R}=3.900 min in 0-60AB_7 min chromatography (Xtimate 3 um, C18, 2.1*30 mm), MS (ESI) m/z 476.2 [M+H]⁺.

### 3-(2-imino-3-methyl-8-(1-methyl-2,5-dihydro-1H-pyrrol-3-yl)-2,3-dihydro-1H-imidazo[4,5-c]quinolin-1-yl)-4-methylbenzonitrile (60)

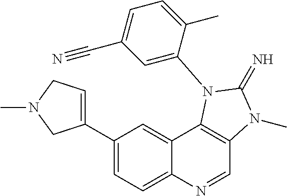

LCMS: t_{R}=1.572 min in 0-60AB_7.0 MIN chromatography (Xtimate 3 um, C18, 2.1*30 mm), MS (ESI) m/z 395.2[M+H]⁺.

¹H NMR (400 MHz, DMSO-d₆) δ=10.81-10.58 (m, 1H), 9.39 (s, 1H), 9.12 (s, 2H), 8.34 (s, 1H), 8.30-8.24 (m, 1H), 8.21 (d, J=9.0 Hz, 1H), 8.02-7.92 (m, 2H), 6.48-6.36 (m, 2H), 4.46 (br d, J=14.3 Hz, 1H), 4.22-4.01 (m, 3H), 3.95 (s, 3H), 2.96 (s, 3H), 2.22 (s, 3H).

### 3-(2-imino-3-methyl-8-(o-tolyl)-2,3-dihydro-1H-imidazo[4,5-c]quinolin-1-yl)-4-methylbenzonitrile (61)

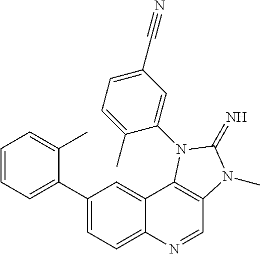 [SMILES: Cc1ccccc1-c1ccc2ncc3c(c2c1)n(-c1cc(C#N)ccc1C)c(=N)n3C]

LCMS: t_{R}=3.678 min in 0-60 AB_7MIN_220&254 chromatography (Xtimate 3 um, C18, 2.1*30 mm), MS (ESI) m/z 404.1 [M+H]⁺.

¹H NMR: (400 MHz, DMSO-d₆) δ=9.42 (s, 1H), 9.01 (br s, 2H), 8.36 (d, J=1.5 Hz, 1H), 8.26 (d, J=8.6 Hz, 1H), 8.20 (dd, J=1.7, 8.0 Hz, 1H), 7.89 (d, J=8.4 Hz, 1H), 7.75 (dd, J=2.0, 8.8 Hz, 1H), 7.30-7.26 (m, 2H), 7.26-7.21 (m, 1H), 7.09 (d, J=7.1 Hz, 1H), 6.69 (d, J=1.5 Hz, 1H), 3.96 (s, 3H), 2.22 (s, 3H), 2.04 (s, 3H).

### 3-(8-(2-chlorophenyl)-2-imino-3-methyl-2,3-dihydro-1H-imidazo[4,5-c]quinolin-1-yl)-4-methylbenzonitrile (62)

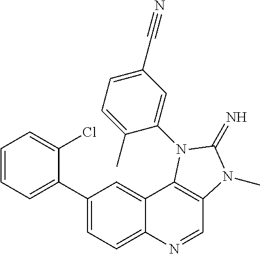 [SMILES: Cc1ccc(C#N)cc1-n1c(=N)n(C)c2cnc3ccc(-c4ccccc4Cl)cc3c21]

LCMS: t_{R}=3.723 min in 0-60AB_7.0 MIN chromatography (Xtimate 3 um, C18, 2.1*30 mm), MS (ESI) m/z 424.0[M+H]⁺.

¹H NMR (400 MHz, DMSO-d₆) δ=9.69 (s, 1H), 8.40 (d, J=9.0 Hz, 1H), 8.25 (d, J=1.8 Hz, 1H), 8.08 (ddd, J=1.8, 8.5, 16.4 Hz, 2H), 7.88 (d, J=8.2 Hz, 1H), 7.54-7.47 (m, 1H), 7.45-7.38 (m, 2H), 7.37-7.31 (m, 1H), 7.14 (d, J=1.5 Hz, 1H), 4.08 (s, 3H), 2.34 (s, 3H).

### 8-(4-chlorophenyl)-3-methyl-1-phenyl-1,3-dihydro-2H-imidazo[4,5-c]quinolin-2-imine (63)

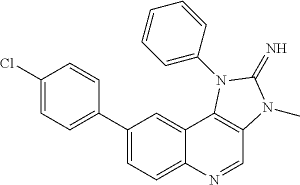 [SMILES: Cn1c(=N)n(-c2ccccc2)c2c3cc(-c4ccc(Cl)cc4)ccc3ncc21]

LCMS: t_{R}=4.500 min in 0-60AB_7 min_220&254 (Xtimate 3 um, C18, 2.1*30 mm), MS (ESI) m/z 385.0 [M+H]⁺.

¹H NMR (400 MHz, DMSO-d₆) δ=9.33 (s, 1H), 8.87 (s, 2H), 8.21 (d, J=9.0 Hz, 1H), 7.99 (dd, J=2.1, 8.9 Hz, 1H), 7.89-7.82 (m, 4H), 7.49-7.44 (m, 2H), 7.32-7.28 (m, 2H), 6.90 (d, J=1.8 Hz, 1H), 3.91 (s, 3H).

### 8-(4-chlorophenyl)-1-(3-methoxyphenyl)-3-methyl-1,3-dihydro-2H-imidazo[4,5-c]quinolin-2-imine (64)

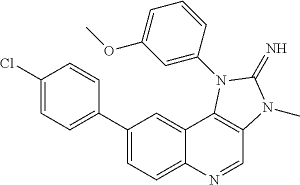 [SMILES: COc1cccc(-n2c(=N)n(C)c3cnc4ccc(-c5ccc(Cl)cc5)cc4c32)c1]

LCMS: t_{R}=4.489 min in 0-60 AB_7MIN_220&254 chromatography (Xtimate 3 um, C18, 2.1*30 mm), MS (ESI) m/z 415.0 [M+H]⁺.

¹H NMR: (400 MHz, DMSO-d₆) δ=9.35 (s, 1H), 8.92 (s, 2H), 8.24 (d, J=8.8 Hz, 1H), 8.03 (dd, J=2.1, 8.9 Hz, 1H), 7.77 (t, J=8.2 Hz, 1H), 7.53-7.49 (m, 3H), 7.49-7.42 (m, 2H), 7.39-7.35 (m, 2H), 7.08 (d, J=1.8 Hz, 1H), 3.94 (s, 3H), 3.86 (s, 3H).

### 4-(8-(4-chlorophenyl)-2-imino-3-methyl-2,3-dihydro-1H-imidazo[4,5-c]quinolin-1-yl)-N-cyclopropylbenzamide (65)

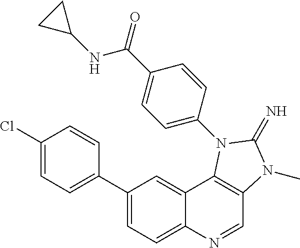 [SMILES: Cn1c(=N)n(-c2ccc(C(=O)NC3CC3)cc2)c2c3cc(-c4ccc(Cl)cc4)ccc3ncc21]

LCMS: t_{R}=3.792 min in 0-60AB_7.0 min chromatography (Xtimate, 2.1*30 mm, 3 um), MS (ESI) m/z 468.1 [M+H]⁺.

¹H NMR: (400 MHz, DMSO-d₆): δ=8.79 (s, 1H), 8.68 (s, 1H), 8.10 (d, J=8.4 Hz, 2H), 7.97 (s, 1H), 7.76-7.69 (m, 3H), 7.33 (d, J=8.4 Hz, 2H), 7.24 (d, J=8.8 Hz, 2H), 6.84 (s, 1H), 3.50 (s, 3H), 2.93-2.92 (m, 1H), 0.75-0.72 (m, 2H), 0.62-0.60 (m, 2H).

### 8-(4-chlorophenyl)-3-methyl-1-(m-tolyl)-1,3-dihydro-2H-imidazo[4,5-c]quinolin-2-imine (66)

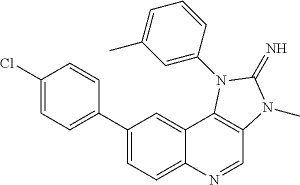 [SMILES: Cc1cccc(-n2c(=N)n(C)c3cnc4ccc(-c5ccc(Cl)cc5)cc4c32)c1]

LCMS: t_{R}=4.154 min in 0-60AB_7 min_220&254 (Xtimate 3 um, C18, 2.1*30 mm), MS (ESI) m/z 399.0 [M+H]⁺.

¹H NMR (400 MHz, DMSO-d₆) δ=9.32 (s, 1H), 8.89 (s, 2H), 8.20 (d, J=9.0 Hz, 1H), 8.00 (dd, J=2.1, 8.9 Hz, 1H), 7.74-7.67 (m, 3H), 7.66-7.59 (m, 1H), 7.52-7.44 (m, 2H), 7.36-7.28 (m, 2H), 6.98 (d, J=1.8 Hz, 1H), 3.91 (s, 3H), 2.49 (s, 3H).

### 1-(3-(8-(4-chlorophenyl)-2-imino-3-methy-2,3-dihydro-H-imidazo[4,5-c]quinolin-1-yl)phenyl)-N-methylmethanamine (67)

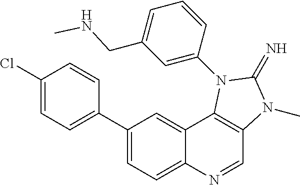 [SMILES: CNCc1cccc(-n2c(=N)n(C)c3cnc4ccc(-c5ccc(Cl)cc5)cc4c32)c1]

LCMS: t_{R}=2.861 min in 0-60AB_7 min_220&254 (Xtimate C18 2.1*30 mm, 3 um), MS (ESI) m/z 427.9 [M+H]⁺.

¹H NMR (400 MHz, DMSO-d₆) δ=9.37 (s, 1H), 9.32-9.15 (m, 2H), 9.02 (br s, 2H), 8.25 (d, J=9.0 Hz, 1H), 8.03 (dd, J=1.6, 8.9 Hz, 1H), 8.00-7.88 (m, 4H), 7.51 (d, J=8.5 Hz, 2H), 7.35 (d, J=8.5 Hz, 2H), 6.99 (d, J=1.3 Hz, 1H), 4.40-4.25 (m, 2H), 3.96 (s, 3H), 2.58-2.53 (m, 3H).

### 2-(8-(4-chlorophenyl)-2-imino-3-methyl-2,3-dihydro-1H-imidazo[4,5-c]quinolin-1-yl)benzonitrile (68)

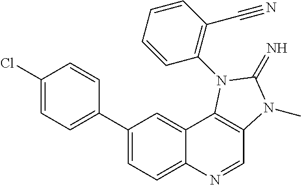 [SMILES: Cn1c(=N)n(-c2ccccc2C#N)c2c3cc(-c4ccc(Cl)cc4)ccc3ncc21]

LCMS: t_{R}=3.965 min in 0-60AB_7 min_220&254 chromatography (Xtimate, 2.1*30 mm, 3 um), MS (ESI) m/z 409.9 [M+H]⁺.

¹H NMR: (400 MHz, DMSO-d₆) δ=10.15 (br s, 1H), 9.91 (br s, 1H), 9.61 (s, 1H), 8.79 (br d, J=7.8 Hz, 1H), 8.58 (br d, J=5.3 Hz, 1H), 8.50 (br s, 1H), 8.35 (br d, J=8.8 Hz, 1H), 8.21 (br t, J=7.7 Hz, 1H), 8.12 (br d, J=8.3 Hz, 1H), 7.90 (br t, J=7.5 Hz, 1H), 7.77 (br s, 2H), 7.57 (br d, J=7.8 Hz, 2H), 4.08 (s, 3H).

### 3-(8-(4-chlorophenyl)-2-imino-3-methyl-2,3-dihydro-1H-imidazo[4,5-c]quinolin-1-yl)-2-methylbenzonitrile (69)

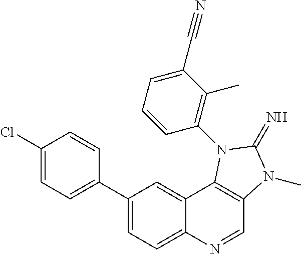 [SMILES: Cc1c(C#N)cccc1-n1c(=N)n(C)c2cnc3ccc(-c4ccc(Cl)cc4)cc3c21]

LCMS: t_{R}=4.062 min in 0-60AB_7.0 min chromatography (Xtimate, 2.1*30 mm, 3 um), MS (ESI) m/z 424.0 [M+H]⁺.

¹H NMR: (400 MHz, DMSO-d₆): δ=8.82 (s, 1H), 8.11 (s, 1H), 8.01 (s, 1H), 7.90-7.89 (m, 1H), 7.78-7.75 (m, 1H), 7.74-7.68 (m, 1H), 7.43 (d, J=8.4 Hz, 2H), 7.27 (d, J=8.8 Hz, 2H), 6.79 (s, 1H), 3.52 (s, 3H), 2.28 (s, 3H).

### 5-(8-(4-chlorophenyl)-2-imino-3-methyl-2,3-dihydro-1H-imidazo[4,5-c]quinolin-1-yl)-2-methylbenzonitrile (70)

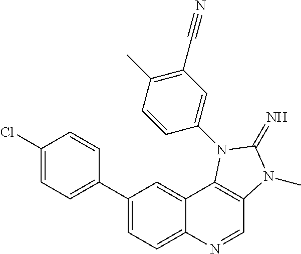 [SMILES: Cc1ccc(-n2c(=N)n(C)c3cnc4ccc(-c5ccc(Cl)cc5)cc4c32)cc1C#N]

LCMS: t_{R}=4.081 min in 0-60AB_7 min chromatography (Xtimate 3 um, C18, 2.1*30 mm), MS (ESI) m/z 424.1 [M+H]⁺.

¹H NMR (400 MHz, DMSO-d₆) δ=9.36 (s, 1H), 9.00 (s, 2H), 8.42 (d, J=2.21 Hz, 1H), 8.26 (d, J=9.04 Hz, 1H), 8.13 (dd, J=8.16, 2.21 Hz, 1H), 8.06 (dd, J=8.93, 2.09 Hz, 1H), 7.97 (d, J=8.16 Hz, 1H), 7.51-7.55 (m, 2H), 7.35-7.39 (m, 2H), 6.99 (d, J=1.98 Hz, 1H), 3.94 (s, 3H), 2.75 (s, 3H).

### 3-(8-(4-chlorophenyl)-2-imino-3-methyl-2,3-dihydro-1H-imidazo[4,5-c]quinolin-1-yl)-5-methylbenzonitrile (71)

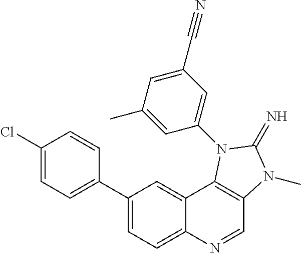 [SMILES: Cc1cc(C#N)cc(-n2c(=N)n(C)c3cnc4ccc(-c5ccc(Cl)cc5)cc4c32)c1]

LCMS: t_{R}=4.664 min in 0-60AB_7 min chromatography (Xtimate 3 um, C18, 2.1*30 mm), MS (ESI) m/z 424.0 [M+H]⁺.

¹H NMR (400 MHz, DMSO) δ=9.52 (s, 1H), 8.34 (d, J=9.2 Hz, 1H), 8.29 (s, 1H), 8.23 (s, 1H), 8.13-8.09 (m, 2H), 7.51 (d, J=8.8 Hz, 2H), 7.36 (d, J=8.8 Hz, 2H), 7.04 (d, J=2.0 Hz, 1H), 4.03 (s, 3H), 2.54 (s, 3H).

### 8-(4-chlorophenyl)-3-methyl-1-(4-(piperazin-1-yl)-3-(trifluoromethyl)phenyl)-1,3-dihydro-2H-imidazo[4,5-c]quinolin-2-imine (72)

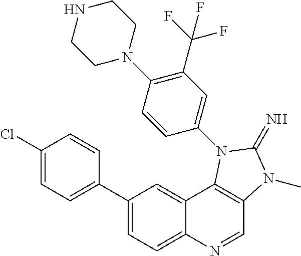 [SMILES: Cn1c(=N)n(-c2ccc(N3CCNCC3)c(C(F)(F)F)c2)c2c3cc(-c4ccc(Cl)cc4)ccc3ncc21]

LCMS: t_{R}=3.819 min in 0-60AB_7 min 220&254 (Xtimate 3 um, C18, 2.1*30 mm), MS (ESI) m/z 537.0 [M+H]⁺.

¹H NMR (400 MHz, DMSO-d₆) δ=9.36 (s, 1H), 9.03 (s, 2H), 8.98-8.26 (m, 4H), 8.21 (d, J=8.8 Hz, 1H), 8.05 (dd, J=2.0, 8.8 Hz, 1H), 7.97 (d, J=8.6 Hz, 1H), 7.48 (d, J=8.6 Hz, 2H), 7.35 (d, J=8.4 Hz, 2H), 6.92 (d, J=2.0 Hz, 1H), 3.94 (s, 3H), 3.35-3.30 (m, 4H), 3.23-3.20 (m, 4H).

### 2-(3-(8-(4-chlorophenyl)-2-imino-3-methyl-2,3-dihydro-1H-imidazo[4,5-c]quinolin-1-yl)phenyl)acetonitrile (73)

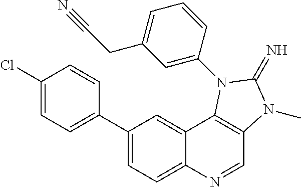 [SMILES: Cn1c(=N)n(-c2cccc(CC#N)c2)c2c3cc(-c4ccc(Cl)cc4)ccc3ncc21]

LCMS: t_{R}=4.025 min in 0-60AB_7 min_220&254 (Xtimate C18 2.1*30 mm, 3 um), MS (ESI) m/z 424.0 [M+H]⁺.

¹H NMR (400 MHz, DMSO-d₆) δ=9.33 (s, 1H), 8.90 (s, 2H), 8.22 (d, J=8.8 Hz, 1H), 8.01 (dd, J=2.0, 8.8 Hz, 1H), 7.92-7.77 (m, 4H), 7.51-7.46 (m, 2H), 7.37-7.32 (m, 2H), 6.91 (d, J=1.8 Hz, 1H), 4.30 (s, 2H), 3.91 (s, 3H).

### 1-(3-(8-(4-chlorophenyl)-2-imino-3-methyl-2,3-dihydro-1H-imidazo[4,5-c]quinolin-1-yl)phenyl)ethan-1-one (74)

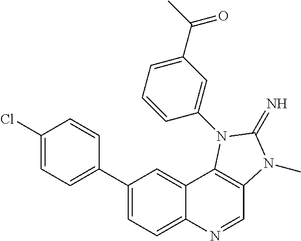 [SMILES: CC(=O)c1cccc(-n2c(=N)n(C)c3cnc4ccc(-c5ccc(Cl)cc5)cc4c32)c1]

LCMS: t_{R}=3.727 min in 0-60AB_7 min_220&254 (Xtimate C18 2.1*30 mm, 3 um), MS (ESI) m/z 426.9 [M+H]⁺.

¹H NMR (400 MHz, DMSO-d₆) δ=9.34 (s, 1H), 8.96 (s, 2H), 8.47-8.40 (m, 2H), 8.22 (d, J=9.0 Hz, 1H), 8.16-8.10 (m, 1H), 8.03-7.95 (m, 2H), 7.48-7.41 (m, 2H), 7.32-7.24 (m, 2H), 6.93 (d, J=1.8 Hz, 1H), 3.92 (s, 3H), 2.67 (s, 3H).

### 8-(4-chlorophenyl)-3-methyl-1-(3-(methylsulfonyl)phenyl)-1,3-dihydro-2H-imidazo[4,5-c]quinolin-2-imine (75)

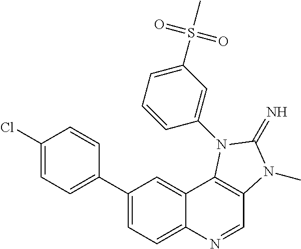 [SMILES: Cn1c(=N)n(-c2cccc(S(C)(=O)=O)c2)c2c3cc(-c4ccc(Cl)cc4)ccc3ncc21]

LCMS: t_{R}=3.563 min in 0-60AB_7 min_220&254 (Xtimate 3 um, C18, 2.1*30 mm), MS (ESI) m/z 462.9 [M+H]⁺.

¹H NMR (400 MHz, DMSO-d₆) δ=9.39 (s, 1H), 9.21 (s, 2H), 8.52 (t, J=1.8 Hz, 1H), 8.42 (td, J=1.2, 8.2 Hz, 1H), 8.25 (d, J=9.0 Hz, 1H), 8.23-8.20 (m, 1H), 8.11-8.06 (m, 1H), 8.03 (dd, J=2.1, 8.9 Hz, 1H), 7.46-7.42 (m, 2H), 7.33-7.29 (m, 2H), 6.85 (d, J=1.8 Hz, 1H), 3.95 (s, 3H), 3.31 (s, 3H).

### 8-(4-chlorophenyl)-3-methyl-1-(3-(piperazin-1-yl)phenyl)-1,3-dihydro-2H-imidazo[4,5-c]quinolin-2-imine (76)

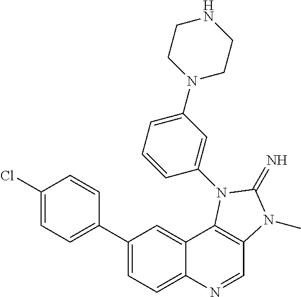 [SMILES: Cn1c(=N)n(-c2cccc(N3CCNCC3)c2)c2c3cc(-c4ccc(Cl)cc4)ccc3ncc21]

LCMS: t_{R}=3.965 min in 0-60 AB_7 min chromatography (Xtimate 3 um, C18, 2.1*30 mm), MS (ESI) m/z 469.0 [M+H]⁺.

¹H NMR: (400 MHz, DMSO-d₆) δ=9.31 (s, 1H), 9.08-8.97 (m, 2H), 8.93 (s, 2H), 8.20 (d, J=8.8 Hz, 1H), 8.00 (dd, J=2.1, 8.9 Hz, 1H), 7.70-7.63 (m, 1H), 7.52-7.43 (m, 3H), 7.35 (d, J=8.6 Hz, 2H), 7.28-7.22 (m, 1H), 7.04 (d, J=1.8 Hz, 1H), 3.91 (s, 3H), 3.44 (br t, J=5.0 Hz, 4H), 3.24 (br s, 4H).

### 3-(8-(4-chlorophenyl)-2-imino-3-methyl-2,3-dihydro-1H-imidazo[4,5-c]quinolin-1-yl)benzenesulfonamide (77)

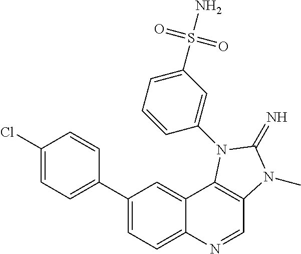 [SMILES: Cn1c(=N)n(-c2cccc(S(N)(=O)=O)c2)c2c3cc(-c4ccc(Cl)cc4)ccc3ncc21]

LCMS: t_{R}=3.416 min in 0-60AB_7 mm 220&254 (Xtimate 3 um, C18, 2.1*30 mm), MS (ESI) m/z 463.9 [M+H]⁺.

¹H NMR (400 MHz, DMSO-d₆) δ=9.32 (s, 1H), 8.96 (s, 2H), 8.34 (s, 1H), 8.23 (dd, J=8.4, 17.4 Hz, 2H), 8.08-8.04 (m, 1H), 8.03-7.96 (m, 2H), 7.80 (s, 2H), 7.45 (d, J=8.6 Hz, 2H), 7.35 (br d, J=8.4 Hz, 2H), 6.85 (s, 1H), 3.89 (s, 3H).

### 8-(4-chlorophenyl)-1-(3-fluorophenyl)-3-methyl-1,3-dihydro-2H-imidazo[4,5-c]quinolin-2-imine (78)

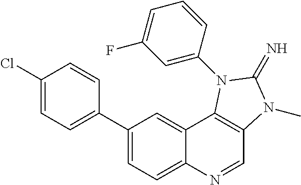 [SMILES: Cn1c(=N)n(-c2cccc(F)c2)c2c3cc(-c4ccc(Cl)cc4)ccc3ncc21]

LCMS: t_{R}=4.048 min in 0-60AB_7 min_220&254 (Xtimate 3 um, C18, 2.1*30 mm), MS (ESI) m/z 403.0 [M+H]⁺.

¹H NMR (400 MHz, DMSO-d₆) δ=9.32 (s, 1H), 8.95 (s, 2H), 8.22 (d, J=8.8 Hz, 1H), 8.00 (dd, J=2.0, 8.8 Hz, 1H), 7.92-7.83 (m, 2H), 7.78-7.70 (m, 2H), 7.52-7.44 (m, 2H), 7.37-7.29 (m, 2H), 6.98 (d, J=1.8 Hz, 1H), 3.90 (s, 3H).

### 3-(8-(4-chlorophenyl)-2-imino-3-methyl-2,3-dihydro-1H-imidazo[4,5-c]quinolin-1-yl)phenol (79)

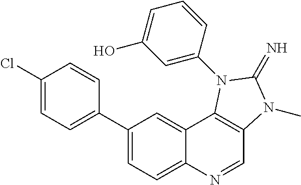 [SMILES: Cn1c(=N)n(-c2cccc(O)c2)c2c3cc(-c4ccc(Cl)cc4)ccc3ncc21]

LCMS: t_{R}=0.717 min in 5-95AB_1.5 MIN_220&254 chromatography (RP-18e, 25-2 mm), MS (ESI) m/z 399.0 [M+H]⁺.

¹H NMR: (400 MHz, DMSO-d₆) δ=10.42 (br s, 1H), 9.33 (s, 1H), 8.90 (br s, 2H), 8.23 (d, J=8.8 Hz, 1H), 8.02 (dd, J=2.1, 8.9 Hz, 1H), 7.65 (t, J=8.0 Hz, 1H), 7.53-7.48 (m, 2H), 7.42-7.37 (m, 2H), 7.29-7.24 (m, 2H), 7.23-7.21 (m, 1H), 7.12 (d, J=1.8 Hz, 1H), 3.92 (s, 3H).

### 8-(4-chlorophenyl)-1-(2-fluorophenyl)-3-methyl-1,3-dihydro-2H-imidazo[4,5-c]quinolin-2-imine (80)

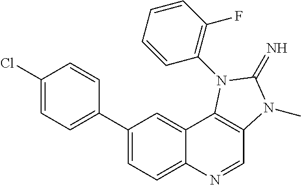 [SMILES: Cn1c(=N)n(-c2ccccc2F)c2c3cc(-c4ccc(Cl)cc4)ccc3ncc21]

LCMS: t_{R}=3.975 min in 0-60 AB_7MIN_220&254 chromatography (Xtimate 3 um, C18, 2.1*30 mm), MS (ESI) m/z 403.0 [M+H]⁺.

¹H NMR: (400 MHz, DMSO-d₆) δ=8.83 (br s, 1H), 8.03 (br d, J=9.0 Hz, 1H), 7.80 (br d, J=8.6 Hz, 3H), 7.65 (br d, J=9.5 Hz, 1H), 7.56 (br d, J=7.1 Hz, 1H), 7.46 (br d, J=8.4 Hz, 2H), 7.32 (br d, J=8.4 Hz, 2H), 7.04 (br s, 1H), 3.55 (s, 3H).

### 8-(4-chlorophenyl)-3-methyl-1-(o-tolyl)-1,3-dihydro-2H-imidazo[4,5-c]quinolin-2-imine (81)

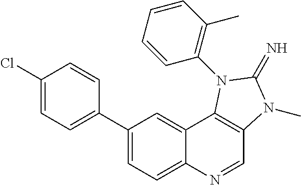 [SMILES: Cc1ccccc1-n1c(=N)n(C)c2cnc3ccc(-c4ccc(Cl)cc4)cc3c21]

LCMS: t_{R}=3.944 min in 0-60AB_7 min_220&254 (Xtimate 3 um, C18, 2.1*30 mm), MS (ESI) m/z 398.9 [M+H]⁺.

¹H NMR (400 MHz, DMSO-d₆) δ=9.34 (s, 1H), 8.96 (s, 2H), 8.21 (d, J=8.8 Hz, 1H), 7.99 (dd, J=2.1, 8.9 Hz, 1H), 7.79-7.69 (m, 3H), 7.65-7.59 (m, 1H), 7.48-7.42 (m, 2H), 7.31-7.25 (m, 2H), 6.81 (d, J=1.8 Hz, 1H), 3.92 (s, 3H), 2.11 (s, 3H).

### 8-(4-chlorophenyl)-1-(2-methoxyphenyl)-3-methyl-1,3-dihydro-2H-imidazo[4,5-c]quinolin-2-imine (82)

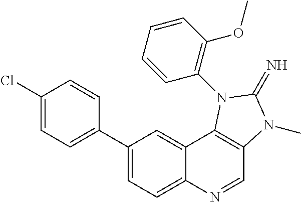 [SMILES: COc1ccccc1-n1c(=N)n(C)c2cnc3ccc(-c4ccc(Cl)cc4)cc3c21]

LCMS: t_{R}=4.626 min in 0-60AB_7 min_220&254 (Xtimate 3 um, C18, 2.1*30 mm), MS (ESI) m/z 414.9 [M+H]⁺.

¹H NMR (400 MHz, DMSO-d₆) δ=9.30 (s, 1H), 8.96 (s, 2H), 8.20 (d, J=9.0 Hz, 1H), 8.00 (dd, J=2.1, 8.9 Hz, 1H), 7.87-7.82 (m, 1H), 7.78 (dd, J=1.5, 7.7 Hz, 1H), 7.51 (d, J=7.7 Hz, 1H), 7.49-7.45 (m, 2H), 7.37-7.30 (m, 3H), 7.01 (d, J=1.8 Hz, 1H), 3.92 (s, 3H), 3.73 (s, 3H).

### 2-(8-(4-chlorophenyl)-2-imino-3-methyl-2,3-dihydro-1H-imidazo[4,5-c]quinolin-1-yl)phenol (83)

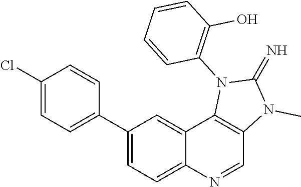 [SMILES: Cn1c(=N)n(-c2ccccc2O)c2c3cc(-c4ccc(Cl)cc4)ccc3ncc21]

LCMS: t_{R}=3.838 min in 0-60AB_7 min_220&254 (Xtimate 3 um, C18, 2.1*30 mm), MS (ESI) m/z 401.9 [M+H]⁺.

¹H NMR (400 MHz, DMSO-d₆) δ=10.37 (br s, 2H), 9.26 (s, 1H), 8.23-8.17 (m, 1H), 8.11 (dd, J=1.8, 9.0 Hz, 1H), 7.61-7.47 (m, 4H), 7.40-7.35 (m, 3H), 7.23-7.18 (m, 1H), 7.12 (dt, J=1.1, 7.6 Hz, 1H), 3.65 (s, 3H).

### 5-(8-(4-chlorophenyl)-2-imino-3-methyl-2,3-dihydro-1H-imidazo[4,5-c]quinolin-1-yl)-2-methoxybenzonitrile (84)

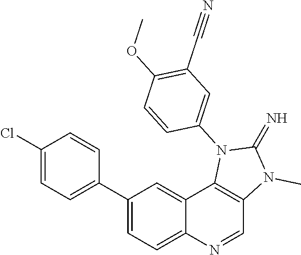 [SMILES: COc1ccc(-n2c(=N)n(C)c3cnc4ccc(-c5ccc(Cl)cc5)cc4c32)cc1C#N]

LCMS: t_{R}=0.682 min in 5-95AB_1.5 MIN_220&254 chromatography (RP-18e, 25-2 mm), MS (ESI) m/z 400.0 [M+H]⁺.

¹H NMR: (400 MHz, DMSO-d₆) δ=9.36 (s, 1H), 8.98 (s, 2H), 8.38 (d, J=2.6 Hz, 1H), 8.26 (d, J=9.0 Hz, 1H), 8.21 (dd, J=2.6, 9.0 Hz, 1H), 8.05 (dd, J=2.1, 8.9 Hz, 1H), 7.74 (d, J=9.0 Hz, 1H), 7.54-7.50 (m, 2H), 7.45-7.40 (m, 2H), 7.09 (d, J=1.8 Hz, 1H), 4.13 (s, 3H), 3.93 (s, 3H).

### 8-(4-chlorophenyl)-3-methyl-1-(3-(piperazin-1-ylmethyl)phenyl)-1,3-dihydro-2H-imidazo[4,5-c]quinolin-2-imine (85)

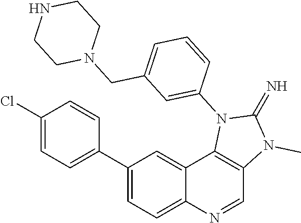 [SMILES: Cn1c(=N)n(-c2cccc(CN3CCNCC3)c2)c2c3cc(-c4ccc(Cl)cc4)ccc3ncc21]

LCMS: t_{R}=2.995 min in 0-60AB_7 min 220&254 (Xtimate C18 2.1*30 mm, 3 um), MS (ESI) m/z 483.0 [M+H]⁺.

¹H NMR (400 MHz, DMSO-d₆) δ=9.33 (s, 1H), 9.27-8.86 (m, 4H), 8.22 (d, J=8.8 Hz, 1H), 7.99 (dd, J=2.1, 8.9 Hz, 1H), 7.92-7.81 (m, 4H), 7.53-7.43 (m, 2H), 7.31-7.25 (m, 2H), 6.89 (d, J=1.8 Hz, 1H), 4.06-4.03 (m, 2H), 3.93 (s, 3H), 3.14-3.02 (m, 4H), 2.95-2.82 (m, 4H).

### 3-(8-(2-hydroxyphenyl)-2-imino-3-methyl-2,3-dihydro-1H-imidazo[4,5-c]quinolin-1-yl)-4-methylbenzonitrile (86)

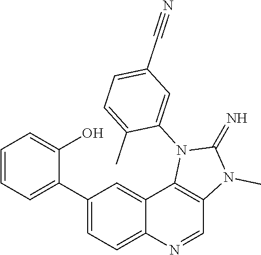 [SMILES: Cc1ccc(C#N)cc1-n1c(=N)n(C)c2cnc3ccc(-c4ccccc4O)cc3c21]

LCMS: t_{R}=3.101 min in 0-60AB_7.0 MIN chromatography (Xtimate 3 um, C18, 2.1*30 mm), MS (ESI) m/z 406.1[M+H]⁺.

¹H NMR (400 MHz, DMSO-d₆) δ=9.61 (s, 1H), 9.36 (s, 1H), 8.95 (s, 2H), 8.33 (d, J=1.5 Hz, 1H), 8.19 (d, J=8.8 Hz, 2H), 7.95 (dd, J=1.9, 8.9 Hz, 1H), 7.88 (d, J=8.2 Hz, 1H), 7.21-7.13 (m, 1H), 7.11-7.04 (m, 2H), 6.92 (d, J=7.3 Hz, 1H), 6.84 (t, J=7.4 Hz, 1H), 3.95 (s, 3H), 2.23 (s, 3H).

### methyl (E)-(8-(4-chlorophenyl)-1-(5-cyano-2-methylphenyl)-3-methyl-1,3-dihydro-2H-imidazo[4,5-c]quinolin-2-ylidene)carbamate (87)

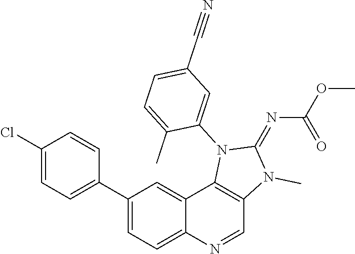 [SMILES: COC(=O)/N=c1\n(C)c2cnc3ccc(-c4ccc(Cl)cc4)cc3c2n1-c1cc(C#N)ccc1C]

LCMS: t_{R}=0.691 min in 5-95AB_1.5 min chromatography (RP-18e, 25-2 mm), MS (ESI) m/z 482.1 [M+H]⁺.

¹H NMR (400 MHz, CHLOROFORM-d) δ=8.98 (s, 1H), 8.25 (d, J=9.04 Hz, 1H), 7.82-7.87 (m, 2H), 7.80 (s, 1H), 7.64 (br d, J=7.94 Hz, 1H), 7.35-7.41 (m, 2H), 7.17-7.22 (m, 2H), 6.98 (d, J=1.76 Hz, 1H), 3.83 (s, 3H), 3.56 (s, 3H), 2.27 (s, 3H).

### tert-butyl (E)-(8-(4-chlorophenyl)-1-(5-cyano-2-methylphenyl)-3-methyl-1,3-dihydro-2H-imidazo[4,5-c]quinolin-2-ylidene)carbamate (88)

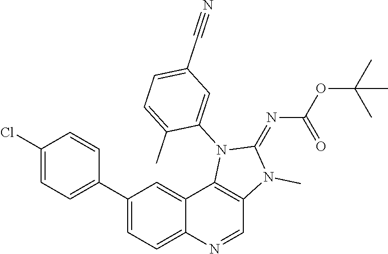 [SMILES: Cc1ccc(C#N)cc1-n1/c(=N/C(=O)OC(C)(C)C)n(C)c2cnc3ccc(-c4ccc(Cl)cc4)cc3c21]

LCMS: t_{R}=1.586 min in 0-60AB_2 min chromatography (XBridge Shield 2.1*50 mm), MS (ESI) m/z 524.3 [M+H]⁺.

¹H NMR (400 MHz, CHLOROFORM-d) δ=8.93 (s, 1H), 8.23 (d, J=8.8 Hz, 1H), 7.84-7.82 (m, 2H), 7.81-7.79 (m, 1H), 7.62 (d, J=7.5 Hz, 1H), 7.40-7.36 (m, 2H), 7.22-7.19 (m, 2H), 7.01 (d, J=1.5 Hz, 1H), 3.80 (s, 3H), 2.31 (s, 3H), 1.29 (s, 9H).

butyl (E)-(8-(4-chlorophenyl)-1-(5-cyano-2-methylphenyl)-3-methyl-1,3-dihydro-2H-imidazo[4,5-c]quinolin-2-ylidene)carbamate (89)

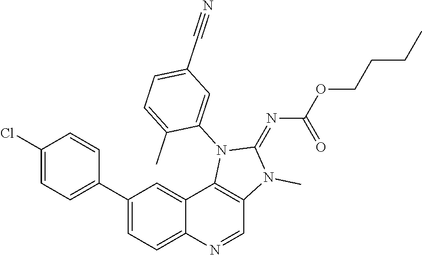 [SMILES: CCCCOC(=O)/N=c1\n(C)c2cnc3ccc(-c4ccc(Cl)cc4)cc3c2n1-c1cc(C#N)ccc1C]

LCMS: t_{R}=5.934 min in 0-60AB_7 min chromatography (Xtimate 3 um, C18, 2.1*30 mm), MS (ESI) m/z 524.1 [M+H]⁺.

¹H NMR (400 MHz, CHLOROFORM-d) δ=8.96 (s, 1H), 8.24 (d, J=8.8 Hz, 1H), 7.85-7.83 (m, 1H), 7.83-7.82 (m, 1H), 7.81-7.80 (m, 1H), 7.63 (d, J=7.9 Hz, 1H), 7.39-7.36 (m, 2H), 7.22-7.19 (m, 2H), 7.00 (d, J=2.0 Hz, 1H), 3.93-3.83 (m, 2H), 3.82 (s, 3H), 2.29 (s, 3H), 1.54-1.48 (m, 2H), 1.36-1.29 (m, 2H), 0.90 (t, J=7.3 Hz, 3H).

### 4-(8-(4-chlorophenyl)-2-imino-3-methyl-2,3-dihydro-1H-imidazo[4,5-c]quinolin-1-yl)picolinonitrile (90)

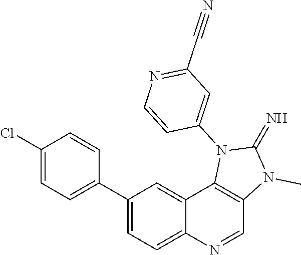 [SMILES: Cn1c(=N)n(-c2ccnc(C#N)c2)c2c3cc(-c4ccc(Cl)cc4)ccc3ncc21]

LCMS: t_{R}=3.708 min in 0-60AB_7 min 220&254 (Xtimate 3 um, C18, 2.1*30 mm), MS (ESI) m/z 410.9 [M+H]⁺.

¹H NMR (400 MHz, DMSO-d₆) δ=9.40 (s, 1H), 9.25 (d, J=5.3 Hz, 1H), 9.15 (br s, 2H), 8.72 (d, J=1.5 Hz, 1H), 8.37 (dd, J=1.8, 5.3 Hz, 1H), 8.29 (d, J=8.8 Hz, 1H), 8.06 (dd, J=1.6, 8.9 Hz, 1H), 7.56-7.49 (m, 2H), 7.49-7.44 (m, 2H), 7.13 (s, 1H), 3.95 (s, 3H).

### 3-(2-imino-3-methyl-8-(pyridin-2-yl)-2,3-dihydro-1H-imidazo[4,5-c]quinolin-1-yl)-4-methylbenzonitrile (91)

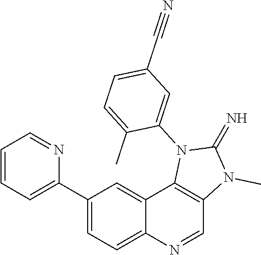 [SMILES: Cc1ccc(C#N)cc1-n1c(=N)n(C)c2cnc3ccc(-c4ccccn4)cc3c21]

LCMS: t_{R}=2.970 min in 0-60AB_7.0 min chromatography (Xtimate, 2.1*30 mm, 3 um), MS (ESI) m/z 390.9 [M+H]⁺.

¹H NMR: (400 MHz, DMSO-d₆): δ=8.82 (s, 1H), 8.53-8.52 (m, 1H), 8.15-8.13 (m, 3H), 8.02-8.00 (m, 1H), 7.81-7.79 (m, 2H), 7.47-7.44 (m, 2H), 7.29 (s, 1H), 3.53 (s, 3H), 2.13 (s, 3H).

### 3-(8-(4-chlorophenyl)-2-imino-3-methyl-2,3-dihydro-1H-imidazo[4,5-c]quinolin-1-yl)-4-methylbenzamide (92)

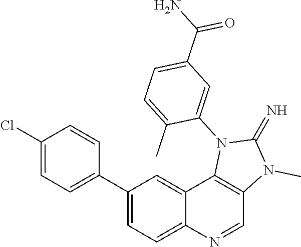 [SMILES: Cc1ccc(C(N)=O)cc1-n1c(=N)n(C)c2cnc3ccc(-c4ccc(Cl)cc4)cc3c21]

LCMS: t_{R}=3.497 min in 0-60AB_7.0 min chromatography (Xtimate, 2.1*30 mm, 3 um), MS (ESI) m/z 441.9 [M+H]⁺.

¹H NMR: (400 MHz, DMSO-d₆): δ=8.81 (s, 1H), 8.10-8.08 (m, 2H), 8.02-7.98 (m, 2H), 7.76-7.73 (m, 1H), 7.67-7.65 (m, 1H), 7.48 (s, 1H), 7.38 (d, J=8.8 Hz, 2H), 7.22 (d, J=8.4 Hz, 2H), 6.82 (s, 1H), 3.53 (s, 3H), 2.10 (s, 3H).

### 3-(8-(4-chlorophenyl)-3-ethyl-2-imino-2,3-dihydro-1H-imidazo[4,5-c]quinolin-1-yl)-4-methylbenzonitrile (93)

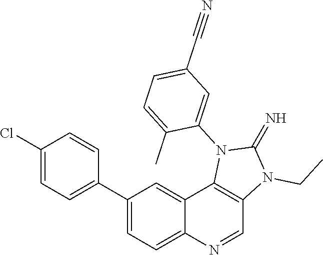 [SMILES: CCn1c(=N)n(-c2cc(C#N)ccc2C)c2c3cc(-c4ccc(Cl)cc4)ccc3ncc21]

LCMS: t_{R}=4.087 min in 0-60 AB_7 min chromatography (Xtimate 3 um, C18, 2.1*30 mm), MS (ESI) m/z 438.0 [M+H]⁺.

¹H NMR: (400 MHz, DMSO-d₆) δ=9.47 (s, 1H), 9.36-9.25 (m, 2H), 8.43 (d, J=1.5 Hz, 1H), 8.31-8.21 (m, 2H), 8.06 (dd, J=2.0, 9.0 Hz, 1H), 7.92 (d, J=8.2 Hz, 1H), 7.48 (d, J=8.4 Hz, 2H), 7.32 (d, J=8.6 Hz, 2H), 6.84 (d, J=2.0 Hz, 1H), 4.54 (q, J=7.1 Hz, 2H), 2.19 (s, 3H), 1.45 (t, J=7.2 Hz, 3H).

### 3-(3-benzyl-8-(4-chlorophenyl)-2-imino-2,3-dihydro-1H-imidazo[4,5-c]quinolin-1-yl)-4-methylbenzonitrile (94)

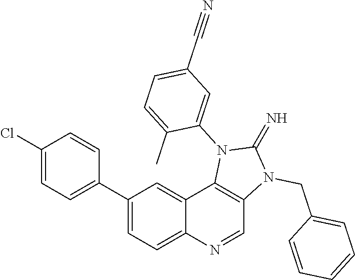 [SMILES: Cc1ccc(C#N)cc1-n1c(=N)n(Cc2ccccc2)c2cnc3ccc(-c4ccc(Cl)cc4)cc3c21]

LCMS: t_{R}=4.593 min in 0-60 AB_7 min chromatography (Xtimate 3 um, C18, 2.1*30 mm), MS (ESI) m/z 500.0 [M+H]⁺.

¹H NMR: (400 MHz, DMSO-d₆) δ=9.56 (br s, 2H), 9.22 (s, 1H), 8.57 (d, J=1.5 Hz, 1H), 8.28 (dd, J=1.8, 7.9 Hz, 1H), 8.23 (d, J=8.8 Hz, 1H), 8.05 (dd, J=2.0, 8.8 Hz, 1H), 7.95 (d, J=8.2 Hz, 1H), 7.53-7.47 (m, 4H), 7.46-7.40 (m, 2H), 7.40-7.36 (m, 1H), 7.35-7.31 (m, 2H), 6.86 (d, J=1.8 Hz, 1H), 5.93-5.81 (m, 2H), 2.23 (s, 3H).

### 3-(8-(4-chlorophenyl)-3-(2-hydroxyethyl)-2-imino-2,3-dihydro-1H-imidazo[4,5-c]quinolin-1-yl)-4-methylbenzonitrile (95)

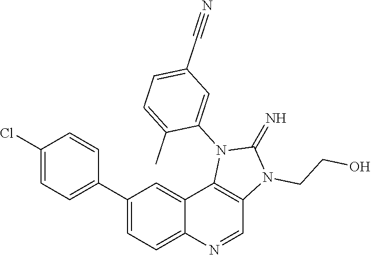 [SMILES: Cc1ccc(C#N)cc1-n1c(=N)n(CCO)c2cnc3ccc(-c4ccc(Cl)cc4)cc3c21]

LCMS: t_{R}=4.707 min in 0-60AB_7 min chromatography (Xtimate 3 um, C18, 2.1*30 mm), MS (ESI) m/z 454.1 [M+H]⁺.

### 8-(4-chlorophenyl)-3-methyl-1-((1r,4r)-4-(piperazin-1-yl)cyclohexyl)-1,3-dihydro-2H-imidazo[4,5-c]quinolin-2-imine (96)

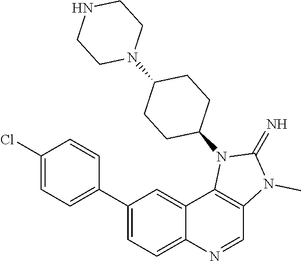 [SMILES: Cn1c(=N)n([C@H]2CC[C@H](N3CCNCC3)CC2)c2c3cc(-c4ccc(Cl)cc4)ccc3ncc21]

LCMS: t_{R}=4.703 min in 0-60AB_7 min chromatography (Xtimate 3 um, C18, 2.1*30 mm), MS (ESI) m/z 475.2 [M+H]⁺.

### 1-([1,4′-bipiperidin]-4-yl)-8-(4-chlorophenyl)-3-methyl-1,3-dihydro-2H-imidazo[4,5-c]quinolin-2-imine (97)

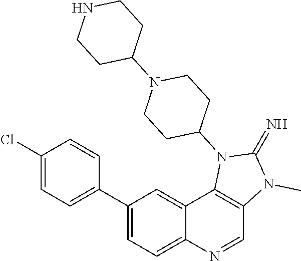 [SMILES: Cn1c(=N)n(C2CCN(C3CCNCC3)CC2)c2c3cc(-c4ccc(Cl)cc4)ccc3ncc21]

LCMS: t_{R}=3.555 min in 0-30AB_7 min_220&254 (Xtimate 3 um, C18, 2.1*30 mm), MS (ESI) m/z 475.1 [M+H]⁺.

¹H NMR (400 MHz, DMSO-d₆) δ=11.32 (br s, 1H), 9.41-9.24 (m, 4H), 9.16 (br d, J=9.0 Hz, 1H), 8.50 (s, 1H), 8.36 (d, J=8.8 Hz, 1H), 8.16 (dd, J=1.5, 8.8 Hz, 1H), 8.00 (d, J=8.6 Hz, 2H), 7.63 (d, J=8.6 Hz, 1H), 7.68-7.58 (m, 1H), 5.85-5.68 (m, 1H), 3.88 (s, 3H), 3.71 (br s, 2H), 3.53-3.42 (m, 6H), 3.29 (br d, J=11.9 Hz, 2H), 2.97 (br d, J=11.9 Hz, 2H), 2.39-2.27 (m, 2H), 2.18-1.94 (m, 2H).

### 8-(4-chlorophenyl)-3-methyl-1-(3-(piperazin-1-yl)cyclobutyl)-1,3-dihydro-2H-imidazo[4,5-c]quinolin-2-imine (98)

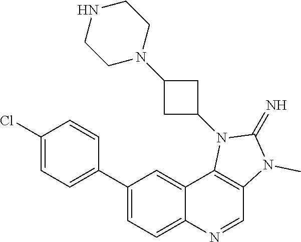 [SMILES: Cn1c(=N)n(C2CC(N3CCNCC3)C2)c2c3cc(-c4ccc(Cl)cc4)ccc3ncc21]

LCMS: t_{R}=4.189 min in 0-30AB_7 min_220&254 (Xtimate 3 um, C18, 2.1*30 mm), MS (ESI) m/z 447.1 [M+H]⁺.

¹H NMR (400 MHz, DMSO-d₆) δ=9.63 (br s, 2H), 9.33 (s, 1H), 9.25 (br s, 2H), 8.36 (s, 1H), 8.30 (d, J=8.8 Hz, 1H), 8.14 (dd, J=1.8, 9.0 Hz, 1H), 8.17-8.08 (m, 1H), 7.98 (d, J=8.5 Hz, 2H), 7.63 (d, J=8.5 Hz, 2H), 5.50 (br t, J=7.8 Hz, 1H), 3.89 (s, 5H), 3.69-3.34 (m, 4H), 3.33-2.91 (m, 7H).

### 8-(4-chlorophenyl)-1-(2,6-dimethylphenyl)-3-methyl-1,3-dihydro-2H-imidazo[4,5-c]quinolin-2-imine (99)

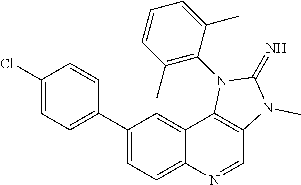 [SMILES: Cc1cccc(C)c1-n1c(=N)n(C)c2cnc3ccc(-c4ccc(Cl)cc4)cc3c21]

LCMS: t_{R}=4.643 min in 0-60AB_7 min_220&254 chromatography (Xtimate, 2.1*30 mm, 3 um), MS (ESI) m/z 412.9 [M+H]⁺.

¹H NMR (400 MHz, DMSO-d₆) δ=9.37 (s, 1H), 9.07 (s, 2H), 8.23 (d, J=9.0 Hz, 1H), 8.02 (dd, J=2.0, 9.0 Hz, 1H), 7.72-7.62 (m, 1H), 7.55-7.42 (m, 4H), 7.33-7.24 (m, 2H), 6.81 (d, J=1.8 Hz, 1H), 3.96 (s, 3H), 2.03 (s, 6H).

### (8-(4-chlorophenyl)-1-(5-cyano-2-methylphenyl)-3-methyl-1,3-dihydro-2H-imidazo[4,5-c]quinolin-2-ylidene)sulfonamide

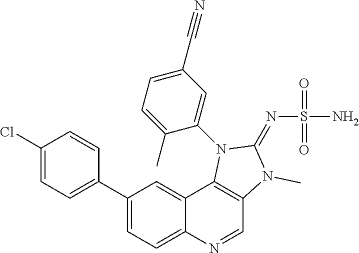 [SMILES: Cc1ccc(C#N)cc1-n1/c(=N/S(N)(=O)=O)n(C)c2cnc3ccc(-c4ccc(Cl)cc4)cc3c21]

LCMS: t_{R}=4.374 min in 0-60AB_7 min chromatography (Xtimate 3 um, C18, 2.1*30 mm), MS (ESI) m/z 502.9 [M+H]⁺.

¹H NMR (400 MHz, DMSO-d₆) δ=9.21 (s, 1H), 8.27 (d, J=1.8 Hz, 1H), 8.19 (d, J=8.8 Hz, 1H), 8.13 (dd, J=1.7, 8.0 Hz, 1H), 7.98 (dd, J=2.0, 8.8 Hz, 1H), 7.82 (d, J=8.2 Hz, 1H), 7.52-7.49 (m, 2H), 7.34-7.31 (m, 2H), 6.82 (d, J=1.5 Hz, 1H), 6.51 (s, 2H), 4.08 (s, 3H), 2.17 (s, 3H).

### (8-(6-aminopyridin-3-yl)-1-(5-cyano-2-methylphenyl)-3-methyl-1,3-dihydro-2H-imidazo[4,5-c]quinolin-2-ylidene)sulfonamide

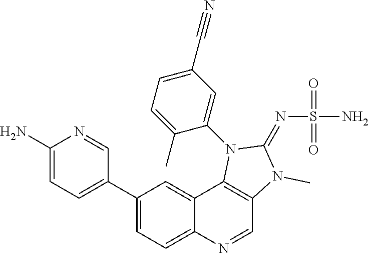 [SMILES: Cc1ccc(C#N)cc1-n1/c(=N/S(N)(=O)=O)n(C)c2cnc3ccc(-c4ccc(N)nc4)cc3c21]

LCMS: t_{R}=2.309 min in 10-80CD_7MIN_220&254 (XBrige Shield RP18 2.1*50 mm), MS (ESI) m/z 485.1 [M+H]⁺.

¹H NMR (400 MHz, DMSO-d₆) δ=9.14 (s, 1H), 8.28 (d, J=1.5 Hz, 1H), 8.16-8.07 (m, 2H), 7.90 (dd, J=1.9, 8.9 Hz, 1H), 7.86-7.79 (m, 2H), 7.38 (dd, J=2.5, 8.7 Hz, 1H), 6.72 (d, J=2.0 Hz, 1H), 6.53-6.42 (m, 3H), 6.25 (s, 2H), 4.07 (s, 3H), 2.17 (s, 3H).

### 8-(4-chlorophenyl)-1-cyclohexyl-3-methyl-1,3-dihydro-2H-imidazo[4,5-c]quinolin-2-imine (102)

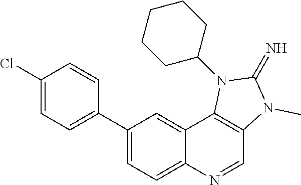 [SMILES: Cn1c(=N)n(C2CCCCC2)c2c3cc(-c4ccc(Cl)cc4)ccc3ncc21]

LCMS: t_{R}=4.231 min in 0-60AB_7 min_220&254 chromatography (Xtimate, 2.1*30 mm, 3 um), MS (ESI) m/z 391.0 [M+H]⁺.

¹H NMR (400 MHz, DMSO-d₆) δ=9.26 (s, 1H), 9.00 (br s, 2H), 8.61-8.31 (m, 1H), 8.27 (d, J=8.8 Hz, 1H), 8.06 (dd, J=1.8, 8.8 Hz, 1H), 7.83 (d, J=8.4 Hz, 2H), 7.68-7.59 (m, 2H), 4.94 (br s, 1H), 3.85 (s, 3H), 2.48-2.38 (m, 2H), 2.13-1.91 (m, 4H), 1.74 (br d, J=11.0 Hz, 1H), 1.56 (q, J=12.8 Hz, 2H), 1.46-1.33 (m, 1H).

### 3-(2-imino-8-(6-methoxypyridin-3-yl)-3-methyl-2,3-dihydro-1H-imidazo[4,5-c]quinolin-1-yl)-4-methylbenzonitrile (103)

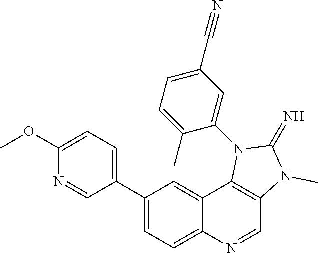

LCMS: t_{R}=3.078 min in 0-60AB_7 min_220&254 (Xtimate 3 um, C18, 2.1*30 mm), MS (ESI) m/z 421.0 [M+H]⁺.

¹H NMR (400 MHz, DMSO-d₆) δ=8.84 (s, 1H), 8.19 (br s, 1H), 8.10 (br d, J=2.6 Hz, 2H), 8.03 (d, J=9.0 Hz, 1H), 7.87-7.76 (m, 3H), 7.61 (dd, J=2.5, 8.7 Hz, 1H), 6.86 (d, J=8.6 Hz, 1H), 6.80 (br d, J=1.5 Hz, 1H), 3.88 (s, 3H), 3.55 (s, 3H), 2.18 (s, 3H).

### 1-(4-(8-(4-chlorophenyl)-2-imino-3-methyl-2,3-dihydro-1H-imidazo[4,5-c]quinolin-1-yl)piperidin-1-yl)propan-1-one (104)

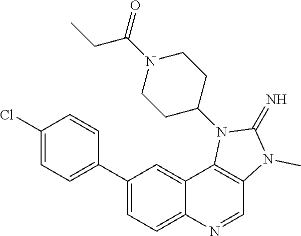 [SMILES: CCC(=O)N1CCC(n2c(=N)n(C)c3cnc4ccc(-c5ccc(Cl)cc5)cc4c32)CC1]

LCMS: t_{R}=3.816 min in 0-60AB_7 min_220&254 (Xtimate 3 um, C18, 2.1*30 mm), MS (ESI) m/z 448.0 [M+H]⁺.

¹H NMR (400 MHz, DMSO-d₆) δ=8.74-8.66 (m, 1H), 8.34 (br s, 1H), 8.06 (d, J=8.8 Hz, 1H), 7.85 (br d, J=9.0 Hz, 1H), 7.78 (br s, 2H), 7.62-7.50 (m, 2H), 5.85 (br s, 1H), 5.18 (s, 1H), 4.79-4.60 (m, 1H), 4.17-3.97 (m, 1H), 3.49-3.42 (m, 3H), 3.26 (br s, 1H), 3.21 (br d, J=11.5 Hz, 1H), 2.83-2.64 (m, 2H), 2.44-2.26 (m, 2H), 1.95-1.80 (m, 2H), 1.07-0.91 (m, 3H).

### 3-(2-imino-3-methyl-8-(quinolin-3-yl)-2,3-dihydro-1H-imidazo[4,5-c]quinolin-1-yl)-4-methylbenzonitrile (105)

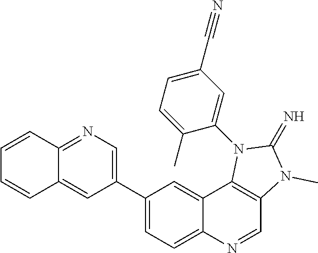 [SMILES: Cc1ccc(C#N)cc1-n1c(=N)n(C)c2cnc3ccc(-c4cnc5ccccc5c4)cc3c21]

LCMS: t_{R}=2.312 min in 10-80AB_7 min_220&254 (Xtimate 3 um, C18, 2.1*30 mm), MS (ESI) m/z 441.0 [M+H]⁺.

¹H NMR (400 MHz, DMSO-d₆) δ=9.41 (s, 1H), 9.05 (s, 2H), 8.78 (d, J=2.4 Hz, 1H), 8.40-8.39 (m, 2H), 8.34-8.30 (m, 2H), 8.27-8.24 (m, 1H), 8.04 (d, J=8.0 Hz, 1H), 8.01-7.96 (m, 2H), 7.82-7.78 (m, 1H), 7.70-7.66 (m, 1H), 7.05 (d, J=2.0 Hz, 1H), 3.94 (s, 3H), 2.24 (s, 3H).

### 3-(8-(6-(3-(dimethylamino)propoxy)pyridin-3-yl)-2-imino-3-methyl-2,3-dihydro-1H-imidazo[4,5-c]quinolin-1-yl)-4-methylbenzonitrile (106)

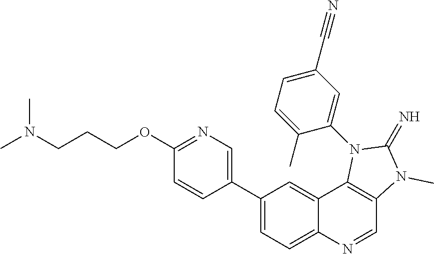

LCMS: t_{R}=2.470 min in 0-60AB_7 min_220&254 chromatography (Xtimate C18 2.1*30 mm, 3 um), MS (ESI) m/z 492.1 [M+H]⁺.

¹H NMR: (400 MHz, DMSO-d₆) δ=10.82 (br s, 1H), 9.69-9.55 (m, 3H), 8.43-8.35 (m, 2H), 8.25 (dd, J=1.5, 7.9 Hz, 1H), 8.16 (dd, J=2.0, 9.0 Hz, 1H), 8.11 (d, J=2.4 Hz, 1H), 7.93 (d, J=8.2 Hz, 1H), 7.66 (dd, J=2.6, 8.6 Hz, 1H), 6.89 (d, J=8.6 Hz, 1H), 6.84 (d, J=1.5 Hz, 1H), 4.33 (t, J=6.2 Hz, 2H), 4.07-3.95 (m, 3H), 3.19-3.14 (m, 2H), 2.73 (d, J=4.9 Hz, 6H), 2.22 (s, 3H), 2.23-2.10 (m, 2H).

### 3-(2-imino-8-(1H-indazol-5-yl)-3-methyl-2,3-dihydro-1H-imidazo[4,5-c]quinolin-1-yl)-4-methylbenzonitrile (107)

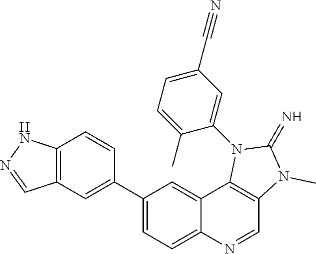 [SMILES: Cc1ccc(C#N)cc1-n1c(=N)n(C)c2cnc3ccc(-c4ccc5[nH]ncc5c4)cc3c21]

LCMS: t_{R}=2.881 min in 0-60AB_7 min_220&254 (Xtimate 3 um, C18, 2.1*30 mm), MS (ESI) m/z 430.0 [M+H]⁺.

¹H NMR (400 MHz, DMSO-d₆): δ=13.16 (br s, 1H), 8.80 (br s, 1H), 8.25 (br s, 1H), 8.16-8.06 (m, 2H), 8.01 (d, J=9.0 Hz, 1H), 7.87-7.78 (m, 2H), 7.68 (s, 1H), 7.53 (d, J=8.6 Hz, 1H), 7.24 (dd, J=1.7, 8.7 Hz, 1H), 6.86 (s, 1H), 3.54 (s, 3H), 2.16 (s, 3H).

### 5-(8-(4-chlorophenyl)-2-imino-3-methyl-2,3-dihydro-1H-imidazo[4,5-c]quinolin-1-yl)-4-methyl-2-(4-methylpiperazin-1-yl)benzonitrile (108)

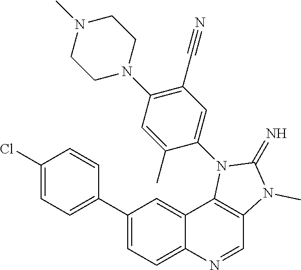 [SMILES: Cc1cc(N2CCN(C)CC2)c(C#N)cc1-n1c(=N)n(C)c2cnc3ccc(-c4ccc(Cl)cc4)cc3c21]

LCMS: t_{R}=3.099 min in 0-60AB_7 min chromatography (Xtimate 3 um, C18, 2.1*30 mm), MS (ESI) m/z 522.1 [M+H]⁺.

¹H NMR: (DMSO-d₆ 400 MHz): δ=9.39 (s, 1H), 9.22 (s, 2H), 8.28 (s, 1H), 8.26 (s, 1H), 8.08 (dd, J=2.1, 8.9 Hz, 1H), 7.61 (s, 1H), 7.54 (d, J=8.6 Hz, 2H), 7.39 (d, J=8.8 Hz, 2H), 7.00 (d, J=1.8 Hz, 1H), 3.97 (s, 3H), 3.77-3.65 (m, 2H), 3.64-3.55 (m, 2H), 3.36 (br d, J=12.1 Hz, 2H), 3.33-3.23 (m, 2H), 2.88 (d, J=4.2 Hz, 3H), 2.16 (s, 3H).

### 2-(4-acetylpiperazin-1-yl)-5-(8-(4-chlorophenyl)-2-imino-3-methyl-2,3-dihydro-1H-imidazo[4,5-c]quinolin-1-yl)-4-methylbenzonitrile (109)

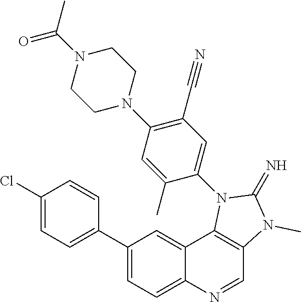 [SMILES: CC(=O)N1CCN(c2cc(C)c(-n3c(=N)n(C)c4cnc5ccc(-c6ccc(Cl)cc6)cc5c43)cc2C#N)CC1]

LCMS: t_{R}=3.929 min in 0-60AB_7 min chromatography (Xtimate 3 um, C18, 2.1*30 mm), MS (ESI) m/z 551.0 [M+H]⁺.

¹H NMR: (DMSO-d₆ 400 MHz): δ=9.04 (s, 1H), 8.14 (s, 1H), 8.12 (s, 1H), 8.09 (s, 1H), 7.92 (dd, J=2.0, 9.0 Hz, 1H), 7.51-7.47 (m, 2H), 7.41-7.38 (m, 3H), 7.06 (d, J=1.8 Hz, 1H), 3.73-3.68 (m, 4H), 3.63 (s, 3H), 3.39 (br s, 4H), 2.12 (s, 3H), 2.11 (s, 3H).

### 2-(azetidin-1-yl)-5-(8-(4-chlorophenyl)-2-imino-3-methyl-2,3-dihydro-1H-imidazo[4,5-c]quinolin-1-yl)-4-methylbenzonitrile (110)

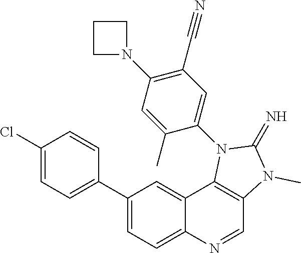 [SMILES: Cc1cc(N2CCC2)c(C#N)cc1-n1c(=N)n(C)c2cnc3ccc(-c4ccc(Cl)cc4)cc3c21]

LCMS: t_{R}=4.295 min in 0-60AB_7 min_220&254 (Xtimate C18 2.1*30 mm, 3 um), MS (ESI) m/z 479.0 [M+H]⁺.

¹H NMR (400 MHz, DMSO-d₆) δ=9.36 (s, 1H), 9.03 (s, 2H), 8.26 (d, J=8.8 Hz, 1H), 8.08 (dd, J=2.0, 8.8 Hz, 1H), 7.98 (s, 1H), 7.58 (d, J=8.5 Hz, 2H), 7.48 (d, J=7.9 Hz, 2H), 7.10 (d, J=2.0 Hz, 1H), 6.85 (s, 1H), 4.30 (sxt, J=8.1 Hz, 4H), 3.94 (s, 3H), 2.55-2.52 (m, 2H), 2.06 (s, 3H).

### 5-(8-(4-chlorophenyl)-2-imino-3-methyl-2,3-dihydro-1H-imidazo[4,5-c]quinolin-1-yl)-4-methyl-2-(pyrrolidin-1-yl)benzonitrile (111)

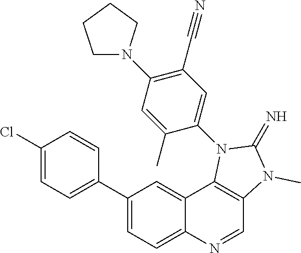 [SMILES: Cc1cc(N2CCCC2)c(C#N)cc1-n1c(=N)n(C)c2cnc3ccc(-c4ccc(Cl)cc4)cc3c21]

LCMS: t_{R}=4.436 min in 0-60AB_7 min_220&254 (Xtimate C18 2.1*30 mm, 3 um), MS (ESI) m/z 493.0 [M+H]⁺.

¹H NMR (400 MHz, DMSO-d₆) δ=9.40 (s, 1H), 9.19 (s, 2H), 8.27 (d, J=9.0 Hz, 1H), 8.09 (d, J=2.0 Hz, 1H), 7.97 (s, 1H), 7.50-7.46 (m, 2H), 7.45-7.40 (m, 2H), 7.10 (d, J=2.0 Hz, 1H), 7.05 (s, 1H), 3.94 (s, 3H), 3.65 (br d, J=5.1 Hz, 4H), 2.09-1.96 (m, 7H).

### 5-(8-(4-chlorophenyl)-2-imino-3-methyl-2,3-dihydro-1H-imidazo[4,5-c]quinolin-1-yl)-4-methyl-2-(piperidin-1-yl)benzonitrile (112)

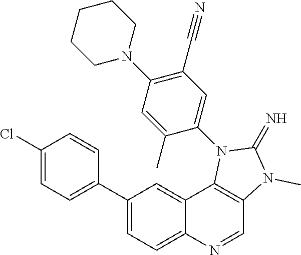 [SMILES: Cc1cc(N2CCCCC2)c(C#N)cc1-n1c(=N)n(C)c2cnc3ccc(-c4ccc(Cl)cc4)cc3c21]

LCMS: t_{R}=4.721 min in 0-60AB_7 min_220&254 (Xtimate C18 2.1*30 mm, 3 um), MS (ESI) m/z 507.0 [M+H]⁺.

¹H NMR (400 MHz, DMSO-d₆) δ=9.34 (s, 1H), 9.01 (br s, 2H), 8.23 (d, J=8.6 Hz, 1H), 8.12 (s, 1H), 8.03 (d, J=9.3 Hz, 1H), 7.48-7.44 (m, 2H), 7.42-7.37 (m, 3H), 6.97 (s, 1H), 3.91 (s, 3H), 3.44-3.39 (m, 4H), 2.11 (s, 3H), 1.69 (br d, J=19.2 Hz, 6H).

### 5-(8-(4-chlorophenyl)-2-imino-3-methyl-2,3-dihydro-1H-imidazo[4,5-c]quinolin-1-yl)-2-((2-(dimethylamino)ethyl)amino)-4-methylbenzonitrile (113)

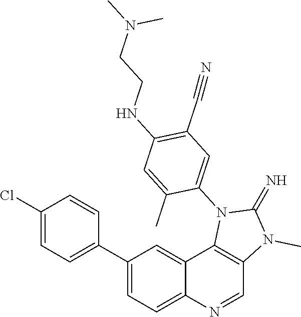 [SMILES: Cc1cc(NCCN(C)C)c(C#N)cc1-n1c(=N)n(C)c2cnc3ccc(-c4ccc(Cl)cc4)cc3c21]

LCMS: t_{R}=4.606 min in 10-80CD_7MIN_220&254 (Xtimate 2.1*30 mm, 3 um), MS (ESI) m/z 510.0 [M+H]⁺.

¹H NMR (400 MHz, DMSO-d₆) δ=9.72 (br, 1H), 9.34 (s, 1H), 9.00 (s, 2H), 8.24 (d, J=8.8 Hz, 1H), 8.05 (d, J=8.8 Hz, 1H), 7.99 (s, 1H), 7.52 (d, J=8.4 Hz, 2H), 7.43 (d, J=8.4 Hz, 2H), 7.16 (s, 1H), 7.12 (s, 1H), 6.99-6.94 (m, 1H), 3.91 (s, 3H), 3.73-3.66 (m, 2H), 3.31-3.24 (m, 2H), 2.89 (br s, 6H), 2.08 (s, 3H).

### 5-(8-(4-chlorophenyl)-2-imino-3-methyl-2,3-dihydro-1H-imidazo[4,5-c]quinolin-1-yl)-2-(4-hydroxypiperidin-1-yl)-4-methylbenzonitrile (114)

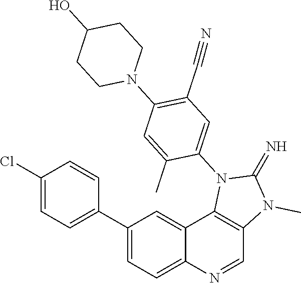 [SMILES: Cc1cc(N2CCC(O)CC2)c(C#N)cc1-n1c(=N)n(C)c2cnc3ccc(-c4ccc(Cl)cc4)cc3c21]

LCMS: t_{R}=3.841 min in 10-80CD_7.0 min chromatography (Xtimate 3 um, C18, 2.1*30 mm), MS (ESI) m/z 523.0 [M+H]⁺.

¹H NMR: (DMSO-d₆ 400 MHz): δ=9.46 (s, 1H), 9.30 (br s, 2H), 8.32 (d, J=8.8 Hz, 1H), 8.16 (s, 1H), 8.12 (dd, J=2.0, 8.8 Hz, 1H), 7.53-7.48 (m, 2H), 7.47 (s, 1H), 7.44-7.40 (m, 2H), 7.03 (d, J=1.8 Hz, 1H), 3.99 (s, 3H), 3.81 (dt, J=3.9, 8.2 Hz, 1H), 3.69 (br s, 1H), 3.66-3.65 (m, 1H), 3.67-3.64 (m, 1H), 3.53-3.52 (m, 1H), 3.48-3.40 (m, 1H), 3.29-3.13 (m, 2H), 2.14 (s, 3H), 1.96 (br d, J=8.5 Hz, 2H), 1.71-1.56 (m, 2H).

### 5-(8-(4-chlorophenyl)-2-imino-3-methyl-2,3-dihydro-1H-imidazo[4,5-c]quinolin-1-yl)-2-(4-(2-hydroxyethyl)piperazin-1-yl)-4-methylbenzonitrile (115)

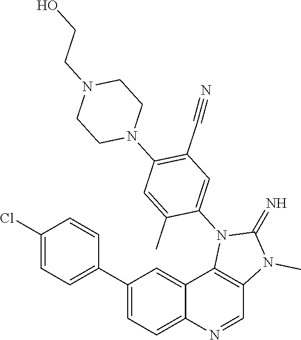 [SMILES: Cc1cc(N2CCN(CCO)CC2)c(C#N)cc1-n1c(=N)n(C)c2cnc3ccc(-c4ccc(Cl)cc4)cc3c21]

LCMS: t_{R}=2.989 in 0-60AB_7.0 min chromatography (Xtimate 3 um, C18, 2.1*30 mm), MS (ESI) m/z 552.1 [M+H]⁺.

¹H NMR: (DMSO-d6 400 MHz): 9.43 (s, 1H), 9.29 (br s, 2H), 8.33-8.27 (m, 2H), 8.10 (d, J=2.0 Hz, 1H), 8.13 (br d, J=2.0 Hz, 1H), 7.62 (s, 1H), 7.57 (d, J=8.5 Hz, 2H), 7.42 (d, J=8.5 Hz, 2H), 7.04 (d, J=1.8 Hz, 1H), 4.00 (s, 3H), 3.97-3.73 (m, 8H), 2.19 (s, 3H).

### (1-(5-cyano-2-methylphenyl)-8-(3-fluorophenyl)-3-methyl-1,3-dihydro-2H-imidazo[4,5-c]quinolin-2-ylidene)sulfonamide

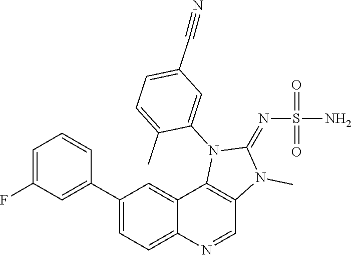 [SMILES: Cc1ccc(C#N)cc1-n1/c(=N/S(N)(=O)=O)n(C)c2cnc3ccc(-c4cccc(F)c4)cc3c21]

LCMS: t_{R}=3.969 min in 0-60AB_7 min chromatography (Xtimate 3 um, C18, 2.1*30 mm), MS (ESI) m/z 487.0 [M+H]⁺.

¹H NMR: (DMSO-d₆ 400 MHz): δ=9.46 (s, 1H), 8.36 (d, J=8.8 Hz, 1H), 8.31 (d, J=1.5 Hz, 1H), 8.18 (ddd, J=1.8, 8.5, 12.6 Hz, 2H), 7.87 (d, J=8.0 Hz, 1H), 7.54-7.47 (m, 1H), 7.30-7.23 (m, 1H), 7.19-7.13 (m, 2H), 6.90 (d, J=1.8 Hz, 1H), 4.11 (s, 3H), 2.22 (s, 3H).

### 5-(8-(4-chlorophenyl)-2-imino-3-methyl-2,3-dihydro-1H-imidazo[4,5-c]quinolin-1-yl)-2-(2,6-dimethylmorpholino)-4-methylbenzonitrile (117)

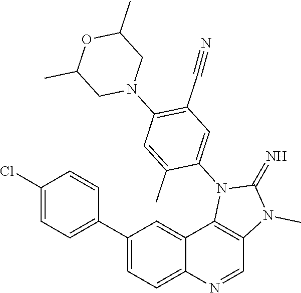 [SMILES: Cc1cc(N2CC(C)OC(C)C2)c(C#N)cc1-n1c(=N)n(C)c2cnc3ccc(-c4ccc(Cl)cc4)cc3c21]

LCMS: t_{R}=4.794 min in 10-80CD_7 min_220&254 (Xtimate 2.1*30 mm, 3 um), MS (ESI) m/z 537.2 [M+H]⁺.

¹H NMR (400 MHz, METHANOL-d₄) δ=8.78 (s, 1H), 8.04 (d, J=9.0 Hz, 1H), 7.84 (s, 1H), 7.79 (dd, J=2.0, 9.0 Hz, 1H), 7.39-7.34 (m, 2H), 7.34-7.25 (m, 3H), 6.91 (d, J=1.5 Hz, 1H), 3.98-3.86 (m, 2H), 3.65 (s, 3H), 3.64-3.54 (m, 2H), 2.72 (dd, J=10.6, 11.2 Hz, 1H), 2.63-2.56 (m, 1H), 2.11 (s, 3H), 1.28 (t, J=6.4 Hz, 6H).

### 5-(8-(4-chlorophenyl)-2-imino-3-methyl-2,3-dihydro-1H-imidazo[4,5-c]quinolin-1-yl)-4-methyl-2-(4-(methylsulfonyl)piperazin-1-yl)benzonitrile (118)

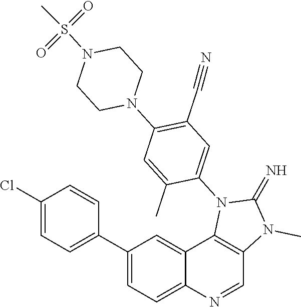 [SMILES: Cc1cc(N2CCN(S(C)(=O)=O)CC2)c(C#N)cc1-n1c(=N)n(C)c2cnc3ccc(-c4ccc(Cl)cc4)cc3c21]

LCMS: t_{R}=4.046 min in 0-60AB_7 min_220&254 (Xtimate C18 2.1*30 mm, 3 um), MS (ESI) m/z 586.0 [M+H]⁺.

¹H NMR (400 MHz, DMSO-d₆) δ=9.48 (s, 1H), 9.39 (br s, 2H), 8.34 (d, J=9.0 Hz, 1H), 8.26 (s, 1H), 8.13 (dd, J=2.0, 9.0 Hz, 1H), 7.58-7.52 (m, 3H), 7.41 (d, J=8.5 Hz, 2H), 7.02 (d, J=1.8 Hz, 1H), 4.01 (s, 3H), 3.55-3.50 (m, 2H), 3.46-3.38 (m, 6H), 3.02 (s, 3H), 2.18 (s, 3H).

### 5-(8-(4-chlorophenyl)-2-imino-3-methyl-2,3-dihydro-1H-imidazo[4,5-c]quinolin-1-yl)-4-methyl-2-(2-methylmorpholino)benzonitrile (119)

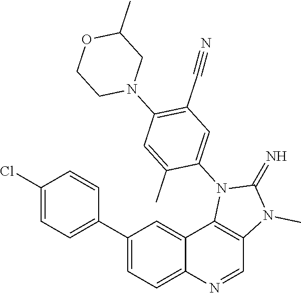 [SMILES: Cc1cc(N2CCOC(C)C2)c(C#N)cc1-n1c(=N)n(C)c2cnc3ccc(-c4ccc(Cl)cc4)cc3c21]

LCMS: t_{R}=4.267 min in 0-60AB_7 min_220&254 (Xtimate C18 2.1*30 mm, 3 um), MS (ESI) m/z 523.0 [M+H]⁺.

¹H NMR (400 MHz, DMSO-d₆) δ=9.39 (s, 1H), 9.21 (d, J=7.2 Hz, 2H), 8.27 (d, J=8.8 Hz, 1H), 8.19 (d, J=6.6 Hz, 1H), 8.06 (ddd, J=2.2, 4.8, 8.9 Hz, 1H), 7.52-7.43 (m, 3H), 7.40-7.36 (m, 2H), 7.01-6.87 (m, 1H), 4.06-3.93 (m, 4H), 3.82-3.62 (m, 2H), 3.49-3.41 (m, 2H), 3.18-2.96 (m, 1H), 2.88-2.66 (m, 1H), 2.13 (d, J=2.4 Hz, 3H), 1.20 (t, J=6.5 Hz, 3H).

### 5-(8-(4-chlorophenyl)-2-imino-3-methyl-2,3-dihydro-1H-imidazo[4,5-c]quinolin-1-yl)-4-methyl-2-((4aS,7aS)-octahydro-6H-pyrrolo[3,4-b]pyridin-6-yl)benzonitrile (120)

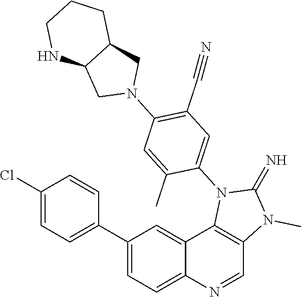 [SMILES: Cc1cc(N2C[C@@H]3CCCN[C@@H]3C2)c(C#N)cc1-n1c(=N)n(C)c2cnc3ccc(-c4ccc(Cl)cc4)cc3c21]

LCMS: t_{R}=3.271 min in 0-60AB_7 min chromatography (Xtimate 3 um, C18, 2.1*30 mm), MS (ESI) m/z 548.1 [M+H]⁺.

¹H NMR: (DMSO-d₆ 400 MHz): δ=9.37 (s, 1H), 8.99 (br d, J=16.3 Hz, 2H), 8.66 (br s, 1H), 8.27 (d, J=8.8 Hz, 1H), 8.12-8.01 (m, 2H), 7.58-7.52 (m, 2H), 7.50-7.42 (m, 2H), 7.24-7.10 (m, 2H), 4.12-4.01 (m, 2H), 3.94 (d, J=2.5 Hz, 3H), 3.90-3.71 (m, 4H), 3.09 (br s, 1H), 2.76 (br s, 1H), 2.11 (d, J=7.3 Hz, 3H), 1.77 (br s, 4H).

### 5-(8-(4-chlorophenyl)-2-imino-3-methyl-2,3-dihydro-1H-imidazo[4,5-c]quinolin-1-yl)-4-methyl-2-(2-oxa-6-azaspiro[3.3]heptan-6-yl)benzonitrile (121)

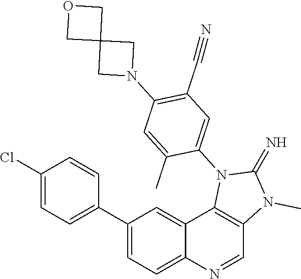 [SMILES: Cc1cc(N2CC3(COC3)C2)c(C#N)cc1-n1c(=N)n(C)c2cnc3ccc(-c4ccc(Cl)cc4)cc3c21]

LCMS: t_{R}=3.984 min in 0-60AB_7.0 min chromatography (Xtimate 3 um, C18, 2.1*30 mm), MS (ESI) m/z 521.0 [M+H]⁺.

¹H NMR: (DMSO-d₆ 400 MHz): δ=8.80 (br s, 1H), 7.99 (br s, 1H), 7.79 (br s, 2H), 7.57-7.26 (m, 6H), 7.01-6.91 (m, 1H), 6.76 (br s, 1H), 4.79 (br s, 4H), 4.39 (br d, J=9.3 Hz, 4H), 3.51 (br s, 3H), 1.97 (br s, 3H).

### 8-(4-chlorophenyl)-3-methyl-1-(2-methyl-5-(methylsulfonyl)phenyl)-1,3-dihydro-2H-imidazo[4,5-c]quinolin-2-imine (122)

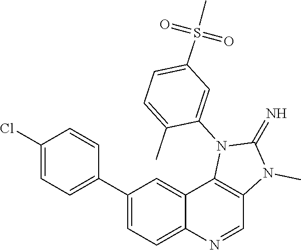 [SMILES: Cc1ccc(S(C)(=O)=O)cc1-n1c(=N)n(C)c2cnc3ccc(-c4ccc(Cl)cc4)cc3c21]

LCMS: t_{R}=3.672 min in 0-60 AB_7 min chromatography (Xtimate 3 um, C18, 2.1*30 mm), MS (ESI) m/z 476.9 [M+H]⁺.

¹H NMR (400 MHz, DMSO-d₆) δ=9.36 (s, 1H), 9.11 (br s, 2H), 8.43 (s, 1H), 8.33 (br d, J=9.7 Hz, 1H), 8.24 (d, J=9.0 Hz, 1H), 7.99 (dd, J=8.7, 12.9 Hz, 2H), 7.45 (d, J=8.4 Hz, 2H), 7.30 (d, J=8.6 Hz, 2H), 6.78 (s, 1H), 3.93 (s, 3H), 3.27 (s, 3H), 2.22 (s, 3H).

### N-(4-(8-(4-chlorophenyl)-2-imino-3-methyl-2,3-dihydro-1H-imidazo[4,5-c]quinolin-1-yl)-2-cyano-5-methylphenyl)methanesulfonamide (123)

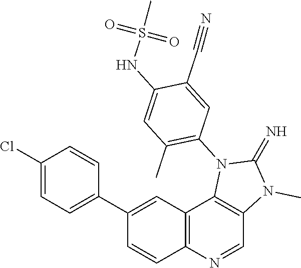 [SMILES: Cc1cc(NS(C)(=O)=O)c(C#N)cc1-n1c(=N)n(C)c2cnc3ccc(-c4ccc(Cl)cc4)cc3c21]

LCMS: t_{R}=3.930 min in 0-60AB_7.0 MIN chromatography (Xtimate C18 2.1*30 mm, 3 um), MS (ESI) m/z 516.9 [M+H]⁺.

¹H NMR (400 MHz, DMSO-d₆) δ=9.31 (s, 1H), 8.96 (br s, 2H), 8.28-8.18 (m, 1H), 8.09 (dd, J=2.0, 9.0 Hz, 1H), 7.71 (s, 1H), 7.56-7.45 (m, 5H), 7.27 (s, 1H), 3.91 (s, 3H), 2.79 (s, 3H), 1.97 (s, 3H).

### 5-(8-(4-chlorophenyl)-2-imino-3-methyl-2,3-dihydro-1H-imidazo[4,5-c]quinolin-1-yl)-4-methyl-2-(3-methylpiperazin-1-yl)benzonitrile (124)

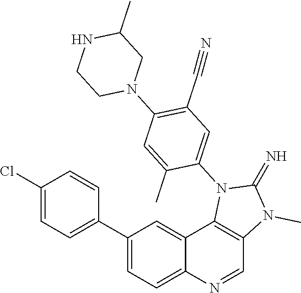 [SMILES: Cc1cc(N2CCNC(C)C2)c(C#N)cc1-n1c(=N)n(C)c2cnc3ccc(-c4ccc(Cl)cc4)cc3c21]

LCMS: t_{R}=4.204 min in 10-80CD_7MIN_220&254 (Xtimate 2.1*30 mm, 3 um), MS (ESI) m/z 522.2 [M+H]⁺.

¹H NMR (400 MHz, DMSO-d₆) δ=8.79 (s, 1H), 8.00 (d, J=8.8 Hz, 1H), 7.97 (br d, J=8.6 Hz, 1H), 7.78 (ddd, J=2.2, 3.6, 9.0 Hz, 1H), 7.43-7.38 (m, 2H), 7.34-7.30 (m, 3H), 6.90 (d, J=1.8 Hz, 1H), 3.60-3.46 (m, 4H), 3.06-2.97 (m, 1H), 2.93-2.81 (m, 3H), 2.62-2.55 (m, 1H), 2.46-2.39 (m, 1H), 2.05 (s, 3H), 1.05 (dd, J=6.4, 10.8 Hz, 3H).

### methyl (E)-(1-(5-cyano-2-methylphenyl)-3-methyl-8-(6-methylpyridin-3-yl)-1,3-dihydro-2H-imidazo[4,5-c]quinolin-2-ylidene)carbamate (125)

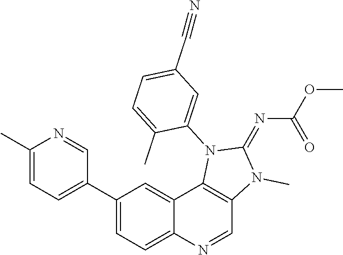

LCMS: t_{R}=2.838 min in 10-80CD_7MIN_220&25 (Xtimate 3 um, C18, 2.1*30 mm), MS (ESI) m/z 463.2 [M+H]⁺.

¹H NMR (400 MHz, METHANOL-d₄) δ=9.18 (s, 1H), 8.30 (d, J=2.2 Hz, 1H), 8.25 (d, J=8.8 Hz, 1H), 8.05 (s, 1H), 8.03-7.96 (m, 2H), 7.81-7.73 (m, 2H), 7.36 (d, J=8.2 Hz, 1H), 7.11 (d, J=1.8 Hz, 1H), 3.84 (s, 3H), 3.50 (s, 3H), 2.55 (s, 3H), 2.21 (s, 3H).

### 4-(4-(8-(4-chlorophenyl)-2-imino-3-methyl-2,3-dihydro-1H-imidazo[4,5-c]quinolin-1-yl)-2-cyano-5-methylphenyl)piperazine-1-carboxamide (126)

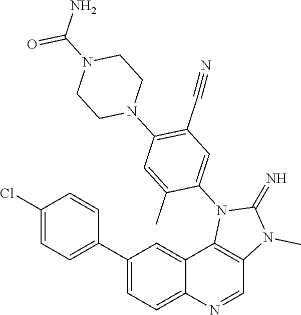 [SMILES: Cc1cc(N2CCN(C(N)=O)CC2)c(C#N)cc1-n1c(=N)n(C)c2cnc3ccc(-c4ccc(Cl)cc4)cc3c21]

LCMS: t_{R}=3.478 min in 10-80CD_7MIN (Xtimate 2.1*30 mm, 3 um), MS (ESI) m/z 551.2 [M+H]⁺.

¹H NMR (400 MHz, DMSO-d₆) δ=8.80 (s, 1H), 8.00 (d, J=8.8 Hz, 2H), 7.79 (dd, J=2.1, 8.9 Hz, 1H), 7.44-7.40 (m, 2H), 7.37 (s, 1H), 7.34-7.30 (m, 2H), 6.91 (d, J=1.3 Hz, 1H), 6.14 (s, 2H), 3.54-3.50 (m, 7H), 3.28 (br d, J=5.1 Hz, 2H), 3.24-3.19 (m, 2H), 2.07 (s, 3H).

### 1-(4-(8-(4-chlorophenyl)-2-imino-3-methyl-2,3-dihydro-1H-imidazo[4,5-c]quinolin-1-yl)-[1,4′-bipiperidin]-1′-yl)ethan-1-one (127)

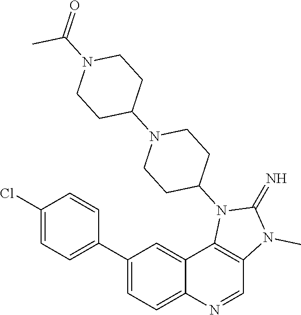 [SMILES: CC(=O)N1CCC(N2CCC(n3c(=N)n(C)c4cnc5ccc(-c6ccc(Cl)cc6)cc5c43)CC2)CC1]

LCMS: t_{R}=4.513 min in 0-30AB_7 min_220&254 (Xtimate 3 um, C18, 2.1*30 mm), MS (ESI) m/z 517.1 [M+H]⁺.

¹H NMR (400 MHz, DMSO-d₆) δ=11.11 (br s, 1H), 9.40-9.24 (m, 3H), 8.50 (s, 1H), 8.36 (d, J=9.0 Hz, 1H), 8.17 (d, J=10.0 Hz, 1H), 7.99 (d, J=8.5 Hz, 2H), 7.64 (d, J=8.5 Hz, 2H), 5.85-5.71 (m, 1H), 5.85-5.71 (m, 1H), 4.57 (br d, J=13.6 Hz, 1H), 4.02 (br d, J=13.8 Hz, 1H), 3.87 (s, 3H), 3.77 (br s, 2H), 3.46 (br d, J=11.8 Hz, 2H), 3.38-3.20 (m, 2H), 3.16-3.03 (m, 1H), 2.57-2.54 (m, 2H), 2.48-2.39 (m, 2H), 2.17 (br t, J=13.1 Hz, 2H), 2.04 (s, 3H), 1.81-1.66 (m, 1H), 1.66-1.48 (m, 1H).

### 1-(4-(8-(4-chlorophenyl)-2-imino-3-methyl-2,3-dihydro-1H-imidazo[4,5-c]quinolin-1-yl)-[1,4′-bipiperidin]-1′-yl)propan-1-one (128)

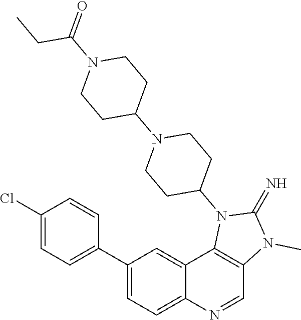 [SMILES: CCC(=O)N1CCC(N2CCC(n3c(=N)n(C)c4cnc5ccc(-c6ccc(Cl)cc6)cc5c43)CC2)CC1]

LCMS: t_{R}=4.853 min in 0-30AB_7 min_220&254 (Xtimate 3 um, C18, 2.1*30 mm), MS (ESI) m/z 531.1 [M+H]⁺. 1H NMR (400 MHz, DMSO-d₆) δ=11.11 (br s, 1H), 9.43-9.23 (m, 3H), 8.50 (s, 1H), 8.36 (d, J=8.8 Hz, 1H), 8.17 (d, J=9.0 Hz, 1H), 8.00 (d, J=8.5 Hz, 2H), 7.64 (d, J=8.5 Hz, 2H), 5.86-5.65 (m, 1H), 4.59 (br d, J=12.8 Hz, 1H), 4.07 (br d, J=14.1 Hz, 1H), 3.87 (s, 3H), 3.64-3.57 (m, 2H), 3.46 (br d, J=9.0 Hz, 2H), 3.35-3.21 (m, 2H), 3.05 (br t, J=12.5 Hz, 1H), 2.63-2.53 (m, 2H), 2.46 (br s, 2H), 2.37 (q, J=7.7 Hz, 2H), 2.17 (br t, J=9.8 Hz, 2H), 1.82-1.47 (m, 2H), 1.00 (t, J=7.3 Hz, 3H).

### 1-(4-((1r,3r)-3-(8-(4-chlorophenyl)-2-imino-3-methyl-2,3-dihydro-1H-imidazo[4,5-c]quinolin-1-yl)cyclobutyl)piperazin-1-yl)ethan-1-one (129)

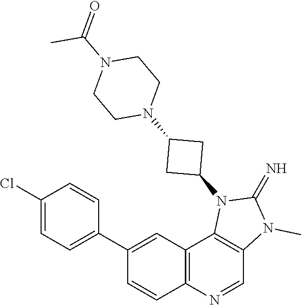 [SMILES: CC(=O)N1CCN([C@H]2C[C@H](n3c(=N)n(C)c4cnc5ccc(-c6ccc(Cl)cc6)cc5c43)C2)CC1]

LCMS: t_{R}=4.466 min in 0-30AB_7 min_220&254 chromatography (Xtimate C18 2.1*30 mm, 3 um), MS (ESI) m/z 489.0 [M+H]⁺.

¹H NMR (400 MHz, DMSO-d₆) δ=11.99 (br s, 1H), 12.05-11.91 (m, 1H), 9.44-9.23 (m, 3H), 8.38 (s, 1H), 8.33 (d, J=9.0 Hz, 1H), 8.16 (br d, J=8.8 Hz, 1H), 7.98 (d, J=8.5 Hz, 2H), 7.63 (d, J=8.5 Hz, 2H), 5.60-5.48 (m, 1H), 4.42 (br d, J=12.5 Hz, 1H), 3.95 (br d, J=15.1 Hz, 1H), 3.89 (s, 3H), 3.45-3.33 (m, 3H), 3.18 (br dd, J=8.3, 17.6 Hz, 4H), 2.87 (br d, J=13.6 Hz, 2H), 2.78 (br d, J=9.5 Hz, 1H), 2.43 (br s, 1H), 2.02 (s, 3H).

### 2-(4-acetylpiperazin-1-yl)-5-(8-(4-chlorophenyl)-2-imino-3-methyl-2,3-dihydro-1H-imidazo[4,5-c]quinolin-1-yl)-6-methylnicotinonitrile (130)

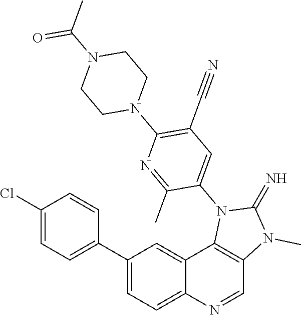 [SMILES: CC(=O)N1CCN(c2nc(C)c(-n3c(=N)n(C)c4cnc5ccc(-c6ccc(Cl)cc6)cc5c43)cc2C#N)CC1]

LCMS: t_{R}=4.005 min in 0-60AB_7.0 min chromatography (Xtimate, 2.1*30 mm, 3 um), MS (ESI) m/z 551.1 [M+H]⁺.

¹H NMR: (400 MHz, DMSO-d₆): δ=9.43 (s, 1H), 9.36 (s, 2H), 8.55 (s, 1H), 8.31 (d, J=8.8 Hz, 1H), 8.10 (dd, J=2.0, 8.8 Hz, 1H), 7.54-7.48 (m, 4H), 7.23 (d, J=2.0 Hz, 1H), 3.99 (s, 3H), 3.97-3.95 (m, 4H), 3.68-3.65 (m, 4H), 2.28 (s, 3H), 2.12 (s, 3H).

### N-(1-(4-(8-(4-chlorophenyl)-2-imino-3-methyl-2,3-dihydro-H-imidazo[4,5-c]quinolin-1-yl)-2-cyano-5-methylphenyl)piperidin-4-yl)methanesulfonamide (131)

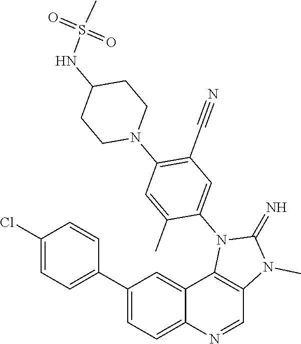 [SMILES: Cc1cc(N2CCC(NS(C)(=O)=O)CC2)c(C#N)cc1-n1c(=N)n(C)c2cnc3ccc(-c4ccc(Cl)cc4)cc3c21]

LCMS: t_{R}=4.150 min in 0-60AB_7.0 MIN chromatography (Xtimate C18 2.1*30 mm, 3 um), MS (ESI) m/z 600.1 [M+H]⁺.

¹H NMR (400 MHz, DMSO-d₆) δ=9.34 (s, 1H), 9.00 (br s, 2H), 8.23 (d, J=8.8 Hz, 1H), 8.14 (s, 1H), 8.04 (dd, J=2.1, 8.9 Hz, 1H), 7.50-7.45 (m, 2H), 7.43 (s, 1H), 7.38 (d, J=8.4 Hz, 2H), 7.26 (d, J=7.5 Hz, 1H), 6.98 (d, J=1.8 Hz, 1H), 3.91 (s, 3H), 3.79-3.59 (m, 2H), 3.30-3.26 (m, 1H), 3.18-3.08 (m, 2H), 2.98 (s, 3H), 2.10 (s, 3H), 2.09-2.00 (m, 2H), 1.66 (br d, J=18.3 Hz, 2H).

### N-(1-(4-(8-(4-chlorophenyl)-2-imino-3-methyl-2,3-dihydro-1H-imidazo[4,5-c]quinolin-1-yl)-2-cyano-5-methylphenyl)piperidin-4-yl)acetamide (132)

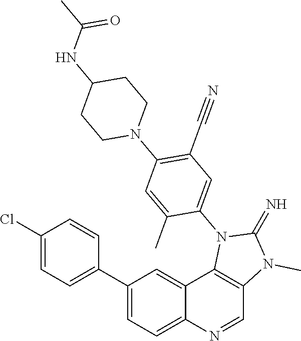 [SMILES: CC(=O)NC1CCN(c2cc(C)c(-n3c(=N)n(C)c4cnc5ccc(-c6ccc(Cl)cc6)cc5c43)cc2C#N)CC1]

LCMS: t_{R}=3.838 min in 0-60AB_7.0 MIN chromatography (Xtimate C18 2.1*30 mm, 3 um), MS (ESI) m/z 564.1 [M+H]⁺.

¹H NMR (400 MHz, DMSO-d₆) δ=9.35 (s, 1H), 9.02 (s, 2H), 8.24 (d, J=9.0 Hz, 1H), 8.15 (s, 1H), 8.05 (dd, J=2.0, 8.8 Hz, 1H), 7.95 (d, J=7.5 Hz, 1H), 7.52-7.43 (m, 3H), 7.39 (d, J=8.6 Hz, 2H), 6.98 (d, J=1.8 Hz, 1H), 3.92 (s, 3H), 3.84-3.81 (m, 1H), 3.79-3.60 (m, 2H), 3.20-3.04 (m, 2H), 2.11 (s, 3H), 1.96 (br d, J=8.2 Hz, 2H), 1.83 (s, 3H), 1.71-1.48 (m, 2H).

### 5-(8-(4-chlorophenyl)-2-imino-3-methyl-2,3-dihydro-1H-imidazo[4,5-c]quinolin-1-yl)-4-methyl-2-(3-oxopiperazin-1-yl)benzonitrile (133)

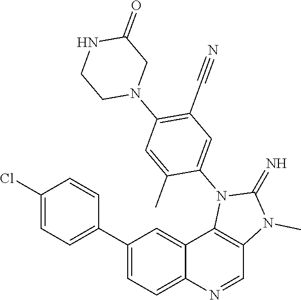 [SMILES: Cc1cc(N2CCNC(=O)C2)c(C#N)cc1-n1c(=N)n(C)c2cnc3ccc(-c4ccc(Cl)cc4)cc3c21]

LCMS: t_{R}=3.689 min in 0-60AB_7.0 MIN chromatography (Xtimate C18 2.1*30 mm, 3 um), MS (ESI) m/z 522.0 [M+H]⁺.

¹H NMR (400 MHz, DMSO-d₆) δ=9.37 (s, 1H), 9.06 (s, 2H), 8.30-8.24 (m, 2H), 8.22 (s, 1H), 8.07 (dd, J=2.1, 8.9 Hz, 1H), 7.54-7.49 (m, 3H), 7.44-7.40 (m, 2H), 7.02 (d, J=2.2 Hz, 1H), 4.10-4.06 (m, 1H), 3.96-3.92 (m, 4H), 3.86-3.73 (m, 1H), 3.70-3.61 (m, 1H), 3.46-3.45 (m, 2H), 2.15 (s, 3H).

### 5-(8-(4-chlorophenyl)-2-imino-3-methyl-2,3-dihydro-1H-imidazo[4,5-c]quinolin-1-yl)-2-fluoro-4-methylbenzonitrile (134)

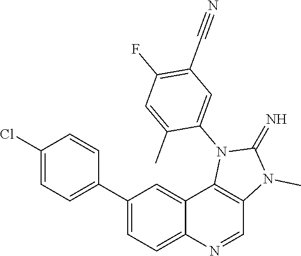 [SMILES: Cc1cc(F)c(C#N)cc1-n1c(=N)n(C)c2cnc3ccc(-c4ccc(Cl)cc4)cc3c21]

LCMS: t_{R}=4.302 min in 0-60AB_7 min chromatography (Xtimate 3 um, C18, 2.1*30 mm), MS (ESI) m/z 441.9 [M+H]⁺.

¹H NMR: DMSO-d₆ 400 MHz): δ=9.41 (s, 1H), 9.11 (br s, 2H), 8.52 (d, J=6.0 Hz, 1H), 8.29 (d, J=8.8 Hz, 1H), 8.11-8.00 (m, 2H), 7.57-7.51 (m, 2H), 7.44 (d, J=8.5 Hz, 2H), 7.01 (d, J=1.5 Hz, 1H), 3.96 (s, 3H), 2.25 (s, 3H).

### 4-(8-(4-chlorophenyl)-2-imino-3-methyl-2,3-dihydro-1H-imidazo[4,5-c]quinolin-1-yl)-5-methylphthalonitrile (135)

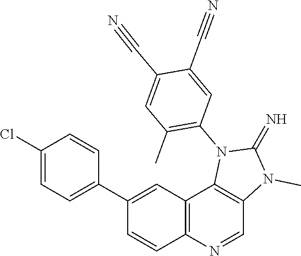 [SMILES: Cc1cc(C#N)c(C#N)cc1-n1c(=N)n(C)c2cnc3ccc(-c4ccc(Cl)cc4)cc3c21]

LCMS: t_{R}=4.941 min in 0-60AB_7 min chromatography (Xtimate 3 um, C18, 2.1*30 mm), MS (ESI) m/z 449.1 [M+H]⁺.

### 3-(2-imino-3-methyl-8-(1-methyl-1H-pyrazol-4-yl)-2,3-dihydro-1H-imidazo[4,5-c]quinolin-1-yl)-4-methylbenzonitrile (136)

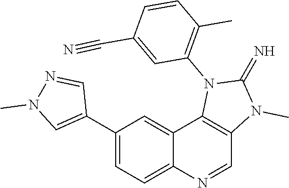

LCMS: t_{R}=2.502 min in 0-60AB_7 min_220&254 chromatography (Xtimate 3 um, C18, 2.1*30 mm), MS (ESI) m/z 394.0 [M+H]⁺.

¹H NMR (400 MHz, DMSO-d₆) δ=9.27 (s, 1H), 9.04 (s, 2H), 8.39-8.30 (m, 2H), 8.12 (d, J=8.8 Hz, 1H), 7.98 (d, J=7.9 Hz, 1H), 7.94 (s, 1H), 7.88 (dd, J=2.0, 8.8 Hz, 1H), 7.17 (s, 1H), 6.68 (d, J=1.5 Hz, 1H), 3.91 (s, 3H), 3.82 (s, 3H), 2.20 (s, 3H).

### 3-(8-(4-chlorophenyl)-2-imino-3-methyl-2,3-dihydro-1H-imidazo[4,5-c]quinolin-1-yl)-4-methylbenzonitrile (137)

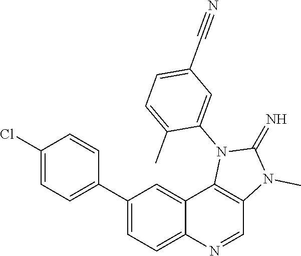 [SMILES: Cc1ccc(C#N)cc1-n1c(=N)n(C)c2cnc3ccc(-c4ccc(Cl)cc4)cc3c21]

LCMS: t_{R}=3.841 min in 0-60AB_7 min_220&254 chromatography (Xtimate 3 um, C18, 2.1*30 mm), MS (ESI) m/z 424.0 [M+H]⁺.

¹H NMR (400 MHz, DMSO-d₆) δ=9.37 (s, 1H), 9.08 (s, 2H), 8.36 (d, J=1.3 Hz, 1H), 8.31-8.20 (m, 2H), 8.02 (dd, J=2.0, 8.8 Hz, 1H), 7.93 (d, J=7.9 Hz, 1H), 7.48 (d, J=8.6 Hz, 2H), 7.32 (d, J=8.6 Hz, 2H), 6.83 (d, J=1.8 Hz, 1H), 3.93 (s, 3H), 2.20 (s, 3H).

### (Z)—N-(9-(4-chlorophenyl)-1-(4-(4-(2-(dimethylamino)ethyl)piperazine-1-carbonyl)phenyl)benzo[h][1,6]naphthyridin-2(1H)-ylidene)cyanamide (138)

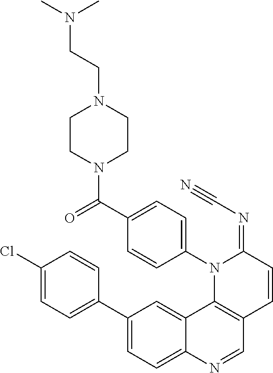 [SMILES: CN(C)CCN1CCN(C(=O)c2ccc(-n3/c(=N\C#N)ccc4cnc5ccc(-c6ccc(Cl)cc6)cc5c43)cc2)CC1]

LCMS: t_{R}=3.794 min in 0-60AB_7 min_220&254 (Xtimate 3 um, C18, 2.1*30 mm), MS (ESI) m/z 612.1 [M+Na]⁺.

¹H NMR (400 MHz, DMSO-d₆) δ=9.31 (s, 1H), 8.56 (d, J=9.3 Hz, 1H), 8.20-8.16 (m, 1H), 8.10-8.05 (m, 1H), 7.76-7.69 (m, 4H), 7.66 (d, J=9.3 Hz, 1H), 7.53 (d, J=8.5 Hz, 2H), 7.30 (d, J=1.0 Hz, 1H), 7.21 (d, J=8.3 Hz, 2H), 3.75-3.62 (m, 2H), 3.25-3.13 (m, 2H), 2.45-2.25 (m, 8H), 2.16 (s, 6H).

### 3-(9-(4-chlorophenyl)-2-iminobenzo[h][1,6]naphthyridin-1(2H)-yl)benzonitrile (139)

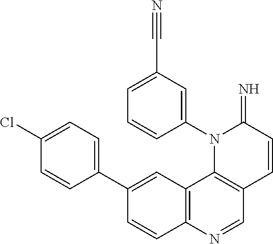 [SMILES: N#Cc1cccc(-n2c(=N)ccc3cnc4ccc(-c5ccc(Cl)cc5)cc4c32)c1]

LCMS: t_{R}=4.299 min in 0-60 AB_7MIN_220&254 chromatography (Xtimate 3 um, C18, 2.1*30 mm), MS (ESI) m/z 406.9 [M+H]⁺.

¹H NMR: (400 MHz, DMSO-d₆) δ=10.05 (s, 2H), 8.79 (s, 1H), 7.98 (br d, J=8.8 Hz, 1H), 7.75 (br dd, J=2.0, 8.8 Hz, 1H), 7.50 (br t, J=7.8 Hz, 1H), 7.36 (br dd, J=5.5, 8.8 Hz, 2H), 7.21 (br t, J=8.8 Hz, 2H), 7.13 (br d, J=1.5 Hz, 1H), 7.06 (br d, J=7.9 Hz, 1H), 7.01 (br d, J=6.6 Hz, 1H), 6.93 (br s, 1H), 3.50 (s, 3H).

### 9-(4-chlorophenyl)-1-(3-(trifluoromethyl)phenyl)benzo[h][1,6]naphthyridin-2(1H)-imine (140)

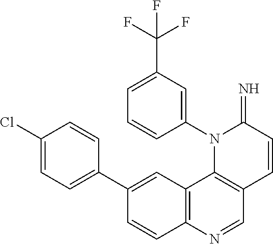 [SMILES: N=c1ccc2cnc3ccc(-c4ccc(Cl)cc4)cc3c2n1-c1cccc(C(F)(F)F)c1]

LCMS: t_{R}=4.930 min in 0-60 AB_7 min chromatography (Xtimate 3 um, C18, 2.1*30 mm), MS (ESI) m/z 449.9 [M+H]⁺.

¹H NMR: (400 MHz, DMSO-d₆) δ=8.82 (br s, 1H), 8.02 (br s, 1H), 7.96 (br d, J=8.6 Hz, 2H), 7.86 (br d, J=8.2 Hz, 1H), 7.78 (br s, 1H), 7.67 (br d, J=6.4 Hz, 1H), 7.54 (br d, J=9.0 Hz, 1H), 7.37 (br d, J=7.7 Hz, 2H), 7.05 (br d, J=8.2 Hz, 2H), 6.96 (br s, 1H), 6.85 (br d, J=9.3 Hz, 1H).

### 1-(3-chlorophenyl)-9-(4-chlorophenyl)benzo[h][1,6]naphthyridin-2(1H)-imine (141)

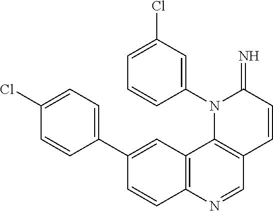 [SMILES: N=c1ccc2cnc3ccc(-c4ccc(Cl)cc4)cc3c2n1-c1cccc(Cl)c1]

LCMS: t_{R}=4.729 min in 0-60AB_7 min_220&254 chromatography (Xtimate, 2.1*30 mm, 3 um), MS (ESI) m/z 415.9 [M+H]⁺.

¹H NMR (400 MHz, DMSO-d₆) δ=8.82 (s, 1H), 8.00-7.93 (m, 1H), 7.92-7.84 (m, 1H), 7.76-7.67 (m, 2H), 7.62 (br t, J=7.9 Hz, 1H), 7.55 (br d, J=9.3 Hz, 1H), 7.46-7.35 (m, 3H), 7.20-7.05 (m, 3H), 6.84 (d, J=9.5 Hz, 1H).

### 5-(8-(3-fluorophenyl)-2-imino-3-methyl-2,3-dihydro-1H-imidazo[4,5-c]quinolin-1-yl)-6-methyl-2-morpholinonicotinonitrile (142)

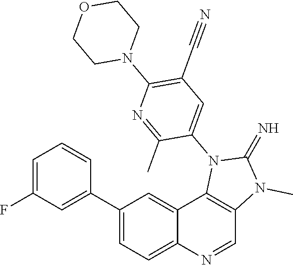 [SMILES: Cc1nc(N2CCOCC2)c(C#N)cc1-n1c(=N)n(C)c2cnc3ccc(-c4cccc(F)c4)cc3c21]

LCMS: t_{R}=4.262 min in 0-60AB_7 min chromatography (Xtimate 3 um, C18, 2.1*30 mm), MS (ESI) m/z 494.0 [M+H]⁺.

¹HNMR: (DMSO-d₆ 400 MHz): δ=9.43 (s, 1H), 9.36 (br s, 2H), 8.55 (s, 1H), 8.31 (d, J=8.8 Hz, 1H), 8.16 (dd, J=2.0, 9.0 Hz, 1H), 7.58-7.49 (m, 1H), 7.38-7.23 (m, 4H), 7.22 (br s, 1H), 3.99 (s, 3H), 3.95-3.77 (m. 8H), 2.28 (s, 3H).

### 5-(8-(3-fluorophenyl)-2-imino-3-methyl-2,3-dihydro-1H-imidazo[4,5-c]quinolin-1-yl)-6-methyl-2-(4-methylpiperazin-1-yl)nicotinonitrile (143)

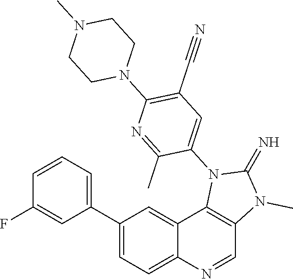 [SMILES: Cc1nc(N2CCN(C)CC2)c(C#N)cc1-n1c(=N)n(C)c2cnc3ccc(-c4cccc(F)c4)cc3c21]

LCMS: t_{R}=4.380 min in 0-30AB_7.0 min chromatography (Xtimate, 2.1*30 mm, 3 um), MS (ESI) m/z 507.1 [M+H]⁺.

¹H NMR: (400 MHz, DMSO-d₆): δ=11.79 (s, 1H), 9.61 (s, 2H), 9.48 (s, 1H), 8.68 (s, 1H), 8.34 (d, J=8.0 Hz, 1H), 8.18 (d, J=8.8 Hz, 2H), 7.61-7.59 (m, 1H), 7.35-7.26 (m, 4H), 4.54-4.49 (m, 2H), 4.02 (s, 3H), 3.67-3.60 (m, 4H), 3.33-3.21 (m, 2H), 2.85 (s, 3H), 2.32 (s, 3H).

### 5-(8-(3-fluorophenyl)-2-imino-3-methyl-2,3-dihydro-1H-imidazo[4,5-c]quinolin-1-yl)-4-methyl-2-(4-methylpiperazin-1-yl)benzonitrile (144)

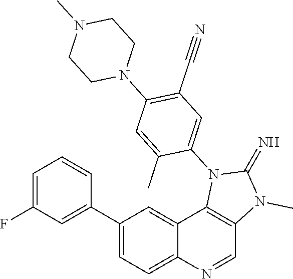 [SMILES: Cc1cc(N2CCN(C)CC2)c(C#N)cc1-n1c(=N)n(C)c2cnc3ccc(-c4cccc(F)c4)cc3c21]

LCMS: t_{R}=2.824 min in 0-60AB_7 min chromatography (Xtimate 3 um, C18, 2.1*30 mm), MS (ESI) m/z 506.1 [M+H]⁺.

¹H NMR: (DMSO-d₆ 400 MHz): δ=11.70 (br s, 1H), 9.41 (s, 1H), 9.31 (br s, 2H), 8.30-8.26 (m, 2H), 8.12 (dd, J=1.9, 8.9 Hz, 1H), 7.63 (s, 1H), 7.60-7.54 (m, 1H), 7.27-7.16 (m, 2H), 7.21-7.15 (m, 1H), 7.01 (d, J=1.8 Hz, 1H), 3.99 (s, 3H), 3.81-3.70 (m, 2H), 3.64-3.61 (m, 2H), 3.51-3.43 (m, 2H), 3.33-3.20 (m, 2H), 2.86 (br d, J=4.2 Hz, 3H), 2.31 (s, 1H), 2.16 (s, 3H).

### 5-(2-imino-3-methyl-8-(p-tolyl)-2,3-dihydro-1H-imidazo[4,5-c]quinolin-1-yl)-4-methyl-2-morpholinobenzonitrile (145)

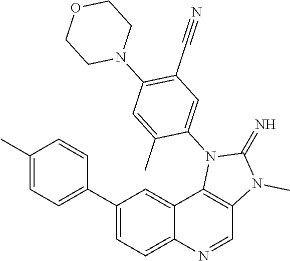

LCMS: t_{R}=4.133 min in 0-60AB_7.0 MIN chromatography (Xtimate 3 um, C18, 2.1*30 mm), MS (ESI) m/z 489.1[M+H]⁺.

¹H NMR (400 MHz, DMSO-d₆) δ=9.34 (s, 1H), 9.04 (s, 2H), 8.23 (t, J=4.4 Hz, 2H), 8.06 (dd, J=1.8, 9.0 Hz, 1H), 7.51 (s, 1H), 7.33-7.21 (m, 4H), 6.99 (s, 1H), 3.94 (s, 3H), 3.87 (t, J=4.5 Hz, 4H), 3.36-3.29 (m, 4H), 2.35 (s, 3H), 2.16 (s, 3H).

### Methyl (E)-(1-(5-cyano-2-methyl-4-(4-methylpiperazin-1-yl)phenyl)-8-(3-fluorophenyl)-3-methyl-1,3-dihydro-2H-imidazo[4,5-c]quinolin-2-ylidene)carbamate (146)

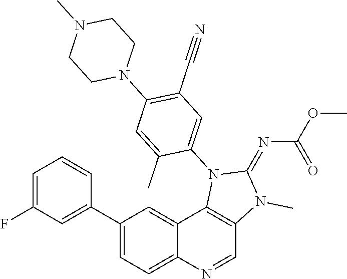 [SMILES: COC(=O)/N=c1\n(C)c2cnc3ccc(-c4cccc(F)c4)cc3c2n1-c1cc(C#N)c(N2CCN(C)CC2)cc1C]

LCMS: t_{R}=3.133 min in 0-60AB_7.0 MIN chromatography (Xtimate 3 um, C18, 2.1*30 mm), MS (ESI) m/z 564.1 [M+H]⁺.

¹H NMR (400 MHz, DMSO-d₆) δ=9.23 (s, 1H), 8.19 (d, J=8.8 Hz, 1H), 8.08-7.99 (m, 2H), 7.52-7.43 (m, 1H), 7.39 (s, 1H), 7.29-7.21 (m, 2H), 7.16 (br d, J=10.8 Hz, 1H), 7.09 (d, J=2.0 Hz, 1H), 3.73 (s, 3H), 3.39 (s, 3H), 3.32-3.28 (m, 4H), 2.56 (br s, 4H), 2.28 (s, 3H), 2.09 (s, 3H).

### Methyl (E)-(1-(5-cyano-2-methyl-4-(4-methylpiperazin-1-yl)phenyl)-3-methyl-8-(p-tolyl)-1,3-dihydro-2H-imidazo[4,5-c]quinolin-2-ylidene)carbamate (147)

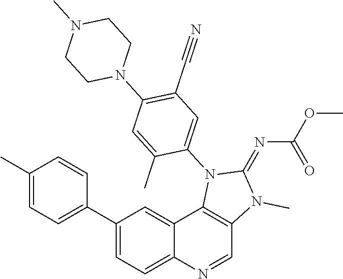

LCMS: t_{R}=3.241 min in 0-60AB_7 min_220&254 (Xtimate 3 um, C18, 2.1*30 mm), MS (ESI) m/z 560.1 [M+H]⁺.

¹H NMR (400 MHz, DMSO-d₆) δ=9.16 (s, 1H), 8.13 (d, J=8.8 Hz, 1H), 8.03 (s, 1H), 7.94 (dd, J=2.0, 9.0 Hz, 1H), 7.35 (s, 1H), 7.27-7.22 (m, 2H), 7.21-7.16 (m, 2H), 7.03 (d, J=1.8 Hz, 1H), 3.69 (s, 3H), 3.36 (s, 3H), 3.31-3.25 (m, 4H), 2.57-2.52 (m, 4H), 2.31 (s, 3H), 2.26 (s, 3H), 2.05 (s, 3H).

### Methyl (E)-(1-(5-cyano-2-methyl-4-morpholinophenyl)-8-(3-fluorophenyl)-3-methyl-1,3-dihydro-2H-imidazo[4,5-c]quinolin-2-ylidene)carbamate (148)

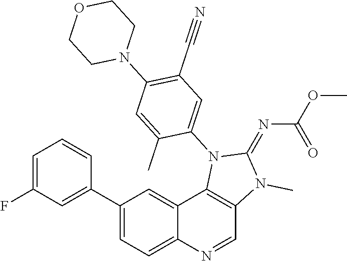 [SMILES: COC(=O)/N=c1\n(C)c2cnc3ccc(-c4cccc(F)c4)cc3c2n1-c1cc(C#N)c(N2CCOCC2)cc1C]

LCMS: t_{R}=4.216 min in 0-60AB_7.0 MIN chromatography (Xtimate C18 2.1*30 mm, 3 um), MS (ESI) m/z 551.1 [M+H]⁺.

¹H NMR (400 MHz, DMSO-d₆) δ=9.21 (s, 1H), 8.17 (d, J=9.0 Hz, 1H), 8.08 (s, 1H), 8.01 (dd, J=2.1, 8.9 Hz, 1H), 7.50-7.42 (m, 1H), 7.40 (s, 1H), 7.26-7.19 (m, 2H), 7.14 (br d, J=10.1 Hz, 1H), 7.06 (d, J=1.8 Hz, 1H), 3.81 (t, J=4.6 Hz, 4H), 3.71 (s, 3H), 3.37 (s, 3H), 3.29-3.22 (m, 4H), 2.09 (s, 3H).

### (E)-4-(9-(4-chlorophenyl)-2-(tosylimino)benzo[h][1,6]naphthyridin-1(2H)-yl)-N-cyclopropylbenzamide (149)

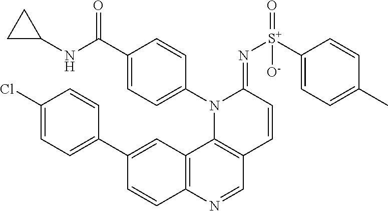 [SMILES: Cc1ccc([S+](=O)([O-])/N=c2\ccc3cnc4ccc(-c5ccc(Cl)cc5)cc4c3n2-c2ccc(C(=O)NC3CC3)cc2)cc1]

LCMS: t_{R}=5.485 min in 0-60AB_7MIN 220&254 (Xtimate, 2.1*30 mm, 3 um), MS (ESI) m/z 619.0 [M+H]⁺.

¹H NMR (400 MHz, DMSO-d₆) δ=9.23 (s, 1H), 8.75 (d, J=4.0 Hz, 1H), 8.53 (d, J=9.7 Hz, 1H), 8.17-8.10 (m, 3H), 8.03 (dd, J=2.0, 8.6 Hz, 1H), 7.89 (d, J=9.5 Hz, 1H), 7.69 (d, J=8.6 Hz, 2H), 7.43 (d, J=8.4 Hz, 2H), 7.32 (d, J=8.6 Hz, 2H), 7.21 (d, J=8.2 Hz, 2H), 7.06 (d, J=8.4 Hz, 2H), 6.89 (d, J=1.8 Hz, 1H), 3.02-2.90 (m, 1H), 2.30 (s, 3H), 0.83-0.75 (m, 2H), 0.67-0.60 (m, 2H).

### (Z)-4-(9-(4-chlorophenyl)-2-(propionylimino)benzo[h][1,6]naphthyridin-1(2H)-yl)-N-cyclopropylbenzamide (150)

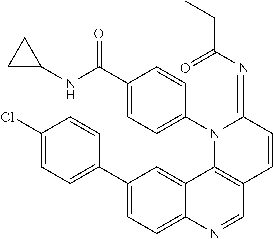 [SMILES: CCC(=O)/N=c1/ccc2cnc3ccc(-c4ccc(Cl)cc4)cc3c2n1-c1ccc(C(=O)NC2CC2)cc1]

LCMS: t_{R}=4.104 min in 10-80CD_7MIN_220&254 (XBridge Shield RP18 2.1*50 mm, 5 um), MS (ESI) m/z 521.2 [M+H]⁺.

¹H NMR (400 MHz, DMSO-d₆) δ=9.06 (s, 1H), 8.71 (d, J=4.0 Hz, 1H), 8.13-8.02 (m, 4H), 7.95 (dd, J=1.9, 8.7 Hz, 1H), 7.62 (d, J=8.6 Hz, 2H), 7.35 (d, J=9.5 Hz, 1H), 7.33-7.28 (m, 2H), 7.06-7.01 (m, 2H), 6.89 (d, J=1.5 Hz, 1H), 2.99-2.87 (m, 1H), 2.17 (q, J=7.5 Hz, 2H), 0.88 (t, J=7.4 Hz, 3H), 0.81-0.73 (m, 2H), 0.65-0.59 (m, 2H).

### (E)-4-(9-(4-chlorophenyl)-2-(methylimino)benzo[h][1,6]naphthyridin-1(2H)-yl)-N-cyclopropylbenzamide (151)

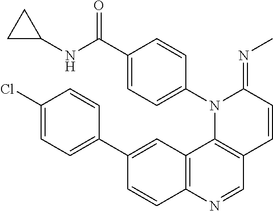 [SMILES: C/N=c1\ccc2cnc3ccc(-c4ccc(Cl)cc4)cc3c2n1-c1ccc(C(=O)NC2CC2)cc1]

LCMS: t_{R}=3.973 min in 0-60AB_7.0 min chromatography (Xtimate, 2.1*30 mm, 3 um), MS (ESI) m/z 479.1 [M+H]⁺.

¹H NMR: (400 MHz, DMSO-d₆): δ=8.34 (d, J=4.0 Hz, 1H), 8.12 (s, 1H), 7.90-7.89 (m, 1H), 7.88-7.87 (m, 2H), 7.83-7.81 (m, 1H), 7.63-7.61 (m, 1H), 7.28-7.26 (m, 3H), 7.14-7.12 (m, 2H), 6.77 (d, J=8.4 Hz, 2H), 6.63 (brs, 1H), 3.76 (s, 3H), 2.89-2.86 (m, 1H), 0.73-0.69 (m, 2H), 0.59-0.56 (m, 2H).

### 5-(2-imino-1-(3-(trifluoromethyl)phenyl)-1,2-dihydrobenzo[h][1,6]naphthyridin-9-yl)pyridin-2-amine (152)

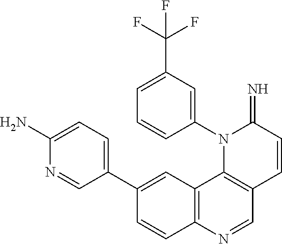 [SMILES: N=c1ccc2cnc3ccc(-c4ccc(N)nc4)cc3c2n1-c1cccc(C(F)(F)F)c1]

LCMS: t_{R}=2.230 min in 0-60AB_7 min chromatography (Xtimate 3 um, C18, 2.1*30 mm), MS (ESI) m/z 432.0[M+H]⁺.

¹H NMR: (DMSO-d₆ 400 MHz): δ 10.13 (s, 1H), 9.41 (s, 1H), 8.75 (d, J=9.3 Hz, 1H), 8.51 (s, 1H), 8.35-8.08 (m, 7H), 7.84 (d, J=2.0 Hz, 1H), 7.58 (d, J=9.3 Hz, 1H), 7.38 (dd, J=2.1, 9.2 Hz, 1H), 6.90 (d, J=9.3 Hz, 1H), 6.58 (d, J=1.3 Hz, 1H).

### 4-(9-(4-chlorophenyl)-2-iminobenzo[h][1,6]naphthyridin-1(2H)-yl)-N-cyclopropylbenzamide (153)

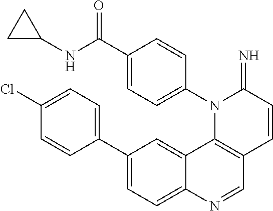 [SMILES: N=c1ccc2cnc3ccc(-c4ccc(Cl)cc4)cc3c2n1-c1ccc(C(=O)NC2CC2)cc1]

LCMS: t_{R}=2.803 min in 10-80AB_7 min_220&254 chromatography (Xtimate 3 um, C18, 2.1*30 mm), MS (ESI) m/z 465.1 [M+H]⁺.

¹H NMR (400 MHz, DMSO-d₆) δ=9.29 (s, 1H), 8.93 (br d, J=3.7 Hz, 1H), 8.56 (br d, J=9.3 Hz, 1H), 8.35 (br d, J=8.4 Hz, 2H), 8.17 (d, J=8.6 Hz, 1H), 8.06 (br d, J=8.6 Hz, 1H), 7.91 (br d, J=8.4 Hz, 2H), 7.55 (br d, J=9.3 Hz, 1H), 7.33 (br d, J=8.4 Hz, 2H), 7.09 (br d, J=8.4 Hz, 2H), 6.74 (s, 1H), 3.03-2.92 (m, 1H), 0.85-0.64 (m, 4H).

### 3-(2-imino-3-methyl-8-(phenylethynyl)-2,3-dihydro-H-imidazo[4,5-c]quinolin-1-yl)-4-methylbenzonitrile (154)

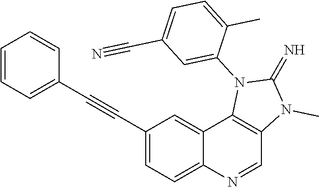 [SMILES: Cc1ccc(C#N)cc1-n1c(=N)n(C)c2cnc3ccc(C#Cc4ccccc4)cc3c21]

LCMS: t_{R}=4.677 min in 0-60 AB_7MIN_220&254 chromatography (Xtimate 3 um, C18, 2.1*30 mm), MS (ESI) m/z 414.1 [M+H]⁺.

¹H NMR: (400 MHz, DMSO-d₆) δ=8.86 (br s, 1H), 8.13 (br s, 3H), 7.97 (br d, J=9.3 Hz, 1H), 7.85 (br s, 1H), 7.56 (br d, J=9.0 Hz, 1H), 7.44 (s, 4H), 6.77 (br s, 1H), 3.55 (s, 3H), 2.16 (br s, 3H).

### 3-(8-((4-chlorophenyl)ethynyl)-2-imino-3-methyl-2,3-dihydro-1H-imidazo[4,5-c]quinolin-1-yl)-4-methylbenzonitrile (155)

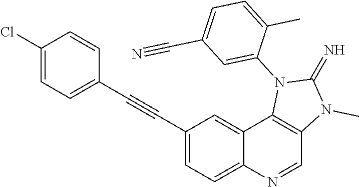 [SMILES: Cc1ccc(C#N)cc1-n1c(=N)n(C)c2cnc3ccc(C#Cc4ccc(Cl)cc4)cc3c21]

LCMS: t_{R}=4.919 min in 0-60 AB_7MIN_220&254 chromatography (Xtimate 3 um, C18, 2.1*30 mm), MS (ESI) m/z 448.0 [M+H]⁺.

¹H NMR: (400 MHz, DMSO-d₆) δ=8.87 (s, 1H), 8.19 (br s, 1H), 8.11 (br d, J=7.9 Hz, 1H), 7.98 (d, J=8.8 Hz, 1H), 7.84 (br d, J=7.7 Hz, 1H), 7.65 (br d, J=8.6 Hz, 1H), 7.56 (dd, J=1.8, 8.8 Hz, 1H), 7.52-7.45 (m, 4H), 6.77 (s, 1H), 3.54 (s, 3H), 2.15 (s, 3H).

### 8-(4-chlorophenyl)-3-methyl-1-(pyrimidin-5-yl)-1,3-dihydro-2H-imidazo[4,5-c]quinolin-2-imine

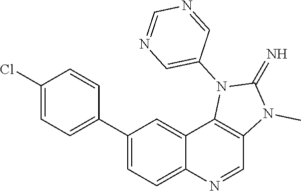 [SMILES: Cn1c(=N)n(-c2cncnc2)c2c3cc(-c4ccc(Cl)cc4)ccc3ncc21]

LCMS: t_{R}=3.411 min in 0-60AB_7.0 MIN chromatography (Xtimate C18 2.1*30 mm, 3 um), MS (ESI) m/z 387.1 [M+H]⁺.

¹H NMR (400 MHz, DMSO-d₆) δ=9.66 (s, 1H), 9.41 (d, J=6.4 Hz, 5H), 8.29 (d, J=8.8 Hz, 1H), 8.04 (dd, J=2.0, 8.8 Hz, 1H), 7.51 (d, J=8.6 Hz, 2H), 7.37 (d, J=8.6 Hz, 2H), 6.93 (d, J=1.7 Hz, 1H), 3.99 (s, 3H)

### 8-(4-fluorophenyl)-3-methyl-1-(pyrimidin-5-yl)-1,3-dihydro-2H-imidazo[4,5-c]quinolin-2-imine

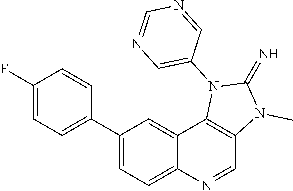 [SMILES: Cn1c(=N)n(-c2cncnc2)c2c3cc(-c4ccc(F)cc4)ccc3ncc21]

LCMS: t_{R}=3.010 min in 0-60AB_7.0 MIN chromatography (Xtimate C18 2.1*30 mm, 3 um), MS (ESI) m/z 371.1 [M+H]⁺.

¹H NMR (400 MHz, DMSO-d₆) δ=9.68 (s, 1H), 9.49-9.33 (m, 5H), 8.30 (d, J=8.8 Hz, 1H), 8.06 (dd, J=2.0, 9.0 Hz, 1H), 7.49-7.21 (m, 4H), 6.93 (d, J=1.7 Hz, 1H), 4.01 (s, 3H)

### 5-(2-imino-3-methyl-1-(pyrimidin-5-yl)-2,3-dihydro-1H-imidazo[4,5-c]quinolin-8-yl)pyridin-2-amine

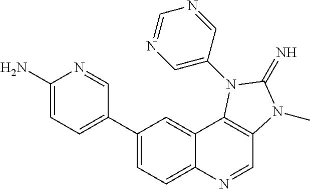 [SMILES: Cn1c(=N)n(-c2cncnc2)c2c3cc(-c4ccc(N)nc4)ccc3ncc21]

LCMS: t_{R}=1.507 min in 0-60AB_7 MIN chromatography (Xtimate C18 2.1*30 mm, 3 um), MS (ESI) m/z 369.1 [M+H]⁺.

¹H NMR (400 MHz, DMSO-d₆) δ=9.67 (s, 1H), 9.48-9.33 (m, 5H), 8.30 (d, J=8.8 Hz, 2H), 8.13-7.95 (m, 2H), 7.86 (dd, J=2.3, 9.3 Hz, 1H), 7.07 (d, J=9.3 Hz, 1H), 6.92 (d, J=1.8 Hz, 1H), 4.01 (s, 3H)

### 3-methyl-1-(pyrimidin-5-yl)-8-(p-tolyl)-1,3-dihydro-2H-imidazo[4,5-c]quinolin-2-imine

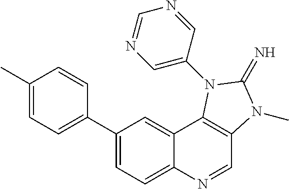

LCMS: t_{R}=3.269 min in 0-60AB_7 MIN chromatography (Xtimate C18 2.1*30 mm, 3 um), MS (ESI) m/z 367.1 [M+H]⁺.

¹H NMR (400 MHz, DMSO-d₆) δ=9.70 (s, 1H), 9.49-9.29 (m, 5H), 8.39-8.19 (m, 1H), 8.11-7.95 (m, 1H), 7.26 (s, 3H), 6.94 (d, J=1.8 Hz, 1H), 4.00 (s, 3H), 2.33 (s, 3H)

### 8-(3-fluorophenyl)-3-methyl-1-(pyrimidin-5-yl)-1,3-dihydro-2H-imidazo[4,5-c]quinolin-2-imine

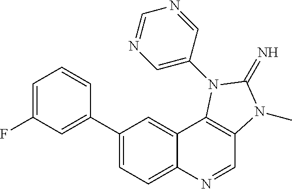 [SMILES: Cn1c(=N)n(-c2cncnc2)c2c3cc(-c4cccc(F)c4)ccc3ncc21]

LCMS: t_{R}=3.027 min in 0-60AB_7 MIN chromatography (Xtimate C18 2.1*30 mm, 3 um), MS (ESI) m/z 371.1 [M+H]⁺.

¹H NMR (400 MHz, DMSO-d₆) δ=9.70 (s, 1H), 9.49-9.31 (m, 5H), 8.30 (d, J=9.0 Hz, 1H), 8.10 (dd, J=2.0, 9.0 Hz, 1H), 7.51 (q, J=7.5 Hz, 1H), 7.33-7.17 (m, 3H), 6.99 (d, J=1.8 Hz, 1H), 4.00 (s, 3H)

### 8-(4-chlorophenyl)-3-methyl-1-(4-methylpyrimidin-5-yl)-1,3-dihydro-2H-imidazo[4,5-c]quinolin-2-imine

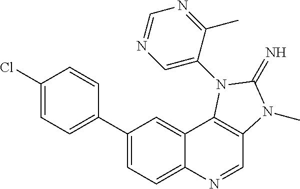 [SMILES: Cc1ncncc1-n1c(=N)n(C)c2cnc3ccc(-c4ccc(Cl)cc4)cc3c21]

LCMS: t_{R}=3.482 min in 0-60AB_7.0 MIN chromatography (Xtimate C18 2.1*30 mm, 3 um), MS (ESI) m/z 401.1 [M+H]⁺.

¹H NMR (400 MHz, METHANOL-d₄) δ=9.61 (s, 1H), 9.52 (s, 1H), 9.21 (s, 1H), 8.38 (d, J=9.0 Hz, 1H), 8.23 (dd, J=2.0, 9.0 Hz, 1H), 7.53-7.41 (m, 2H), 7.40-7.31 (m, 2H), 7.06 (d, J=1.5 Hz, 1H), 4.06 (s, 3H), 2.55 (s, 3H)

### 8-(4-fluorophenyl)-3-methyl-1-(4-methylpyrimidin-5-yl)-1,3-dihydro-2H-imidazo[4,5-c]quinolin-2-imine

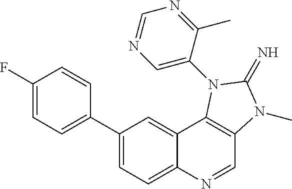 [SMILES: Cc1ncncc1-n1c(=N)n(C)c2cnc3ccc(-c4ccc(F)cc4)cc3c21]

LCMS: t_{R}=3.138 min in 0-60AB_7.0 MIN chromatography (Xtimate C18 2.1*30 mm, 3 um), MS (ESI) m/z 385.1 [M+H]⁺.

¹H NMR (400 MHz, METHANOL-d₄) δ=9.74 (s, 1H), 9.53 (s, 1H), 9.24 (s, 1H), 8.45-8.37 (m, 1H), 8.31 (dd, J=1.7, 9.0 Hz, 1H), 7.46-7.35 (m, 2H), 7.21 (t, J=8.8 Hz, 2H), 7.08 (d, J=1.7 Hz, 1H), 4.08 (s, 3H), 2.57 (s, 3H)

### 5-(2-imino-3-methyl-1-(4-methylpyrimidin-5-yl)-2,3-dihydro-1H-imidazo[4,5-c]quinolin-8-yl)pyridin-2-amine

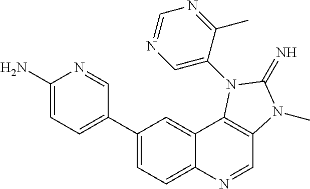 [SMILES: Cc1ncncc1-n1c(=N)n(C)c2cnc3ccc(-c4ccc(N)nc4)cc3c21]

LCMS: t_{R}=1.627 min in 0-60AB_7.0 MIN chromatography (Xtimate C18 2.1*30 mm, 3 um), MS (ESI) m/z 383.2 [M+H]⁺.

¹H NMR (400 MHz, DMSO-d₆) δ=9.59-9.34 (m, 4H), 9.19 (s, 1H), 8.59-8.15 (m, 3H), 8.11-7.97 (m, 2H), 7.81 (br d, J=9.3 Hz, 1H), 7.18-7.00 (m, 1H), 6.82 (s, 1H), 4.01 (s, 3H), 2.45 (s, 3H)

### 3-methyl-1-(4-methylpyrimidin-5-yl)-8-(p-tolyl)-1,3-dihydro-2H-imidazo[4,5-c]quinolin-2-imine

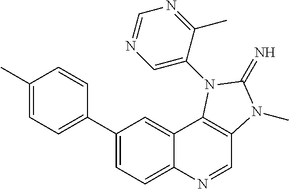

LCMS: t_{R}=3.358 min in 0-60AB_7.0 MIN chromatography (Xtimate C18 2.1*30 mm, 3 um), MS (ESI) m/z 381.2 [M+H]⁺.

¹H NMR (400 MHz, METHANOL-d₄) δ=9.63 (s, 1H), 9.57-9.51 (m, 1H), 9.23 (s, 1H), 8.42-8.33 (m, 1H), 8.28 (dd, J=1.7, 9.0 Hz, 1H), 7.32-7.24 (m, 4H), 7.08 (d, J=1.5 Hz, 1H), 4.13-4.03 (m, 3H), 2.57 (s, 3H), 2.39 (s, 3H)

### 8-(3-fluorophenyl)-3-methyl-1-(4-methylpyrimidin-5-yl)-1,3-dihydro-2H-imidazo[4,5-c]quinolin-2-imine

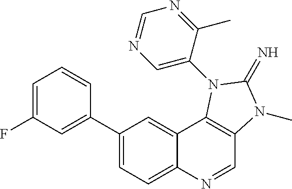 [SMILES: Cc1ncncc1-n1c(=N)n(C)c2cnc3ccc(-c4cccc(F)c4)cc3c21]

LCMS: t_{R}=3.178 min in 0-60AB_7.0 MIN chromatography (Xtimate C18 2.1*30 mm, 3 um), MS (ESI) m/z 385.1 [M+H]⁺.

¹H NMR (400 MHz, METHANOL-d₄) δ=9.90-9.73 (m, 1H), 9.54 (s, 1H), 9.31 (s, 1H), 8.54-8.29 (m, 2H), 7.63-7.44 (m, 1H), 7.29-7.04 (m, 4H), 4.17-4.04 (m, 3H), 2.60 (s, 3H)

### 1-(4-(5-(8-(4-chlorophenyl)-2-imino-3-methyl-2,3-dihydro-H-imidazo[4,5-c]quinolin-1-yl)pyrimidin-2-yl)piperazin-1-yl)ethan-1-one

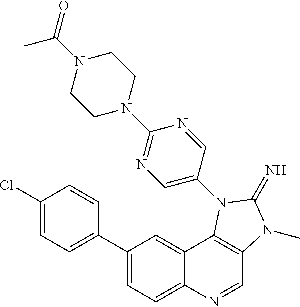 [SMILES: CC(=O)N1CCN(c2ncc(-n3c(=N)n(C)c4cnc5ccc(-c6ccc(Cl)cc6)cc5c43)cn2)CC1]

LCMS: t_{R}=4.267 min in 0-60AB_7.0 MIN chromatography (Xtimate C18 2.1*30 mm, 3 um), MS (ESI) m/z 513.2 [M+H]⁺.

### (E)-8-(3-fluorophenyl)-3-methyl-N,1-di(pyrimidin-5-yl)-1,3-dihydro-2H-imidazo[4,5-c]quinolin-2-imine

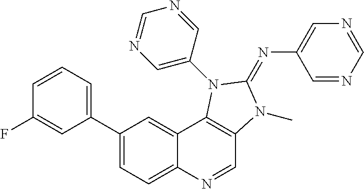 [SMILES: Cn1/c(=N\c2cncnc2)n(-c2cncnc2)c2c3cc(-c4cccc(F)c4)ccc3ncc21]

LCMS: t_{R}=3.799 min in 0-60AB_7.0 MIN chromatography (Xtimate C18 2.1*30 mm, 3 um), MS (ESI) m/z 449.0 [M+H]⁺.

### 3-methyl-8-(1-methyl-1H-pyrazol-5-yl)-1-(pyrimidin-5-yl)-1,3-dihydro-2H-imidazo[4,5-c]quinolin-2-imine

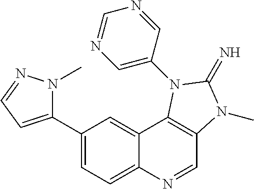 [SMILES: Cn1nccc1-c1ccc2ncc3c(c2c1)n(-c1cncnc1)c(=N)n3C]

LCMS: t_{R}=2.965 min in 0-60AB_7.0 MIN chromatography (Xtimate C18 2.1*30 mm, 3 um), MS (ESI) m/z 357.1 [M+H]⁺.

### 8-(4-chloro-3-methoxyphenyl)-3-methyl-1-(4-methylpyrimidin-5-yl)-1,3-dihydro-2H-imidazo[4,5-c]quinolin-2-imine

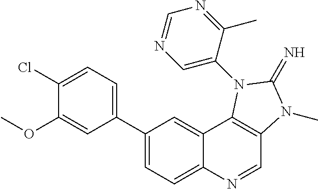

LCMS: t_{R}=4.151 min in 0-60AB_7.0 min chromatography (Xtimate, 2.1*30 mm, 3 um), MS (ESI) m/z 431.2 [M+H]⁺.

¹H NMR: (400 MHz, DMSO-d₆): δ=9.66 (brs, 2H), 9.51-9.45 (m, 2H), 9.22 (s, 1H), 8.34 (d, J=9.2 Hz, 1H), 8.15-8.12 (m, 1H), 7.49 (d, J=8.0 Hz, 1H), 7.01-7.00 (m, 1H), 6.99-6.96 (m, 1H), 6.91 (td, J=1.6 Hz, 1H), 4.02 (s, 3H), 3.89 (s, 3H), 2.44 (s, 3H).

### 8-(3-fluoro-4-methoxyphenyl)-3-methyl-1-(4-methylpyrimidin-5-yl)-1,3-dihydro-2H-imidazo[4,5-c]quinolin-2-imine

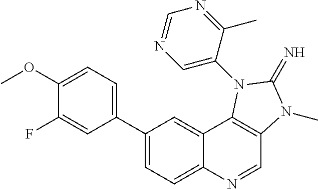

LCMS: t_{R}=3.783 min in 0-60AB_7.0 min chromatography (Xtimate, 2.1*30 mm, 3 um), MS (ESI) m/z 415.2 [M+H]⁺.

¹H NMR: (400 MHz, DMSO-d₆): δ=9.58 (brs, 2H), 9.51 (s, 1H), 9.45 (s, 1H), 9.23 (s, 1H), 8.29 (d, J=8.8 Hz, 1H), 8.10-8.07 (m, 1H), 7.28-7.23 (m, 2H), 7.12-7.10 (m, 1H), 6.84 (d, J=1.6 Hz, 1H), 4.02 (s, 3H), 3.88 (s, 3H), 2.45 (s, 3H).

### 8-(4-fluoro-3-methoxyphenyl)-3-methyl-1-(4-methylpyrimidin-5-yl)-1,3-dihydro-2H-imidazo[4,5-c]quinolin-2-imine

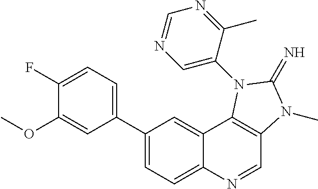

LCMS: t_{R}=3.847 min in 0-60AB_7.0 min chromatography (Xtimate, 2.1*30 mm, 3 um), MS (ESI) m/z 415.2 [M+H]⁺.

¹H NMR: (400 MHz, DMSO-d₆): δ=9.86 (brs, 2H), 9.59 (s, 1H), 9.48 (s, 1H), 9.24 (s, 1H), 8.39 (d, J=8.8 Hz, 1H), 8.18-8.16 (m, 1H), 7.32-7.27 (m, 2H), 7.01-6.97 (m, 2H), 6.89 (d, J=2.0 Hz, 1H), 4.04 (s, 3H), 3.87 (s, 3H), 2.45 (s, 3H).

### 8-(3,4-dimethoxyphenyl)-3-methyl-1-(pyrimidin-5-yl)-1,3-dihydro-2H-imidazo[4,5-c]quinolin-2-imine

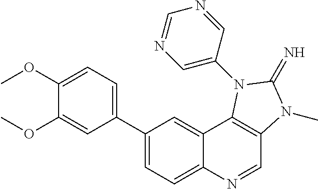

LCMS: t_{R}=3.495 min in 0-60AB_7.0 MIN chromatography (Xtimate C18 2.1*30 mm, 3 um), MS (ESI) m/z 413.0 [M+H]⁺.

### 8-(4-chloro-3-(trifluoromethyl)phenyl)-3-methyl-1-(4-methylpyrimidin-5-yl)-1,3-dihydro-2H-imidazo[4,5-c]quinolin-2-imine

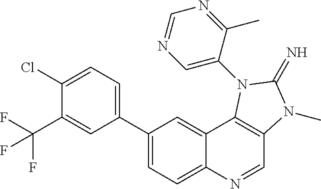 [SMILES: Cc1ncncc1-n1c(=N)n(C)c2cnc3ccc(-c4ccc(Cl)c(C(F)(F)F)c4)cc3c21]

LCMS: t_{R}=4.560 min in 0-60AB_7.0 MIN chromatography (Xtimate C18 2.1*30 mm, 3 um), MS (ESI) m/z 469.0 [M+H]⁺.

### 2-chloro-5-(2-imino-3-methyl-1-(pyrimidin-5-yl)-2,3-dihydro-1H-imidazo[4,5-c]quinolin-8-yl)benzonitrile

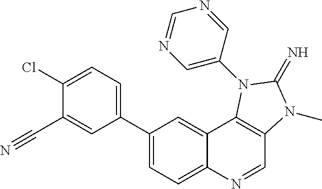 [SMILES: Cn1c(=N)n(-c2cncnc2)c2c3cc(-c4ccc(Cl)c(C#N)c4)ccc3ncc21]

LCMS: t_{R}=4.019 min in 0-60AB_7.0 MIN chromatography (Xtimate C18 2.1*30 mm, 3 um), MS (ESI) m/z 412.1 [M+H]⁺.

### 8-(4-chloro-3-(trifluoromethoxy)phenyl)-3-methyl-1-(4-methylpyrimidin-5-yl)-1,3-dihydro-2H-imdazo[4,5-c]quinolin-2-imine

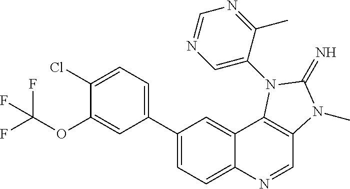 [SMILES: Cc1ncncc1-n1c(=N)n(C)c2cnc3ccc(-c4ccc(Cl)c(OC(F)(F)F)c4)cc3c21]

LCMS: t_{R}=4.747 min in 0-60AB_7.0 MIN chromatography (Xtimate C18 2.1*30 mm, 3 um), MS (ESI) m/z 485.1 [M+H]⁺.

### 8-(4-chloro-3-(trifluoromethoxy)phenyl)-3-methyl-1-(pyrimidin-5-yl)-1,3-dihydro-2H-imidazo[4,5-c]quinolin-2-imine

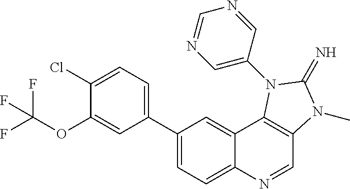 [SMILES: Cn1c(=N)n(-c2cncnc2)c2c3cc(-c4ccc(Cl)c(OC(F)(F)F)c4)ccc3ncc21]

LCMS: t_{R}=4.702 min in 0-60AB_7.0 MIN chromatography (Xtimate C18 2.1*30 mm, 3 um), MS (ESI) m/z 471.1 [M+H]⁺.

### 8-(3,4-dimethoxyphenyl)-3-methyl-1-(4-methylpyrimidin-5-yl)-1,3-dihydro-2H-imidazo[4,5-c]quinolin-2-imine

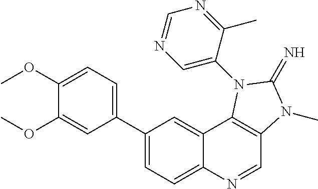

LCMS: t_{R}=3.562 min in 0-60AB_7.0 MIN chromatography (Xtimate C18 2.1*30 mm, 3 um), MS (ESI) m/z 427.1 [M+H]⁺.

### 3-methyl-1-(pyrimidin-5-yl)-8-(6-(trifluoromethyl)pyridin-3-yl)-1,3-dihydro-2H-imidazo[4,5-c]quinolin-2-imine

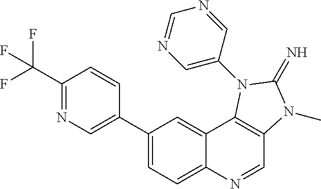 [SMILES: Cn1c(=N)n(-c2cncnc2)c2c3cc(-c4ccc(C(F)(F)F)nc4)ccc3ncc21]

LCMS: t_{R}=3.823 min in 0-60AB_7.0 MIN chromatography (Xtimate C18 2.1*30 mm, 3 um), MS (ESI) m/z 422.2 [M+H]⁺.

### 3-methyl-8-(6-methylpyridin-3-yl)-1-(pyrimidin-5-yl)-1,3-dihydro-2H-imidazo [4,5-c]quinolin-2-imine

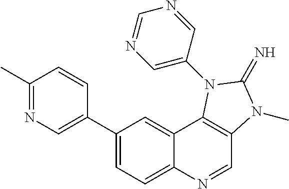

LCMS: t_{R}=2.418 min in 0-60AB_7.0 MIN chromatography (Xtimate C18 2.1*30 mm, 3 um), MS (ESI) m/z 368.2 [M+H]⁺.

### 8-(6-methoxypyridin-3-yl)-3-methyl-1-(pyrimidin-5-yl)-1,3-dihydro-2H-imidazo[4,5-c]quinolin-2-imine

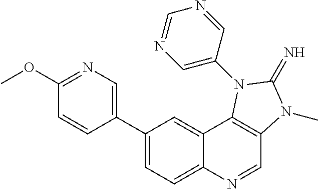

LCMS: t_{R}=3.503 min in 0-60AB_7.0 MIN chromatography (Xtimate C18 2.1*30 mm, 3 um), MS (ESI) m/z 384.2 [M+H]⁺.

### 4-(8-(4-chlorophenyl)-2-imino-3-methyl-2,3-dihydro-1H-imidazo[4,5-c]quinolin-1-yl)pyrimidin-2-amine

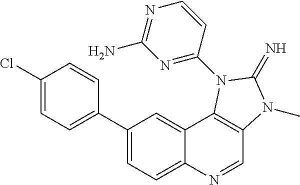 [SMILES: Cn1c(=N)n(-c2ccnc(N)n2)c2c3cc(-c4ccc(Cl)cc4)ccc3ncc21]

LCMS: t_{R}=3.299 min in 0-60AB_7 min_220&254_Shimadzu chromatography (Xtimate C18 2.1*30 mm), MS (ESI) m/z 402.0 [M+H]⁺.

¹H NMR (400 MHz, DMSO-d₆) δ=9.54 (br s, 2H), 9.48 (s, 1H), 8.76 (d, J=4.8 Hz, 1H), 8.35 (d, J=8.9 Hz, 1H), 8.14 (br d, J=9.0 Hz, 1H), 7.65-7.52 (m, 6H), 7, 7.14 (d, J=4.9 Hz, 1H), 3.98 (s, 3H).

### 4-(8-(3-fluorophenyl)-2-imino-3-methyl-2,3-dihydro-1H-imidazo[4,5-c]quinolin-1-yl)pyrimidin-2-amine

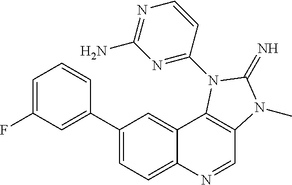 [SMILES: Cn1c(=N)n(-c2ccnc(N)n2)c2c3cc(-c4cccc(F)c4)ccc3ncc21]

LCMS: t_{R}=3.024 min in 0-60AB_7 min_220&254_Shimadzu chromatography (Xtimate C18 2.1*30 mm), MS (ESI) m/z 386.0 [M+H]⁺.

¹H NMR (400 MHz, DMSO-d₆) δ=9.96 (br s, 2H), 9.68 (s, 1H), 8.79 (br d, J=4.6 Hz, 1H), 8.46 (br d, J=8.9 Hz, 1H), 8.26 (br d, J=8.9 Hz, 1H), 7.61 (s, 1H), 7.53 (q, J=7.3 Hz, 1H), 7.38 (br d, J=8.5 Hz, 2H), 7.29 (br t, J=8.4 Hz, 1H), 7.19 (br d, J=4.6 Hz, 1H), 4.05 (s, 3H).

### 8-(4-chlorophenyl)-3-methyl-1-(4-(trifluoromethyl)pyrimidin-2-yl)-1,3-dihydro-2H-imidazo[4,5-c]quinolin-2-imine

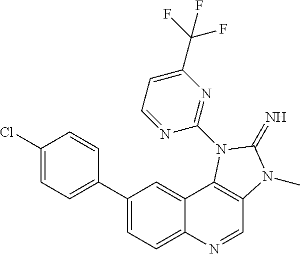 [SMILES: Cn1c(=N)n(-c2nccc(C(F)(F)F)n2)c2c3cc(-c4ccc(Cl)cc4)ccc3ncc21]

LCMS: t_{R}=4.207 min in 0-60AB_7 min_220&254_Shimadzu chromatography (Xtimate C18 2.1*30 mm, 3 um), MS (ESI) m/z 455.2 [M+H]⁺.

¹H NMR (400 MHz, DMSO-d₆) δ=9.63 (d, J=5.1 Hz, 1H), 9.54 (br s, 2H), 9.38 (s, 1H), 8.50 (d, J=5.1 Hz, 1H), 8.26 (d, J=8.8 Hz, 1H), 8.06 (dd, J=2.0, 8.8 Hz, 1H), 7.63-7.55 (m, 3H), 7.47 (d, J=8.6 Hz, 2H), 3.94 (s, 3H).

### 8-(3-fluorophenyl)-3-methyl-1-(4-(trifluoromethyl)pyrimidin-2-yl)-1,3-dihydro-2H-imidazo[4,5-c]quinolin-2-imine

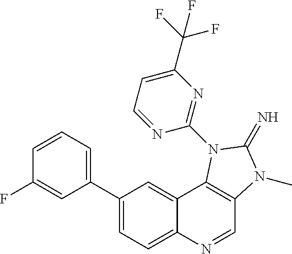 [SMILES: Cn1c(=N)n(-c2nccc(C(F)(F)F)n2)c2c3cc(-c4cccc(F)c4)ccc3ncc21]

LCMS: t_{R}=4.372 min in 0-60AB_7 min_220&254_Shimadzu chromatography (Xtimate C18 2.1*30 mm, 3 um), MS (ESI) m/z 439.2 [M+H]⁺.

¹H NMR (400 MHz, DMSO-d₆) δ=9.63 (d, J=5.1 Hz, 1H), 9.59 (br s, 2H), 9.40 (s, 1H), 8.52 (d, J=5.1 Hz, 1H), 8.27 (d, J=8.8 Hz, 1H), 8.10 (dd, J=2.0, 9.0 Hz, 1H), 7.67 (d, J=1.7 Hz, 1H), 7.51-7.36 (m, 3H), 7.21 (br t, J=8.2 Hz, 1H), 3.95 (s, 3H).

### 8-(4-chlorophenyl)-1-(4-methoxypyrimidin-2-yl)-3-methyl-1,3-dihydro-2H-imidazo[4,5-c]quinolin-2-imine

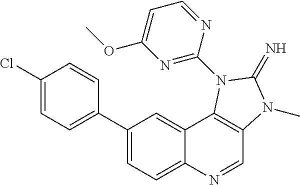 [SMILES: COc1ccnc(-n2c(=N)n(C)c3cnc4ccc(-c5ccc(Cl)cc5)cc4c32)n1]

LCMS: t_{R}=4.031 min in 0-60AB_7 min_220&254_Shimadzu (Xtimate C18 2.1*30 mm, 3 um), MS (ESI) m/z 417.2 [M+H]⁺.

¹H NMR: (400 MHz, DMSO-d₆) δ=9.58-9.44 (m, 1H), 9.47 (br s, 1H), 9.39 (s, 1H), 8.96 (d, J=5.8 Hz, 1H), 8.29 (d, J=8.8 Hz, 1H), 8.14-8.07 (m, 1H), 7.57 (q, J=8.7 Hz, 5H), 7.42 (d, J=6.0 Hz, 1H), 3.97 (s, 3H), 3.95 (s, 3H)

### 2-(8-(4-chlorophenyl)-2-imino-3-methyl-2,3-dihydro-1H-imidazo[4,5-c]quinolin-1-yl)pyrimidin-4-ol

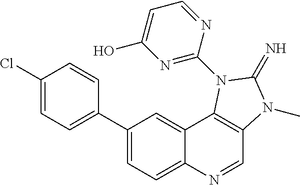 [SMILES: Cn1c(=N)n(-c2nccc(O)n2)c2c3cc(-c4ccc(Cl)cc4)ccc3ncc21]

LCMS: t_{R}=3.692 min in 0-60AB_7 min_220&254_Shimadzu (Xtimate C18 2.1*30 mm, 3 um), MS (ESI) m/z 403.2 [M+H]⁺.

¹H NMR: (400 MHz, DMSO-d₆) δ=9.54-9.40 (m, 2H), 9.37 (s, 1H), 8.81 (d, J=5.9 Hz, 1H), 8.28 (d, J=8.8 Hz, 1H), 8.08 (dd, J=2.0, 9.0 Hz, 1H), 7.70-7.59 (m, 3H), 7.58-7.53 (m, 2H), 7.16 (d, J=6.1 Hz, 1H), 3.94 (s, 3H)

### 8-(3-fluorophenyl)-1-(4-methoxypyrimidin-2-yl)-3-methyl-1,3-dihydro-2H-imidazo[4,5-c]quinolin-2-imine

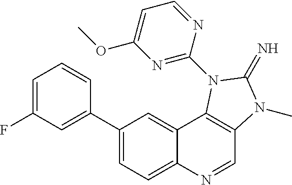 [SMILES: COc1ccnc(-n2c(=N)n(C)c3cnc4ccc(-c5cccc(F)c5)cc4c32)n1]

LCMS: t_{R}=5.051 min in 0-60AB_7 min_220&254_Shimadzu (Xtimate C18 2.1*30 mm, 3 um), MS (ESI) m/z 401.2 [M+H]⁺.

¹H NMR: (400 MHz, DMSO-d₆) δ=9.41 (br s, 2H), 9.40 (s, 1H), 8.96 (d, J=6.0 Hz, 1H), 8.29 (d, J=8.8 Hz, 1H), 8.13 (dd, J=2.0, 9.0 Hz, 1H), 7.65 (s, 1H), 7.56-7.50 (m, 1H), 7.47-7.40 (m, 3H), 7.30-7.24 (m, 1H), 3.96 (s, 3H), 3.95 (s, 3H)

### 2-(8-(3-fluorophenyl)-2-imino-3-methyl-2,3-dihydro-1H-imidazo[4,5-c]quinolin-1-yl)pyrimidin-4-ol

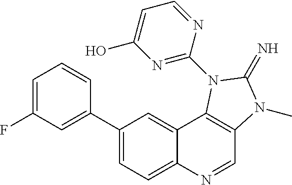 [SMILES: Cn1c(=N)n(-c2nccc(O)n2)c2c3cc(-c4cccc(F)c4)ccc3ncc21]

LCMS: t_{R}=3.187 min in 0-60AB_7.0 min_220&254_Shimadzu (Xtimate C18 2.1*30 mm, 3 um), MS (ESI) m/z 387.1 [M+H]⁺.

¹H NMR: (400 MHz, DMSO-d₆) δ=9.31 (s, 2H), 8.24 (d, J=9.0 Hz, 1H), 8.17-7.97 (m, 4H), 7.57-7.47 (m. 3H), 7.25 (s, 1H), 3.90 (s, 3H)

### 2-(8-(4-chlorophenyl)-2-imino-3-methyl-2,3-dihydro-1H-imidazo[4,5-c]quinolin-1-yl)pyrimidin-4-amine

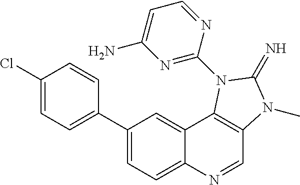 [SMILES: Cn1c(=N)n(-c2nccc(N)n2)c2c3cc(-c4ccc(Cl)cc4)ccc3ncc21]

LCMS: t_{R}=3.400 min in 0-60AB_7 min_220&254_Shimadzu chromatography (Xtimate C18 2.1*30 mm), MS (ESI) m/z 402.0 [M+H]⁺.

¹H NMR (400 MHz, DMSO-d₆) δ=9.62 (br s, 2H), 9.45 (s, 1H), 8.39 (br d, J=5.6 Hz, 1H), 8.32 (br d, J=8.8 Hz, 1H), 8.12 (br d, J=8.9 Hz, 1H), 8.03 (br s, 1H), 7.82 (br s, 1H), 7.76 (s, 1H), 7.67-7.61 (m, 2H), 7.60-7.52 (m, 2H), 6.82 (br d, J=5.9 Hz, 1H), 3.98 (s, 3H).

### 2-(8-(3-fluorophenyl)-2-imino-3-methyl-2,3-dihydro-1H-imidazo[4,5-c]quinolin-1-yl)pyrimidin-4-amine

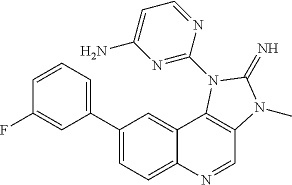 [SMILES: Cn1c(=N)n(-c2nccc(N)n2)c2c3cc(-c4cccc(F)c4)ccc3ncc21]

LCMS: t_{R}=3.130 min in 0-60AB_7 min_220&254_Shimadzu chromatography (Xtimate C18 2.1*30 mm), MS (ESI) m/z 386.0 [M+H]⁺.

¹H NMR (400 MHz, DMSO-d₆) δ=9.34 (br d, J=3.6 Hz, 3H), 8.39 (br d, J=5.6 Hz, 1H), 8.24 (br d, J=8.8 Hz, 1H), 8.08 (br d, J=8.8 Hz, 1H), 7.96 (br s, 1H), 7.77 (s, 2H), 7.53 (q, J=7.1 Hz, 1H), 7.49-7.40 (m, 2H), 7.35-7.22 (m, 1H), 6.79 (br d, J=5.8 Hz, 1H), 3.92 (s, 3H).

### 8-(3,4-dimethoxyphenyl)-3-methyl-1-(3-methylpyrazin-2-yl)-1,3-dihydro-2H-imidazo[4,5-c]quinolin-2-imine

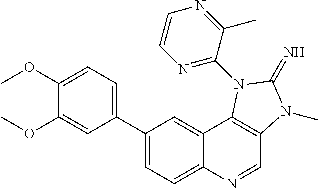

LCMS: t_{R}=3.768 min in 0-60AB_7 min_220&254_Shimadzu chromatography (Xtimate C18 2.1*30 mm), MS (ESI) m/z 427.1[M+H]⁺.

### 3-methyl-8-(6-methylpyridin-3-yl)-1-(4-(methylsulfonyl)phenyl)-1,3-dihydro-2H-imidazo[4,5-c]quinolin-2-imine

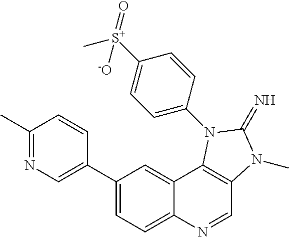

LCMS: t_{R}=2.739 min in 0-60AB_7 min_220&254_Shimadzu chromatography (Xtimate C18 2.1*30 mm), MS (ESI) m/z 444.2 [M+H]⁺.

### 8-(3,4-dimethoxyphenyl)-1-(pyridazin-4-yl)-1,3-dihydro-2H-imidazo[4,5-c]quinolin-2-imine

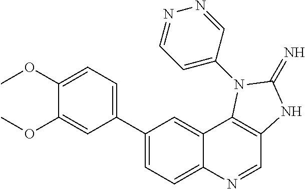 [SMILES: COc1ccc(-c2ccc3ncc4[nH]c(=N)n(-c5ccnnc5)c4c3c2)cc1OC]

LCMS: t_{R}=3.409 min in 0-60AB_7 min_220&254_Shimadzu chromatography (Xtimate C18 2.1*30 mm), MS (ESI) m/z 399.2 [M+H]⁺.

### 3-methyl-1-(4-methylpyrimidin-5-yl)-8-(3-(methylsulfonyl)phenyl)-1,3-dihydro-2H-imidazo[4,5-c]quinolin-2-imine

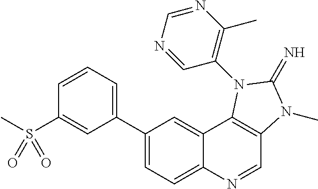

LCMS: t_{R}=3.268 min in 0-60AB_7 min_220&254_Shimadzu chromatography (Xtimate C18 2.1*30 mm), MS (ESI) m/z 445.0 [M+H]⁺.

### 8-(3-fluoro-4,5-dimethoxyphenyl)-3-methyl-1-(4-methylpyrimidin-5-yl)-1,3-dihydro-2H-imidazo[4,5-c]quinolin-2-imine

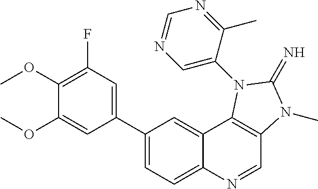

LCMS: t_{R}=4.074 min in 0-60AB_7 min_220&254_Shimadzu chromatography (Xtimate C18 2.1*30 mm), MS (ESI) m/z 445.2 [M+H]+.

### Example 4

This example demonstrates the gametocytocidal activity and activity against asexual parasites in accordance with an embodiment of the invention.

Compounds were screened against gametocytes and asexual parasites as described in Examples 1 and 2. The results are set forth in Tables 1-3.

TABLE 1

| | | | Microsomal | | |
|---|---|---|---|---|---|
| | | Asexual | Stability | Solubility | PAMPA |
| | | EC50 | (rat) T1/2 | pH 7.4 | Permeability |
| # | Structure | (nM) | (min) | (ug/mL) | (x106 cm/s) |
| 1 | 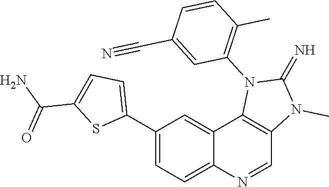 [SMILES: Cc1ccc(C#N)cc1-n1c(=N)n(C)c2cnc3ccc(-c4ccc(C(N)=O)s4)cc3c21] | 8.36 | >30 | 14.2 | |
| 2 | 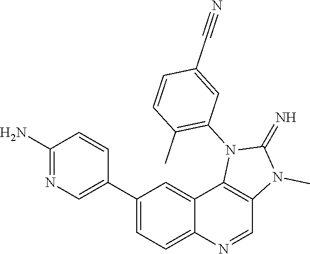 [SMILES: Cc1ccc(C#N)cc1-n1c(=N)n(C)c2cnc3ccc(-c4ccc(N)nc4)cc3c21] | 5.31 | >30 | >60 | |
| 3 | 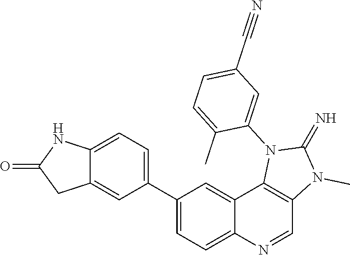 [SMILES: Cc1ccc(C#N)cc1-n1c(=N)n(C)c2cnc3ccc(-c4ccc5c(c4)CC(=O)N5)cc3c21] | 20.31 | >30 | 3.6 | |
| 4 | 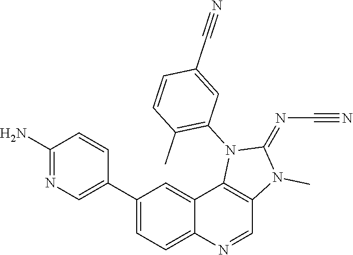 [SMILES: Cc1ccc(C#N)cc1-n1/c(=N/C#N)n(C)c2cnc3ccc(-c4ccc(N)nc4)cc3c21] | 11.89 | >30 | 3.7 | 34.1 |
| 5 | 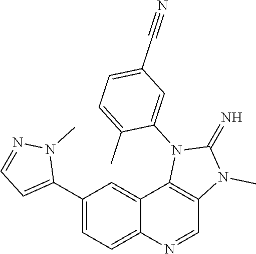 [SMILES: Cc1ccc(C#N)cc1-n1c(=N)n(C)c2cnc3ccc(-c4ccnn4C)cc3c21] | 153.8 | >30 | 42.5 | 219 |
| 6 | 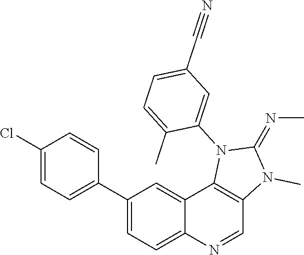 [SMILES: C/N=c1\n(C)c2cnc3ccc(-c4ccc(Cl)cc4)cc3c2n1-c1cc(C#N)ccc1C] | 279.3 | >30 | 9.6 | 921 |
| 7 | 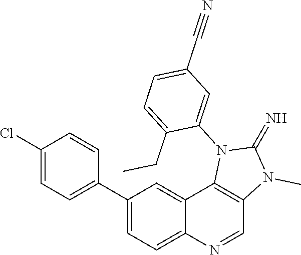 [SMILES: CCc1ccc(C#N)cc1-n1c(=N)n(C)c2cnc3ccc(-c4ccc(Cl)cc4)cc3c21] | 360 | >30 | 3.4 | |
| 8 | 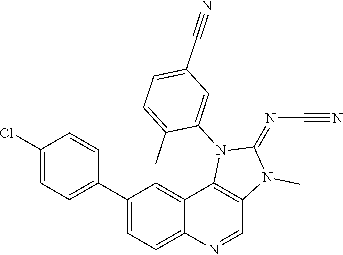 [SMILES: Cc1ccc(C#N)cc1-n1/c(=N/C#N)n(C)c2cnc3ccc(-c4ccc(Cl)cc4)cc3c21] | 59.84 | >30 | <1 | 1187.2 |
| 9 | 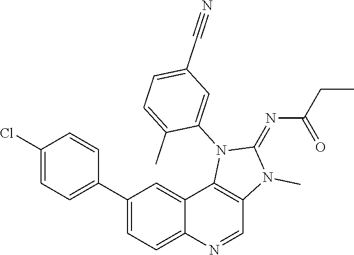 [SMILES: CCC(=O)/N=c1\n(C)c2cnc3ccc(-c4ccc(Cl)cc4)cc3c2n1-c1cc(C#N)ccc1C] | 38.52 | >30 | <1 | 526 |
| 10 | 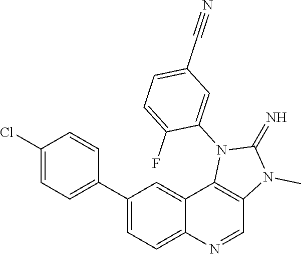 [SMILES: Cn1c(=N)n(-c2cc(C#N)ccc2F)c2c3cc(-c4ccc(Cl)cc4)ccc3ncc21] | 24.45 | >30 | <1 | |
| 11 | 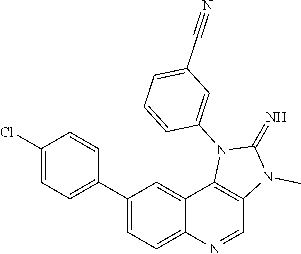 [SMILES: Cn1c(=N)n(-c2cccc(C#N)c2)c2c3cc(-c4ccc(Cl)cc4)ccc3ncc21] | 48.25 | >30 | 3.1 | 1004.7 |
| 12 | 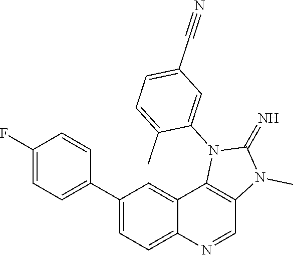 [SMILES: Cc1ccc(C#N)cc1-n1c(=N)n(C)c2cnc3ccc(-c4ccc(F)cc4)cc3c21] | 64.43 | >30 | 3.9 | 1025.6 |
| 13 | 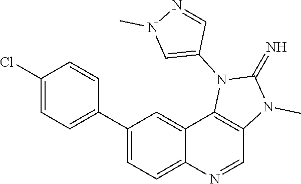 [SMILES: Cn1cc(-n2c(=N)n(C)c3cnc4ccc(-c5ccc(Cl)cc5)cc4c32)cn1] | 477.2 | >30 | >38 | |
| 14 | 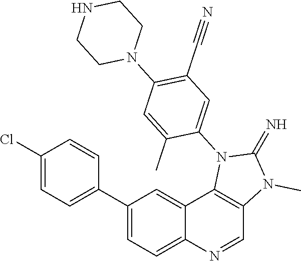 [SMILES: Cc1cc(N2CCNCC2)c(C#N)cc1-n1c(=N)n(C)c2cnc3ccc(-c4ccc(Cl)cc4)cc3c21] | 175 | >30 | <1 | |
| 15 | 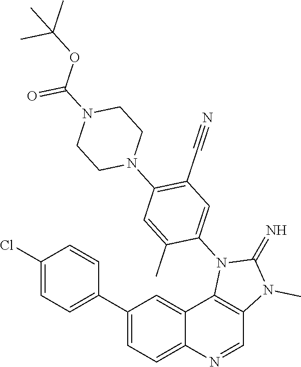 [SMILES: Cc1cc(N2CCN(C(=O)OC(C)(C)C)CC2)c(C#N)cc1-n1c(=N)n(C)c2cnc3ccc(-c4ccc(Cl)cc4)cc3c21] | 591 | >30 | <1 | |
| 16 | 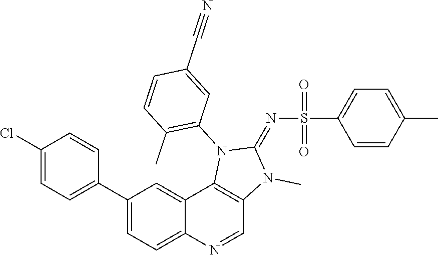 [SMILES: Cc1ccc(S(=O)(=O)/N=c2\n(C)c3cnc4ccc(-c5ccc(Cl)cc5)cc4c3n2-c2cc(C#N)ccc2C)cc1] | 239.7 | 10.4 | <1 | |
| 17 | 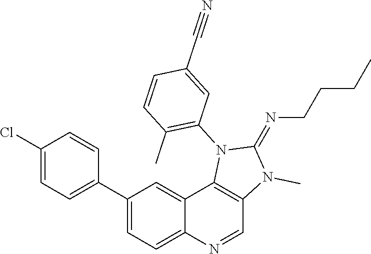 [SMILES: CCCC/N=c1\n(C)c2cnc3ccc(-c4ccc(Cl)cc4)cc3c2n1-c1cc(C#N)ccc1C] | 542.1 | >30 | 1.1 | |
| 18 | 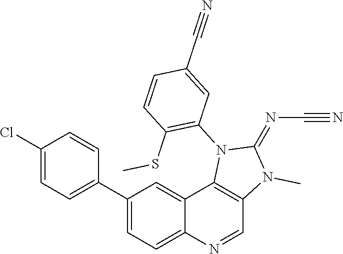 [SMILES: CSc1ccc(C#N)cc1-n1/c(=N/C#N)n(C)c2cnc3ccc(-c4ccc(Cl)cc4)cc3c21] | 563.7 | 16.9 | <1 | 246.6 |
| 19 | 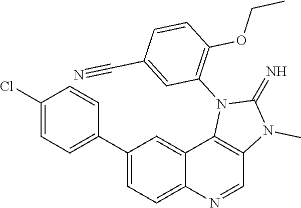 [SMILES: CCOc1ccc(C#N)cc1-n1c(=N)n(C)c2cnc3ccc(-c4ccc(Cl)cc4)cc3c21] | 778 | >30 | 26 | |
| 20 | 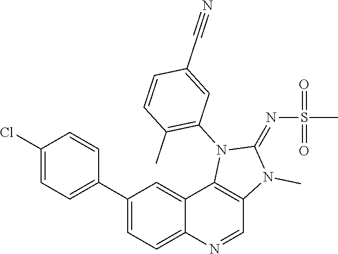 [SMILES: Cc1ccc(C#N)cc1-n1/c(=N/S(C)(=O)=O)n(C)c2cnc3ccc(-c4ccc(Cl)cc4)cc3c21] | 6.28 | >30 | <1 | |
| 21 | 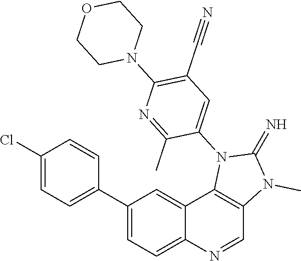 [SMILES: Cc1nc(N2CCOCC2)c(C#N)cc1-n1c(=N)n(C)c2cnc3ccc(-c4ccc(Cl)cc4)cc3c21] | 317.3 | >30 | 4.4 | 1564.7 |
| 22 | 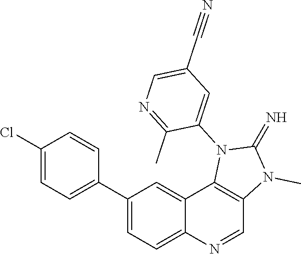 [SMILES: Cc1ncc(C#N)cc1-n1c(=N)n(C)c2cnc3ccc(-c4ccc(Cl)cc4)cc3c21] | 62.3 | >30 | 3.5 | |
| 23 | 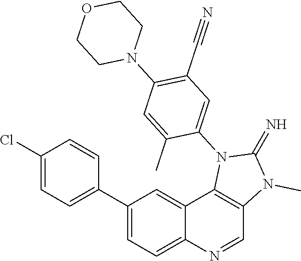 [SMILES: Cc1cc(N2CCOCC2)c(C#N)cc1-n1c(=N)n(C)c2cnc3ccc(-c4ccc(Cl)cc4)cc3c21] | 63.9 | >30 | <1 | 339.8 |
| 24 | 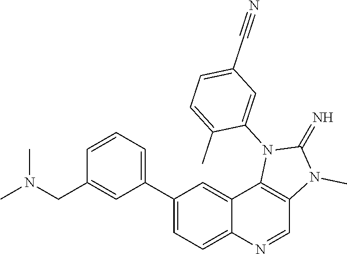 | 979.7 | >30 | >44 | 404.7 |
| 25 | 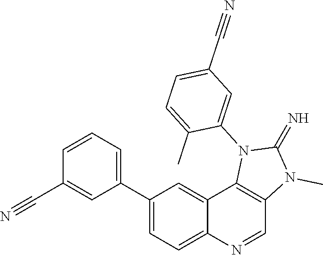 [SMILES: Cc1ccc(C#N)cc1-n1c(=N)n(C)c2cnc3ccc(-c4cccc(C#N)c4)cc3c21] | 118.4 | >30 | >41 | 1428.9 |
| 26 | 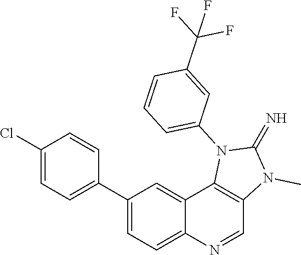 [SMILES: Cn1c(=N)n(-c2cccc(C(F)(F)F)c2)c2c3cc(-c4ccc(Cl)cc4)ccc3ncc21] | 529 | >30 | <1 | 1669.5 |
| 27 | 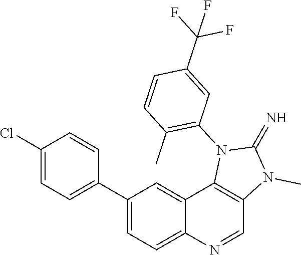 [SMILES: Cc1ccc(C(F)(F)F)cc1-n1c(=N)n(C)c2cnc3ccc(-c4ccc(Cl)cc4)cc3c21] | 137.7 | >30 | <1 | 1606.7 |
| 28 | 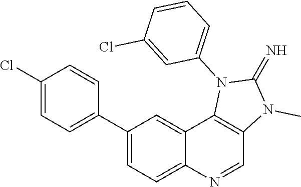 [SMILES: Cn1c(=N)n(-c2cccc(Cl)c2)c2c3cc(-c4ccc(Cl)cc4)ccc3ncc21] | 185.9 | >30 | <1 | 226 |
| 29 | 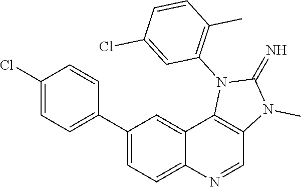 [SMILES: Cc1ccc(Cl)cc1-n1c(=N)n(C)c2cnc3ccc(-c4ccc(Cl)cc4)cc3c21] | 136 | >30 | <1 | 1684 |
| 30 | 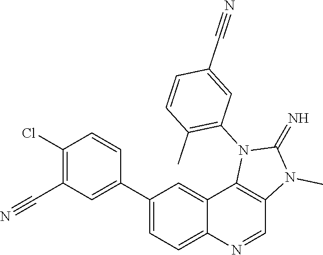 [SMILES: Cc1ccc(C#N)cc1-n1c(=N)n(C)c2cnc3ccc(-c4ccc(Cl)c(C#N)c4)cc3c21] | 60.3 | >30 | 7.3 | 1206.4 |
| 31 | 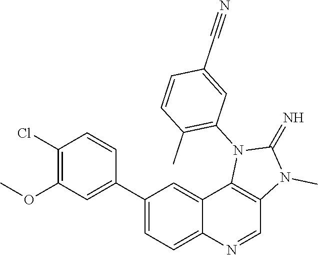 | 27.2 | >30 | <1 | 432.9 |
| 32 | 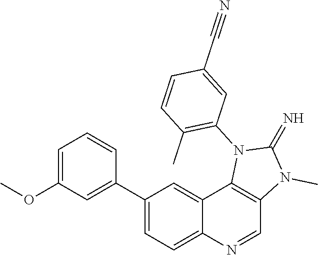 | 420 | >30 | >42 | 678.4 |
| 33 | 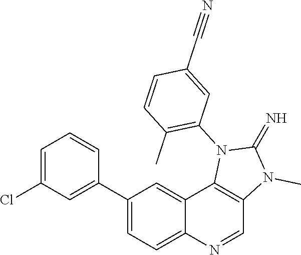 [SMILES: Cc1ccc(C#N)cc1-n1c(=N)n(C)c2cnc3ccc(-c4cccc(Cl)c4)cc3c21] | 297.1 | >30 | 14.5 | 1824.1 |
| 34 | 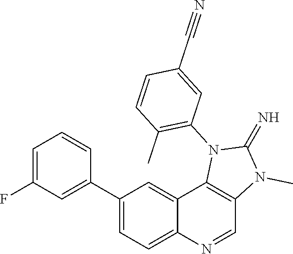 [SMILES: Cc1ccc(C#N)cc1-n1c(=N)n(C)c2cnc3ccc(-c4cccc(F)c4)cc3c21] | 47.3 | >30 | >40 | 852 |
| 35 | 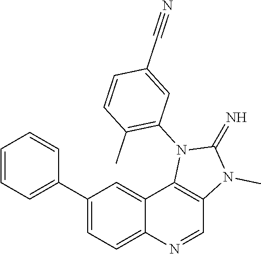 [SMILES: Cc1ccc(C#N)cc1-n1c(=N)n(C)c2cnc3ccc(-c4ccccc4)cc3c21] | 34 | >30 | >39 | 716 |
| 36 | 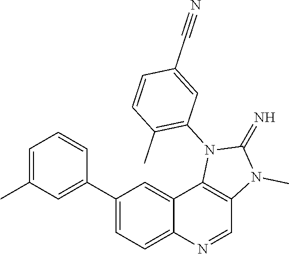 | 158 | 27 | >40 | 1151.3 |
| 37 | 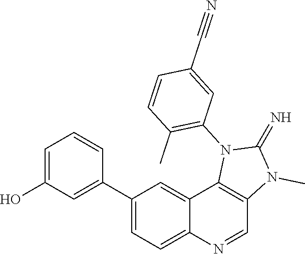 [SMILES: Cc1ccc(C#N)cc1-n1c(=N)n(C)c2cnc3ccc(-c4cccc(O)c4)cc3c21] | 46.1 | >30 | >40 | 10.3 |
| 38 | 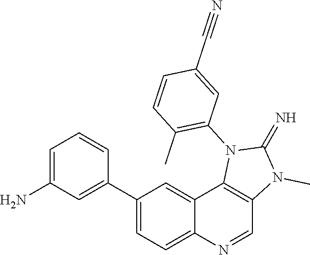 [SMILES: Cc1ccc(C#N)cc1-n1c(=N)n(C)c2cnc3ccc(-c4cccc(N)c4)cc3c21] | 83 | >30 | >40 | 435 |
| 39 | 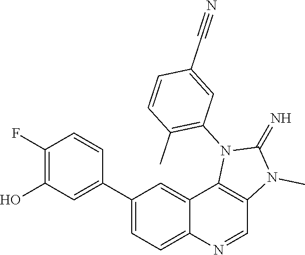 [SMILES: Cc1ccc(C#N)cc1-n1c(=N)n(C)c2cnc3ccc(-c4ccc(F)c(O)c4)cc3c21] | 54.5 | >30 | >42 | 147.1 |
| 40 | 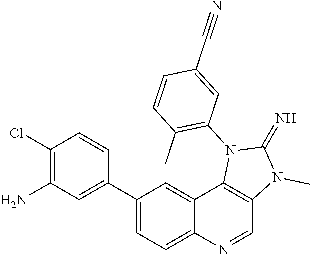 [SMILES: Cc1ccc(C#N)cc1-n1c(=N)n(C)c2cnc3ccc(-c4ccc(Cl)c(N)c4)cc3c21] | 30.8 | >30 | >43 | |
| 41 | 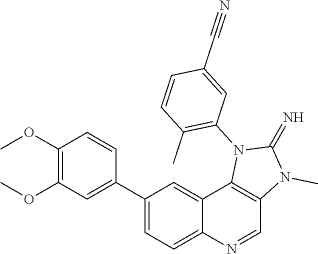 | 46.2 | >30 | >44 | 987.5 |
| 42 | 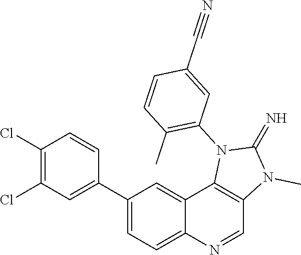 [SMILES: Cc1ccc(C#N)cc1-n1c(=N)n(C)c2cnc3ccc(-c4ccc(Cl)c(Cl)c4)cc3c21] | 573.6 | >30 | <1 | 1645.7 |
| 43 | 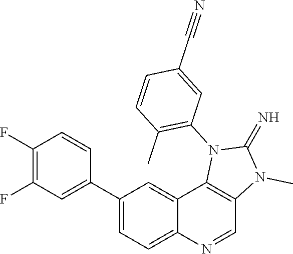 [SMILES: Cc1ccc(C#N)cc1-n1c(=N)n(C)c2cnc3ccc(-c4ccc(F)c(F)c4)cc3c21] | 118 | >30 | 8.3 | 836.7 |
| 44 | 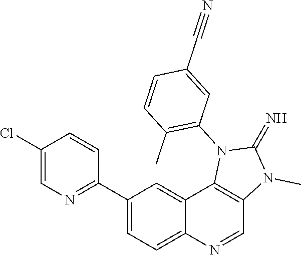 [SMILES: Cc1ccc(C#N)cc1-n1c(=N)n(C)c2cnc3ccc(-c4ccc(Cl)cn4)cc3c21] | 119.1 | >30 | >42 | 1561.8 |
| 45 | 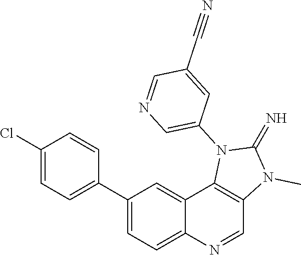 [SMILES: Cn1c(=N)n(-c2cncc(C#N)c2)c2c3cc(-c4ccc(Cl)cc4)ccc3ncc21] | 129.6 | >30 | 7.8 | |
| 46 | 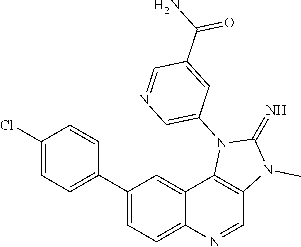 [SMILES: Cn1c(=N)n(-c2cncc(C(N)=O)c2)c2c3cc(-c4ccc(Cl)cc4)ccc3ncc21] | 1759 | >30 | 19.9 | |
| 47 | 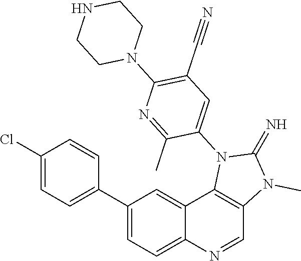 [SMILES: Cc1nc(N2CCNCC2)c(C#N)cc1-n1c(=N)n(C)c2cnc3ccc(-c4ccc(Cl)cc4)cc3c21] | 69.66 | >30 | 6 | |
| 48 | 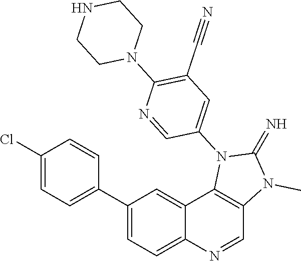 [SMILES: Cn1c(=N)n(-c2cnc(N3CCNCC3)c(C#N)c2)c2c3cc(-c4ccc(Cl)cc4)ccc3ncc21] | 125.2 | >30 | <1 | 30.5 |
| 49 | 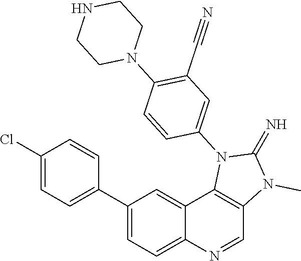 [SMILES: Cn1c(=N)n(-c2ccc(N3CCNCC3)c(C#N)c2)c2c3cc(-c4ccc(Cl)cc4)ccc3ncc21] | 58.2 | >30 | 1 | 4.9 |
| 50 | 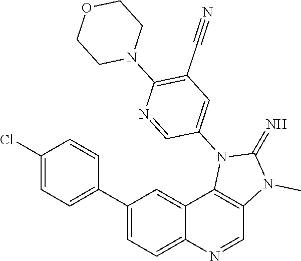 [SMILES: Cn1c(=N)n(-c2cnc(N3CCOCC3)c(C#N)c2)c2c3cc(-c4ccc(Cl)cc4)ccc3ncc21] | 309.4 | >30 | <1 | |
| 51 | 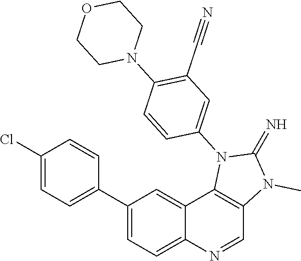 [SMILES: Cn1c(=N)n(-c2ccc(N3CCOCC3)c(C#N)c2)c2c3cc(-c4ccc(Cl)cc4)ccc3ncc21] | 185.9 | >30 | <1 | 81.1 |
| 52 | 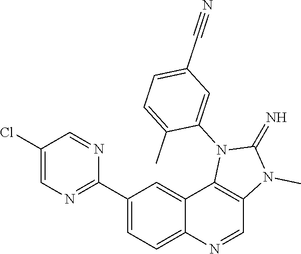 [SMILES: Cc1ccc(C#N)cc1-n1c(=N)n(C)c2cnc3ccc(-c4ncc(Cl)cn4)cc3c21] | 443.8 | >30 | >42 | 1431.7 |
| 53 | 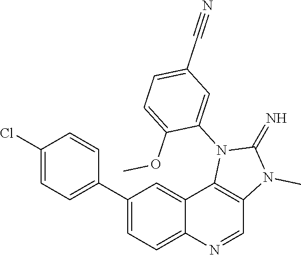 [SMILES: COc1ccc(C#N)cc1-n1c(=N)n(C)c2cnc3ccc(-c4ccc(Cl)cc4)cc3c21] | 119.6 | >30 | 12.2 | 850.5 |
| 54 | 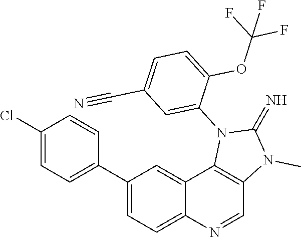 [SMILES: Cn1c(=N)n(-c2cc(C#N)ccc2OC(F)(F)F)c2c3cc(-c4ccc(Cl)cc4)ccc3ncc21] | 1423 | >30 | <1 | |
| 55 | 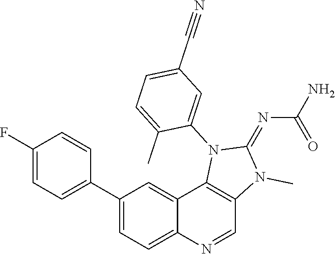 [SMILES: Cc1ccc(C#N)cc1-n1/c(=N/C(N)=O)n(C)c2cnc3ccc(-c4ccc(F)cc4)cc3c21] | 95.14 | >30 | 1.3 | 466.2 |
| 56 | 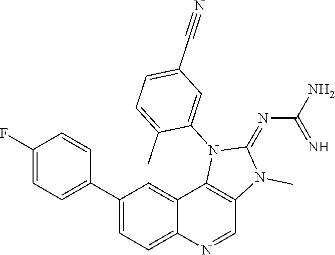 [SMILES: Cc1ccc(C#N)cc1-n1/c(=N/C(=N)N)n(C)c2cnc3ccc(-c4ccc(F)cc4)cc3c21] | 1460 | >30 | >44 | 1.7 |
| 57 | 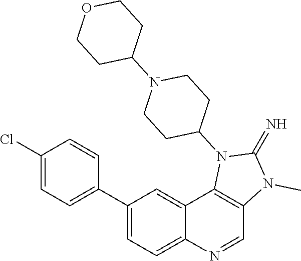 [SMILES: Cn1c(=N)n(C2CCN(C3CCOCC3)CC2)c2c3cc(-c4ccc(Cl)cc4)ccc3ncc21] | 2523 | >30 | >47 | |
| 58 | 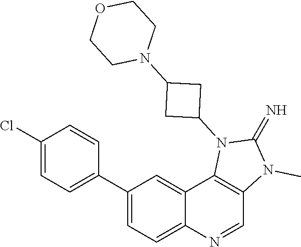 [SMILES: Cn1c(=N)n(C2CC(N3CCOCC3)C2)c2c3cc(-c4ccc(Cl)cc4)ccc3ncc21] | 2674 | >30 | >44 | 816.5 |
| 59 | 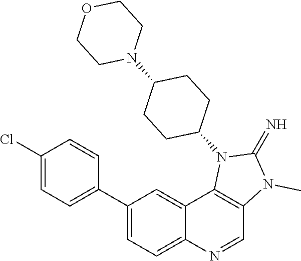 [SMILES: Cn1c(=N)n([C@H]2CC[C@@H](N3CCOCC3)CC2)c2c3cc(-c4ccc(Cl)cc4)ccc3ncc21] | 13200 | >30 | >47 | |
| 60 | 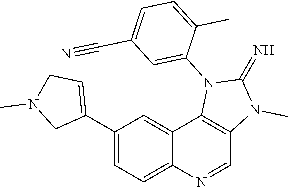 | 481.3 | >30 | >39 | 2 |
| 61 | 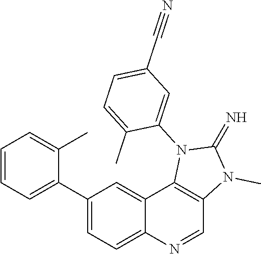 [SMILES: Cc1ccccc1-c1ccc2ncc3c(c2c1)n(-c1cc(C#N)ccc1C)c(=N)n3C] | 1720 | 10 | 19.2 | |
| 62 | 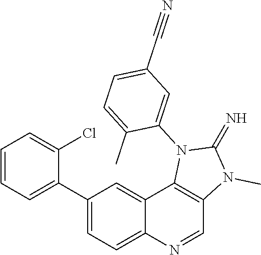 [SMILES: Cc1ccc(C#N)cc1-n1c(=N)n(C)c2cnc3ccc(-c4ccccc4Cl)cc3c21] | 704.4 | 18.5 | 7.8 | |
| 63 | 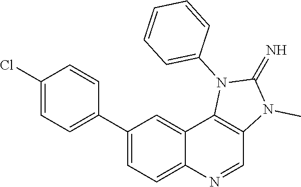 [SMILES: Cn1c(=N)n(-c2ccccc2)c2c3cc(-c4ccc(Cl)cc4)ccc3ncc21] | 1045 | >30 | 5.7 | |
| 64 | 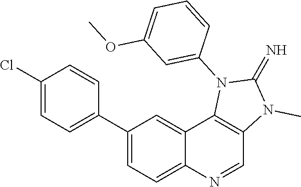 [SMILES: COc1cccc(-n2c(=N)n(C)c3cnc4ccc(-c5ccc(Cl)cc5)cc4c32)c1] | 1303 | >30 | 20.8 | |
| 65 | 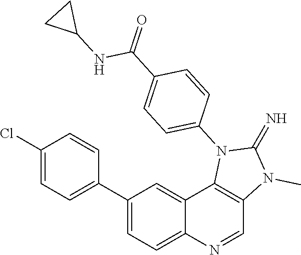 [SMILES: Cn1c(=N)n(-c2ccc(C(=O)NC3CC3)cc2)c2c3cc(-c4ccc(Cl)cc4)ccc3ncc21] | 579.6 | >30 | 6.1 | 10.2 |
| 66 | 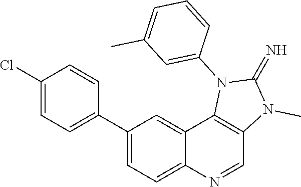 [SMILES: Cc1cccc(-n2c(=N)n(C)c3cnc4ccc(-c5ccc(Cl)cc5)cc4c32)c1] | 381.8 | >30 | 4 | |
| 67 | 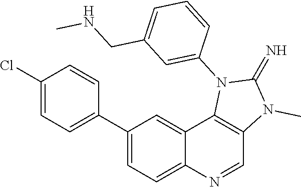 [SMILES: CNCc1cccc(-n2c(=N)n(C)c3cnc4ccc(-c5ccc(Cl)cc5)cc4c32)c1] | 2990 | >30 | >42 | 48.1 |
| 68 | 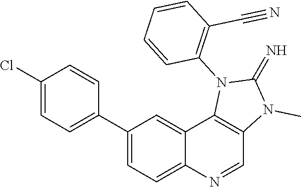 [SMILES: Cn1c(=N)n(-c2ccccc2C#N)c2c3cc(-c4ccc(Cl)cc4)ccc3ncc21] | 1450 | >30 | >41 | 141.6 |
| 69 | 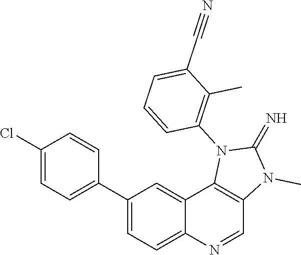 [SMILES: Cc1c(C#N)cccc1-n1c(=N)n(C)c2cnc3ccc(-c4ccc(Cl)cc4)cc3c21] | 207.2 | >30 | <1 | |
| 70 | 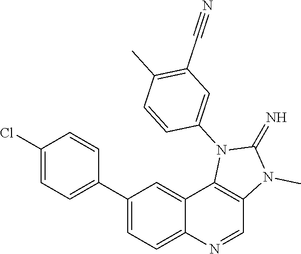 [SMILES: Cc1ccc(-n2c(=N)n(C)c3cnc4ccc(-c5ccc(Cl)cc5)cc4c32)cc1C#N] | 442.7 | >30 | <1 | |
| 71 | 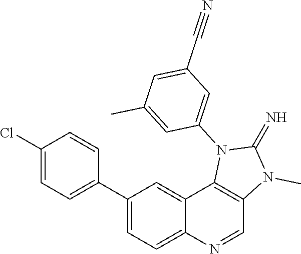 [SMILES: Cc1cc(C#N)cc(-n2c(=N)n(C)c3cnc4ccc(-c5ccc(Cl)cc5)cc4c32)c1] | 510.7 | >30 | <1 | |
| 72 | 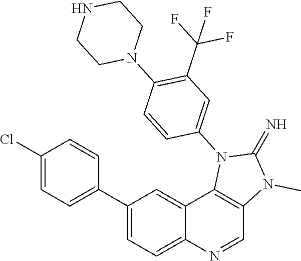 [SMILES: Cn1c(=N)n(-c2ccc(N3CCNCC3)c(C(F)(F)F)c2)c2c3cc(-c4ccc(Cl)cc4)ccc3ncc21] | 257.9 | >30 | <1 | |
| 73 | 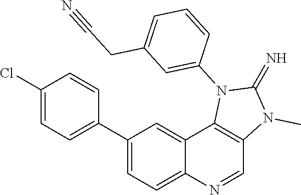 [SMILES: Cn1c(=N)n(-c2cccc(CC#N)c2)c2c3cc(-c4ccc(Cl)cc4)ccc3ncc21] | 118.1 | | | |
| 74 | 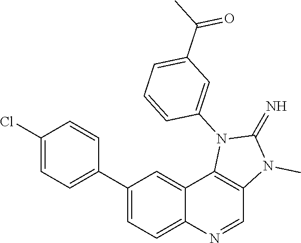 [SMILES: CC(=O)c1cccc(-n2c(=N)n(C)c3cnc4ccc(-c5ccc(Cl)cc5)cc4c32)c1] | 444.6 | >30 | >42 | |
| 75 | 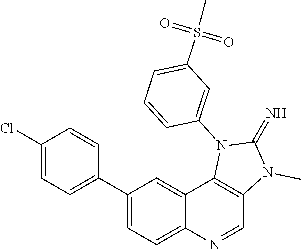 [SMILES: Cn1c(=N)n(-c2cccc(S(C)(=O)=O)c2)c2c3cc(-c4ccc(Cl)cc4)ccc3ncc21] | 1024 | >30 | 11.7 | |
| 76 | 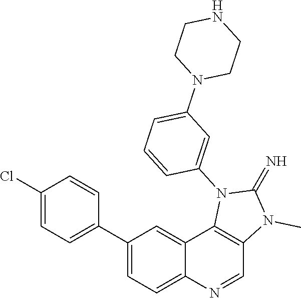 [SMILES: Cn1c(=N)n(-c2cccc(N3CCNCC3)c2)c2c3cc(-c4ccc(Cl)cc4)ccc3ncc21] | 802.4 | >30 | >46 | 28.3 |
| 77 | 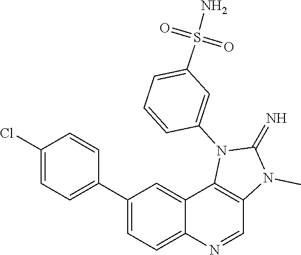 [SMILES: Cn1c(=N)n(-c2cccc(S(N)(=O)=O)c2)c2c3cc(-c4ccc(Cl)cc4)ccc3ncc21] | 1396 | >30 | 6.2 | |
| 78 | 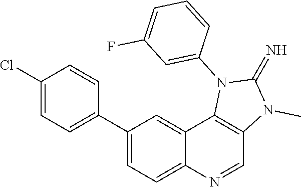 [SMILES: Cn1c(=N)n(-c2cccc(F)c2)c2c3cc(-c4ccc(Cl)cc4)ccc3ncc21] | 309.4 | >30 | <1 | 629.3 |
| 79 | 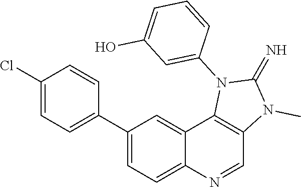 [SMILES: Cn1c(=N)n(-c2cccc(O)c2)c2c3cc(-c4ccc(Cl)cc4)ccc3ncc21] | 1420 | >30 | 9.7 | 42 |
| 80 | 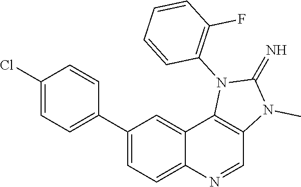 [SMILES: Cn1c(=N)n(-c2ccccc2F)c2c3cc(-c4ccc(Cl)cc4)ccc3ncc21] | 955 | >30 | 12.4 | |
| 81 | 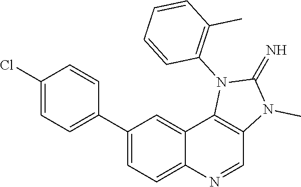 [SMILES: Cc1ccccc1-n1c(=N)n(C)c2cnc3ccc(-c4ccc(Cl)cc4)cc3c21] | 1642 | >30 | >39 | |
| 82 | 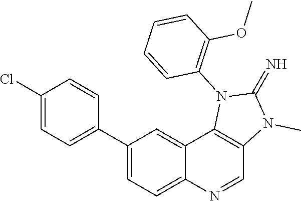 [SMILES: COc1ccccc1-n1c(=N)n(C)c2cnc3ccc(-c4ccc(Cl)cc4)cc3c21] | 1668 | >30 | >41 | |
| 83 | 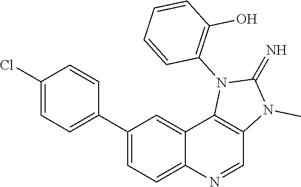 [SMILES: Cn1c(=N)n(-c2ccccc2O)c2c3cc(-c4ccc(Cl)cc4)ccc3ncc21] | 592.6 | | <1 | 859.1 |
| 84 | 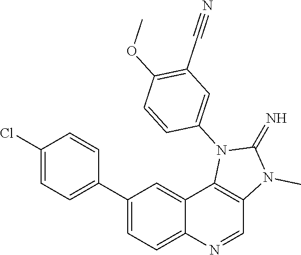 [SMILES: COc1ccc(-n2c(=N)n(C)c3cnc4ccc(-c5ccc(Cl)cc5)cc4c32)cc1C#N] | 658.4 | >30 | <1 | |
| 85 | 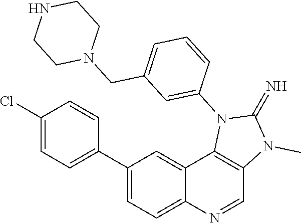 [SMILES: Cn1c(=N)n(-c2cccc(CN3CCNCC3)c2)c2c3cc(-c4ccc(Cl)cc4)ccc3ncc21] | 701.9 | >30 | >48 | 11.2 |
| 86 | 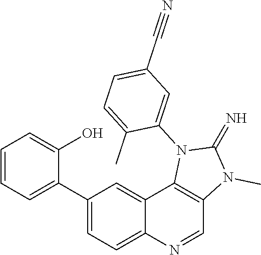 [SMILES: Cc1ccc(C#N)cc1-n1c(=N)n(C)c2cnc3ccc(-c4ccccc4O)cc3c21] | 695.9 | >30 | >40 | 7.9 |
| 87 | 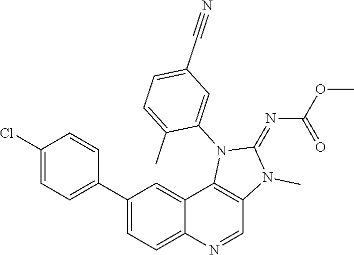 [SMILES: COC(=O)/N=c1\n(C)c2cnc3ccc(-c4ccc(Cl)cc4)cc3c2n1-c1cc(C#N)ccc1C] | 57.55 | >30 | <1 | 95.6 |
| 88 | 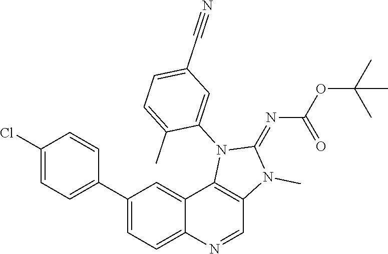 [SMILES: Cc1ccc(C#N)cc1-n1/c(=N/C(=O)OC(C)(C)C)n(C)c2cnc3ccc(-c4ccc(Cl)cc4)cc3c21] | 1619 | >30 | <1 | 127.9 |
| 89 | 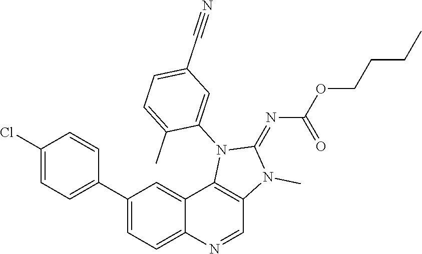 [SMILES: CCCCOC(=O)/N=c1\n(C)c2cnc3ccc(-c4ccc(Cl)cc4)cc3c2n1-c1cc(C#N)ccc1C] | 601.2 | 6.2 | <1 | |
| 90 | 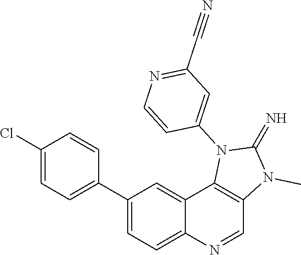 [SMILES: Cn1c(=N)n(-c2ccnc(C#N)c2)c2c3cc(-c4ccc(Cl)cc4)ccc3ncc21] | 224.4 | | | |
| 91 | 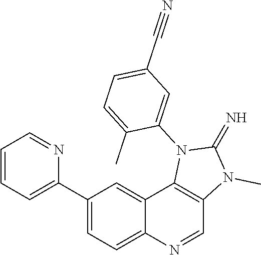 [SMILES: Cc1ccc(C#N)cc1-n1c(=N)n(C)c2cnc3ccc(-c4ccccn4)cc3c21] | 422.7 | >30 | >39 | 1370.7 |
| 92 | 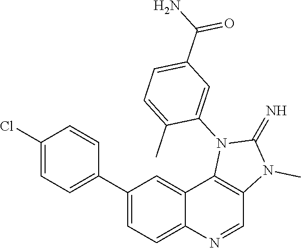 [SMILES: Cc1ccc(C(N)=O)cc1-n1c(=N)n(C)c2cnc3ccc(-c4ccc(Cl)cc4)cc3c21] | 0 | >30 | >44 | 2 |
| 93 | 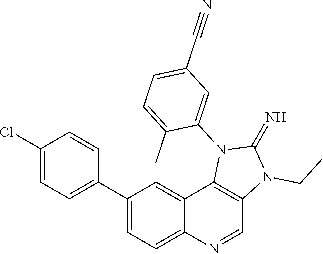 [SMILES: CCn1c(=N)n(-c2cc(C#N)ccc2C)c2c3cc(-c4ccc(Cl)cc4)ccc3ncc21] | 813.1 | >30 | 13.6 | |
| 94 | 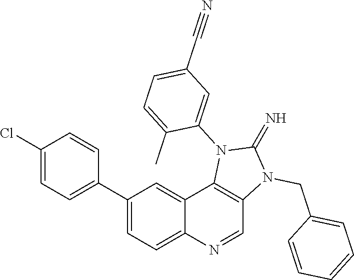 [SMILES: Cc1ccc(C#N)cc1-n1c(=N)n(Cc2ccccc2)c2cnc3ccc(-c4ccc(Cl)cc4)cc3c21] | 9166 | >30 | <1 | |
| 95 | 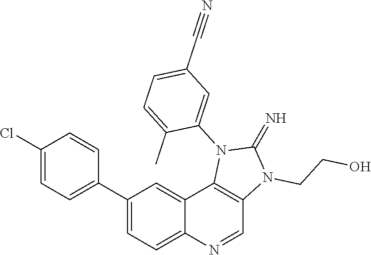 [SMILES: Cc1ccc(C#N)cc1-n1c(=N)n(CCO)c2cnc3ccc(-c4ccc(Cl)cc4)cc3c21] | 2578 | >30 | 27.4 | |
| 96 | 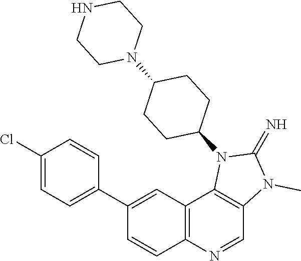 [SMILES: Cn1c(=N)n([C@H]2CC[C@H](N3CCNCC3)CC2)c2c3cc(-c4ccc(Cl)cc4)ccc3ncc21] | 2631 | >30 | >47 | |
| 97 | 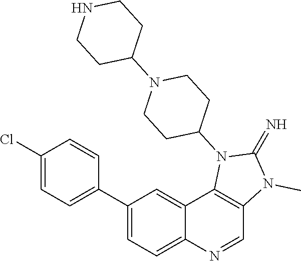 [SMILES: Cn1c(=N)n(C2CCN(C3CCNCC3)CC2)c2c3cc(-c4ccc(Cl)cc4)ccc3ncc21] | 5098 | >30 | >47 | |
| 98 | 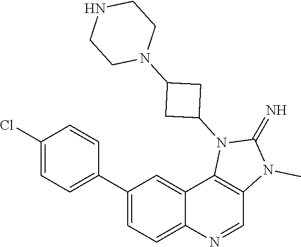 [SMILES: Cn1c(=N)n(C2CC(N3CCNCC3)C2)c2c3cc(-c4ccc(Cl)cc4)ccc3ncc21] | 2546 | >30 | >44 | |
| 99 | 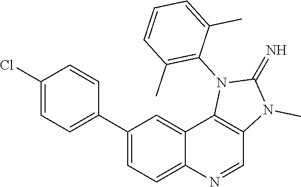 [SMILES: Cc1cccc(C)c1-n1c(=N)n(C)c2cnc3ccc(-c4ccc(Cl)cc4)cc3c21] | 3180 | >30 | >41 | |
| 100 | 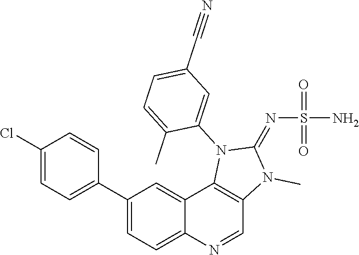 [SMILES: Cc1ccc(C#N)cc1-n1/c(=N/S(N)(=O)=O)n(C)c2cnc3ccc(-c4ccc(Cl)cc4)cc3c21] | 12.87 | >30 | <1 | 235.9 |
| 101 | 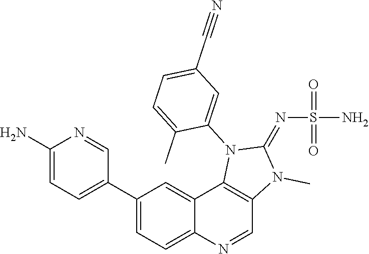 [SMILES: Cc1ccc(C#N)cc1-n1/c(=N/S(N)(=O)=O)n(C)c2cnc3ccc(-c4ccc(N)nc4)cc3c21] | 3.07 | >30 | 2.2 | |
| 102 | 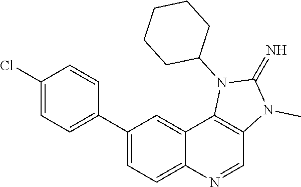 [SMILES: Cn1c(=N)n(C2CCCCC2)c2c3cc(-c4ccc(Cl)cc4)ccc3ncc21] | 4782 | >30 | 22.3 | |
| 103 | 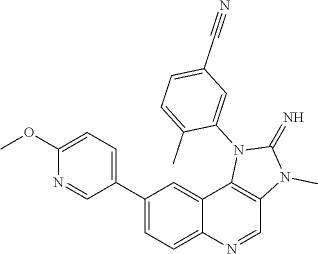 | 162.6 | >30 | >42 | |
| 104 | 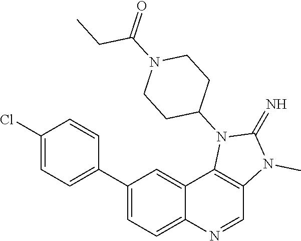 [SMILES: CCC(=O)N1CCC(n2c(=N)n(C)c3cnc4ccc(-c5ccc(Cl)cc5)cc4c32)CC1] | 411 | >30 | >44 | |
| 105 | 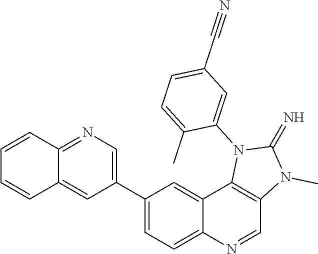 [SMILES: Cc1ccc(C#N)cc1-n1c(=N)n(C)c2cnc3ccc(-c4cnc5ccccc5c4)cc3c21] | 713.9 | >30 | >44 | 1145.6 |
| 106 | 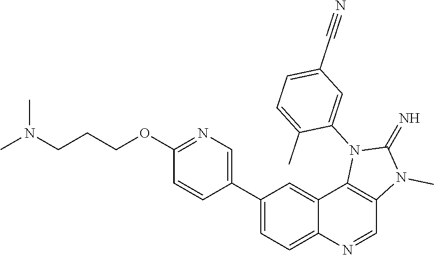 | 140.4 | >30 | >49 | 1.2 |
| 107 | 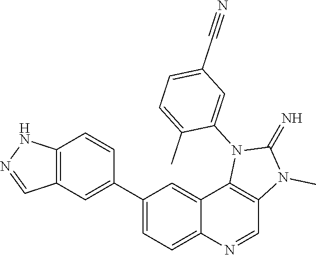 [SMILES: Cc1ccc(C#N)cc1-n1c(=N)n(C)c2cnc3ccc(-c4ccc5[nH]ncc5c4)cc3c21] | 12.95 | 22.9 | >42 | 22.6 |
| 108 | 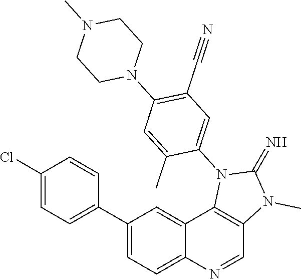 [SMILES: Cc1cc(N2CCN(C)CC2)c(C#N)cc1-n1c(=N)n(C)c2cnc3ccc(-c4ccc(Cl)cc4)cc3c21] | 463.9 | >30 | 37.6 | 255.3 |
| 109 | 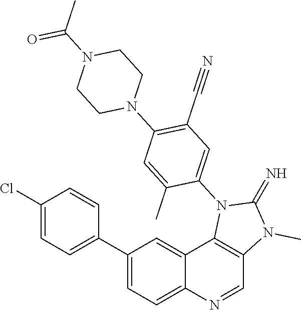 [SMILES: CC(=O)N1CCN(c2cc(C)c(-n3c(=N)n(C)c4cnc5ccc(-c6ccc(Cl)cc6)cc5c43)cc2C#N)CC1] | 4.33 | >30 | <1 | 838.2 |
| 110 | 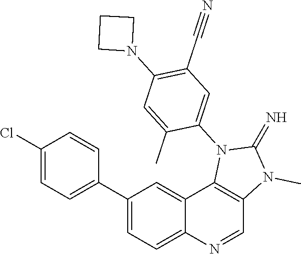 [SMILES: Cc1cc(N2CCC2)c(C#N)cc1-n1c(=N)n(C)c2cnc3ccc(-c4ccc(Cl)cc4)cc3c21] | 373 | >30 | <1 | 176.2 |
| 111 | 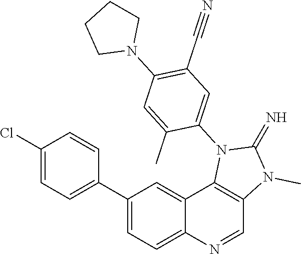 [SMILES: Cc1cc(N2CCCC2)c(C#N)cc1-n1c(=N)n(C)c2cnc3ccc(-c4ccc(Cl)cc4)cc3c21] | 800.9 | | | |
| 112 | 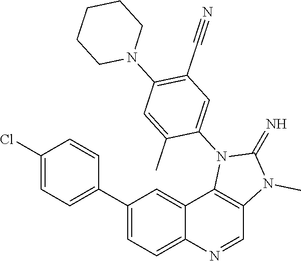 [SMILES: Cc1cc(N2CCCCC2)c(C#N)cc1-n1c(=N)n(C)c2cnc3ccc(-c4ccc(Cl)cc4)cc3c21] | 1834 | >30 | <1 | 1280.3 |
| 113 | 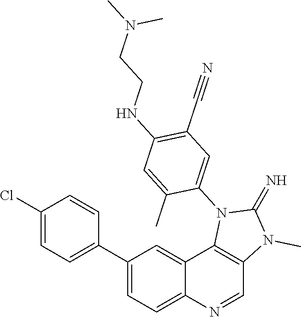 [SMILES: Cc1cc(NCCN(C)C)c(C#N)cc1-n1c(=N)n(C)c2cnc3ccc(-c4ccc(Cl)cc4)cc3c21] | 545.5 | >30 | >50 | |
| 114 | 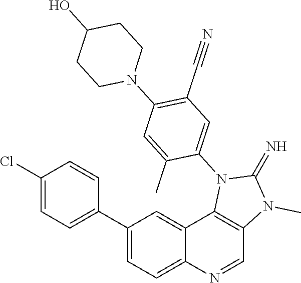 [SMILES: Cc1cc(N2CCC(O)CC2)c(C#N)cc1-n1c(=N)n(C)c2cnc3ccc(-c4ccc(Cl)cc4)cc3c21] | 55.25 | >30 | <1 | |
| 115 | 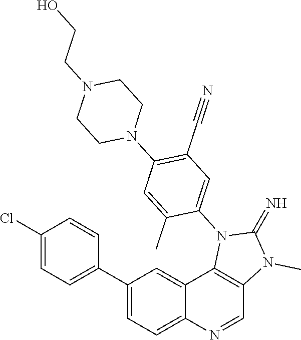 [SMILES: Cc1cc(N2CCN(CCO)CC2)c(C#N)cc1-n1c(=N)n(C)c2cnc3ccc(-c4ccc(Cl)cc4)cc3c21] | 125.3 | >30 | >55 | |
| 116 | 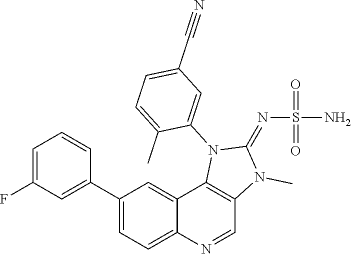 [SMILES: Cc1ccc(C#N)cc1-n1/c(=N/S(N)(=O)=O)n(C)c2cnc3ccc(-c4cccc(F)c4)cc3c21] | 27.83 | 17.9 | <1 | 1071.9 |
| 117 | 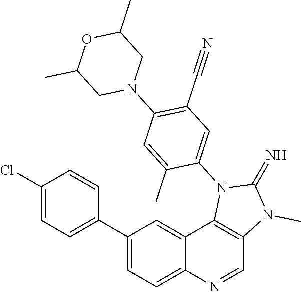 [SMILES: Cc1cc(N2CC(C)OC(C)C2)c(C#N)cc1-n1c(=N)n(C)c2cnc3ccc(-c4ccc(Cl)cc4)cc3c21] | 372.7 | >30 | <1 | |
| 118 | 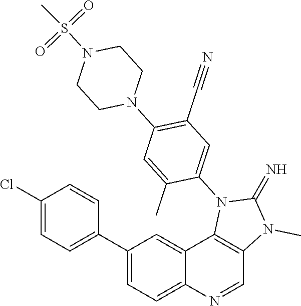 [SMILES: Cc1cc(N2CCN(S(C)(=O)=O)CC2)c(C#N)cc1-n1c(=N)n(C)c2cnc3ccc(-c4ccc(Cl)cc4)cc3c21] | 135.3 | >30 | <1 | |
| 119 | 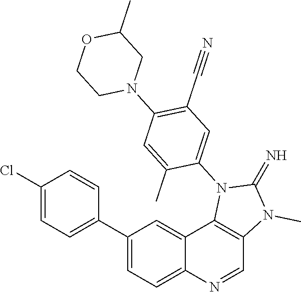 [SMILES: Cc1cc(N2CCOC(C)C2)c(C#N)cc1-n1c(=N)n(C)c2cnc3ccc(-c4ccc(Cl)cc4)cc3c21] | 351.9 | | | |
| 120 | 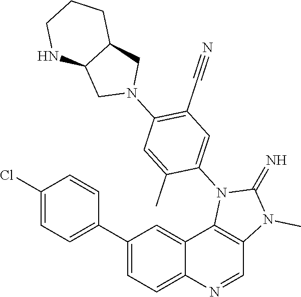 [SMILES: Cc1cc(N2C[C@@H]3CCCN[C@@H]3C2)c(C#N)cc1-n1c(=N)n(C)c2cnc3ccc(-c4ccc(Cl)cc4)cc3c21] | 1470 | | | |
| 121 | 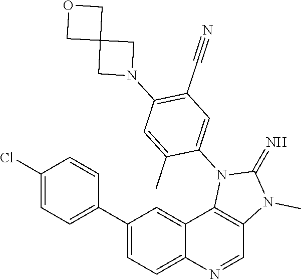 [SMILES: Cc1cc(N2CC3(COC3)C2)c(C#N)cc1-n1c(=N)n(C)c2cnc3ccc(-c4ccc(Cl)cc4)cc3c21] | 148.2 | >30 | 1.3 | 6.2 |
| 122 | 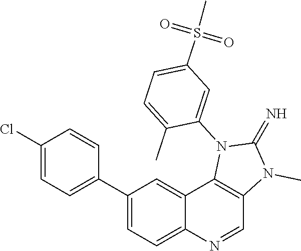 [SMILES: Cc1ccc(S(C)(=O)=O)cc1-n1c(=N)n(C)c2cnc3ccc(-c4ccc(Cl)cc4)cc3c21] | 1607 | >30 | >47 | |
| 123 | 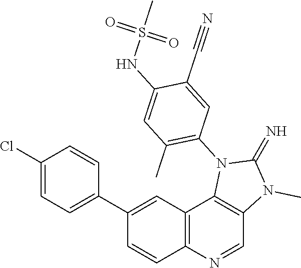 [SMILES: Cc1cc(NS(C)(=O)=O)c(C#N)cc1-n1c(=N)n(C)c2cnc3ccc(-c4ccc(Cl)cc4)cc3c21] | 88.57 | >30 | <1 | |
| 124 | 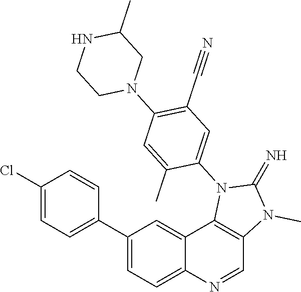 [SMILES: Cc1cc(N2CCNC(C)C2)c(C#N)cc1-n1c(=N)n(C)c2cnc3ccc(-c4ccc(Cl)cc4)cc3c21] | 217.2 | >30 | 1.9 | |
| 125 | 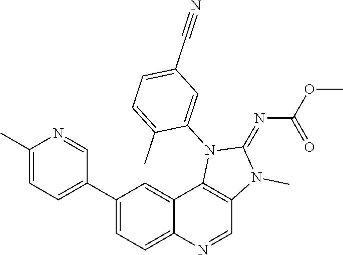 | 1.2 | >30 | 12.5 | 243.4 |
| 126 | 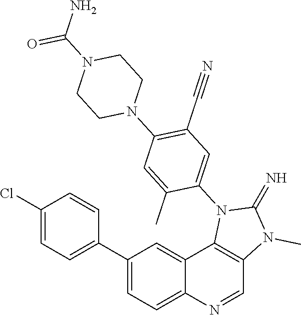 [SMILES: Cc1cc(N2CCN(C(N)=O)CC2)c(C#N)cc1-n1c(=N)n(C)c2cnc3ccc(-c4ccc(Cl)cc4)cc3c21] | 14.48 | >30 | 2.2 | |
| 127 | 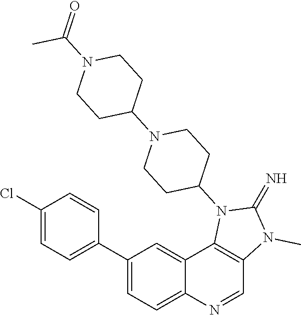 [SMILES: CC(=O)N1CCC(N2CCC(n3c(=N)n(C)c4cnc5ccc(-c6ccc(Cl)cc6)cc5c43)CC2)CC1] | 1495 | | | |
| 128 | 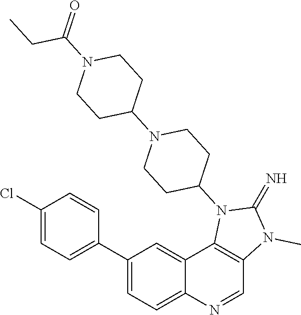 [SMILES: CCC(=O)N1CCC(N2CCC(n3c(=N)n(C)c4cnc5ccc(-c6ccc(Cl)cc6)cc5c43)CC2)CC1] | 626.6 | | | |
| 129 | 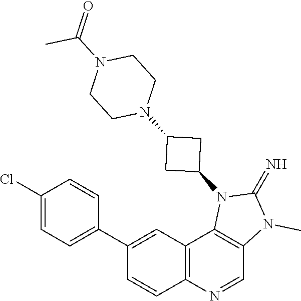 [SMILES: CC(=O)N1CCN([C@H]2C[C@H](n3c(=N)n(C)c4cnc5ccc(-c6ccc(Cl)cc6)cc5c43)C2)CC1] | 12660 | >30 | >48 | 4.6 |
| 130 | 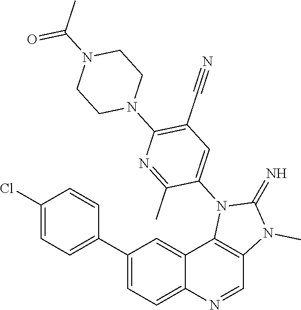 [SMILES: CC(=O)N1CCN(c2nc(C)c(-n3c(=N)n(C)c4cnc5ccc(-c6ccc(Cl)cc6)cc5c43)cc2C#N)CC1] | 22.25 | 20.2 | >54 | 112.1 |
| 131 | 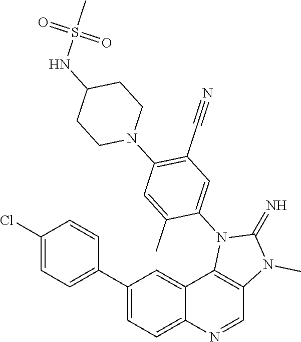 [SMILES: Cc1cc(N2CCC(NS(C)(=O)=O)CC2)c(C#N)cc1-n1c(=N)n(C)c2cnc3ccc(-c4ccc(Cl)cc4)cc3c21] | 16.3 | | | |
| 132 | 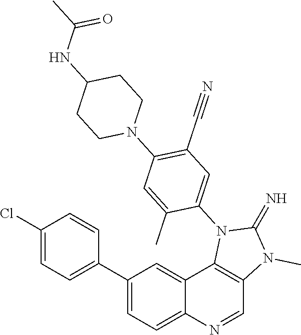 [SMILES: CC(=O)NC1CCN(c2cc(C)c(-n3c(=N)n(C)c4cnc5ccc(-c6ccc(Cl)cc6)cc5c43)cc2C#N)CC1] | 54.88 | >30 | 4.6 | 7 |
| 133 | 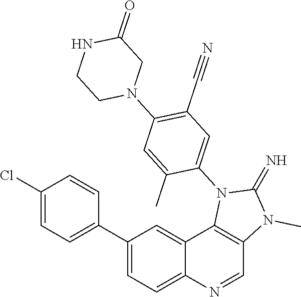 [SMILES: Cc1cc(N2CCNC(=O)C2)c(C#N)cc1-n1c(=N)n(C)c2cnc3ccc(-c4ccc(Cl)cc4)cc3c21] | 6.26 | | | |
| 134 | 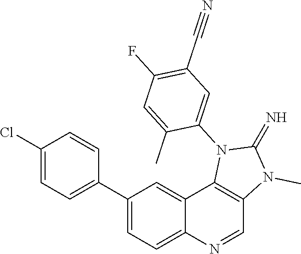 [SMILES: Cc1cc(F)c(C#N)cc1-n1c(=N)n(C)c2cnc3ccc(-c4ccc(Cl)cc4)cc3c21] | 74.3 | | | |
| 135 | 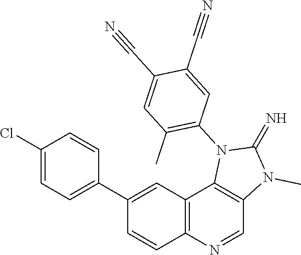 [SMILES: Cc1cc(C#N)c(C#N)cc1-n1c(=N)n(C)c2cnc3ccc(-c4ccc(Cl)cc4)cc3c21] | 26.98 | >30 | <1 | 397.1 |
| 136 | 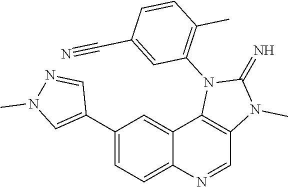 | 56.15 | >30 | 40.4 | 89.5 |
| 137 | 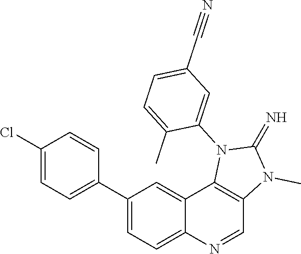 [SMILES: Cc1ccc(C#N)cc1-n1c(=N)n(C)c2cnc3ccc(-c4ccc(Cl)cc4)cc3c21] | 43.15 | >30 | 3.6 | 1257.3 |
| 142 | 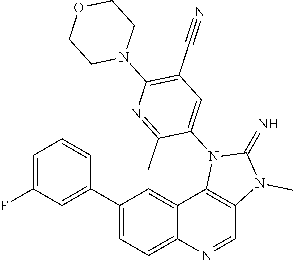 [SMILES: Cc1nc(N2CCOCC2)c(C#N)cc1-n1c(=N)n(C)c2cnc3ccc(-c4cccc(F)c4)cc3c21] | 224.8 | >30 | >49 | |
| 143 | 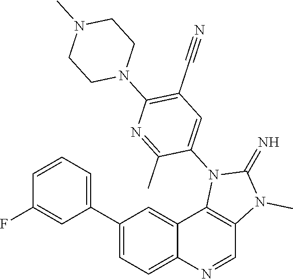 [SMILES: Cc1nc(N2CCN(C)CC2)c(C#N)cc1-n1c(=N)n(C)c2cnc3ccc(-c4cccc(F)c4)cc3c21] | 374.1 | >30 | >50 | 775.8 |
| 144 | 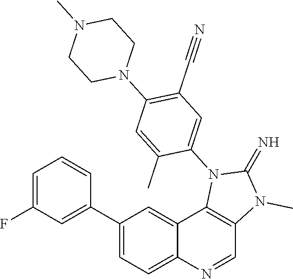 [SMILES: Cc1cc(N2CCN(C)CC2)c(C#N)cc1-n1c(=N)n(C)c2cnc3ccc(-c4cccc(F)c4)cc3c21] | 159.2 | >30 | >50 | |
| 145 | 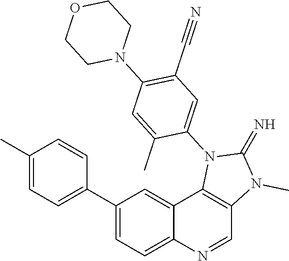 | 142.1 | >30 | 16.7 | 1140.7 |
| 146 | 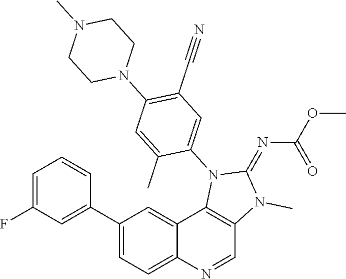 [SMILES: COC(=O)/N=c1\n(C)c2cnc3ccc(-c4cccc(F)c4)cc3c2n1-c1cc(C#N)c(N2CCN(C)CC2)cc1C] | 62.69 | 21.2 | 14.5 | |
| 147 | 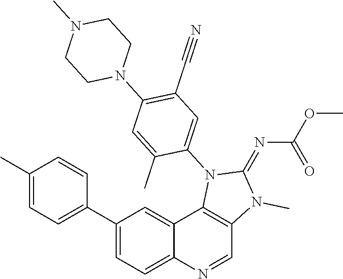 | 28.4 | 24.4 | 4.3 | 656.4 |
| 148 | 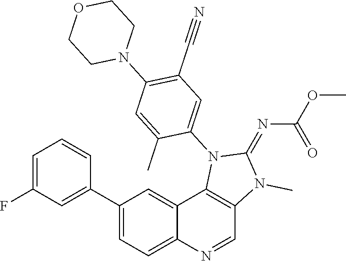 [SMILES: COC(=O)/N=c1\n(C)c2cnc3ccc(-c4cccc(F)c4)cc3c2n1-c1cc(C#N)c(N2CCOCC2)cc1C] | 236.3 | | | |
| 154 | 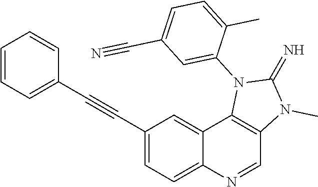 [SMILES: Cc1ccc(C#N)cc1-n1c(=N)n(C)c2cnc3ccc(C#Cc4ccccc4)cc3c21] | 1749 | >30 | 1.4 | |
| 155 | 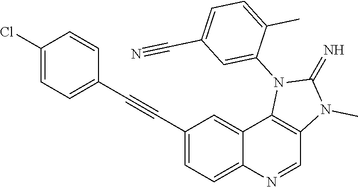 [SMILES: Cc1ccc(C#N)cc1-n1c(=N)n(C)c2cnc3ccc(C#Cc4ccc(Cl)cc4)cc3c21] | 8977 | >30 | <1 | 916.3 |

TABLE 2

| | | Microsomal | Solubility | |
|---|---|---|---|---|
| | | Stability | pH 7.4 | |
| | | (rat) T1/2 | (ug/mL) | PAMPA |
| | Asexual | (min) green | green for | Permeability |
| Structure | EC50 (nM) | for >30 | >1.0 | (×106 cm/s) |
| 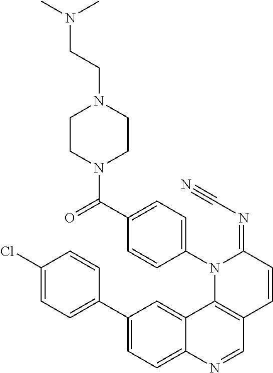 [SMILES: CN(C)CCN1CCN(C(=O)c2ccc(-n3/c(=N\C#N)ccc4cnc5ccc(-c6ccc(Cl)cc6)cc5c43)cc2)CC1] | 45.42 | >30 | 3.2 | 8.8 |
| 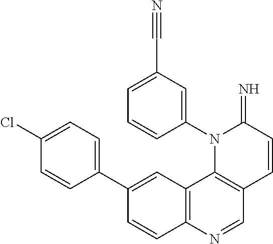 [SMILES: N#Cc1cccc(-n2c(=N)ccc3cnc4ccc(-c5ccc(Cl)cc5)cc4c32)c1] | 186.5 | >30 | 25.9 | |
| 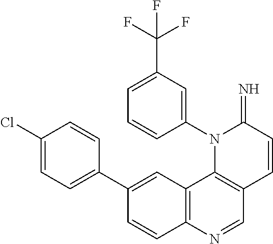 [SMILES: N=c1ccc2cnc3ccc(-c4ccc(Cl)cc4)cc3c2n1-c1cccc(C(F)(F)F)c1] | 212.9 | >30 | 11.5 | |
| 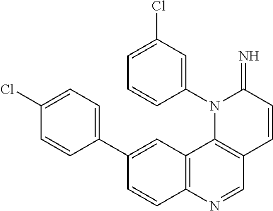 [SMILES: N=c1ccc2cnc3ccc(-c4ccc(Cl)cc4)cc3c2n1-c1cccc(Cl)c1] | 177.7 | >30 | 20.7 | 1242.4 |
| 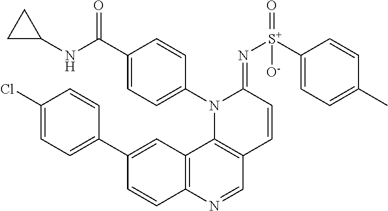 [SMILES: Cc1ccc([S+](=O)([O-])/N=c2\ccc3cnc4ccc(-c5ccc(Cl)cc5)cc4c3n2-c2ccc(C(=O)NC3CC3)cc2)cc1] | 58.97 | 11.5 | <1 | |
| 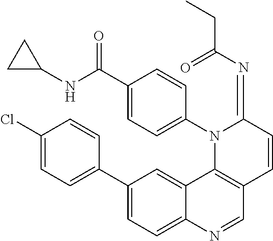 [SMILES: CCC(=O)/N=c1/ccc2cnc3ccc(-c4ccc(Cl)cc4)cc3c2n1-c1ccc(C(=O)NC2CC2)cc1] | 47.5 | 4.8 | <1 | 444.8 |
| 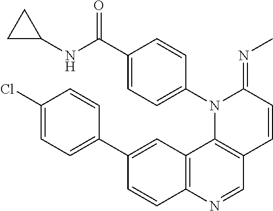 [SMILES: C/N=c1\ccc2cnc3ccc(-c4ccc(Cl)cc4)cc3c2n1-c1ccc(C(=O)NC2CC2)cc1] | 1210 | 17.4 | <1 | 129.7 |
| 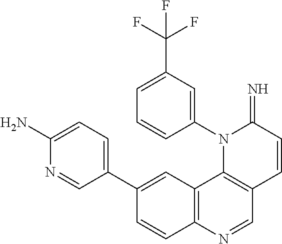 [SMILES: N=c1ccc2cnc3ccc(-c4ccc(N)nc4)cc3c2n1-c1cccc(C(F)(F)F)c1] | 21.78 | >30 | >64 | 227.8 |
| 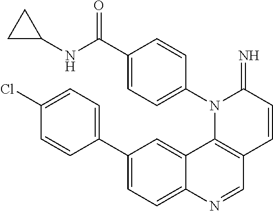 [SMILES: N=c1ccc2cnc3ccc(-c4ccc(Cl)cc4)cc3c2n1-c1ccc(C(=O)NC2CC2)cc1] | 85.67 | >30 | >69 | |

TABLE 3

| | | | Solubility | |
|---|---|---|---|---|
| | | Microsomal | pH 7.4 | |
| | | Stability (rat) | (ug/mL) | PAMPA |
| | Asexual | T1/2 (min) | green for | Permeability |
| Structure | EC50 (nM) | green for >30 | >1.0 | (×106 cm/s) |
| 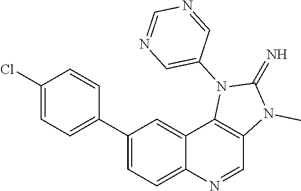 [SMILES: Cn1c(=N)n(-c2cncnc2)c2c3cc(-c4ccc(Cl)cc4)ccc3ncc21] | 220.1 | >30 | 16.3 | 396.8 |
| 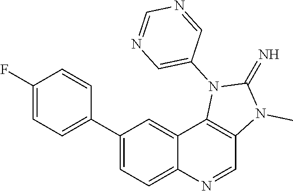 [SMILES: Cn1c(=N)n(-c2cncnc2)c2c3cc(-c4ccc(F)cc4)ccc3ncc21] | 194.3 | >30 | 2 | 26.2 |
| 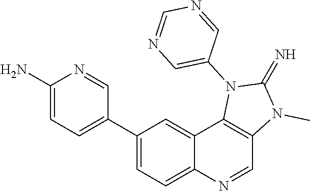 [SMILES: Cn1c(=N)n(-c2cncnc2)c2c3cc(-c4ccc(N)nc4)ccc3ncc21] | 143.3 | >30 | 33.9 | 12.9 |
| 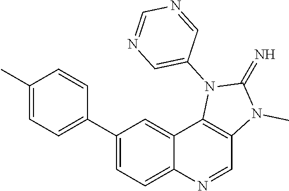 | 229.6 | >30 | 18.5 | 32.3 |
| 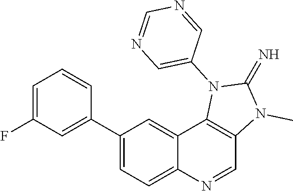 [SMILES: Cn1c(=N)n(-c2cncnc2)c2c3cc(-c4cccc(F)c4)ccc3ncc21] | 168.6 | >30 | 24.9 | 60.6 |
| 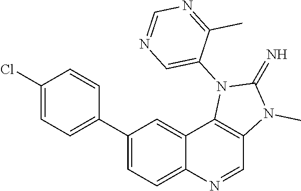 [SMILES: Cc1ncncc1-n1c(=N)n(C)c2cnc3ccc(-c4ccc(Cl)cc4)cc3c21] | 720.9 | >30 | 24.7 | 1288.1 |
| 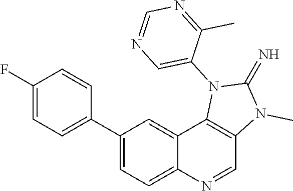 [SMILES: Cc1ncncc1-n1c(=N)n(C)c2cnc3ccc(-c4ccc(F)cc4)cc3c21] | 537.8 | >30 | 6.2 | 1181.6 |
| 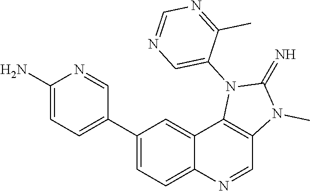 [SMILES: Cc1ncncc1-n1c(=N)n(C)c2cnc3ccc(-c4ccc(N)nc4)cc3c21] | 164.1 | >30 | >38 | |
| 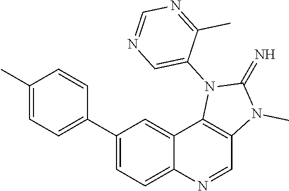 | 1150 | >30 | >38 | 1241.8 |
| 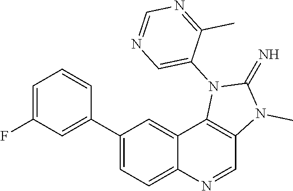 [SMILES: Cc1ncncc1-n1c(=N)n(C)c2cnc3ccc(-c4cccc(F)c4)cc3c21] | 1245 | >30 | >38 | 1096.8 |
| 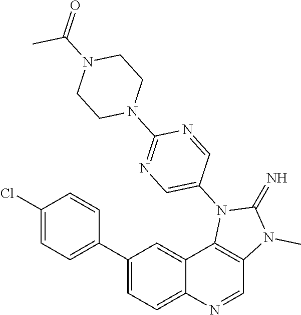 [SMILES: CC(=O)N1CCN(c2ncc(-n3c(=N)n(C)c4cnc5ccc(-c6ccc(Cl)cc6)cc5c43)cn2)CC1] | 391.3 | >30 | 22.9 | 292.5 |
| 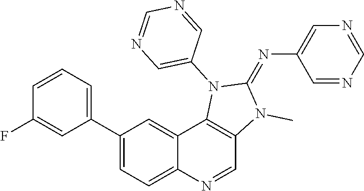 [SMILES: Cn1/c(=N\c2cncnc2)n(-c2cncnc2)c2c3cc(-c4cccc(F)c4)ccc3ncc21] | 193.2 | | | |
| 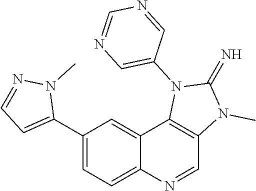 [SMILES: Cn1nccc1-c1ccc2ncc3c(c2c1)n(-c1cncnc1)c(=N)n3C] | 570 | | 36.3 | |
| 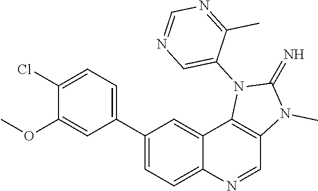 | 50.81 | | 1.3 | |
| 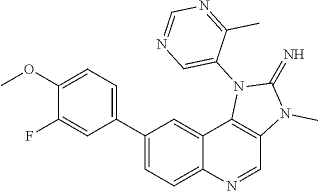 | 228.7 | | >61 | |
| 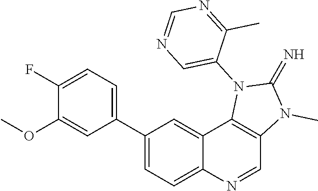 | 52.45 | | <1 | |
| 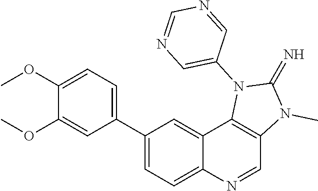 | 17 | >30 | >61 | 510.4 |
| 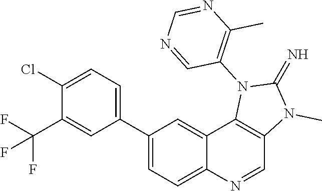 [SMILES: Cc1ncncc1-n1c(=N)n(C)c2cnc3ccc(-c4ccc(Cl)c(C(F)(F)F)c4)cc3c21] | 160 | >30 | 10.6 | |
| 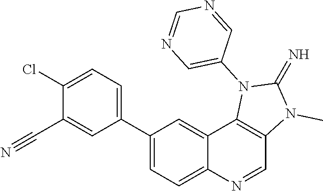 [SMILES: Cn1c(=N)n(-c2cncnc2)c2c3cc(-c4ccc(Cl)c(C#N)c4)ccc3ncc21] | 135.8 | | | |
| 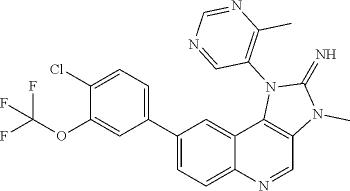 [SMILES: Cc1ncncc1-n1c(=N)n(C)c2cnc3ccc(-c4ccc(Cl)c(OC(F)(F)F)c4)cc3c21] | 157.6 | >30 | 16 | |
| 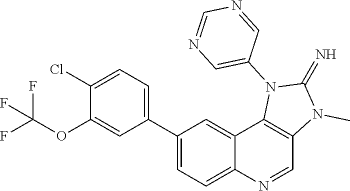 [SMILES: Cn1c(=N)n(-c2cncnc2)c2c3cc(-c4ccc(Cl)c(OC(F)(F)F)c4)ccc3ncc21] | 315.2 | >30 | <1 | |
| 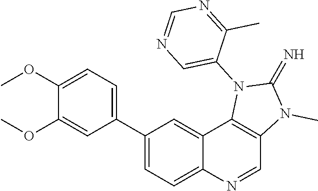 | 25.75 | >30 | | |
| 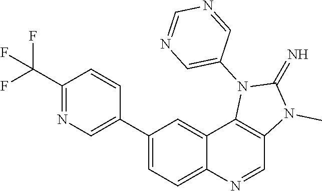 [SMILES: Cn1c(=N)n(-c2cncnc2)c2c3cc(-c4ccc(C(F)(F)F)nc4)ccc3ncc21] | 560.2 | >30 | 31 | 541.1 |
| 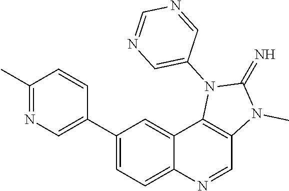 | 190.5 | >30 | >54 | 135.8 |
| 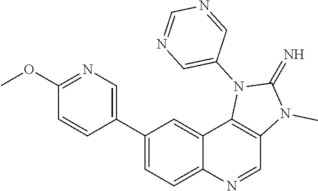 | 1051 | >30 | 34.5 | 684.3 |
| 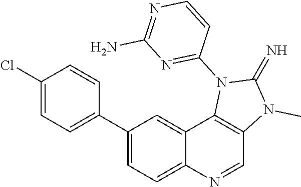 [SMILES: Cn1c(=N)n(-c2ccnc(N)n2)c2c3cc(-c4ccc(Cl)cc4)ccc3ncc21] | 139.9 | >30 | 3.4 | |
| 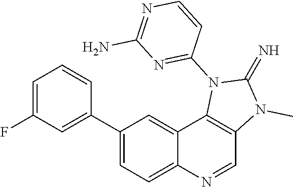 [SMILES: Cn1c(=N)n(-c2ccnc(N)n2)c2c3cc(-c4cccc(F)c4)ccc3ncc21] | 523.1 | >30 | 20.9 | |
| 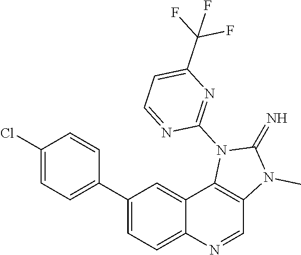 [SMILES: Cn1c(=N)n(-c2nccc(C(F)(F)F)n2)c2c3cc(-c4ccc(Cl)cc4)ccc3ncc21] | 450.2 | | <1 | |
| 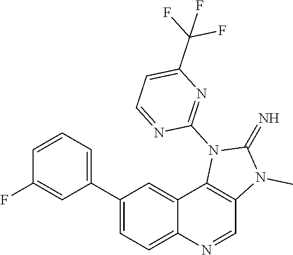 [SMILES: Cn1c(=N)n(-c2nccc(C(F)(F)F)n2)c2c3cc(-c4cccc(F)c4)ccc3ncc21] | 201.7 | | <1 | |
| 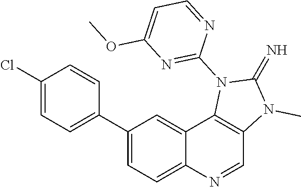 [SMILES: COc1ccnc(-n2c(=N)n(C)c3cnc4ccc(-c5ccc(Cl)cc5)cc4c32)n1] | 2113 | | <1 | |
| 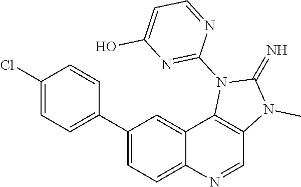 [SMILES: Cn1c(=N)n(-c2nccc(O)n2)c2c3cc(-c4ccc(Cl)cc4)ccc3ncc21] | 43080 | >30 | <1 | |
| 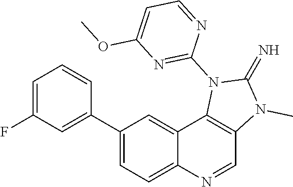 [SMILES: COc1ccnc(-n2c(=N)n(C)c3cnc4ccc(-c5cccc(F)c5)cc4c32)n1] | 1306 | | 6.5 | |
| 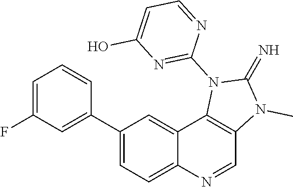 [SMILES: Cn1c(=N)n(-c2nccc(O)n2)c2c3cc(-c4cccc(F)c4)ccc3ncc21] | 18750 | >30 | <1 | |
| 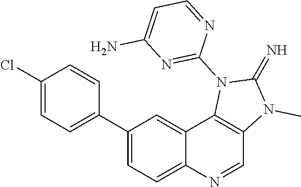 [SMILES: Cn1c(=N)n(-c2nccc(N)n2)c2c3cc(-c4ccc(Cl)cc4)ccc3ncc21] | 1864 | >30 | <1 | |
| 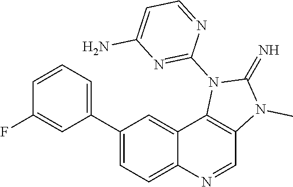 [SMILES: Cn1c(=N)n(-c2nccc(N)n2)c2c3cc(-c4cccc(F)c4)ccc3ncc21] | 1570 | >30 | 5.2 | |
| 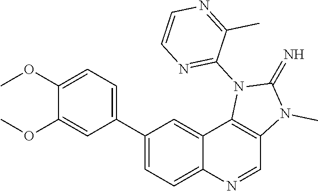 | 58 | | >63 | 1240 |
| 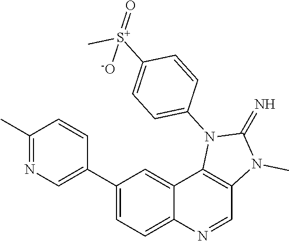 | 46 | | 33.2 | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I): 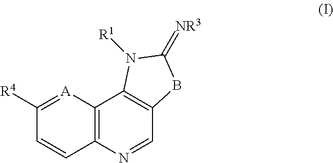
wherein A is CR⁵ or N,
B is CR⁸═CR⁹ or NR²,
R⁸ and R⁹ are independently selected from hydrogen, hydroxyl, OR⁰, halogen, optionally substituted C₆₋₁₀ aryl, and optionally substituted C₁₋₆ alkyl,
R¹⁰ is hydrogen, C₁₋₁₂ alkyl, C₃₋₈cycloalkyl, CH₂COOR¹³, or H₂N(CH₂)ₙ— wherein n is an integer of 2-6,
R¹ is C₆₋₁₀ aryl or heteroaryl optionally substituted with at least one substituent selected from —CN, halo, —CF₃, —CONH₂, —OCF₃, C₁-C₆ alkyl, C₁-C₆ alkylcarbonyl, —OH, C₁-C₆ alkylaminocarbonyl, C₃-C₈ cycloalkylaminocarbonyl, C₁-C₆ alkylaminoalkyl, cyanomethyl, piperazinomethyl, C₁-C₆ alkylsulfonyl, C₁-C₆ alkylsulfonylamino, dialkylaminoalkylamino, optionally substituted piperazinylcarbonyl, and C₁-C₆ alkoxy, a heterocyclyl group selected from the group consisting of piperazin-1-yl, 4-(C₁-C₆ alkylcarbonyl)piperazin-1-yl, morpholinyl optionally substituted with C₁-C₆ alkyl, azetidin-1-yl, pyrrolidin-1-yl, piperidin-1-yl, octahydropyrrolo[3,4-b]pyrrolyl, and 2-oxa-6-azaspiro[3.3]heptyl, C₃-C₈ cycloalkyl or C₃-C₈ azacycloalkyl, each optionally substituted with morpholino, optionally substituted piperidinyl, or optionally substituted piperazinyl,
R² is C₁-C₆ alkyl, hydroxyl C₁-C₆ alkyl, C₁-C₆ alkylcarbonyl, C₁-C₆ alkylsulfonyl, or optionally substituted benzyl,
R³ is H, —CN, C₁-C₆ alkylcarbonyl, C₁-C₆ alkoxycarbonyl, phenylsulfonyl, 4-methylphenylsulfonyl, C₁-C₆ alkylsulfonyl, aminocarbonyl, aminosulfonyl, optionally substituted benzyl, —OH, —OR, —SR, —(S═O)R (R═C₁-C₆ alkyl), guanidino, or pyrimidin-5-yl,
R⁴ is phenyl, heteroaryl, 1-phenyl-2-ethynyl, or heterocyclyl, wherein the phenyl, heteroaryl, heterocyclyl, or phenyl of the 1-phenyl-2-ethynyl is optionally substituted with one or more substituents selected from the group consisting of halo, C₁-C₆ alkyl, amino, oxo, dialkylaminoalkyl, dialkylaminoalkoxy, —CN, aminocarbonyl, —OR⁶, CF₃, and C₁-C₆ alkylsulfonyl,
R⁶ is H or C₁-C₆ alkyl,
R⁵ is hydrogen, C₁-C₆ alkyl, C₆-C₁₀ aryl, halogen, hydroxyl, or OR⁷,
R⁷ is C₁-C₆ alkyl, formyl, C₁-C₆ acyl, or C₆-C₁₀ aryl,
or a pharmaceutically acceptable salt thereof.

2. (canceled)

3. The compound or salt of claim 1, wherein A is CH, B is NR² and R¹ is selected from the group consisting of

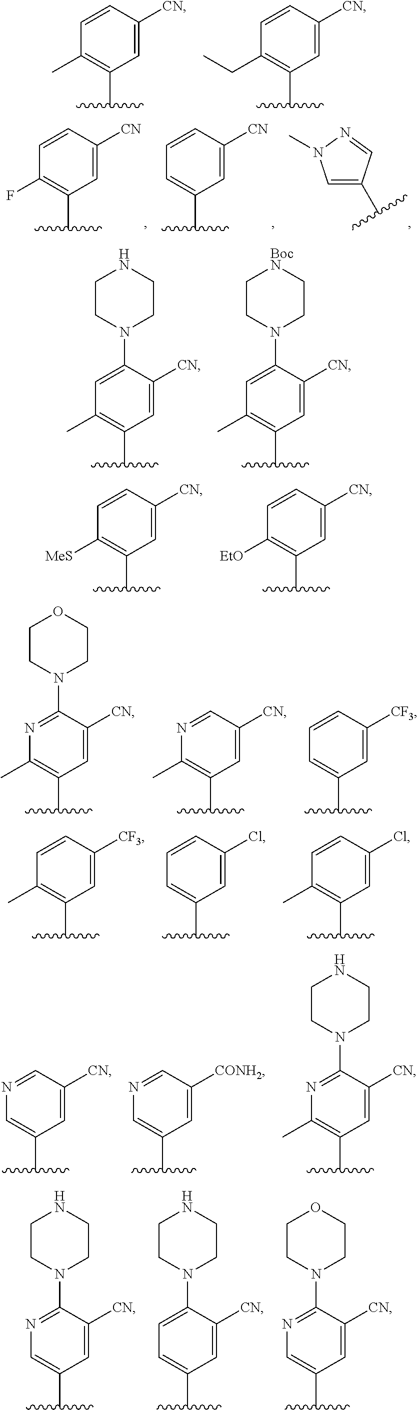

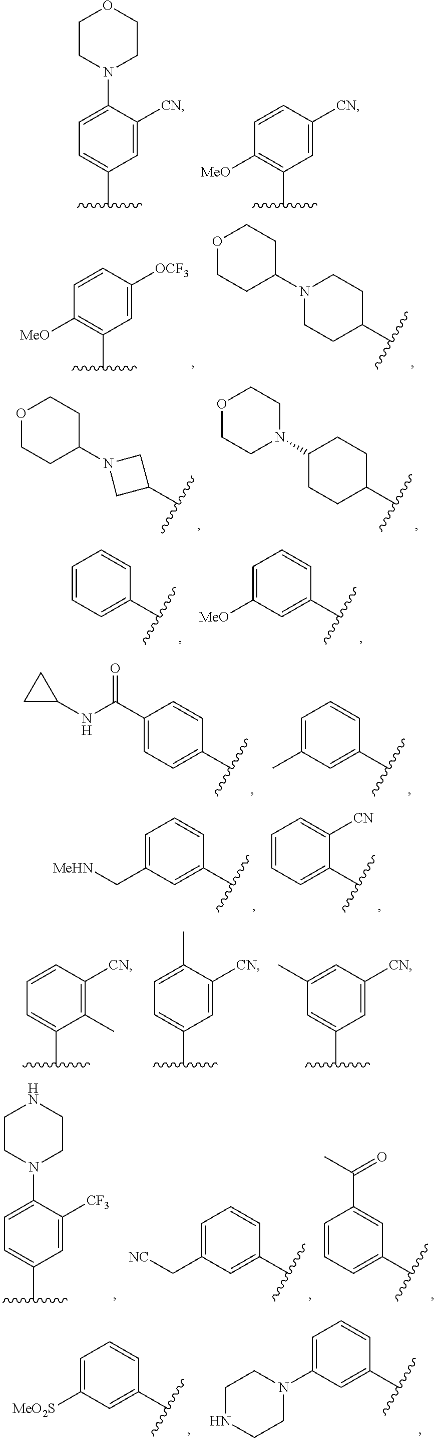

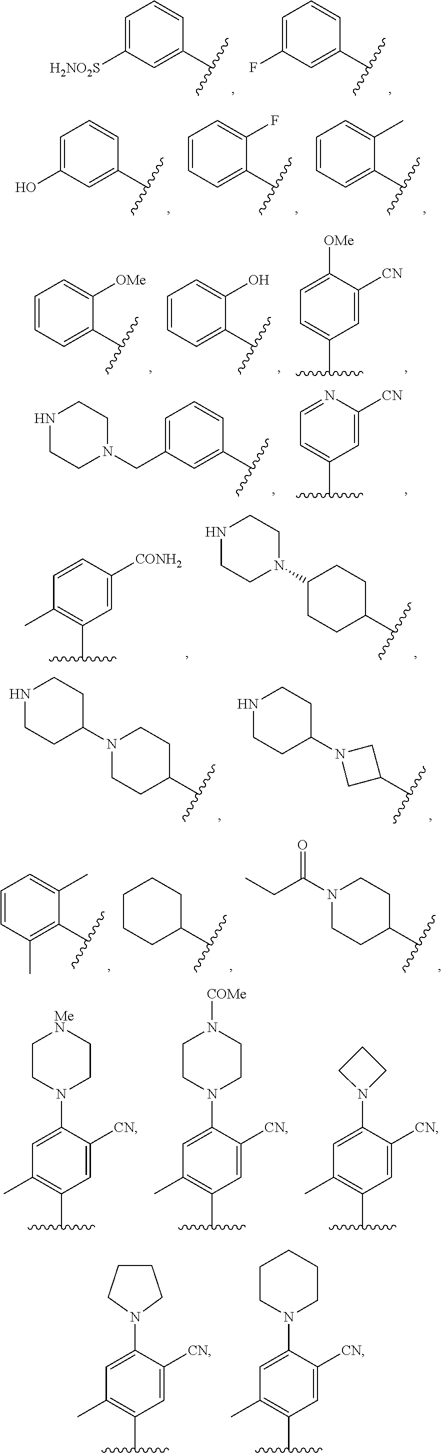

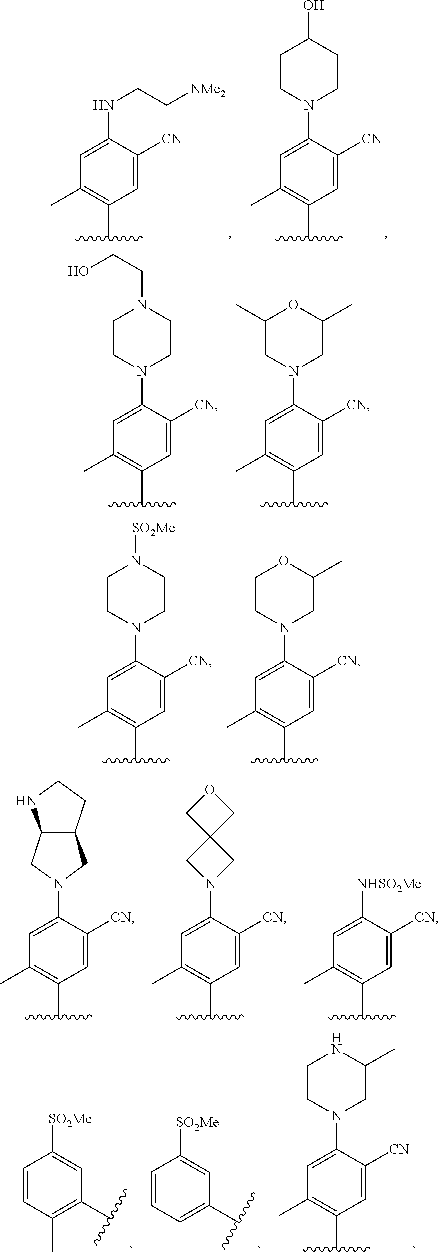

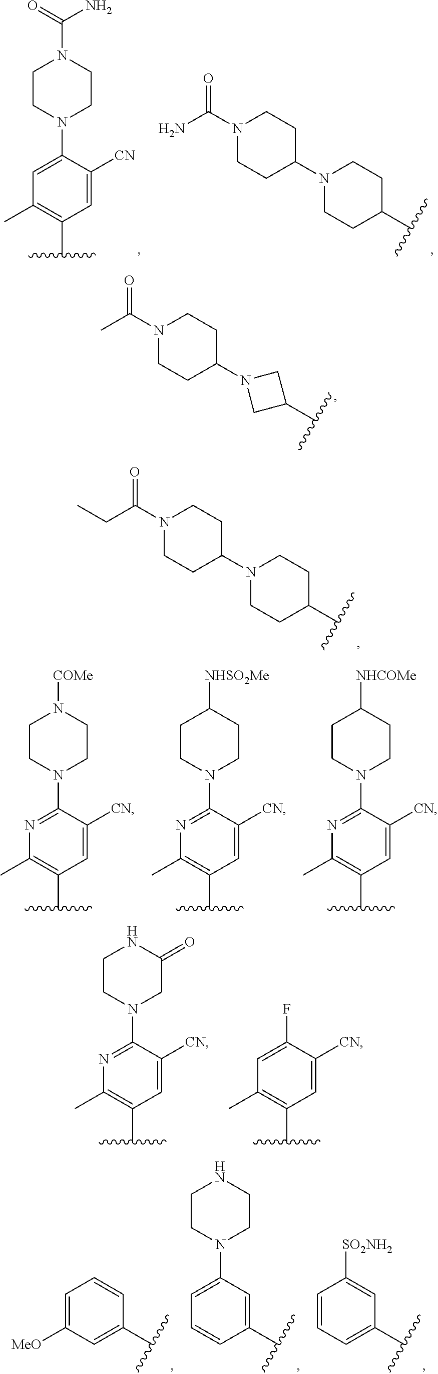

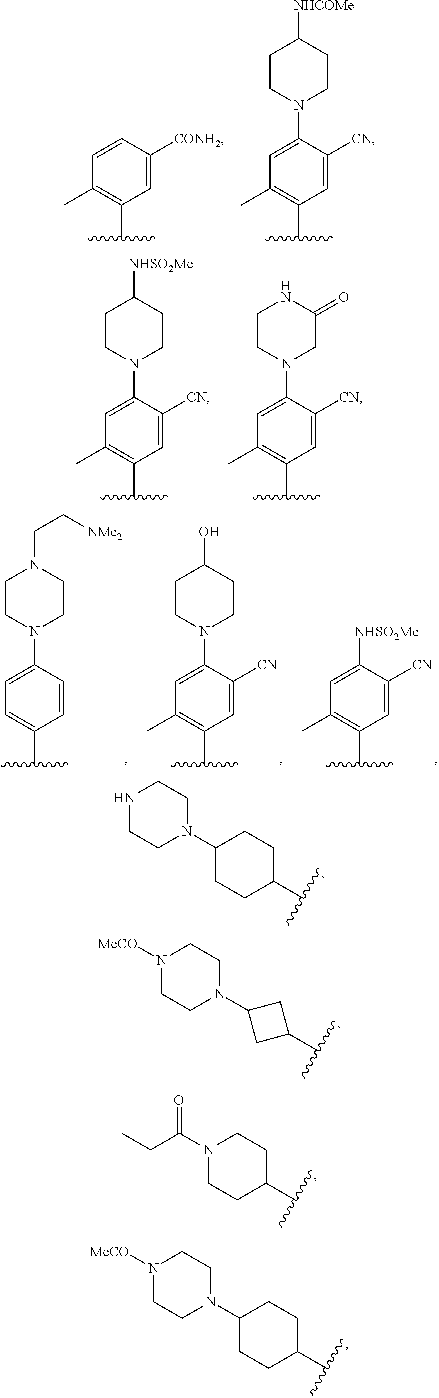

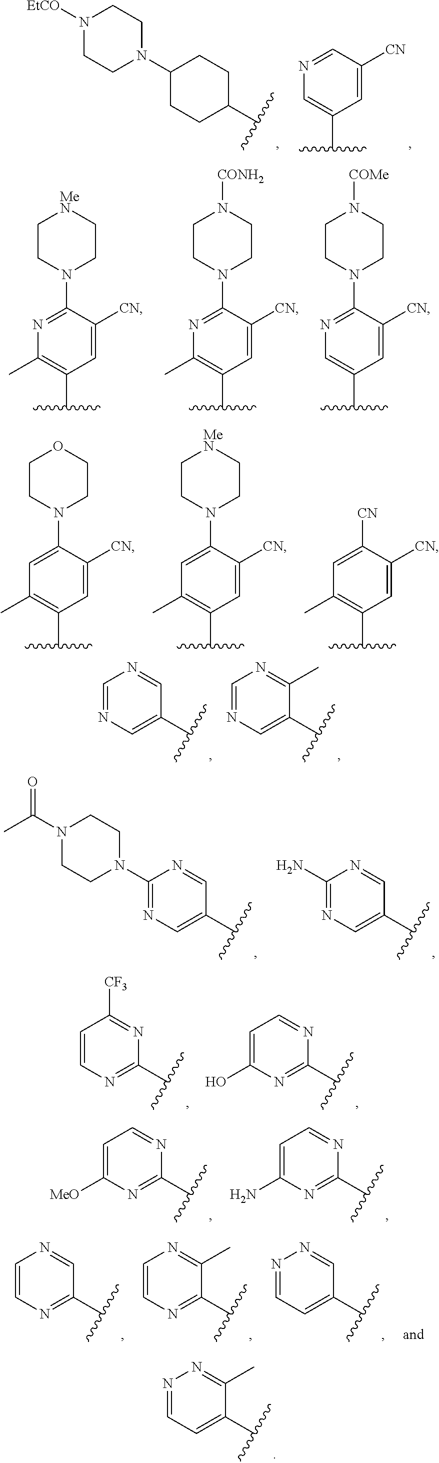

4. The compound or salt of claim 3, wherein R² is methyl, and R³ is H.

5.-6. (canceled)

7. The compound or salt of claim 3, wherein R² is methyl, R³ is H, R¹ is 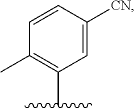 [SMILES: *c1cc(C#N)ccc1C]
and R⁴ is 4-fluorophenyl, 3-dimethylaminomethylphenyl, 3-cyanophenyl, 3-cyano-4-chlorophenyl, 3-methoxy-4-chlorophenyl, 3-chlorophenyl, 3-fluorophenyl, phenyl, 3-methylphenyl, 3-hydroxyphenyl, 3-aminophenyl, 3-hydroxy-4-fluorophenyl, 3,4-dimethoxyphenyl, 3,4-dichlorophenyl, 3,4-difluorophenyl, 2-methylphenyl, 2-chlorophenyl, or 2-hydroxyphenyl.

8. The compound or salt of claim 3, wherein R² is methyl, R³ is H, R⁴ is 4-chlorophenyl, and R¹ is

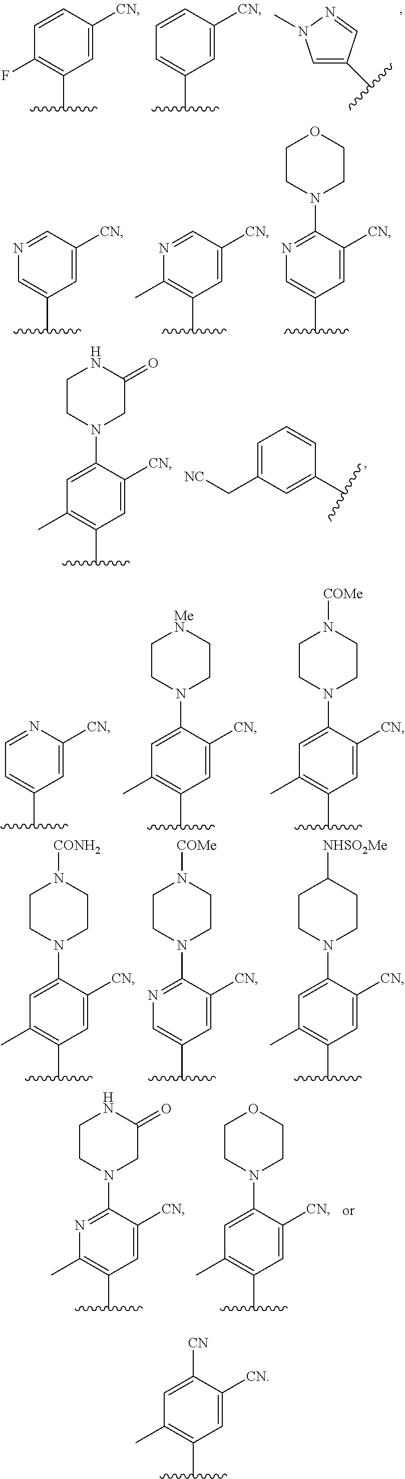

9. The compound or salt of claim 3, wherein R² is methyl, R¹ is 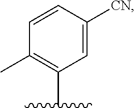 [SMILES: *c1cc(C#N)ccc1C]
R⁴ is 4-chlorophenyl, and R³ is —CN, ethylcarbonyl, 4-methylphenylsulfonyl, methyl, n-butyl, methylsulfonyl, guanidine, methoxycarbonyl, t-butyloxycarbonyl, n-butyloxycarbonyl, or aminosulfonyl.

10. (canceled)

11. The compound or salt of claim 3, wherein R² is methyl, R³ is aminosulfonyl or methoxycarbonyl, R⁴ is 2-amino-5-pyridyl, and R¹ is

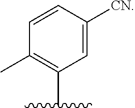 [SMILES: *c1cc(C#N)ccc1C]

12. The compound or salt of claim 3, wherein R² is methyl, R³ is H, R⁴ is 4-chlorophenyl, and R¹ is

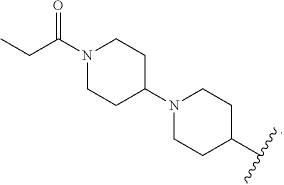 [SMILES: *C1CCN(C2CCN(C(=O)CC)CC2)CC1]

13. The compound or salt of claim 3, wherein R² is methyl, R³ is H, R⁴ is 3-fluorophenyl, and R¹ is

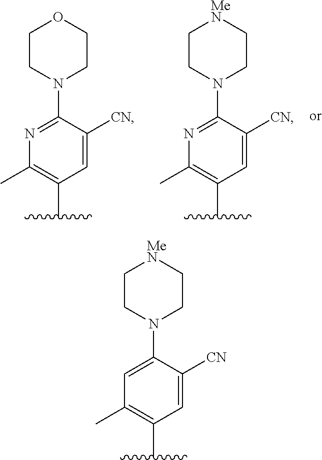 [SMILES: *c1cc(C#N)c(N2CCOCC2)nc1C ; *c1cc(C#N)c(N2CCN(C)CC2)nc1C ; "or" ; *c1cc(C#N)c(N2CCN(C)CC2)cc1C]

14. (canceled)

15. The compound or salt of claim 1, wherein B is CR⁸═CR⁹, A is CH, and R⁸ and R⁹ are both H.

16. The compound or salt of claim 15, wherein R¹ is

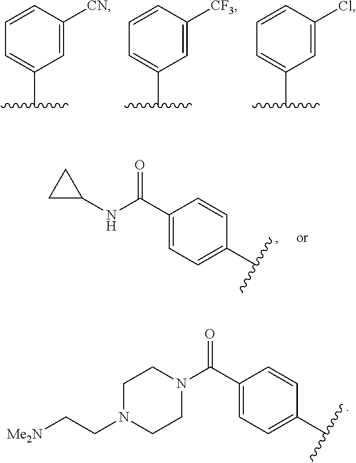

17. The compound or salt of claim 15, wherein R⁴ is 4-chlorophenyl or 2-amino-5-pyridyl.

18. The compound or salt of claim 15, wherein R³ is H, R⁴ is 4-chlorophenyl, and R¹ is 3-trifluorophenyl, 3-chlorophenyl, or

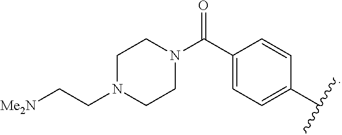 [SMILES: *c1ccc(C(=O)N2CCN(CCN(C)C)CC2)cc1]

19.-21. (canceled)

22. The compound or salt of claim 1, wherein A is CH, B is NR², and R¹ is selected from the group consisting of

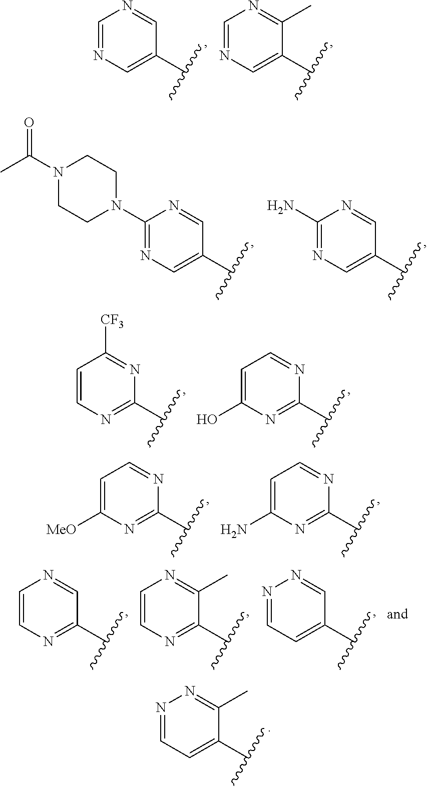

23. The compound or salt of claim 22, wherein R¹ is 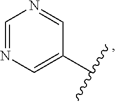 [SMILES: *c1cncnc1]
R² is methyl, R³ is H, and R⁴ is 4-chlorophenyl, 4-fluorophenyl, 2-aminopyrid-5-yl, 4-methylphenyl, 3-fluorophenyl, 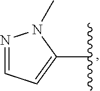 [SMILES: *c1ccnn1C]
3,4-dimethoxyphenyl, 3-cyano-4-chlorophenyl, 3-trifluoromethoxy-4-chlorophenyl, 2-trifluoromethylpyrid-5-yl, 2-methylpyrid-5-yl, or 2-methoxypyrid-5-yl.

24. The compound or salt of claim 22, wherein R¹ is 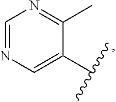 [SMILES: *c1cncnc1C]
R² is methyl, R³ is H, and R⁴ is 4-chlorophenyl, 4-fluorophenyl, 2-aminopyrid-5-yl, 4-methylphenyl, 3-fluorophenyl, 3-methoxy-4-chlorophenyl, 3-fluoro-4-methoxyphenyl, 3-methoxy-4-fluorophenyl, 3-trifluoromethyl-4-chlorophenyl, 3-trifluoromethoxy-4-chlorophenyl, 3,4-dimethoxylphenyl, 3,5-dimethoxylphenyl, 3,4-dimethoxy-5-fluorophenyl, 3,4-dimethoxy-5-chlorophenyl, and 3,4 5-trimethoxyphenyl.

25. The compound or salt of claim 22, wherein:
R² is methyl, R³ is H, and wherein:
R¹ is 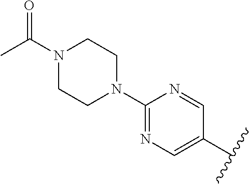 [SMILES: *c1cnc(N2CCN(C(C)=O)CC2)nc1]
and R⁴ is 4-chlorophenyl,
R¹ is 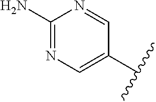 [SMILES: *c1cnc(N)nc1]
and R⁴ is 3-fluorophenyl,
R¹ is 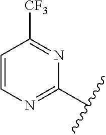 [SMILES: *c1nccc(C(F)(F)F)n1]
and R⁴ is 4-chlorophenyl or 3-fluorophenyl,
R¹ is 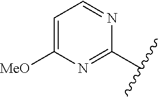 [SMILES: *c1nccc(OC)n1]
and R⁴ is 4-chlorophenyl or 3-fluororophenyl,
R¹ is 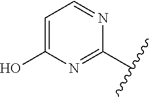 [SMILES: *c1nccc(O)n1]
and R⁴ is 4-chlorophenyl or 3-fluororophenyl,
R¹ is 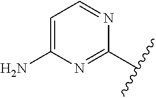 [SMILES: *c1nccc(N)n1]
and R⁴ is 4-chlorophenyl or 3-fluoro,
R¹ is 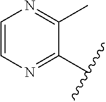 [SMILES: *c1nccnc1C]
and R⁴ is 3,4-dimethoxyphenyl, or
R¹ is 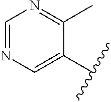 [SMILES: *c1cncnc1C]
and R⁴ is 3,4-dimethoxyphenyl.

26. The compound or salt of claim 22, wherein:
R¹ is 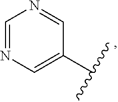 [SMILES: *c1cncnc1]
R² is methyl, R³ is 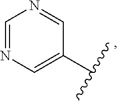 [SMILES: *c1cncnc1]
and R⁴ is 3-fluorophenyl, or
R¹ is 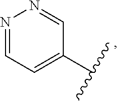 [SMILES: *c1ccnnc1]
R² is H, R³ is H, and R⁴ is 3,4-dimethoxyphenyl.

27. A pharmaceutical composition comprising a compound or salt of claim 1 and a pharmaceutically acceptable carrier.

28. A method of blocking transmission of a Plasmodium parasite in a mammal in need of thereof or for treating malaria by killing or arresting the growth of Plasmodium organisms in a mammal, wherein the Plasmodium organisms are in a liver stage or an asexual stage, the method comprising administering to a mammal a therapeutically effective amount of a compound or salt of claim 1.
